SEQUENCE LISTING (286 sequences)

SEQ ID NO: 1 - RNA, 26 nt, synthetic construct
  misc_feature 1..26: note = Synthetic
  source 1..26: mol_type = other RNA
ggctcgagatactgttcccgatccaa

SEQ ID NO: 2 - RNA, 28 nt, synthetic construct
  misc_feature 1..28: note = Synthetic
  source 1..28: mol_type = other RNA
ggtgctcgagatactgttcccgatccaa

SEQ ID NO: 3 - RNA, 28 nt, synthetic construct
  misc_feature 1..28: note = Synthetic
  source 1..28: mol_type = other RNA
ggatctcgagatactgttcccgatccaa

SEQ ID NO: 4 - RNA, 29 nt, synthetic construct
  misc_feature 1..29: note = Synthetic
  source 1..29: mol_type = other RNA
ggtaactcgagatactgttcccgatccaa

SEQ ID NO: 5 - RNA, 29 nt, synthetic construct
  misc_feature 1..29: note = Synthetic
  source 1..29: mol_type = other RNA
gggatctcgagatactgttcccgatccaa

SEQ ID NO: 6 - DNA, 36 bp, synthetic construct
  misc_feature 1..36: note = Synthetic
  source 1..36: mol_type = other DNA
taatacgactcactatatccctatcagtgatagaga

SEQ ID NO: 7 - DNA, 37 bp, synthetic construct
  misc_feature 1..37: note = Synthetic
  source 1..37: mol_type = other DNA
aatacgactcactataggtccctatcagtgtatagag

SEQ ID NO: 8 - DNA, 36 bp, synthetic construct
  misc_feature 1..36: note = Synthetic
  source 1..36: mol_type = other DNA
atacgactcactatagggatccctatcagtgataga

SEQ ID NO: 9 - DNA, 36 bp, synthetic construct
  misc_feature 1..36: note = Synthetic
  source 1..36: mol_type = other DNA
tacgactcactatagggagatccctatcagtgatag

SEQ ID NO: 10 - DNA, 36 bp, synthetic construct
  misc_feature 1..36: note = Synthetic
  source 1..36: mol_type = other DNA
acgactcactatagggagacctccctatcagtgata

SEQ ID NO: 11 - DNA, 36 bp, synthetic construct
  misc_feature 1..36: note = Synthetic
  source 1..36: mol_type = other DNA
cgactcactatagggagaccactccctatcagtgat

SEQ ID NO: 12 - RNA, 45 nt, synthetic construct
  misc_feature 1..45: note = Synthetic
  source 1..45: mol_type = other RNA
ggtccctatcagtgatagagactcgagatactgttcccgatccaa

SEQ ID NO: 13 - RNA, 54 nt, synthetic construct
  misc_feature 1..54: note = Synthetic
  source 1..54: mol_type = other RNA
ggtatttacaaacaaccatgaatgtaagtactcgagatactgttcccgatccaa

SEQ ID NO: 14 - RNA, 61 nt, synthetic construct
  misc_feature 1..61: note = Synthetic
  source 1..61: mol_type = other RNA
gacacatgaacagttattcagataaataactgttcaactcgagatactgttcccgatccaa

SEQ ID NO: 15 - RNA, 25 nt, synthetic construct
  misc_feature 1..25: note = Synthetic
  source 1..25: mol_type = other RNA
ggacctagcccttgtcatagagctc

SEQ ID NO: 16 - DNA, 26 bp, synthetic construct
  misc_feature 1..26: note = Synthetic
  source 1..26: mol_type = other DNA
ggaacctgcccttgtcatagaagctc

SEQ ID NO: 17 - RNA, 26 nt, synthetic construct
  misc_feature 1..26: note = Synthetic
  source 1..26: mol_type = other RNA
ggaacctagcccttgtcatagagctc

SEQ ID NO: 18 - RNA, 26 nt, synthetic construct
  misc_feature 1..26: note = Synthetic
  source 1..26: mol_type = other RNA
ggaacctagcccttgtcatagagctc

SEQ ID NO: 19 - RNA, 73 nt, synthetic construct
  misc_feature 1..73: note = Synthetic
  source 1..73: mol_type = other RNA
ggctcgagatactgttcccgatccaatagcataaccccttggggcctctaaacgggtcttgaggggttttttg

SEQ ID NO: 20 - RNA, 48 nt, synthetic construct
  misc_feature 1..48: note = Synthetic
  source 1..48: mol_type = other RNA
gacacatgaacagttattcagatactcgagatactgttcccgatccaa

SEQ ID NO: 21 - RNA, 72 nt, synthetic construct
  misc_feature 1..72: note = Synthetic
  source 1..72: mol_type = other RNA
gacacatgaacagttattcagatactcgagatactgttcccgatccaactcgagatactgttcccgatccaa

SEQ ID NO: 22 - RNA, 66 nt, synthetic construct
  misc_feature 1..66: note = Synthetic
  source 1..66: mol_type = other RNA
gacacatgaacagttattcagatatttttctgaataactgttctcgagatactgttcccgatccaa

SEQ ID NO: 23 - RNA, 60 nt, synthetic construct
  misc_feature 1..60: note = Synthetic
  source 1..60: mol_type = other RNA
gacacatgaacagttattcagataaataactgttcactcgagatactgttcccgatccaa

SEQ ID NO: 24 - RNA, 60 nt, synthetic construct; identical to SEQ ID NO: 23
  misc_feature 1..60: note = Synthetic
  source 1..60: mol_type = other RNA

SEQ ID NO: 25 - DNA, 55 bp, synthetic construct
  misc_feature 1..55: note = Synthetic
  source 1..55: mol_type = other DNA
tgagatgcatccattccactcgttctgtttctatgttctgtattcgttctgcctt

SEQ ID NO: 26 - DNA, 67 bp, synthetic construct
  misc_feature 1..67: note = Synthetic
  source 1..67: mol_type = other DNA
gacaaatgaaacagacaaaggcagaacgaatacacaacatagactgtgaacgagtgcaatccatgca

SEQ ID NO: 27 - DNA, 29 bp, synthetic construct
  misc_feature 1..29: note = Synthetic
  source 1..29: mol_type = other DNA
gggacaatgaaacagaaaaaggcagaacg

SEQ ID NO: 28 - RNA, 60 nt, synthetic construct
  misc_feature 1..60: note = Synthetic
  source 1..60: mol_type = other RNA
ggcgttctgcctttttctgtttcatttgtcttttttgacaaatgaaacagaaaaagggag

SEQ ID NO: 29 - RNA, 37 nt, synthetic construct
  misc_feature 1..37: note = Synthetic
  source 1..37: mol_type = other RNA
ggaaaaaaagacaaatgaaacagaaaaaggcagaacg

SEQ ID NO: 30 - RNA, 58 nt, synthetic construct
  misc_feature 1..58: note = Synthetic
  source 1..58: mol_type = other RNA
ggcgttctgcctttttctgtttcatttgtcttttttgacaaatgaaacagaaaaaggc

SEQ ID NO: 31 - RNA, 59 nt, synthetic construct
  misc_feature 1..59: note = Synthetic
  source 1..59: mol_type = other RNA
ggacgttctgcctttttctgtttcatttgtcttttttgacaaatgaaacagaaaaaggc

SEQ ID NO: 32 - RNA, 80 nt, synthetic construct
  misc_feature 1..80: note = Synthetic
  source 1..80: mol_type = other RNA
ggcacatgaacagttattcagatacgttctgcctttttctgtttcatttgtcttttttgacaaatgaaacagaaaaaggc

SEQ ID NO: 33 - RNA, 93 nt, synthetic construct
  misc_feature 1..93: note = Synthetic
  source 1..93: mol_type = other RNA
ggcacatgaacagttattcagatagttcatgtgccttcgttctgcctttttctgtttcatttgtcttttttgacaaatgaaacagaaaaaggc

SEQ ID NO: 34 - DNA, 316 bp, synthetic construct
  misc_feature 1..316: note = Synthetic
  source 1..316: mol_type = other DNA
gcggataacaatttcacacaggaaacagctatgaccatgattacgccaagcttgcatgcctgcaggtcgactctagataatacgactcactatagggatccctatcagtgatagagacccacatactctgatgatccgagacggtcgggtccagatattcgtatctgtcgagtagagtgtgggctcggatcattcatggcaagagacggtcgggtccagatattcgtatctgtcgagtagagtgtgggctcttgccatgtgtatgtgggtagcataaccccttggggcctctaaacgggtcttgaggggttttttg

SEQ ID NO: 35 - DNA, 43 bp, synthetic construct
  misc_feature 1..43: note = Synthetic
  source 1..43: mol_type = other DNA
taatacgactcactataggaacctagcccttgtcatagagctc

SEQ ID NO: 36 - DNA, 90 bp, synthetic construct
  misc_feature 1..90: note = Synthetic
  source 1..90: mol_type = other DNA
taatacgactcactataggaacctagcccttgtcatagagctctagcataaccccttggggcctctaaacgggtcttgaggggttttttg

SEQ ID NO: 37 - DNA, 43 bp, synthetic construct
  misc_feature 1..43: note = Synthetic
  source 1..43: mol_type = other DNA
taatacgactcactataggctcgagatactgttcccgatccaa

SEQ ID NO: 38 - DNA, 90 bp, synthetic construct
  misc_feature 1..90: note = Synthetic
  source 1..90: mol_type = other DNA
taatacgactcactataggctcgagatactgttcccgatccaatagcataaccccttggggcctctaaacgggtcttgaggggttttttg

SEQ ID NO: 39 - DNA, 45 bp, synthetic construct
  misc_feature 1..45: note = Synthetic
  source 1..45: mol_type = other DNA
taatacgactcactataggtgctcgagatactgttcccgatccaa

SEQ ID NO: 40 - DNA, 45 bp, synthetic construct
  misc_feature 1..45: note = Synthetic
  source 1..45: mol_type = other DNA
taatacgactcactataggatctcgagatactgttcccgatccaa

SEQ ID NO: 41 - DNA, 46 bp, synthetic construct
  misc_feature 1..46: note = Synthetic
  source 1..46: mol_type = other DNA
taatacgactcactataggtaactcgagatactgttcccgatccaa

SEQ ID NO: 42 - DNA, 46 bp, synthetic construct
  misc_feature 1..46: note = Synthetic
  source 1..46: mol_type = other DNA
taatacgactcactatagggatctcgagatactgttcccgatccaa

SEQ ID NO: 43 - DNA, 60 bp, synthetic construct
  misc_feature 1..60: note = Synthetic
  source 1..60: mol_type = other DNA
taatacgactcactatatccctatcagtgatagagactcgagatactgttcccgatccaa

SEQ ID NO: 44 - DNA, 62 bp, synthetic construct
  misc_feature 1..62: note = Synthetic
  source 1..62: mol_type = other DNA
taatacgactcactataggtccctatcagtgatagagactcgagatactgttcccgatccaa

SEQ ID NO: 45 - DNA, 64 bp, synthetic construct
  misc_feature 1..64: note = Synthetic
  source 1..64: mol_type = other DNA
taatacgactcactatagggatccctatcagtgatagagactcgagatactgttcccgatccaa

SEQ ID NO: 46 - DNA, 66 bp, synthetic construct
  misc_feature 1..66: note = Synthetic
  source 1..66: mol_type = other DNA
taatacgactcactatagggagatccctatcagtgatagagactcgagatactgttcccgatccaa

SEQ ID NO: 47 - DNA, 68 bp, synthetic construct
  misc_feature 1..68: note = Synthetic
  source 1..68: mol_type = other DNA
taatacgactcactatagggagacctccctatcagtgatagagactcgagatactgttcccgatccaa

SEQ ID NO: 48 - DNA, 70 bp, synthetic construct
  misc_feature 1..70: note = Synthetic
  source 1..70: mol_type = other DNA
taatacgactcactatagggagaccactccctatcagtgatagagactcgagatactgttcccgatccaa

SEQ ID NO: 49 - DNA, 71 bp, synthetic construct
  misc_feature 1..71: note = Synthetic
  source 1..71: mol_type = other DNA
taatacgactcactataggtatttacaaacaaccatgaatgtaagtactcgagatactgttcccgatccaa

SEQ ID NO: 50 - DNA, 65 bp, synthetic construct
  misc_feature 1..65: note = Synthetic
  source 1..65: mol_type = other DNA
taatacgactcactatagacacatgaacagttattcagatactcgagatactgttcccgatccaa

SEQ ID NO: 51 - DNA, 89 bp, synthetic construct
  misc_feature 1..89: note = Synthetic
  source 1..89: mol_type = other DNA
taatacgactcactatagacacatgaacagttattcagatactcgagatactgttcccgatccaactcgagatactgttcccgatccaa

SEQ ID NO: 52 - DNA, 84 bp, synthetic construct
  misc_feature 1..84: note = Synthetic
  source 1..84: mol_type = other DNA
taatacgactcactatagacacatgaacagttattcagatattttttctgaataactgttctcgagatactgttcccgatccaa

SEQ ID NO: 53 - DNA, 77 bp, synthetic construct
  misc_feature 1..77: note = Synthetic
  source 1..77: mol_type = other DNA
taatacgactcactatagacacatgaacagttattcagataaataactgttcactcgagatactgttcccgatccaa

SEQ ID NO: 54 - DNA, 78 bp, synthetic construct
  misc_feature 1..78: note = Synthetic
  source 1..78: mol_type = other DNA
taatacgactcactatagacacatgaacagttattcagataaataactgttcatctcgagatactgttcccgatccaa

SEQ ID NO: 55 - DNA, 79 bp, synthetic construct
  misc_feature 1..79: note = Synthetic
  source 1..79: mol_type = other DNA
taatacgactcactatagacacatgaacagttattcagataaataactgttcatgctcgagatactgttcccgatccaa

SEQ ID NO: 56 - DNA, 78 bp, synthetic construct
  misc_feature 1..78: note = Synthetic
  source 1..78: mol_type = other DNA
taatacgactcactatagacacatgaacagttattcagataaataactgttcaactcgagatactgttcccgatccaa

SEQ ID NO: 57 - DNA, 79 bp, synthetic construct
  misc_feature 1..79: note = Synthetic
  source 1..79: mol_type = other DNA
taatacgactcactatagacacatgaacagttattcagataaataactgttcaaactcgagatactgttcccgatccaa

SEQ ID NO: 58 - DNA, 80 bp, synthetic construct
  misc_feature 1..80: note = Synthetic
  source 1..80: mol_type = other DNA
taatacgactcactatagacacatgaacagttattcagataaataactgttcaaaactcgagatactgttcccgatccaa

SEQ ID NO: 59 - DNA, 47 bp, synthetic construct
  misc_feature 1..47: note = Synthetic
  source 1..47: mol_type = other DNA
taatacgactcactatagggacaaatgaaacagacaaaggcagaacg

SEQ ID NO: 60 - DNA, 65 bp, synthetic construct
  misc_feature 1..65: note = Synthetic
  source 1..65: mol_type = other DNA
taatacgactcactataggtccctatcagtgatagagacagaacgtgaaaccaaacttcatctca

SEQ ID NO: 61 - DNA, 68 bp, synthetic construct
  misc_feature 1..68: note = Synthetic
  source 1..68: mol_type = other DNA
taatacgactcactataggcacatgaacagttattcagatacagaacgagtggaatggatgcatctca

SEQ ID NO: 62 - DNA, 62 bp, synthetic construct
  misc_feature 1..62: note = Synthetic
  source 1..62: mol_type = other DNA
taatacgactcactataggtccctatcagtgatagagaaacgaatacagaacatagaaacag

SEQ ID NO: 63 - DNA, 65 bp, synthetic construct
  misc_feature 1..65: note = Synthetic
  source 1..65: mol_type = other DNA
taatacgactcactataggcacatgaacagttattcagataaacgagtggaatggatgcatctca

SEQ ID NO: 64 - DNA, 47 bp, synthetic construct
  misc_feature 1..47: note = Synthetic
  source 1..47: mol_type = other DNA
taatacgactcactatagggacaaatgaaacagaaaaaggcagaacg

SEQ ID NO: 65 - DNA, 53 bp, synthetic construct
  misc_feature 1..53: note = Synthetic
  source 1..53: mol_type = other DNA
taatacgactcactataggaaaaaagacaaatgaaacagaaaaaggcagaacg

SEQ ID NO: 66 - DNA, 77 bp, synthetic construct
  misc_feature 1..77: note = Synthetic
  source 1..77: mol_type = other DNA
taatacgactcactataggcgttctgcctttttctgtttcatttgtcttttttgacaaatgaaacagaaaaagggag

SEQ ID NO: 67 - DNA, 75 bp, synthetic construct
  misc_feature 1..75: note = Synthetic
  source 1..75: mol_type = other DNA
taatacgactcactataggcgttctgcctttttctgtttcatttgtcttttttgacaaatgaaacagaaaaaggc

SEQ ID NO: 68 - DNA, 76 bp, synthetic construct
  misc_feature 1..76: note = Synthetic
  source 1..76: mol_type = other DNA
taatacgactcactataggacgttctgcctttttctgtttcatttgtcttttttgacaaatgaaacagaaaaaggc

SEQ ID NO: 69 - DNA, 97 bp, synthetic construct
  misc_feature 1..97: note = Synthetic
  source 1..97: mol_type = other DNA
taatacgactcactataggtccctatcagtgatagagaccccgttctgcctttttctgtttcatttgtcttttttgacaaatgaaacagaaaaaggc

SEQ ID NO: 70 - DNA, 97 bp, synthetic construct
  misc_feature 1..97: note = Synthetic
  source 1..97: mol_type = other DNA
taatacgactcactataggtccctatcagtgatagagagttatgtcacgattgtcaatcaattatgaatggatcctcattcgattatcctcttctcg

SEQ ID NO: 71 - DNA, 97 bp, synthetic construct
  misc_feature 1..97: note = Synthetic
  source 1..97: mol_type = other DNA
taatacgactcactataggcacatgaacagttattcagatacgttctgcctttttctgtttcatttgtcttttttgacaaatgaaacagaaaaaggc

SEQ ID NO: 72 - DNA, 110 bp, synthetic construct
  misc_feature 1..110: note = Synthetic
  source 1..110: mol_type = other DNA
taatacgactcactataggcacatgaacagttattcagatagttcatgtgccttcgttctgcctttttctgtttcatttgtcttttttgacaaatgaaacagaaaaaggc

SEQ ID NO: 73 - DNA, 110 bp, synthetic construct
  misc_feature 1..110: note = Synthetic
  source 1..110: mol_type = other DNA
taatacgactcactataggcacatgaacagttattcagataactgtatgcatgcttcatgccacaattgtgcttatagttctaatcgtcgtgttgttcttaattacgtat

SEQ ID NO: 74 - DNA, 66 bp, synthetic construct
  misc_feature 1..66: note = Synthetic
  source 1..66: mol_type = other DNA
taatacgactcactataggtccctatcagtgatagagagacaaatgaaacagacaaaggcagaacg

SEQ ID NO: 75 - DNA, 69 bp, synthetic construct
  misc_feature 1..69: note = Synthetic
  source 1..69: mol_type = other DNA
taatacgactcactataggcacatgaacagttattcagatagacaaatgaaacagacaaaggcagaacg

SEQ ID NO: 76 - DNA, 51 bp, synthetic construct
  misc_feature 1..51: note = Synthetic
  source 1..51: mol_type = other DNA
taatacgactcactataggaaaaacagaacgtgaaaccaaacttcatctca

SEQ ID NO: 77 - DNA, 51 bp, synthetic construct
  misc_feature 1..51: note = Synthetic
  source 1..51: mol_type = other DNA
taatacgactcactataggaaaaacagaacgagtggaatggatgcatctca

SEQ ID NO: 78 - DNA, 89 bp, synthetic construct
  misc_feature 1..89: note = Synthetic
  source 1..89: mol_type = other DNA
taatacgactcactataggtccctatcagtgatagagatgagatgaagtttggtttcacgttctgtttttccagaacgtcaaaccaaac

SEQ ID NO: 79 - DNA, 90 bp, synthetic construct
  misc_feature 1..90: note = Synthetic
  source 1..90: mol_type = other DNA
taatacgactcactataggcacatgaacagttattcagatatgagatgcatccattccactcgttctgtttttccagaacgagtgcaatg

SEQ ID NO: 80 - DNA, 89 bp, synthetic construct
  misc_feature 1..89: note = Synthetic
  source 1..89: mol_type = other DNA
taatacgactcactataggtccctatcagtgatagagacccctgtttctatgttctgtattcgttttttccaacgaatacagaacatag

SEQ ID NO: 81 - DNA, 90 bp, synthetic construct
  misc_feature 1..90: note = Synthetic
  source 1..90: mol_type = other DNA
taatacgactcactataggcacatgaacagttattcagatatgagatgcatccattccactcgtttttttaccaacgagtggaatggatg

SEQ ID NO: 82 - DNA, 47 bp, synthetic construct
  misc_feature 1..47: note = Synthetic
  source 1..47: mol_type = other DNA
taatacgactcactataggaaaaaacgaatacagaacatagaaacag

SEQ ID NO: 83 - DNA, 49 bp, synthetic construct
  misc_feature 1..49: note = Synthetic
  source 1..49: mol_type = other DNA
taatacgactcactataggtaaaaaaacgagtggaatggatgcatctca

SEQ ID NO: 84 - DNA, 16 bp, synthetic construct
  misc_feature 1..16: note = Synthetic
  source 1..16: mol_type = other DNA
ccttgtcatagagctc

SEQ ID NO: 85 - DNA, 24 bp, synthetic construct
  misc_feature 1..24: note = Synthetic
  source 1..24: mol_type = other DNA
gagctctatgacaagggctaggtt

SEQ ID NO: 86 - RNA, 18 nt, synthetic construct
  misc_feature 1..18: note = Synthetic
  misc_feature 1..18: note = 2' O-methylation
  source 1..18: mol_type = other RNA
gagccagacaagggcagg

SEQ ID NO: 87 - DNA, 24 bp, synthetic construct
  misc_feature 1..24: note = Synthetic
  source 1..24: mol_type = other DNA
ttggatcgggaacagtatctcgag

SEQ ID NO: 88 - DNA, 16 bp, synthetic construct
  misc_feature 1..16: note = Synthetic
  source 1..16: mol_type = other DNA
ctcgagatactgttcc

SEQ ID NO: 89 - DNA, 28 bp, synthetic construct
  misc_feature 1..28: note = Synthetic
  source 1..28: mol_type = other DNA
cgttctgcctttgtctgtttcatttgtc

SEQ ID NO: 90 - DNA, 24 bp, synthetic construct
  misc_feature 1..24: note = Synthetic
  source 1..24: mol_type = other DNA
gacaaatgaaacagacaaaggcag

SEQ ID NO: 91 - DNA, 43 bp, synthetic construct
  misc_feature 1..43: note = Synthetic
  source 1..43: mol_type = other DNA
gacaaatgaaacagacaaaggcagaacgtgaaaccaaacttca

SEQ ID NO: 92 - DNA, 27 bp, synthetic construct
  misc_feature 1..27: note = Synthetic
  source 1..27: mol_type = other DNA
tgagatgaagtttggtttcacgttctg

SEQ ID NO: 93 - DNA, 43 bp, synthetic construct
  misc_feature 1..43: note = Synthetic
  source 1..43: mol_type = other DNA
gacaaatgaaacagacaaaggcagaacgagtggaatggatgca

SEQ ID NO: 94 - DNA, 27 bp, synthetic construct
  misc_feature 1..27: note = Synthetic
  source 1..27: mol_type = other DNA
tgagatgcatccattccactcgttctg

SEQ ID NO: 95 - DNA, 63 bp, synthetic construct
  misc_feature 1..63: note = Synthetic
  source 1..63: mol_type = other DNA
gacaaatgaaacagacaaaggcagaacgtgaaaccaaacttcaagatcagtggaatggatgca

SEQ ID NO: 96 - DNA, 47 bp, synthetic construct
  misc_feature 1..47: note = Synthetic
  source 1..47: mol_type = other DNA
tgagatgcatccattccacttgagatgaagtttggtttcacgttctg

SEQ ID NO: 97 - DNA, 67 bp, synthetic construct
  misc_feature 1..67: note = Synthetic
  source 1..67: mol_type = other DNA
gacaaatgaaacagacaaaggcagaacgaatacacaacatagactgtgaacgagtgcaatggatgca

SEQ ID NO: 98 - DNA, 51 bp, synthetic construct
  misc_feature 1..51: note = Synthetic
  source 1..51: mol_type = other DNA
tgagatgcatccattccactcgttctgtttctatgttctgtattcgttctg

SEQ ID NO: 99 - DNA, 53 bp, synthetic construct
  misc_feature 1..53: note = Synthetic
  source 1..53: mol_type = other DNA
tgagatgcatccattccactcgttctgtttctatgttctgtattcgttctgcc

SEQ ID NO: 100 - DNA, 55 bp, synthetic construct; identical to SEQ ID NO: 25
  misc_feature 1..55: note = Synthetic
  source 1..55: mol_type = other DNA

SEQ ID NO: 101 - DNA, 28 bp, synthetic construct
  misc_feature 1..28: note = Synthetic
  source 1..28: mol_type = other DNA
cgttctgcctttgtctgtttcatttgtc

SEQ ID NO: 102 - DNA, 20 bp, synthetic construct
  misc_feature 1..20: note = Synthetic
  source 1..20: mol_type = other DNA
gacaaatgaaacagacaaag

SEQ ID NO: 103 - DNA, 20 bp, synthetic construct
  misc_feature 1..20: note = Synthetic
  source 1..20: mol_type = other DNA
gggttttcccagtcacgacg

SEQ ID NO: 104 - DNA, 18 bp, synthetic construct
  misc_feature 1..18: note = Synthetic
  source 1..18: mol_type = other DNA
ggccacgatgcgtccggc

SEQ ID NO: 105 - DNA, 26 bp, synthetic construct
  misc_feature 1..26: note = Synthetic
  source 1..26: mol_type = other DNA
atccggatatagttcctcctttcagc

SEQ ID NO: 106 - protein, 220 aa, synthetic construct
  REGION 1..220: note = Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSKLAAALEHHHHHH

SEQ ID NO: 107 - protein, 223 aa, synthetic construct
  REGION 1..223: note = Synthetic
MVRRTKEEAQETRAQIIEAAERAFYKRGVARTTLADIAELAGVTRGAIYWHFNNKAELVQALLDSLHETHDHLARASESEDELDPLGCMRKLLLQVFNELVLDARTRRINEILHHKCEFTDDMCEIRQQRQSAVLDCHKGITLALANAVRRGQLPGELDVERAAVAMFAYVDGLIGRWLLLPDSVDLLGDVEKWVDTGLDMLRLSPALRKKLAAALEHHHHHH

SEQ ID NO: 108 - protein, 135 aa, synthetic construct
  REGION 1..135: note = Synthetic
MTKPVLQDGETVVCQGTHAAIASELQAIAPEVAQSLAEFFAVLADPNRLRLLSLLARSELCVGDLAQAIGVSESAVSHQLRSLRNLRLVSYRKQGRHVYYQLQDHHIVALYQNALDHLQECRENLYFQGHHHHHH

SEQ ID NO: 109 - DNA, 43 bp, synthetic construct; identical to SEQ ID NO: 35
  misc_feature 1..43: note = Synthetic
  source 1..43: mol_type = other DNA

SEQ ID NO: 110 - DNA, 128 bp, synthetic construct
  misc_feature 1..128: note = Synthetic
  source 1..128: mol_type = other DNA
taatacgactcactataggaacctagcccttgtcatagagctcgccggccatggtcccagcctcctcgctggcggccggtgggcaacattccgaggggaccgtcccctcggtaatggcgaatgggacc

SEQ ID NO: 111 - DNA, 90 bp, synthetic construct; identical to SEQ ID NO: 36
  misc_feature 1..90: note = Synthetic
  source 1..90: mol_type = other DNA

SEQ ID NO: 112 - DNA, 43 bp, synthetic construct; identical to SEQ ID NO: 37
  misc_feature 1..43: note = Synthetic
  source 1..43: mol_type = other DNA

SEQ ID NO: 113 - DNA, 43 bp, synthetic construct
  misc_feature 1..43: note = Synthetic
  source 1..43: mol_type = other DNA
ttggatcgggaacagtatctcgagcctatagtgagtcgtatta

SEQ ID NO: 114 - DNA, 26 bp, synthetic construct
  misc_feature 1..26: note = Synthetic
  source 1..26: mol_type = other DNA
ggctcgagatactgttcccgatccaa

SEQ ID NO: 115 - DNA, 128 bp, synthetic construct
  misc_feature 1..128: note = Synthetic
  source 1..128: mol_type = other DNA
taatacgactcactataggctcgagatactgttcccgatccaagccggccatggtcccagcctcctcgctggcggccggtgggcaacattccgaggggaccgtcccctcggtaatggcgaatgggacc

SEQ ID NO: 116 - DNA, 90 bp, synthetic construct; identical to SEQ ID NO: 38
  misc_feature 1..90: note = Synthetic
  source 1..90: mol_type = other DNA

SEQ ID NO: 117 - DNA, 45 bp, synthetic construct; identical to SEQ ID NO: 39
  misc_feature 1..45: note = Synthetic
  source 1..45: mol_type = other DNA

SEQ ID NO: 118 - DNA, 130 bp, synthetic construct
  misc_feature 1..130: note = Synthetic
  source 1..130: mol_type = other DNA
taatacgactcactataggtgctcgagatactgttcccgatccaagccggccatggtcccagcctcctcgctggcggccggtgggcaacattccgaggggaccgtcccctcggtaatggcgaatgggacc

SEQ ID NO: 119 - DNA, 45 bp, synthetic construct; identical to SEQ ID NO: 40
  misc_feature 1..45: note = Synthetic
  source 1..45: mol_type = other DNA

SEQ ID NO: 120 - DNA, 130 bp, synthetic construct
  misc_feature 1..130: note = Synthetic
  source 1..130: mol_type = other DNA
taatacgactcactataggatctcgagatactgttcccgatccaagccggccatggtcccagcctcctcgctggcggccggtgggcaacattccgaggggaccgtcccctcggtaatggcgaatgggacc

SEQ ID NO: 121 - DNA, 46 bp, synthetic construct; identical to SEQ ID NO: 41
  misc_feature 1..46: note = Synthetic
  source 1..46: mol_type = other DNA

SEQ ID NO: 122 - DNA, 131 bp, synthetic construct
  misc_feature 1..131: note = Synthetic
  source 1..131: mol_type = other DNA
taatacgactcactataggtaactcgagatactgttcccgatccaagccggccatggtcccagcctcctcgctggcggccggtgggcaacattccgaggggaccgtcccctcggtaatggcgaatgggacc

SEQ ID NO: 123 - DNA, 46 bp, synthetic construct; identical to SEQ ID NO: 42
  misc_feature 1..46: note = Synthetic
  source 1..46: mol_type = other DNA

SEQ ID NO: 124 - DNA, 131 bp, synthetic construct
  misc_feature 1..131: note = Synthetic
  source 1..131: mol_type = other DNA
taatacgactcactatagggatctcgagatactgttcccgatccaagccggccatggtcccagcctcctcgctggcggccggtgggcaacattccgaggggaccgtcccctcggtaatggcgaatgggacc

SEQ ID NO: 125 - DNA, 60 bp, synthetic construct; identical to SEQ ID NO: 43
  misc_feature 1..60: note = Synthetic
  source 1..60: mol_type = other DNA

SEQ ID NO: 126 - DNA, 57 bp, synthetic construct
  misc_feature 1..57: note = Synthetic
  source 1..57: mol_type = other DNA
taatacgactcactataggtccctatcagtgatagagactcgagatactgttcccga

SEQ ID NO: 127 - DNA, 64 bp, synthetic construct; identical to SEQ ID NO: 45
  misc_feature 1..64: note = Synthetic
  source 1..64: mol_type = other DNA

SEQ ID NO: 128 - DNA, 66 bp, synthetic construct; identical to SEQ ID NO: 46
  misc_feature 1..66: note = Synthetic
  source 1..66: mol_type = other DNA

SEQ ID NO: 129 - DNA, 68 bp, synthetic construct; identical to SEQ ID NO: 47
  misc_feature 1..68: note = Synthetic
  source 1..68: mol_type = other DNA

SEQ ID NO: 130 - DNA, 70 bp, synthetic construct; identical to SEQ ID NO: 48
  misc_feature 1..70: note = Synthetic
  source 1..70: mol_type = other DNA

SEQ ID NO: 131 - DNA, 71 bp, synthetic construct; identical to SEQ ID NO: 49
  misc_feature 1..71: note = Synthetic
  source 1..71: mol_type = other DNA

SEQ ID NO: 132 - DNA, 65 bp, synthetic construct; identical to SEQ ID NO: 50
  misc_feature 1..65: note = Synthetic
  source 1..65: mol_type = other DNA

SEQ ID NO: 133 - DNA, 89 bp, synthetic construct; identical to SEQ ID NO: 51
  misc_feature 1..89: note = Synthetic
  source 1..89: mol_type = other DNA

SEQ ID NO: 134 - DNA, 89 bp, synthetic construct
  misc_feature 1..89: note = Synthetic
  source 1..89: mol_type = other DNA
ttggatcgggaacagtatctcgagttggatcgggaacagtatctcgagtatctgaataactgttcatgtgtctatagtgagtcgtatta

SEQ ID NO: 135 - DNA, 84 bp, synthetic construct; identical to SEQ ID NO: 52
  misc_feature 1..84: note = Synthetic
  source 1..84: mol_type = other DNA

SEQ ID NO: 136 - DNA, 77 bp, synthetic construct; identical to SEQ ID NO: 53
  misc_feature 1..77: note = Synthetic
  source 1..77: mol_type = other DNA

SEQ ID NO: 137 - DNA, 78 bp, synthetic construct; identical to SEQ ID NO: 54
  misc_feature 1..78: note = Synthetic
  source 1..78: mol_type = other DNA

SEQ ID NO: 138 - DNA, 79 bp, synthetic construct; identical to SEQ ID NO: 55
  misc_feature 1..79: note = Synthetic
  source 1..79: mol_type = other DNA

SEQ ID NO: 139 - DNA, 78 bp, synthetic construct; identical to SEQ ID NO: 56
  misc_feature 1..78: note = Synthetic
  source 1..78: mol_type = other DNA

SEQ ID NO: 140 - DNA, 79 bp, synthetic construct; identical to SEQ ID NO: 57
  misc_feature 1..79: note = Synthetic
  source 1..79: mol_type = other DNA

SEQ ID NO: 141 - DNA, 80 bp, synthetic construct; identical to SEQ ID NO: 58
  misc_feature 1..80: note = Synthetic
  source 1..80: mol_type = other DNA

SEQ ID NO: 142 - DNA, 47 bp, synthetic construct; identical to SEQ ID NO: 59
  misc_feature 1..47: note = Synthetic
  source 1..47: mol_type = other DNA

SEQ ID NO: 143 - DNA, 47 bp, synthetic construct
  misc_feature 1..47: note = Synthetic
  source 1..47: mol_type = other DNA
cgttctgcctttgtctgtttcatttgtccctatagtgagtcgtatta

SEQ ID NO: 144 - DNA, 65 bp, synthetic construct; identical to SEQ ID NO: 60
  misc_feature 1..65: note = Synthetic
  source 1..65: mol_type = other DNA

SEQ ID NO: 145 - DNA, 68 bp, synthetic construct; identical to SEQ ID NO: 61
  misc_feature 1..68: note = Synthetic
  source 1..68: mol_type = other DNA

SEQ ID NO: 146 - DNA, 60 bp, synthetic construct
  misc_feature 1..60: note = Synthetic
  source 1..60: mol_type = other DNA
aatacgactcactataggtccctatcagtgatagagaaacgaatacagaacatagaaaca

SEQ ID NO: 147 - DNA, 60 bp, synthetic construct
  misc_feature 1..60: note = Synthetic
  source 1..60: mol_type = other DNA
cgactcactataggcacatgaacagttattcagataaacgagtggaatggatgcatctca

SEQ ID NO: 148 - DNA, 47 bp, synthetic construct; identical to SEQ ID NO: 64
  misc_feature 1..47: note = Synthetic
  source 1..47: mol_type = other DNA

SEQ ID NO: 149 - DNA, 47 bp, synthetic construct
  misc_feature 1..47: note = Synthetic
  source 1..47: mol_type = other DNA
cgttctgcctttttctgtttcatttgtccctatagtgagtcgtatta

SEQ ID NO: 150 - DNA, 53 bp, synthetic construct; identical to SEQ ID NO: 65
  misc_feature 1..53: note = Synthetic
  source 1..53: mol_type = other DNA

SEQ ID NO: 151 - DNA, 53 bp, synthetic construct
  misc_feature 1..53: note = Synthetic
  source 1..53: mol_type = other DNA
cgttctgcctttttctgtttcatttgtcttttttcctatagtgagtcgtatta

SEQ ID NO: 152 - DNA, 77 bp, synthetic construct; identical to SEQ ID NO: 66
  misc_feature 1..77: note = Synthetic
  source 1..77: mol_type = other DNA

SEQ ID NO: 153 - DNA, 77 bp, synthetic construct
  misc_feature 1..77: note = Synthetic
  source 1..77: mol_type = other DNA
ctccctttttctgtttcatttgtcaaaaaagacaaatgaaacagaaaaaggcagaacgcctatagtgagtcgtatta

SEQ ID NO: 154 - DNA, 75 bp, synthetic construct; identical to SEQ ID NO: 67
  misc_feature 1..75: note = Synthetic
  source 1..75: mol_type = other DNA

SEQ ID NO: 155 - DNA, 75 bp, synthetic construct
  misc_feature 1..75: note = Synthetic
  source 1..75: mol_type = other DNA
gcctttttctgtttcatttgtcaaaaaagacaaatgaaacagaaaaaggcagaacgcctatagtgagtcgtatta

SEQ ID NO: 156 - DNA, 76 bp, synthetic construct; identical to SEQ ID NO: 68
  misc_feature 1..76: note = Synthetic
  source 1..76: mol_type = other DNA

SEQ ID NO: 157 - DNA, 76 bp, synthetic construct
  misc_feature 1..76: note = Synthetic
  source 1..76: mol_type = other DNA
gcctttttctgtttcatttgtcaaaaaagacaaatgaaacagaaaaaggcagaacgtcctatagtgagtcgtatta

SEQ ID NO: 158 - DNA, 97 bp, synthetic construct; identical to SEQ ID NO: 69
  misc_feature 1..97: note = Synthetic
  source 1..97: mol_type = other DNA

SEQ ID NO: 159 - DNA, 97 bp, synthetic construct
  misc_feature 1..97: note = Synthetic
  source 1..97: mol_type = other DNA
gcctttttctgtttcatttgtcaaaaaagacaaatgaaacagaaaaaggcagaacggggtctctatcactgatagggacctatagtgagtcgtatta

SEQ ID NO: 160 - DNA, 90 bp, synthetic construct
  misc_feature 1..90: note = Synthetic
  source 1..90: mol_type = other DNA
tacgactcactataggtccctatcagtgatagagagttatgtcacgattgtcaatcaattatgaatggatcctcattcgattatcctctt

SEQ ID NO: 161 - DNA, 97 bp, synthetic construct; identical to SEQ ID NO: 71
  misc_feature 1..97: note = Synthetic
  source 1..97: mol_type = other DNA

SEQ ID NO: 162 - DNA, 97 bp, synthetic construct
  misc_feature 1..97: note = Synthetic
  source 1..97: mol_type = other DNA
gcctttttctgtttcatttgtcaaaaaagacaaatgaaacagaaaaaggcagaacgtatctgaataactgttcatgtgcctatagtgagtcgtatta

SEQ ID NO: 163 - DNA, 110 bp, synthetic construct; identical to SEQ ID NO: 72
  misc_feature 1..110: note = Synthetic
  source 1..110: mol_type = other DNA

SEQ ID NO: 164 - DNA, 110 bp, synthetic construct
  misc_feature 1..110: note = Synthetic
  source 1..110: mol_type = other DNA
gcctttttctgtttcatttgtcaaaaaagacaaatgaaacagaaaaaggcagaacgaaggcacatgaactatctgaataactgttcatgtgcctatagtgagtcgtatta

SEQ ID NO: 165 - DNA, 110 bp, synthetic construct; identical to SEQ ID NO: 73
  misc_feature 1..110: note = Synthetic
  source 1..110: mol_type = other DNA

SEQ ID NO: 166 - DNA, 66 bp, synthetic construct; identical to SEQ ID NO: 74
  misc_feature 1..66: note = Synthetic
  source 1..66: mol_type = other DNA

SEQ ID NO: 167 - DNA, 66 bp, synthetic construct
  misc_feature 1..66: note = Synthetic
  source 1..66: mol_type = other DNA
cgttctgcctttgtctgtttcatttgtctctctatcactgatagggacctatagtgagtcgtatta

SEQ ID NO: 168 - DNA, 69 bp, synthetic construct; identical to SEQ ID NO: 75
  misc_feature 1..69: note = Synthetic
  source 1..69: mol_type = other DNA

SEQ ID NO: 169 - DNA, 51 bp, synthetic construct; identical to SEQ ID NO: 76
  misc_feature 1..51: note = Synthetic
  source 1..51: mol_type = other DNA

SEQ ID NO: 170 - DNA, 51 bp, synthetic construct
  misc_feature 1..51: note = Synthetic
  source 1..51: mol_type = other DNA
tgagatgaagtttggtttcacgttctgtttttcctatagtgagtcgtatta

SEQ ID NO: 171 - DNA, 51 bp, synthetic construct; identical to SEQ ID NO: 77
  misc_feature 1..51: note = Synthetic
  source 1..51: mol_type = other DNA

SEQ ID NO: 172 - DNA, 51 bp, synthetic construct
  misc_feature 1..51: note = Synthetic
  source 1..51: mol_type = other DNA
tgagatgcatccattccactcgttctgtttttcctatagtgagtcgtatta

SEQ ID NO: 173 - DNA, 89 bp, synthetic construct; identical to SEQ ID NO: 78
  misc_feature 1..89: note = Synthetic
  source 1..89: mol_type = other DNA

SEQ ID NO: 174 - DNA, 89 bp, synthetic construct
  misc_feature 1..89: note = Synthetic
  source 1..89: mol_type = other DNA
gtttggtttgacgttctggaaaaacagaacgtgaaaccaaacttcatctcatctctatcactgatagggacctatagtgagtcgtatta

SEQ ID NO: 175 - DNA, 90 bp, synthetic construct; identical to SEQ ID NO: 79
  misc_feature 1..90: note = Synthetic
  source 1..90: mol_type = other DNA

SEQ ID NO: 176 - DNA, 90 bp, synthetic construct
  misc_feature 1..90: note = Synthetic
  source 1..90: mol_type = other DNA
cattgcactcgttctggaaaaacagaacgagtggaatggatgcatctcatatctgaataactgttcatgtgcctatagtgagtcgtatta

SEQ ID NO: 177 - DNA, 89 bp, synthetic construct; identical to SEQ ID NO: 80
  misc_feature 1..89: note = Synthetic
  source 1..89: mol_type = other DNA

SEQ ID NO: 178 - DNA, 89 bp, synthetic construct
  misc_feature 1..89: note = Synthetic
  source 1..89: mol_type = other DNA
ctatgttctgtattcgttggaaaaaacgaatacagaacatagaaacaggggtctctatcactgatagggacctatagtgagtcgtatta

SEQ ID NO: 179 - DNA, 90 bp, synthetic construct; identical to SEQ ID NO: 81
  misc_feature 1..90: note = Synthetic
  source 1..90: mol_type = other DNA

SEQ ID NO: 180 - DNA, 90 bp, synthetic construct
  misc_feature 1..90: note = Synthetic
  source 1..90: mol_type = other DNA
catccattccactcgttggtaaaaaaacgagtggaatggatgcatctcatatctgaataactgttcatgtgcctatagtgagtcgtatta

SEQ ID NO: 181 - DNA, 47 bp, synthetic construct; identical to SEQ ID NO: 82
  misc_feature 1..47: note = Synthetic
  source 1..47: mol_type = other DNA

SEQ ID NO: 182 - DNA, 47 bp, synthetic construct
  misc_feature 1..47: note = Synthetic
  source 1..47: mol_type = other DNA
ctgtttctatgttctgtattcgttttttcctatagtgagtcgtatta

SEQ ID NO: 183 - DNA, 49 bp, synthetic construct; identical to SEQ ID NO: 83
  misc_feature 1..49: note = Synthetic
  source 1..49: mol_type = other DNA

SEQ ID NO: 184 - DNA, 49 bp, synthetic construct
  misc_feature 1..49: note = Synthetic
  source 1..49: mol_type = other DNA
tgagatgcatccattccactcgtttttttacctatagtgagtcgtatta

SEQ ID NO: 185 - DNA, 23 bp, synthetic construct
  misc_feature 1..23: note = Synthetic
  source 1..23: mol_type = other DNA
taatacgactcactataggaacc

SEQ ID NO: 186 - DNA, 18 bp, synthetic construct
  misc_feature 1..18: note = Synthetic
  source 1..18: mol_type = other DNA
gagctctatgacaagggc

SEQ ID NO: 187 - DNA, 23 bp, synthetic construct
  misc_feature 1..23: note = Synthetic
  source 1..23: mol_type = other DNA
taatacgactcactataggaacc

SEQ ID NO: 188 - DNA, 19 bp, synthetic construct
  misc_feature 1..19: note = Synthetic
  source 1..19: mol_type = other DNA
caaaaaacccctcaagacc

SEQ ID NO: 189 - DNA, 22 bp, synthetic construct
  misc_feature 1..22: note = Synthetic
  source 1..22: mol_type = other DNA
taatacgactcactataggtgc

SEQ ID NO: 190 - DNA, 20 bp, synthetic construct
  misc_feature 1..20: note = Synthetic
  source 1..20: mol_type = other DNA
taatacgactcactatagga

SEQ ID NO: 191 - DNA, 20 bp, synthetic construct
  misc_feature 1..20: note = Synthetic
  source 1..20: mol_type = other DNA
taatacgactcactataggt

SEQ ID NO: 192 - DNA, 25 bp, synthetic construct
  misc_feature 1..25: note = Synthetic
  source 1..25: mol_type = other DNA
taatacgactcactataggtaactc

SEQ ID NO: 193 - DNA, 24 bp, synthetic construct
  misc_feature 1..24: note = Synthetic
  source 1..24: mol_type = other DNA
taatacgactcactatatcctatc

SEQ ID NO: 194 - DNA, 20 bp, synthetic construct
  misc_feature 1..20: note = Synthetic
  source 1..20: mol_type = other DNA
taatacgactcactataggg

SEQ ID NO: 195 - DNA, 19 bp, synthetic construct
  misc_feature 1..19: note = Synthetic
  source 1..19: mol_type = other DNA
ttggatcgggaacagtatc

SEQ ID NO: 196 - DNA, 27 bp, synthetic construct
  misc_feature 1..27: note = Synthetic
  source 1..27: mol_type = other DNA
taatacgactcactataggtccctatc

SEQ ID NO: 197 - DNA, 22 bp, synthetic construct
  misc_feature 1..22: note = Synthetic
  source 1..22: mol_type = other DNA
ttggatcgggaacagtatctcg

SEQ ID NO: 198 - DNA, 22 bp, synthetic construct
  misc_feature 1..22: note = Synthetic
  source 1..22: mol_type = other DNA
taatacgactcactatagacac

SEQ ID NO: 199 - DNA, 20 bp, synthetic construct
  misc_feature 1..20: note = Synthetic
  source 1..20: mol_type = other DNA
taatacgactcactataggc

SEQ ID NO: 200 - DNA, 17 bp, synthetic construct
  misc_feature 1..17: note = Synthetic
  source 1..17: mol_type = other DNA
cgttctgcctttgtctg

SEQ ID NO: 201 - DNA, 18 bp, synthetic construct
  misc_feature 1..18: note = Synthetic
  source 1..18: mol_type = other DNA
ggtcccattcgccattac

SEQ ID NO: 202 - DNA, 19 bp, synthetic construct
  misc_feature 1..19: note = Synthetic
  source 1..19: mol_type = other DNA
tgagatgaagtttggtttc

SEQ ID NO: 203 - DNA, 19 bp, synthetic construct
  misc_feature 1..19: note = Synthetic
  source 1..19: mol_type = other DNA
tgagatgcatccattccac

SEQ ID NO: 204 - DNA, 16 bp, synthetic construct
  misc_feature 1..16: note = Synthetic
  source 1..16: mol_type = other DNA
tgagatgcatccattc

SEQ ID NO: 205 - DNA, 21 bp, synthetic construct
  misc_feature 1..21: note = Synthetic
  source 1..21: mol_type = other DNA
cgagaagaggataatcgaatg

SEQ ID NO: 206 - DNA, 21 bp, synthetic construct
  misc_feature 1..21: note = Synthetic
  source 1..21: mol_type = other DNA
ctgtttctatgttctgtattc

SEQ ID NO: 207 - DNA, 22 bp, synthetic construct
  misc_feature 1..22: note = Synthetic
  source 1..22: mol_type = other DNA
atacgtaattaagaacaacacg

SEQ ID NO: 208 - RNA, 48 nt, synthetic construct
  misc_feature 1..48: note = Synthetic
  source 1..48: mol_type = other RNA
ggcacatgaacagttattcagataaacgagtggaatggatgcatctca

SEQ ID NO: 209 - RNA, 45 nt, synthetic construct
  misc_feature 1..45: note = Synthetic
  source 1..45: mol_type = other RNA
ggtccctatcagtgatagagaaacgaatacagaacatagaaacag

SEQ ID NO: 210 - DNA, 55 bp, synthetic construct; identical to SEQ ID NO: 25
  misc_feature 1..55: note = Synthetic
  source 1..55: mol_type = other DNA

SEQ ID NO: 211 - DNA, 67 bp, synthetic construct
  misc_feature 1..67: note = Synthetic
  source 1..67: mol_type = other DNA
gacaaatgaaacagacaaaggcagaacgaatacacaacatacactgtgaacgagtgcaatggatgca

SEQ ID NO: 212 - DNA, 28 bp, synthetic construct
  misc_feature 1..28: note = Synthetic
  source 1..28: mol_type = other DNA
cgttctgcctttgtctgtttcatttgtc

SEQ ID NO: 213 - DNA, 24 bp, synthetic construct
  misc_feature 1..24: note = Synthetic
  source 1..24: mol_type = other DNA
gacggaaacagacaaagtaaacag

SEQ ID NO: 214 - RNA, 79 nt, synthetic construct
  misc_feature 1..79: note = Synthetic
  source 1..79: mol_type = other RNA
ggtccctatcagtgatagagaccccgttctgcctttttctgtttcatttgtctttttgacaaatgaaacagaaaaaggc

SEQ ID NO: 215 - RNA, 36 nt, synthetic construct
  misc_feature 1..36: note = Synthetic
  source 1..36: mol_type = other RNA
ggaaaaaagacaaatgaaacagaaaaaggcagaacg

SEQ ID NO: 216 - RNA, 51 nt, synthetic construct
  misc_feature 1..51: note = Synthetic
  source 1..51: mol_type = other RNA
ggcacatgaacagttattcagatacagaagcagtggaatggatgcatctca

SEQ ID NO: 217 - DNA, 27 bp, synthetic construct
  misc_feature 1..27: note = Synthetic
  source 1..27: mol_type = other DNA
tgagatgcatccattccactcgttctg

SEQ ID NO: 218 - DNA, 43 bp, synthetic construct; identical to SEQ ID NO: 93
  misc_feature 1..43: note = Synthetic
  source 1..43: mol_type = other DNA

SEQ ID NO: 219 - DNA, 28 bp, synthetic construct
  misc_feature 1..28: note = Synthetic
  source 1..28: mol_type = other DNA
cgttctgcctttgtctgtttcatttgtc

SEQ ID NO: 220 - DNA, 24 bp, synthetic construct
  misc_feature 1..24: note = Synthetic
  source 1..24: mol_type = other DNA
gacggaaacagacaaagtaaacag

SEQ ID NO: 221 - RNA, 92 nt, synthetic construct
  misc_feature 1..92: note = Synthetic
  source 1..92: mol_type = other RNA
ggcacatgaacagttattcagatagttcatgtgccttcgttctgcctttttctgtttcatttgtctttttgacaaatgaaacagaaaaaggc

SEQ ID NO: 222 - RNA, 36 nt, synthetic construct; identical to SEQ ID NO: 215
  misc_feature 1..36: note = Synthetic
  source 1..36: mol_type = other RNA

SEQ ID NO: 223 - RNA, 49 nt, synthetic construct
  misc_feature 1..49: note = Synthetic
  source 1..49: mol_type = other RNA
ggtccctatcagtgatagagacagaaacgtgaaaccaaacttcatctca

SEQ ID NO: 224 - DNA, 27 bp, synthetic construct
  misc_feature 1..27: note = Synthetic
  source 1..27: mol_type = other DNA
tgagatgaagtttggtttcacgttctg

SEQ ID NO: 225 - DNA, 43 bp, synthetic construct; identical to SEQ ID NO: 91
  misc_feature 1..43: note = Synthetic
  source 1..43: mol_type = other DNA

SEQ ID NO: 226 - DNA, 28 bp, synthetic construct
  misc_feature 1..28: note = Synthetic
  source 1..28: mol_type = other DNA
cgttctgcctttgtctgtttcatttgtc

SEQ ID NO: 227 - DNA, 24 bp, synthetic construct
  misc_feature 1..24: note = Synthetic
  source 1..24: mol_type = other DNA
gacaaatgaaacagacaaaggcag

SEQ ID NO: 228 - RNA, 80 nt, synthetic construct
  misc_feature 1..80: note = Synthetic
  source 1..80: mol_type = other RNA
ggtccctatcagtgatagagaccccgttctgcctttttctgtttcatttgtcttttttgacaaatgaaacagaaaaaggc

SEQ ID NO: 229 - RNA, 36 nt, synthetic construct; identical to SEQ ID NO: 215
  misc_feature 1..36: note = Synthetic
  source 1..36: mol_type = other RNA

SEQ ID NO: 230 - RNA, 52 nt, synthetic construct
  misc_feature 1..52: note = Synthetic
  source 1..52: mol_type = other RNA
ggcactagaacagttattcagatagacaaatgaaacagacaaaggcagaacg

SEQ ID NO: 231 - DNA, 28 bp, synthetic construct
  misc_feature 1..28: note = Synthetic
  source 1..28: mol_type = other DNA
cgttctgcctttgtctgtttcatttgtc

SEQ ID NO: 232 - DNA, 24 bp, synthetic construct
  misc_feature 1..24: note = Synthetic
  source 1..24: mol_type = other DNA
gacaaatgaaacagacaaaggcag

SEQ ID NO: 233 - RNA, 93 nt, synthetic construct; identical to SEQ ID NO: 33
  misc_feature 1..93: note = Synthetic
  source 1..93: mol_type = other RNA

SEQ ID NO: 234 - RNA, 36 nt, synthetic construct
  misc_feature 1..36: note = Synthetic
  source 1..36: mol_type = other RNA
ggaaaaaagacaaatgaaacagaaaaaggcagaagc

SEQ ID NO: 235 - RNA, 49 nt, synthetic construct
  misc_feature 1..49: note = Synthetic
  source 1..49: mol_type = other RNA
ggtccctatcagtgatagagagacaaatgaaacagacaaaggcagaacg

SEQ ID NO: 236 - DNA, 28 bp, synthetic construct
  misc_feature 1..28: note = Synthetic
  source 1..28: mol_type = other DNA
cgttctgcctttgtctgtttcatttgtc

SEQ ID NO: 237 - DNA, 24 bp, synthetic construct
  misc_feature 1..24: note = Synthetic
  source 1..24: mol_type = other DNA
gacaaatgaaacagacaaaggcag

SEQ ID NO: 238 - RNA, 34 nt, synthetic construct
  misc_feature 1..34: note = Synthetic
  source 1..34: mol_type = other RNA
ggaaaaacagaacgtgaaaccaaacttcatctca

SEQ ID NO: 239 - RNA, 72 nt, synthetic construct
  misc_feature 1..72: note = Synthetic
  source 1..72: mol_type = other RNA
ggtccctatcagtgatagagatgagatgaagtttggtttcacgttctgtttttccagaacgtcaaaccaaac

SEQ ID NO: 240 - DNA, 27 bp, synthetic construct
  misc_feature 1..27: note = Synthetic
  source 1..27: mol_type = other DNA
tgagatgaagtttggtttcacgttctg

SEQ ID NO: 241 - DNA, 43 bp, synthetic construct; identical to SEQ ID NO: 91
  misc_feature 1..43: note = Synthetic
  source 1..43: mol_type = other DNA

SEQ ID NO: 242 - RNA, 34 nt, synthetic construct
  misc_feature 1..34: note = Synthetic
  source 1..34: mol_type = other RNA
ggaaaaacagaacgagtggaatggatgcatctca

SEQ ID NO: 243 - RNA, 73 nt, synthetic construct
  misc_feature 1..73: note = Synthetic
  source 1..73: mol_type = other RNA
ggcacatgaacagttattcagatatgagatgcatccattccactcgttctgtttttccagaacgagtgcaatg

SEQ ID NO: 244 - DNA, 27 bp, synthetic construct
  misc_feature 1..27: note = Synthetic
  source 1..27: mol_type = other DNA
tgagatgcatccattccactcgttctg

SEQ ID NO: 245 - DNA, 43 bp, synthetic construct; identical to SEQ ID NO: 93
  misc_feature 1..43: note = Synthetic
  source 1..43: mol_type = other DNA

SEQ ID NO: 246 - DNA, 28 bp, synthetic construct
  misc_feature 1..28: note = Synthetic
  source 1..28: mol_type = other DNA
cgttctgcctttgtctgtttcatttgtc

SEQ ID NO: 247 - DNA, 24 bp, synthetic construct
  misc_feature 1..24: note = Synthetic
  source 1..24: mol_type = other DNA
gacaaatgaaacagacaaaggcag

SEQ ID NO: 248 - RNA, 72 nt, synthetic construct
  misc_feature 1..72: note = Synthetic
  source 1..72: mol_type = other RNA
ggtccctatcagtgatagagacccctgtttctatgttctgtattcgttttttccaacgaatacagaacatag

SEQ ID NO: 249 - RNA, 30 nt, synthetic construct
  misc_feature 1..30: note = Synthetic
  source 1..30: mol_type = other RNA
ggaaaaaacgaatacagaacatagaaacag

SEQ ID NO: 250 - RNA, 48 nt, synthetic construct; identical to SEQ ID NO: 208
  misc_feature 1..48: note = Synthetic
  source 1..48: mol_type = other RNA

SEQ ID NO: 251 - DNA, 55 bp, synthetic construct; identical to SEQ ID NO: 25
  misc_feature 1..55: note = Synthetic
  source 1..55: mol_type = other DNA

SEQ ID NO: 252 - DNA, 66 bp, synthetic construct
  misc_feature 1..66: note = Synthetic
  source 1..66: mol_type = other DNA
gacaaatgaaacagacaaaggcagaacgaatacacaacatacactgtgaagagtgcaatggatgca

SEQ ID NO: 253 - DNA, 28 bp, synthetic construct
  misc_feature 1..28: note = Synthetic
  source 1..28: mol_type = other DNA
cgttctgcctttgtctgtttcatttgtc

SEQ ID NO: 254 - DNA, 24 bp, synthetic construct
  misc_feature 1..24: note = Synthetic
  source 1..24: mol_type = other DNA
gacaaatgaaacagacaaaggcag

SEQ ID NO: 255 - RNA, 73 nt, synthetic construct
  misc_feature 1..73: note = Synthetic
  source 1..73: mol_type = other RNA
ggcacatgaacagttattcagatatgagatgcatccattccactcgtttttttaccaacgagtggaatggatg

SEQ ID NO: 256 - RNA, 32 nt, synthetic construct
  misc_feature 1..32: note = Synthetic
  source 1..32: mol_type = other RNA
ggtaaaaaaacgagtggaatggatgcatctca

SEQ ID NO: 257 - RNA, 45 nt, synthetic construct; identical to SEQ ID NO: 209
  misc_feature 1..45: note = Synthetic
  source 1..45: mol_type = other RNA

SEQ ID NO: 258 - DNA, 55 bp, synthetic construct; identical to SEQ ID NO: 25
  misc_feature 1..55: note = Synthetic
  source 1..55: mol_type = other DNA

SEQ ID NO: 259 - DNA, 67 bp, synthetic construct; identical to SEQ ID NO: 97
  misc_feature 1..67: note = Synthetic
  source 1..67: mol_type = other DNA

SEQ ID NO: 260 - DNA, 28 bp, synthetic construct
  misc_feature 1..28: note = Synthetic
  source 1..28: mol_type = other DNA
cgttctgcctttgtctgtttcatttgtc

SEQ ID NO: 261 - DNA, 24 bp, synthetic construct
  misc_feature 1..24: note = Synthetic
  source 1..24: mol_type = other DNA
gacaaatgaaacagacaaaggcag

SEQ ID NO: 262 - RNA, 80 nt, synthetic construct; identical to SEQ ID NO: 228
  misc_feature 1..80: note = Synthetic
  source 1..80: mol_type = other RNA

SEQ ID NO: 263 - RNA, 92 nt, synthetic construct
  misc_feature 1..92: note = Synthetic
  source 1..92: mol_type = other RNA
ggcacatgaacagttattcagatagttcatgtgccttcgttctgctttttctgtttcatttgtcttttttgacaaatgaaacagaaaaaggc

SEQ ID NO: 264 - RNA, 36 nt, synthetic construct; identical to SEQ ID NO: 215
  misc_feature 1..36: note = Synthetic
  source 1..36: mol_type = other RNA

SEQ ID NO: 265 - DNA, 28 bp, synthetic construct
  misc_feature 1..28: note = Synthetic
  source 1..28: mol_type = other DNA
cgttctgcctttgtctgtttcatttgtc

SEQ ID NO: 266 - DNA, 24 bp, synthetic construct
  misc_feature 1..24: note = Synthetic
  source 1..24: mol_type = other DNA
gacaaatgaaacagacaaaggcag

SEQ ID NO: 267 - RNA, 80 nt, synthetic construct; identical to SEQ ID NO: 228
  misc_feature 1..80: note = Synthetic
  source 1..80: mol_type = other RNA

SEQ ID NO: 268 - RNA, 36 nt, synthetic construct; identical to SEQ ID NO: 215
  misc_feature 1..36: note = Synthetic
  source 1..36: mol_type = other RNA

SEQ ID NO: 269 - DNA, 28 bp, synthetic construct
  misc_feature 1..28: note = Synthetic
  source 1..28: mol_type = other DNA
cgttctgcctttgtctgtttcatttgtc

SEQ ID NO: 270 - DNA, 24 bp, synthetic construct
  misc_feature 1..24: note = Synthetic
  source 1..24: mol_type = other DNA
gacggaaacagacaaagtaaacag

SEQ ID NO: 271 - RNA, 93 nt, synthetic construct
  misc_feature 1..93: note = Synthetic
  source 1..93: mol_type = other RNA
ggcacatgaacagttattcagatagttcatgtgccttcgttctgcctttttcagtttcatttgtcttttttgacaaatgaaacagaaaaaggc

SEQ ID NO: 272 - RNA, 36 nt, synthetic construct; identical to SEQ ID NO: 215
  misc_feature 1..36: note = Synthetic
  source 1..36: mol_type = other RNA

SEQ ID NO: 273 - DNA, 28 bp, synthetic construct
  misc_feature 1..28: note = Synthetic
  source 1..28: mol_type = other DNA
cgttctgcctttgtctgtttcatttgtc

SEQ ID NO: 274 - DNA, 24 bp, synthetic construct
  misc_feature 1..24: note = Synthetic
  source 1..24: mol_type = other DNA
gacaaatgaaacagacaaaggcag

SEQ ID NO: 275 - RNA, 48 nt, synthetic construct
  misc_feature 1..48: note = Synthetic
  source 1..48: mol_type = other RNA
ggtccctatcagtgatagagacagaacgtgaaaccaaacttcatctca

SEQ ID NO: 276 - DNA, 27 bp, synthetic construct
  misc_feature 1..27: note = Synthetic
  source 1..27: mol_type = other DNA
tgagatgaagtttggtttcacgttctg

SEQ ID NO: 277 - DNA, 43 bp, synthetic construct; identical to SEQ ID NO: 91
  misc_feature 1..43: note = Synthetic
  source 1..43: mol_type = other DNA

SEQ ID NO: 278 - RNA, 51 nt, synthetic construct
  misc_feature 1..51: note = Synthetic
  source 1..51: mol_type = other RNA
ggcacatgaacagttattcagatacagaacgagtggaatggatgcatctca

SEQ ID NO: 279 - DNA, 27 bp, synthetic construct
  misc_feature 1..27: note = Synthetic
  source 1..27: mol_type = other DNA
tgagatgcatccattccactcgttctg

SEQ ID NO: 280 - DNA, 43 bp, synthetic construct; identical to SEQ ID NO: 93
  misc_feature 1..43: note = Synthetic
  source 1..43: mol_type = other DNA

SEQ ID NO: 281 - DNA, 28 bp, synthetic construct
  misc_feature 1..28: note = Synthetic
  source 1..28: mol_type = other DNA
cgttctgcctttgtctgtttcatttgtc

SEQ ID NO: 282 - DNA, 24 bp, synthetic construct
  misc_feature 1..24: note = Synthetic
  source 1..24: mol_type = other DNA
gacaaatgaaacagacaaaggcag

SEQ ID NO: 283 - DNA, 23 bp, synthetic construct
  source 1..23: mol_type = other DNA
gagaggatttggatcgggaacag

SEQ ID NO: 284 - DNA, 20 bp, synthetic construct
  source 1..20: mol_type = other DNA
agatactgttcccgatccaa

SEQ ID NO: 285 - DNA, 24 bp, synthetic construct
  source 1..24: mol_type = other DNA
ttggatcgggaacagtatctcgag

SEQ ID NO: 286 - DNA, 16 bp, synthetic construct
  source 1..16: mol_type = other DNA
ctcgagatactgttcc

(19) United States
(12) Patent Application Publication
(10) Pub. No.: US 2023/0332252 A1
(43) Pub. Date: Oct. 19, 2023

(54) CELL-FREE BIOSENSORS WITH DNA STRAND DISPLACEMENT CIRCUITS AND POLYMERASE STRAND RECYCLING (PSR)

(71) Applicant: Northwestern University, Evanston, IL (US)

(72) Inventors: Jaeyoung Kirsten Jung, Evanston, IL (US); Julius Beau Lucks, Evanston, IL (US); Khalid K. Alam, Evanston, IL (US); Yueyi Li, Evanston, IL (US)

(21) Appl. No.: 18/311,030

(22) Filed: May 2, 2023

Related U.S. Application Data
(63) Continuation-in-part of application No. PCT/US2022/018133, filed on Feb. 28, 2022.
(60) Provisional application No. 63/254,824, filed on Oct. 12, 2021.
(60) Provisional application No. 63/154,247, filed on Feb. 26, 2021.
(60) Provisional application No. 63/337,267, filed on May 2, 2022.

(51) Int. Cl. C12Q 1/6897; C12N 15/115

(52) U.S. Cl. CPC C12Q 1/6897; C12N 15/115; C12N 2330/30; C12N 2310/16

(57) ABSTRACT

Disclosed are compositions, systems, kits, and methods for detecting an analyte or target molecule in a sample by regulated in vitro transcription. The compositions, systems, kits, and methods typically comprise and/or utilize one or more components selected from: (a) an RNA polymerase; (b) an allosteric transcription factor (aTF), wherein the aTF binds an analyte or target molecule as a ligand; (c) an engineered transcription template; (d) a dsDNA signal gate molecule; (e) a dsDNA fuel gate molecule; and/or any combination thereof.

### CROSS-REFERENCE TO RELATED APPLICATIONS

This application is a continuation-in-part of PCT/US2022/018133 filed on Feb. 28, 2022, which claims the benefit of U.S. Application No. 63/154,247 filed Feb. 26, 2021, and U.S. Application No. 63/254,824 filed Oct. 12, 2021. This application claims priority to U.S. Application No. 63/337,267 filed May 2, 2022. The entire contents of each of the above-referenced applications are incorporated herein by reference.

### STATEMENT REGARDING FEDERALLY SPONSORED RESEARCH OR DEVELOPMENT

This invention was made with government support under NSF1452441 and NSF1929912 awarded by the National Science Foundation, and T32GM008449 awarded by the Institutes of Health. The government has certain rights in the invention.

### SEQUENCE LISTING

A Sequence Listing accompanies this application and is submitted as an ASCII text file of the sequence listing named “702581_02339_Sequence_Listing.xml” which is 369,446 bytes in size and was created on May 1, 2023. The sequence listing is electronically submitted via EFS-Web with the application and is incorporated herein by reference in its entirety.

### FIELD

The technical field relates to sensors for detecting molecules and metals in aqueous solution. In particular, the technical field relates to low-cost, programmable, and rapid sensors for detecting molecules such as toxins, drugs, contaminants and the like, and metals such as zinc, lead, copper and the like in aqueous solutions.

### BACKGROUND

Cell-free biosensing is emerging as a low-cost, easy-to-use and field-deployable diagnostic technology that can be applied to detect a range of chemical contaminants related to human and environmental health [1]. At their core, these systems consist of two layers: a sensing layer that includes an RNA or protein-based biosensor that can detect a chemical target, and an output layer that includes a reporter construct. By genetically wiring the sensing layer to the output layer, a signal can be generated when the target compound binds to the biosensor and activates the expression of the reporter (FIG. 1). Ultimately, reactions can be assembled by embedding the biosensors and reporter constructs within cell-free reaction environments, freeze-dried for easy storage and transportation, and rehydrated with a sample of interest at the point-of-need [1, 2]. Using this approach, cell-free biosensors have been created for chemical compounds related to human health such as zinc [3] and quorum sensing molecules produced by pathogenic bacteria [4], drugs such as gamma-hydroxy-butyrate [5] and water contaminants such as fluoride [1], atrazine [6], antibiotics and heavy metals [7] among others.

### SUMMARY

The present invention relates to compositions, systems, kits, and methods for detecting analytes and target molecules. The compositions, systems, kits, and methods utilize regulated in vitro transcription in order to detect an analyte or a target molecule in a sample via toehold-mediated strand displacement circuits.

The disclosed compositions, systems, kits, and methods typically comprise and/or utilize one or more components selected from: (a) an RNA polymerase; (b) an allosteric transcription factor (aTF), wherein the aTF binds an analyte or target molecule as a ligand; (c) an engineered transcription template; (d) a dsDNA signal gate molecule; (e) a dsDNA fuel gate molecule; and/or any combination thereof. The engineered transcription template typically comprises a promoter sequence for the RNA polymerase and an operator sequence for the aTF. The promoter sequence and operator sequence are operably linked to a sequence encoding an RNA, wherein the aTF modulates transcription of the encoded RNA when the aTF binds the analyte or target molecule as a ligand. The RNA that is transcribed from the engineered transcription template displaces a DNA strand of the dsDNA signal gate which generates a detectable signal.

In some embodiments of the cell-free sensor systems disclosed herein, polymerase strand recycling components (PSR) are included. By way of example, such components include at least a fuel gate. In embodiments comprising PSR components, the RNA that is transcribed from the engineered transcription template additionally or alternatively displaces a DNA strand (e.g., the “RecycleD” strand) of a double-stranded (ds) DNA fuel gate by hybridizing to the “waste” strand of the ds DNA fuel gate, thereby freeing the RecyleD strand of the double-stranded fuel gate. The RecycleD molecule may then displace a DNA strand of the dsDNA signal gate to form a double-stranded molecule which (a) generates detectable signal, and (b) is used as template for promoter independent T7 RNA polymerase transcription, releasing the RecycleD molecules to displace additional DNA signal gates to achieve signal amplification. Any of the logic gates/logic gate assay cell-free systems disclosed herein (e.g., as exemplified in the figures and detailed description) may include polymerase strand recycling components (PSR) as described herein, to amplify signal and/or to improve assay sensitivity.

The disclosure provides compositions, systems, and/or kits for detecting an analyte comprising as components one or more of (a) an RNA polymerase; (b) an allosteric transcription factor (aTF), wherein the analyte is a ligand to which the aTF binds; (c) an engineered transcription template; (d) a double-stranded DNA (dsDNA) signal gate molecule; and (e) a dsDNA fuel gate molecule. In some embodiments, the engineered transcription template comprises a promoter sequence for the RNA polymerase and an operator sequence for the aTF operably linked to a sequence encoding an RNA. In embodiments of the analyte detection systems disclosed herein, the aTF modulates transcription of the encoded RNA when the aTF binds the analyte as a ligand. The transcribed RNA displaces a strand of the dsDNA fuel gate molecule generating a free single-stranded DNA molecule which displaces a strand of the dsDNA signal gate molecule to produce a detectable signal and a new hybrid signal/fuel gate. The RNA polymerase transcribes the hybrid signal/fuel gate to release the fuel gate strand of DNA which can then displace a strand of another dsDNA signal gate molecule, thus creating a positive feedback loop of signal amplification. In some aspects, the dsDNA fuel gate molecule is a double-stranded nucleic acid comprising a waste strand and a RecycleD strand, and in some further aspects the dsDNA signal gate molecule is a fluorescently labeled double-stranded DNA molecule comprising a top strand having a fluorophore conjugated at its 3′-end and a bottom strand having a quencher conjugated at its 5′ end that quenches the fluorophore in the fluorescently labeled double-stranded DNA molecule and the RecycleD strand displaces the bottom strand of the fluorescently labeled double-stranded DNA molecule which results in dequenching of the fluorophore to generate the detectable signal and hybridization of the RecycleD strand to the top strand generates a double-stranded DNA molecule comprising a 3′ toehold on the top strand wherein the RNA polymerase transcribes the top strand and displaces the RecycleD strand and generates a fluorescent DNA/RNA hybrid, and the released RecycleD strand can displace an additional signal gate generating a positive feedback loop of signal amplification. In some aspects, the top strand is longer than the bottom strand and the transcribed RNA comprises a sequence that is complementary to the full length of the top strand. In further aspects, the waste strand or the bottom strand comprises one or more non-natural modifications that prevent the strand from being utilized as a template for transcription (e.g., 2′-O-methylation). In some aspects, the compositions, systems, and/or kits further comprise a non-labeled double-stranded DNA molecule comprising a top strand that comprises a nucleotide sequence that is identical to the nucleotide sequence of the top strand of the labeled double-stranded DNA molecule, and in other aspects, the top strand of the non-labeled double-stranded DNA molecule is longer than the bottom strand of the non-labeled double-stranded DNA molecule. In some aspects, the bottom strand of the non-labeled double-stranded DNA molecule is shorter in length than the length of the bottom strand of the fluorescently labeled double-stranded DNA molecule.

In another aspect, the transcribed RNA does not form and/or is designed not to form an intramolecular secondary structure. In some aspects, the RNA polymerase is selected from T7 RNA polymerase, T3 RNA polymerase, SP6 RNA polymerase, and Syn5 RNA polymerase or the RNA polymerase is an engineered polymerase. In some aspects, the aTF represses, blocks, or inhibits transcription from the engineered transcription template when the aTF binds the operator, and in other aspects, the aTF activates transcription from the engineered transcription template when the aTF binds the operator. In some aspects, in the presence of the analyte as a ligand the aTF does not bind to the operator sequence or binds to the operator at a lower affinity than in the absence of the analyte as a ligand, while in other aspects, in the absence of the analyte as a ligand the aTF does not bind to the operator sequence or binds to the operator at a lower affinity than in the presence of the analyte as a ligand. In some aspects, the aTF belongs to the TetR, MarR, or ArsR/SmtB class or family of transcription factors or the aTF is an engineered aTF, and in other aspects, the aTF is selected from the group consisting of TetR, MphR, QacR, OtrR, CtcS, SAR2349, MobR, SmtB, CadC, CsoR, AdcR, TtgR, and HucR.

In other aspects, the analyte that is a ligand for the aTF is an antibiotic, and in some further aspects, the analyte that is a ligand for the aTF is a member of the macrolide-family of antibiotics or member of the tetracycline-family of antibiotics. In other aspects, the analyte is a quaternary amine or salts thereof. In other aspects, the analyte is a metal or a cation thereof, and in further aspects, the metal or the cation thereof is Zn, Pb, Cu, Cd, Ni, As, or Mn. In other aspects, the analyte is selected from salicylate, 3-hydroxy benzoic acid, naringenin, and uric acid.

In other aspects, the compositions, systems, and/or kits further comprise (f) one or more components for preparing a reaction mixture for RNA transcription.

In some aspects, the compositions, systems, and/or kits disclosed herein are adapted to detect and/or quantify a range of compounds related to environmental contamination and human health.

The disclosure further provides a method for detecting an analyte in a sample, the method comprising contacting the sample with any one of the compositions, systems, and/or kits disclosed herein and detecting a signal.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Interfacing cell-free biosensors with a DNA strand displacement circuit information processing layer expands and enhances their function. (Top half) A cell-free biosensor typically activates when a target compound (input) binds to a protein transcription factor (sensor layer) that is configured to activate expression of a reporter construct (output layer). This results in the production of a detectable signal such as fluorescence. (Bottom half) Adding a downstream information processing layer before signal generation can enhance the performance and expand the function of cell-free biosensors by adding computational features such as logic processing and signal comparison. Here, this is implemented by wiring the biosensing output layer to produce a single stranded RNA capable of activating toehold-mediated strand displacement circuits that generate the signal.

FIG. 2A-G. Secondary structure of InvadeR impacts strand displacement efficiency. a, Three different variants of InvadeR were designed. Variant 1 includes the two initiating guanines followed by the sequence fully complementary to the fluorophore strand. For variant 2 and 3, two or three additional nucleotides were inserted between the initiating nucleotides and the InvadeR sequence (shaded regions), such that they disrupt the secondary structure at its base. Strengthened versions were created by mutating the additional nucleotides to strengthen the structure, while keeping the number and positions of the nucleotides that interact with the DNA signal gate consistent. Minimum free energies and base pairing probabilities for each structure are predicted using NUPACK at 37° C. [32]. b, When a gel-purified variant is added to the DNA signal gate, a fluorescent signal via toehold-mediated strand displacement is observed. c, 5 μM of gel-purified InvadeR variants were added to an equimolar amount of the DNA signal gate, and fluorescence activation was quantified. Variant 3 generates the highest fluorescent signal followed by variants 2 and 1, while both strengthening mutants show a decrease in signal from their respective variants by the fold reduction indicated above the bars. d. When a DNA template encoding InvadeR is included with T7 RNAP and the DNA signal gate, the RNA output can be tracked in situ by monitoring fluorescence activation from the signal gate. e, Comparison of fluorescence kinetics of the three variants from IVT using an equimolar DNA template (50 nM) or a no template negative control. Comparison of fluorescence kinetics between variants and their strengthening mutants for f, variant 2 and g, variant 3, shows that strengthening base pairs negatively impact fluorescence kinetics. Data shown for n=3 technical replicates as points (c) with bar heights representing the average or n=3 independent biological replicates each shown as line (e-g) with raw fluorescence standardized to MEF (μM fluorescein). Error bars (c) and shading (e-g) indicate the average value of the replicates±standard deviation.

FIG. 3A-F. Transcription of InvadeR can be regulated with an allosteric transcription factor. a, IVTs can be allosterically regulated with a template configured to bind a purified transcription factor (TetR) via operator sequence (tetO) placed downstream of the T7 promoter. A series of spacers in 2-bp intervals was constructed to evaluate the impact of spacer length on the ability of TetR to regulate the transcription of InvadeR. b, End-point data (at 1 h) shown for promoter-operator spacer variants regulated (with 5 μM TetR dimer, 50 nM DNA template) and unregulated (without TetR). c, Induction of a TetR-regulated IVT reaction occurs in the presence of the cognate ligand, anhydrotetracycline (aTc), which binds to TetR and prevents its binding to tetO. This allows transcription to proceed, leading to fluorescence activation via toehold-mediated strand displacement. d, Dose response with aTc, measured at 1 h with 50 nM DNA template and 5 μM TetR dimer. The lowest ligand concentration at which the signal is distinguishable from the background was determined using a two-tailed, heteroscedastic Student's t-test against the no-ligand condition, and its P value range is indicated with an asterisk (***P<0.001, **P=0.001-0.01, *P=0.01-0.05). See Data Availability section below for exact P values along with degrees of freedom for all ligand concentrations tested. Data for no-ligand condition are not shown herein because the x-axis is on a log scale; see Data Availability section. e, The speed of the toehold-mediated strand displacement output is fast and tunable while that of the RNA aptamer output is slow and difficult to alter. f, Comparison of fluorescence kinetics between the TetR-regulated InvadeR and the aptamer outputs when induced with 10 μM aTc. All data shown for n=3 independent biological replicates each shown as point (b, d) or line (f) with raw fluorescence values standardized to MEF (μM fluorescein). Error bars (d) and shading (f) indicate the average value of the replicates±standard deviation.

FIG. 4A-F. Different input molecules can be detected by modularly configuring InvadeR with an aTF operator sequence. DNA templates encoding InvadeR are modified to contain the T7 promoter followed by a 2-bp spacer and an aTF operator sequence immediately upstream of the InvadeR sequence. Secondary structures, minimum free energies and base pairing probabilities of a, GG-tetO-InvadeR, b, GG-ttgO-InvadeR and c, GA-srntO-Hairpin2-InvadeR (1BP spacer variant) are predicted using NUPACK at 37° C. [32]. The GA-smtO-Hairpin2-InvadeR sequence includes an RNA hairpin designed to minimize structural interference of InvadeR with smtO and optimize the signal (FIG. 14h, i). The sequence complementary to the signal gate is denoted with a dotted line and highlighted in green shading. d, TetR can be used to sense tetracycline. e, TtgR, a MarR-family aTF, can be used to sense naringenin. f, SmtB can be used to sense zinc. Variations in activation kinetics match the trend where the predicted secondary structure of the modified InvadeR impacts the induction speed. All data shown for n=3 independent biological replicates each shown as line with raw fluorescence value standardized to MEF (μM fluorescein). Shading indicates the average value of the replicates±standard deviation

FIG. 5A-F. Additional gates can be layered to perform logic computation. a, A two-input OR gate includes two additional DNA OR gates. When transcription is activated by either ligand, synthesized InvadeR molecules can react with their respective DNA OR gates to produce an output strand with a domain (green) that can invade the DNA signal gate. b, When the two-input OR gate is activated by either ZnSO₄, tetracycline or both, fluorescence activation is observed. c, A two-input AND gate contains a designed DNA AND gate which requires both InvadeR variants to dissociate the output strand to reveal the domain (green) complementary to the signal gate. Design features such as thermodynamic drivers (highlighted in red) as well as a clamp domain are implemented to facilitate efficient TMSD with minimal leak (FIG. 16). d, The two-input AND gate activates fluorescence only when both ZnSO₄ and tetracycline are present. e, A NOT gate includes two DNA templates: an unregulated InvadeR template and an aTF-regulated inverter template. Upon transcription, the aTF-regulated inverter forms a hairpin structure that resembles the DNA signal gate to create an RNA NOT gate. The InvadeR molecules transcribed from the unregulated template preferably react with the RNA NOT gate due to a greater number of bp interactions and the mismatch between InvadeR and the DNA signal gate (red). A spacer sequence is included to prevent the tetO sequence from interfering with the RNA NOT gate. f, When implemented with a tetracycline sensor, the NOT gate generates signal in the absence of tetracycline. All data shown for n=3 independent biological replicates each shown as line with raw fluorescence value standardized to MEF (μM fluorescein). Shading indicates the average value of the replicates±standard deviation. Domains with the same color share the same sequence except for the AND gate where the domain highlighted in orange is modified from the orange domain in the OR gate to improve its TMSD efficiency. All nucleic acid gates are drawn according to the secondary structures predicted using NUPACK at 37° C. [32]. Sequence of each domain and each gate architecture can be found in FIGS. 22-25 and FIGS. 30-37.

FIG. 6A-H. Logic component layering allows more complex computation. a, A two-input NOR gate, which is an inversion of an OR gate, is built by layering two RNA NOT gates. The NOT gates are regulated by either TetR or SmtB that sequester the unregulated InvadeR molecules from the DNA signal gate. b, Fluorescence activation is observed only in the absence of both tetracycline and ZnSO₄. c, A two-input IMPLY gate combines a DNA OR gate with an RNA NOT gate. In this specific example (ZnSO₄ IMPLY tetracycline), the OR gate is regulated by TetR, and the NOT gate is regulated by SmtB, thus preventing signal generation in the presence of ZnSO₄ only. d, An expected, fluorescence activation is observed unless only ZnSO₄ is added. Faster signal generation is observed from the tetracycline only input condition since no mismatch is present between the DNA OR gate output strand and the DNA signal gate. e, A two-input NAND gate, which is an inversion of an AND gate, layers two unregulated DNA OR gates with two regulated RNA NOT gates. In this configuration, the presence of both tetracycline and ZnSO₄ is required to hinder signal generation. Thermodynamic drivers (highlighted in red) are incorporated in the NOT gates to favor the interactions with their respective InvadeR strands. f, The expected NAND gate computation is observed. g, A two-input NIMPLY gate is built by combining the DNA AND gate and the RNA NOT gate. In this specific example, tetracycline-induced InvadeR and unregulated InvadeR are required for the AND gate activation. A SmtB-regulated NOT gate sequesters the unregulated InvadeR. h, Fluorescence activation is observed in the presence of tetracycline only as expected. All data shown for n=3 independent biological replicates each shown as line with raw fluorescence value standardized to MEF (μM fluorescein). Shading indicates the average value of the replicates±standard deviation. Domains with the same color share the same sequence except for the AND gate where the domain highlighted in orange is modified from the orange domain in the OR gate to improve its TMSD efficiency. All nucleic acid gates are drawn according to the secondary structures predicted using NUPACK at 37° C. [32]. Sequences of each domain and each gate architecture can be found in FIGS. 22-25 and FIGS. 30-37.

FIG. 7A-E. Quantifying ligand concentration with a molecular analog-to-digital conversion circuit. a, Increasing the length of the DNA gate toehold region can be used to speed the strand invasion process. An unlabeled DNA gate with a longer toehold (8-nt) can then preferentially react with InvadeR, acting as a programmable threshold. InvadeR can only strand-displace the signal gate (4-nt toehold) after the threshold gate is exhausted. b, Titrating the 8-nt toehold threshold gate in different ratios above a fixed signal gate concentration (0×-8×) results in a time delay in fluorescence activation that can be quantitatively modeled with ODE simulations (dotted lines). All data shown for n=3 independent biological replicates each shown as line. Raw fluorescence values were first standardized to MEF (μM fluorescein) and normalized to the maximum MEF among all conditions to accommodate their comparison to the simulations (See Materials and Methods for the normalization method used). Shading indicates the average value of the replicates±standard deviation. c, A molecular analog-to-digital conversion (ADC) circuit is made by constructing a strip of tests of the same sensor, with each test containing a different concentration of the DNA threshold gate. A higher threshold gate concentration requires a higher ligand concentration to activate fluorescence. When the same sample is applied to each tube, a user can obtain semi-quantitative information about the concentration of ligand present in the sample (analog input) by identifying the series of tubes that activate (binary digital output). Characterization of a molecular ADC circuit for zinc using d, ODE simulations and e, end-point experimental data at 100 min generated using the SmtB-regulated zinc sensor. The values on the heatmap represent the average MEF (μM fluorescein) of n=3 independent biological replicates.

FIG. 8. Micromolar Equivalent Fluorescein (MEF) standardization. Arbitrary units of fluorescence were standardized to μM concentrations of fluorescein using a NIST traceable standard (see Materials and Methods section below). In the representative example shown here, a dilution series of fluorescein was prepared in buffer (100 mM sodium borate, pH 9.5) and measured on a plate reader using the same settings for measuring 6′ FAM signal (495 nm excitation, 520 nm emission) from the DNA signal gates. The resulting curve, calculated over the linear range of 0-3.125 μM, was then used to standardize fluorescence measured from ROSALIND reactions with TMSD outputs. The standard curve was generated for each plate reader and each measurement setting. Data shown are for n=9 replicates (3 experimentally independent replicates each with 3 technical replicates). Error bars indicate standard deviation computed over n=9 replicates.

FIG. 9A-E. TMSD by Invading DNA and RNA strands. Titration of a purified a, InvadeR (RNA) and b, InvadeD (DNA) into reactions containing 10 μM of the DNA signal gate in annealing buffer (100 mM potassium acetate, 30 mM HEPES) after 15 minutes. c, Secondary structures, minimum free energies and base pairing probabilities of the InvadeR and InvadeD molecules predicted by NUPACK at 37° C. [1]. d, A urea-PAGE gel of purified InvadeR and InvadeD. The higher molecular weight band in the InvadeR lane likely corresponds to e, a duplex formed by two InvadeR molecules interacting with each other, as predicted by NUPACK. Data shown in a and b for n=3 independent biological replicates as points (a, b) with bar heights representing the average. Error bars indicate the average value of the replicates±standard deviation. See Data Availability section below for the uncropped, unprocessed gel image shown in d.

FIG. 10A-F. Toehold-mediated DNA strand displacement can be used to track RNA output with an appropriately designed DNA gate. a, In the presence of IVT components, T7 RNAP can nonspecifically bind to the toehold region of the DNA signal gate. When the overhanging toehold is on the 3′ end of the gate, this non-specific binding leads to transcription of unwanted RNA side products that can displace the quencher strand. This process is blocked when the overhanging toehold is on the 5′ end of the gate. b, The 3′ toehold DNA signal gate leads to fluorescence activation in the presence of T7 RNAP, while the 5′ toehold DNA signal gate does not get activated by T7 RNAP. c, When the reaction products from b were extracted and run on a polyacrylamide gel, RNA side products appear only when the toehold is located on the 3′ end of the gate. A negative control where no DNA signal gate is present in the reaction (−) was run alongside for both 3′ and 5′ toeholds. d, Modifying the DNA gate with 2′-O-methyl oligonucleotides prevents promoter-independent transcription by T7 RNAP. e, When the DNA signal gate with its toehold on the 3′ end is modified with 2′-O-methyl oligonucleotides, no fluorescence activation is observed in the absence of a T7 RNAP-driven IVT template. f, When the reactions from e were run on a urea-PAGE gel, no RNA side products were observed from the 2′-O-methyl DNA signal gate. A negative control where no DNA signal gate is present in the reaction (−) was run alongside for both 3′ and 5′ toeholds. Data shown in b and e for n=3 independent biological replicates each shown as line with raw fluorescence standardized to MEF (μM fluorescein). Shading indicates the average value of the replicates±standard deviation. See Data Availability section below, for the uncropped, unprocessed gel image shown in c and f.

FIG. 11A-E. Transcription efficiency impacts the speed of TMSD. a, The eight initially transcribed nucleotides of each InvadeR variant in FIG. 3a. Nucleotides that are not part of the InvadeR sequences are bolded. b, Concentrations of each variant from T7 transcription reactions measured by the Qubit RNA HS assay kit (Invitrogen #Q32852). Each variant was produced in situ in the presence of the DNA signal gate for 30 min and extracted (see the RNA extraction from IVT reactions section in Materials and Methods). The concentration of variant 1 was too low for Qubit quantification. c, The samples measured in b were run on a urea-PAGE gel and stained with SYBR gold. Titration of an RNA standard of a similar length was performed to determine the linear range of band peak area quantified by Fiji-ImageJ [2]. d, A calibration curve was constructed by plotting the peak area computed from Fiji-ImageJ quantification against the total amount of standard loaded. e, Using the calibration curve in d, the total amount of RNA for each variant was determined and compared to the measurements made by Qubit in b. Data in b are shown for n=3 independent biological replicates as points with bar heights representing the averages. Error bars indicate the average value of the replicates±standard deviation. See Data Availability section below, for the uncropped, unprocessed gel image shown in c.

FIG. 12A-B. Adding a T7 terminator does not notably improve the initial speed of ROSALIND with TMSD. a, Secondary structure, minimum free energy and base pairing probabilities of the InvadeR variant 1 (dashed backbone) that includes the T7 terminator (solid backbone) as predicted by NUPACK at 37° C. [1]. b, Comparison of the kinetics of InvadeR variant 1 TMSD of the DNA signal gate with and without the T7 terminator. All data shown for n=3 independent biological replicates each shown as line with raw fluorescence standardized to MEF (μM fluorescein). Shading indicates the average value of the replicates±standard deviation (for more information, please reference the Data Availability section, below).

FIG. 13A-C. Dose response curves of ROSALIND with TMSD. The dose response curves of ROSALIND with TMSD induced by a, tetracycline, b, naringenin and c, zinc are presented (1 h end-point data). The amounts of DNA template and aTF used in each panel are configured as described in FIG. 4d-f. All data shown for n=3 independent biological replicates as points with raw fluorescence values standardized to MEF (μM fluorescein). Error bars indicate the average value of the replicates±standard deviation. The ligand concentrations at which the signals are distinguishable from the background were determined using a two-tailed, heteroscedastic Student's t-test against the no-ligand condition, and their P value ranges are indicated with asterisks (***P<0.001, **P=0.001-0.01, *P=0.01-0.05). Please see the Data Availability section below for exact P values along with degrees of freedom. Data for the no-ligand condition were excluded because the x-axis is on the log scale; please see the data availability section.

FIG. 14A-I. Extra nucleotides can be added between the operator and InvadeR sequence to tune the kinetics of the zinc sensor. a, Secondary structure, minimum free energy and base pairing probabilities of srntO-InvadeR predicted by NUPACK at 37° C. [1]. A part of the wild type smtO sequence is complementary to the InvadeR sequence used, forming a strong predicted stem-loop. The nucleotides highlighted in green correspond to the InvadeR sequence that is designed to strand-displace the DNA signal gate. b, Secondary structure, minimum free energy and base pairing probabilities of smtO-InvadeR-InvadeR predicted by NUPACK at 37° C. c, Comparison of the kinetics of the unregulated smtO-InvadeR reaction and the unregulated smtO-InvadeR-InvadeR reaction. d, e, NUPACK-predicted secondary structures, minimum free energies and base pairing probabilities of the two smtO-Hairpin-InvadeR variants designed to internally sequester the smtO sequence and prevent it from binding to the InvadeR sequence. The nucleotides highlighted in grey indicate the added sequestering sequence. f, Comparison of the kinetics of the unregulated reactions of the variants shown in d and e. g, The smtO-Hairpin2-InvadeR variants were built by lengthening either the stem length or the spacer between the added hairpin sequence and the InvadeR sequence. The stem length variants are built with the spacer=0-nt, and the spacer variants are built with the stem length=12BP. h, i, Comparison of the kinetics of the unregulated reactions of the smtO-Hairpin2-InvadeR variants. All data shown for n=3 independent biological replicates each shown as line with raw fluorescence values standardized to MEF (μM fluorescein). Shadings indicate the average value of the replicates±standard deviation (see Data Availability section).

FIG. 15A-L. ODE Modeling of Logic Gates. a-l, Simulations of logic gates discussed in FIGS. 5, 6, and 17 are shown along with the expected computation patterns. The method used to develop the ODE model for each representative logic gate is discussed in the Supplementary Method section, below. In some embodiments, any one of the logic gate systems a-l may include polymerase strand recycling components (PSR) as described herein, to amplify signal and/or to improve assay sensitivity.

FIG. 16A-M. Design Features of Basic Logic Gate Components. a, The sequence and design of the AND gate shown in FIG. 5c. The mismatch in each input domain acts as the thermodynamic driver to run the TMSD reaction forward. The clamp domain prevents the top output strand from being completely strand-displaced only with Input 1 (orange sequence). Design iterations were evaluated with the reaction scheme shown in FIG. 5c with 5 μM of the DNA AND gate and 50 nM of the DNA template(s) encoding either Input 1, 2 or both in the absence of aTFs. b, When no thermodynamic drivers are included, no signal is observed regardless of input. Lengthening the clamp domain reduces the leak in the presence of Input 1 only. The leak observed from a c, 3BP, d, 5BP and e, 7BP clamp. f, The sequences and designs of the unregulated InvadeR molecules and the corresponding RNA NOT gates without any operator sequences in FIG. 5e. There is a mismatch between InvadeR 1 and the DNA signal gate highlighted in red. InvadeR 2 incorporates 6 extra adenines that strengthen its interactions with the NOT gate. NOT gate 1 has a 4-nt toehold while NOT gate 2 and 3 have 6-nt toeholds. NOT gate 3 has an extra adenine immediately following the two initiating guanines that increases its transcription efficiency compared to that of NOT gate 2 [3]. InvadeR and NOT gate designs were evaluated with the reaction scheme shown in FIG. 5e, but in the absence of any aTFs. Titration of the DNA template encoding NOT gate 1 in the presence of 25 nM of the DNA template encoding g, InvadeR 1 and h, InvadeR 2. While a greater amount of the NOT gate template is required to turn off the signal from InvadeR 2, much stronger fluorescence activation is observed from InvadeR 2 in the absence of the NOT gate. Titration of the DNA template encoding NOT gate i, design 2 and j, design 3 in the presence of 25 nM of the DNA template encoding InvadeR 2. Compared to the NOT gate design 1, both NOT gate design 2 and 3 are much more efficient at sequestering InvadeR 2. k, Incorporating the operator sequence into the NOT gate impacts its secondary structure. The smtO sequence (light grey) is predicted to interact with a part of NOT gate 3, blocking the toehold domain required for sequestering InvadeR 2. A spacer sequence can be designed to restore the expected secondary structure of the NOT gate. Nucleotides highlighted in grey are smtO sequence, and the spacer sequences are underlined. Secondary structures indicated are the predictions from NUPACK [1]. Titration of the DNA template encoding 1, smtO-NOT gate 3 and m, smtO-spacer-NOT gate 3 in the presence of 25 nM of the DNA template encoding InvadeR 2. When no spacer sequence is built in, a greater amount of the DNA template encoding the NOT gate is required to efficiently sequester InvadeR 2. All kinetic reactions shown are unregulated. All data shown for n=3 independent biological replicates each shown as line with raw fluorescence standardized to MEF (μM fluorescein). Shading indicates the average value of the replicates±standard deviation (see Data Availability section for additional details).

FIG. 17A-L. Logic gates can be built modularly using ligand-induced RNA inputs. a, Tetracycline-induced NOT gate shown in FIG. 5a. b, A transcription template encoding the shuffled sequence of the TetR-regulated NOT gate was used as a control to demonstrate that the reduction in signal in the presence of tetracycline is not due to the resource limitations from having an extra DNA template. c, ZnSO₄-induced NOT gate can be designed the same way as the tetracycline-induced NOT gate. The spacer sequence was added to prevent the smtO sequence from disrupting the secondary structure of the NOT gate (FIG. 16). d, In the presence of ZnSO₄, the fluorescence signal is deactivated. Similarly, the control reactions that include a transcription template encoding the shuffled sequence of the SmtB-regulated NOT gate demonstrate that the reduction in signal in the presence of ZnSO₄ is not due to the resource limitations. e, A tet IMPLY ZnSO₄ gate designed as described in FIG. 6c. f, Fluorescence activation is observed unless only tetracycline is added. g, A tet IMPLY ZnSO₄ gate can be alternatively built by including a ZnSO₄—inducible transcription template that directly interacts with the DNA signal gate instead of the DNA OR gate. h, The alternative IMPLY gate design performs the expected logic computation. A faster signal generation from the conditions that include ZnSO₄ is observed since the ZnSO₄-induced RNA inputs directly perform TMSD on the DNA signal gate. i, The alternative approach to building the IMPLY gate can also be applied to the ZnSO₄ IMPLY tet gate, and j, the gate performs the expected logic computation. k, A tet NIMPLY ZnSO₄ gate can be designed the same way as the ZnSO₄ NIMPLY tet gate shown in FIG. 6g. l, The tet NIMPLY ZnSO₄ gate performs the expected logic computation. All data shown for n=3 independent biological replicates each shown as line with raw fluorescence standardized to MEF (μM fluorescein). Shading indicates the average value of the replicates±standard deviation (please refer to the Data Availability section for additional detail). In some embodiments, any one of the logic gates and/or logic gate systems a-l may include polymerase strand recycling components (PSR) as described herein, to amplify signal and/or to improve assay sensitivity.

FIG. 18A-B. A molecular analog-to-digital converter circuit enables ligand quantification. a, An electronic analog-to-digital converter (ADC) circuit takes in a numerical voltage value (analog input) and generates a binary output of 1s and 0s (digital output). It is built by configuring a series of comparator circuits, each comparing the input voltage to a variable reference voltage to produce a binary output of 1 if the input exceeds the reference value. By setting the threshold voltages (VTH) of the comparators to be increasing along the series, the input voltage value is converted into an array of bits with the more 1's representing a higher voltage. b, A molecular version of an ADC circuit can be built by implementing TMSD thresholding circuits that compare the input target ligand concentration to a pre-defined threshold value. By titrating the pre-defined threshold value, the molecular ADC circuit can generate different bit arrays to indicate the concentration range of the target ligand

FIG. 19A-H. The semi-quantitative standard generated by the genetic ADC circuit built with the zinc sensor. The kinetic traces corresponding to the data shown in FIG. 7e are presented for the 8-nt threshold gate in different ratios above a fixed signal gate concentration (a, 0×, b, 1×, c, 2×, and d, 3×threshold). The time point (t=100 min) at which the heatmap was created is indicated with a vertical dotted line. The differences in the response speed for different zinc concentrations are essential in creating the standard. e, The 10 μM and 30 μM zinc conditions show no kinetic differences without any threshold gate. f, g, The functional characterization and ODE predictions of the ADC circuit at 100 min from FIG. 7d, e, respectively, including the 30 μM zinc and 4×threshold conditions. The 10 μM and 30 μM zinc conditions behave identically as predicted by the ODE model due to their identical kinetic behavior observed in e. h, The corresponding bar graph data of the semi-quantitative standard shown in f. All data shown for n=3 independent biological replicates each shown as line (a-e) or point (h) with raw fluorescence values standardized to MEF (μM fluorescein). The bars in h and the values on heatmap in f represent averages of the replicates. Shadings in a-e and error bars in h indicate the average value of the replicates±standard deviation (please refer to the Data Availability section for additional detail)

FIG. 20A-I. ROSALIND with TMSD can be freeze-dried. Unregulated reactions were lyophilized overnight with the addition of 50 mM sucrose and 250 mM D-mannitol as the lyoprotectants unless otherwise indicated. The lyophilized reactions were then vacuum-packaged in a light protective bag with a dri-card and kept in a cool, shaded area until usage. Kinetic traces of rehydrated reactions after a, 1 day, b, 4 days and c, 7 days of storage are shown. There is a decrease in overall signal as well as in the response speed over time. To investigate the cause of the signal loss over time, the DNA signal gate alone was lyophilized overnight with or without the lyoprotectants, packaged and stored as described above. The DNA signal gate was rehydrated with the rest of the IVT components after d, 1 day, e, 4 days and f, 7 days. The response speed as well as the magnitude of the signal are maintained, indicating that the signal loss is likely due to instability of certain IVT components. To test this hypothesis, unregulated reactions with Tris-buffered NTPs instead of NaOH-buffered NTPs were lyophilized with the lyoprotectants, packaged and stored as described above. Kinetic traces of rehydrated reactions after g, 1 day, h, 4 days and i, 7 days of storage are shown. The signal loss is somewhat mitigated with Tris-buffered NTPs, but a similar degree of signal loss is observed for a long-term storage of lyophilized reactions. All data shown for n=3 independent biological replicates each shown as line with raw fluorescence values standardized to MEF (μM fluorescein). Shadings indicate the average value of the replicates±standard deviation

FIG. 21A-B. Freeze-dried ROSALIND with TMSD can be rehydrated with real-world water matrices. Zinc-sensing ROSALIND with TMSD reactions that use InvadeR shown in Figure. 14b were freeze-dried and rehydrated with a, tap water and b, Lake Michigan water spiked with a range of concentrations of ZnSO₄. The reactions were then incubated at 37° C. for 1 hour and characterized for fluorescence by plate reader. For each water sample type, the signal was compared to that of the reactions rehydrated with laboratory-grad water spiked with the same amount of ZnSO₄. In each case, the reactions behaved as expected and saw no difference in signal between filtered and unfiltered water samples. All data shown for n=3 independent biological replicates as points with raw fluorescence values standardized to MEF (μM FITC), and bars representing averages of the replicates. Error bars indicate the average value of 3 independent biological replicates±standard deviation.

FIG. 22. Is a table showing the sequences of various oligonucleotides disclosed in the present application.

FIG. 23. Is a table showing the sequences of exemplary gate oligonucleotides, sequencing primers, and the amino acid sequence of three proteins: TetR-6XHis, TtgR-6XHis, and aSmtB-TEV-6XHis.

FIG. 24. Is a table showing the sequence of exemplary template oligonucleotides disclosed herein.

FIG. 25. Is a table showing the sequence of PCR primers disclosed herein.

FIG. 26. Is a diagram of plasmid pJBL701, comprising the T7-lacO-TetR-T insert.

FIG. 27. Is a diagram of plasmid pJBL704, comprising the T7-4BP-tetO-3WJdB-T insert.

FIG. 28. Is a diagram of plasmid pJBL721, comprising the T7-41acO-TtgR-T insert.

FIG. 29. Is a diagram of plasmid pJBL725, comprising the T7-lacO-SmtB-T insert.

FIG. 30. Shows the designs and sequences of the RNA and DNA strands involved in an exemplary AND logic gate as disclosed herein. To make the TMSD cascades more explicit, any RNA secondary structure is not depicted. The description of each sequence domain: Red—thermodynamic driver mismatches; Dark red & pink—toeholds between InvadeR strands and DNA AND gate; Brown—toehold between AND gate output and DNA signal gate; Grey—operator sequences; Orange—AND gate domain 1 adapted from OR gate domain 1; Blue—AND gate domain 2; Green—“reporting strand” (DNA signal gate strand modified with a fluorophore or its complementary sequence on AND gate) Purple—DNA signal gate strand modified with a quencher.

FIG. 31A-B. A. Shows the designs and sequences of the RNA and DNA strands involved in exemplary IMPLY logic gates disclosed herein. To make the TMSD cascades more explicit, the secondary structure of the ZnSO₄-induced InvadeR strand is not depicted. Below is the description of each sequence domain: Red—built-in mismatch between unregulated InvadeR and DNA signal gate; Dark red—toehold between ZnSO₄-induced InvadeR and OR gate 2; Brown—toehold between OR gate 2 output/unregulated InvadeR and DNA signal gate; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Blue—OR gate domain 2; Green—“reporting strand” (DNA signal gate strand modified with a fluorophore, its RNA version on RNA NOT gate, or its complementary sequence on unregulated InvadeR and OR gate 2); Purple—DNA signal gate strand modified with a quencher or its RNA version on RNA NOT gate. B. To make the TMSD cascades more explicit, the secondary structure of the Tet-induced InvadeR strand is not depicted. Below is the description of each sequence domain: Red—built-in mismatch between unregulated InvadeR and DNA signal gate; Dark red—toehold between Tet-induced InvadeR and OR gate 1; Brown—toehold between OR gate 2 output/unregulated InvadeR and DNA signal gate; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Orange—OR gate domain 1; Green—“reporting strand” (DNA signal gate strand modified with a fluorophore, its RNA version on RNA NOT gate, or its complementary sequence on unregulated InvadeR and OR gate 1) Purple—DNA signal gate strand modified with a quencher or its RNA version on RNA NOT gate.

FIG. 32A-B. A. shows the designs and sequences of the RNA and DNA strands involved in exemplary IMPLY logic gates disclosed herein. To make the TMSD cascades more explicit, the secondary structure of the ZnSO₄-induced InvadeR strand is not depicted. Below is the description of each sequence domain: Red—built-in mismatch between unregulated InvadeR and DNA signal gate; Brown—toehold between the InvadeR strands and DNA signal gate/RNA NOT gate; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Green—“reporting strand” (DNA signal gate strand modified with a fluorophore, its RNA version on RNA NOT gate, or its complementary sequence on the InvadeR strands) Purple—DNA signal gate strand modified with a quencher or its RNA version on RNA NOT gate. B. To make the TMSD cascades more explicit, the secondary structure of the Tet-induced InvadeR strand is not depicted. Below is the description of each sequence domain: Red—built-in mismatch between unregulated InvadeR and DNA signal gate; Brown—toehold between the InvadeR strands and DNA signal gate/RNA NOT gate; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Green—“reporting strand” (DNA signal gate strand modified with a fluorophore, its RNA version on RNA NOT gate, or its complementary sequence on the InvadeR strands) Purple—DNA signal gate strand modified with a quencher or its RNA version on RNA NOT gate.

FIG. 33 shows the designs and sequences of the RNA and DNA strands involved in an exemplary NAND logic gate disclosed herein. To make the TMSD cascades more explicit, any RNA secondary structure is not depicted. Below is the description of each sequence domain: Red—thermodynamic driver mismatches; Dark red—toehold between unregulated InvadeR and OR gates/RNA NOT gates; Brown—toehold between OR gate outputs and DNA signal gate; Grey—operator sequences; Dark Grey—built-in loop sequence in RNA NOT gates to increase the number of BP interactions with unregulated inputs; Orange—OR gate domain 1 or its RNA version on Tet-induced RNA NOT gate; Blue—OR gate domain 2 or its RNA version on ZnSO₄-induced RNA NOT gate; Green—“reporting strand” (DNA signal gate strand modified with a fluorophore or its complementary sequence on OR gates) Purple—DNA signal gate strand modified with a quencher.

FIG. 34A-B. A. shows the designs and sequences of the RNA and DNA strands involved in exemplary NIMPLY logic gates as disclosed herein. To make the TMSD cascades more explicit, the secondary structure of the ZnSO₄-induced InvadeR strand is not depicted. Below is the description of each sequence domain: Red—thermodynamic driver mismatches Dark red—toehold between ZnSO₄-induced InvadeR and AND gate; Pink—toehold between unregulated InvadeR and the RNA NOT gate/AND gate; Brown—toehold between AND gate output and DNA signal gate; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Orange—AND gate domain 1; Blue—AND gate domain 2; Green—“reporting strand” (DNA signal gate strand modified with a fluorophore or its complementary sequence on AND gate); Purple—DNA signal gate strand modified with a quencher. B. To make the TMSD cascades more explicit, the secondary structure of the Tet-induced InvadeR strand is not depicted. Below is the description of each sequence domain: Red—thermodynamic driver mismatches; Dark red—toehold between unregulated InvadeR and the RNA NOT gate/AND gate; Pink—toehold between Tet-induced InvadeR and AND gate; Brown—toehold between AND gate output and DNA signal gate; Grey—operator sequence; Dark grey—built-in loop sequence in RNA NOT gate to increase the number of BP interactions with unregulated InvadeR; Orange—AND gate domain 1 adapted from OR gate domain 1Blue—AND gate domain 2; Green—“reporting strand” (DNA signal gate strand modified with a fluorophore or its complementary sequence on AND gate) Purple—DNA signal gate strand modified with a quencher.

FIG. 35. shows the designs and sequences of the RNA and DNA strands involved in an exemplary NOR logic gate disclosed herein. Below is the description of each sequence domain: Red—built-in mismatch between unregulated InvadeR and DNA signal gate; Brown—toehold; Grey—operator sequence; Grey underlined—built-in spacer in RNA NOT gates to sequester the operator sequences away from the downstream signal gate sequence; Dark grey—built-in loop sequence in RNA NOT gates to increase the number of BP interactions with unregulated InvadeR; Green—“reporting strand” (DNA signal gate strand modified with a fluorophore, its RNA version on RNA NOT gates, or its complementary sequence on unregulated InvadeR); Purple—DNA signal gate strand modified with a quencher or its RNA version on RNA NOT gates.

FIG. 36A-B. A. shows the designs and sequences of the RNA and DNA strands involved in exemplary NOT logic gates disclosed herein (Tet). Below is the description of each sequence domain: Red—built-in mismatch between unregulated InvadeR and DNA signal gate; Brown—toehold; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Green—“reporting strand” (DNA signal gate strand modified with a fluorophore, its RNA version on RNA NOT gate, or its complementary sequence on unregulated InvadeR); Purple—DNA signal gate strand modified with a quencher or its RNA version on RNA NOT gate. B. Below is the description of each sequence domain for an exemplary ZnSO₄ NOT logic gate as disclosed herein: Red—built-in mismatch between unregulated InvadeR and DNA signal gate; Brown—toehold; Grey—operator sequence; Grey underlined—built-in spacer in RNA NOT gate to sequester the operator sequence away from the downstream signal gate sequence; Dark grey—built-in loop sequence in RNA NOT gate to increase the number of BP interactions with unregulated InvadeR; Green—“reporting strand” (DNA signal gate strand modified with a fluorophore, its RNA version on RNA NOT gate, or its complementary sequence on unregulated InvadeR); Purple—DNA signal gate strand modified with a quencher or its RNA version on RNA NOT gate.

FIG. 37. shows the designs and sequences of the RNA and DNA strands involved in an exemplary OR logic gate as disclosed herein. To make the TMSD cascades more explicit, any RNA secondary structure is not depicted. Below is the description of each sequence domain: Dark red—toehold between InvadeR strands and OR gates; Brown—toehold between OR gate outputs and DNA signal gate; Grey—operator sequences; Orange—OR gate domain 1; Blue—OR gate domain 2; Green—“reporting strand” (DNA signal gate strand modified with a fluorophore or its complementary sequence on OR gates) Purple—DNA signal gate strand modified with a quencher.

FIG. 38. Polymerase strand recycling (PSR) can be used to create an intermediate layer in cell-free systems that amplifies signals. FIG. 38 provides a schematic of an exemplary cell-free sensor including a fuel gate. Adding a downstream amplification layer before signal generation can recycle the single-stranded RNA output and decrease the amount of input required to fully activate a DNA signal gate. In the system, T7 RNA polymerase provide promoter-independent transcription to recycle InvadeR. In the presence of in vitro transcription components, T7 RNAP can non-specifically bind to the toehold region of the DNA signal gate. When the toehold is on the 3′ end of the gate, this leads to transcription of unwanted RNA side products that can displace the quencher strand. This process is blocked when the toehold is on the 5′ end of the gate. InvaderR is transcribed by T7 TNAP, which strand displaces DNA fuel gate to release RecycleD. Recycle D strand displaces DNA Signal Gate and activates fluorescent output while leaving a 3′ toehold for T7 RNAP to non-specifically bind to. T7 RNAP transcribes from the 3′ Fluorophore toehold and releases Recycle D to strand displace more DNA Signal Gates to achieve signal amplification.

FIG. 39. Exemplary Fuel Gate and Signal Gate designs.

FIG. 40A and FIG. 40B. Fuel gate and T7 RNAP enhance signal generation. A) Recycle D strand displaces DNA Signal Gate and is recycled by T7 RNAP promoter-independent activity. B) Adding T7 RNAP increases signal in the presence of NTPs. T7 Polymerase can displace a range of 3′ toehold lengths (data not shown). SP6 Polymerase cannot displace 3′ toeholds (data not shown), and RNA/DNA duplex cannot be transcribed by T7 RNAP (data not shown).

FIG. 41A and FIG. 41B. DNA template inputs can be reduced by a factor of 100-200 fold (from 50 nm). A) Reaction including catalytic amplification (with T7 RNAP recycling). B) Rection without catalytic amplification (no T7 RNAP recycling).

FIG. 42. Methylation of the quencher strand of the signal gate improves signal/noise ratio (reduces leak due to from T7 RNAP amplifier).

FIG. 43. Methylating the fuel gate, and/or fuel gate and signal gate, helps improve signal/noise rations (reduce leak due to T7 RNAP amplifier).

FIG. 44. Promoter-independent T7 RNAP activity is sensitive to the quality and source of T7 RNAP.

FIG. 45. Data showing tetracycline sensor sensitivity can be increased by polymerase strand recycling (PSR).

FIG. 46. Provides anticipated results of an exemplary cell-free molecular sensors. The sensors, with the fuel gate, will be able to reduce the amount of transcription factor needed for a ROSALIND reaction and increase the sensitivity of more small molecule sensors, including lead and zinc.

### DETAILED DESCRIPTION

The present invention is described herein using several definitions, as set forth below and throughout the application.

Unless otherwise specified or indicated by context, the terms “a”, “an”, and “the” mean “one or more.” For example, “a component,” “a composition,” “a system,” “a kit,” “a method,” “a protein,” “a vector,” “a domain,” “a binding site,” and “an RNA” should be interpreted to mean “one or more components,” “one or more compositions,” “one or more systems,” “one or more kits,” “one or more methods,” “one or more proteins,” “one or more vectors,” “one or more domains,” “one or more binding sites,” and “one or more RNAs,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “regulation” and “modulation” may be utilized interchangeably and may include “promotion” and “induction.” For example, a transcription factor that regulates or modulates expression of a target gene may promote and/or induce expression of the target gene. In addition, the terms “regulation” and “modulation” may be utilized interchangeably and may include “inhibition” and “reduction.” For example, a transcription factor that regulates or modulates expression of a target gene may inhibit and/or reduce expression of the target gene.

As used herein, the term “sample” may include “biological samples” and “non-biological samples.” Biological samples may include samples obtained from a human or non-human subject. Biological samples may include but are not limited to, blood samples and blood product samples (e.g., serum or plasma), urine samples, saliva samples, fecal samples, perspiration samples, and tissue samples. Non-biological samples may include but are not limited to aqueous samples (e.g., watershed samples) and surface swab samples.

### Polynucleotides and Uses Thereof

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Therrnus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, RNA polymerases of bacteriophages (e.g. T3 RNA polymerase, T7 RNA polymerase, SP6 RNA polymerase, Syn5 RNA polymerase), and E. coli RNA polymerase, among others. The foregoing examples of RNA polymerases are also known as DNA-dependent RNA polymerase. The polymerase activity of any of the above enzymes can be determined by means well known in the art.

Also contemplated for us in the disclosed compositions, systems, kits, and methods are engineered RNA polymerase. For example, an engineered polymerase may be a non-naturally occurring RNA polymerase whose amino acid sequence has been engineered to include one or more of an insertion, a deletion, or a substitution relative to the amino acid sequence of a naturally occurring or wild-type RNA polymerase.

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

In the methods contemplated herein, a host cell may be transiently or non-transiently transfected (i.e., stably transfected) with one or more vectors described herein. A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors), and modified through the activity of a complex, in order to establish a new cell line comprising cells containing the modification but lacking any other exogenous sequence.

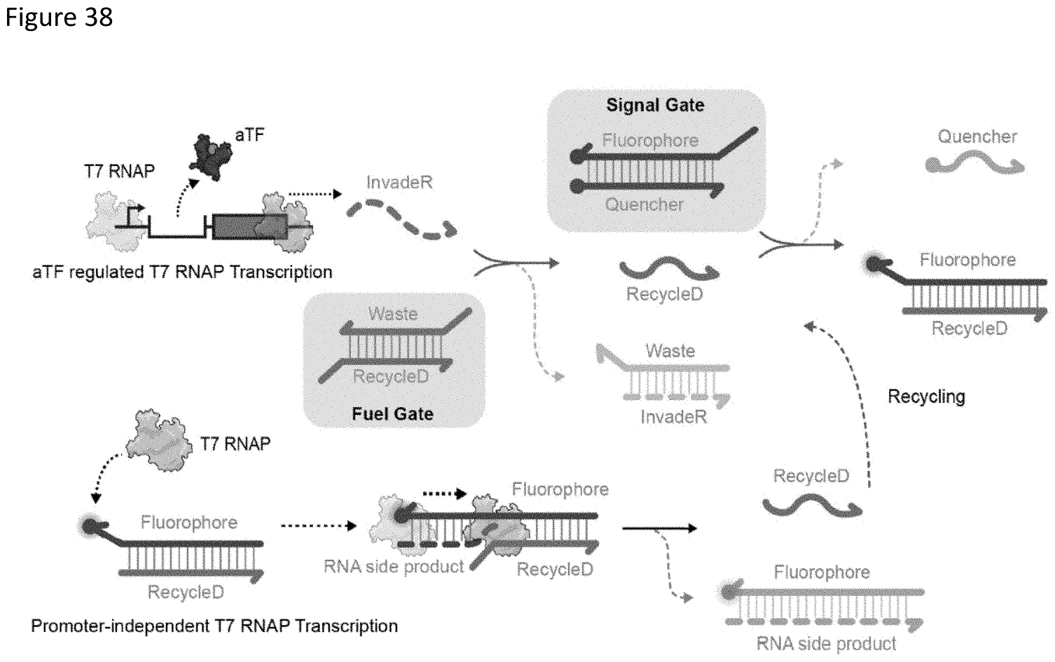

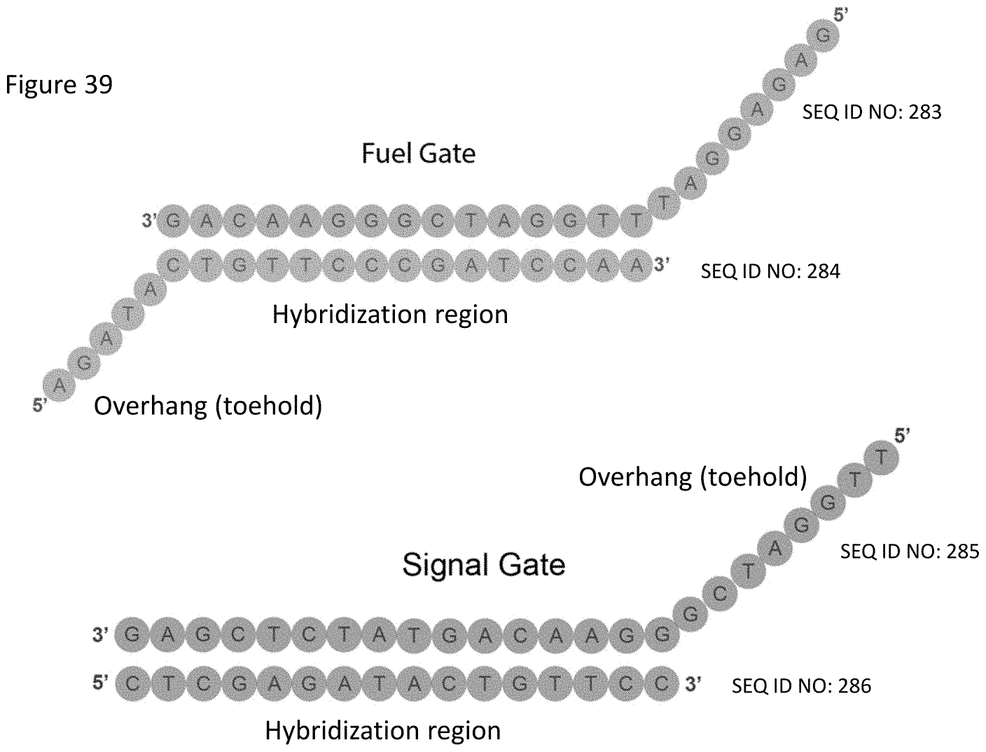

Fuel gates of the present disclosure comprise double-stranded nucleic acid molecules, and may comprise DNA:DNA duplexes, DNA:RNA duplexes, or RNA:RNA duplexes. In some embodiments, one or both nucleic acid molecules of the fuel gate duplex comprise one or more modifications, such as, without limitation, one or more non-natural nucleic acids, one or more methyl groups. In some embodiments, the two nucleic strands of the fuel gate comprise a complementary region or hybridization regions of at least 90%, 91%, 92%, 93%, 94%, 95%, 96%, 97%, 98%, 99%, or 100% that allows the two strands to hybridize with each other. In some embodiments, the region of hybridization regions is about 7, 8, 9, 10, 11, 12, 13, 14, 15, 16, 17, 18, 19, or 20 bases pairs in length. In some embodiments, the complementary regions is about 15 base pairs in length, and has 100% complementarity for all 15 nucleotides. In some embodiments, the double-stranded fuel gate comprises, in addition to the complementary region, one or more overhands (e.g., 5′ overhands). See e.g., FIG. 39. In some embodiments, the one or more 5′ overhangs comprise single stranded regions that are 5, 6, 7, 8, 9, 10, 11, 12, 13, 14, 15 or more nucleotides in length. In embodiments, one strand of the fuel gate is termed a “waste strand”, and one strand of the fuel gate is termed a “Recycle D stand”. In embodiments, the overhang region and the complementary region of waste strand allow for preferential hybridization of an InvadeR RNA molecule; that is, the length of the hybridization region between the InvadeR RNA and the waste strand is greater than the length of the hybridization regions between the two strands of the fuel gate. In some embodiments, the complementary region and the overhand region of a RecycleD strand of a fuel gate hybridizes preferentially to a nucleic acid member of a signal gate duplex, e.g., has a longer hybridization region, with the labeled strand of a signal gate (as opposed to the quencher strand of a signal gate) than to the partially waste strand. In embodiments, the RecycleD strand is 90%, 91%, 92%, 93%, 94%, 95%, 96%, 97%, 98%, 99%, or 100% complementary to the signal gate strand, e.g., the labeled strand. In embodiments, the labeled strand of a RecycleD/signal gate-labeled strand comprises a single-stranded overhang region, wherein the overhang comprise signal gate nucleotides of the labeled strand. In some embodiments, the signal gate overhang is the 3′ end of the signal gate molecule, and comprises 1, 2, 3, 4, 5, 6, 7, 8, 9, 10 or more nucleotides. This 3′ overhang is sufficient for T7RNAP to bind and initiate transcription of the signal strand, thereby displacing the RecycleD strand. See e.g. FIG. 38. The waste strand may comprise RNA, while the RecycleD strand of the fuel gate typically comprises DNA. The quencher strand of the signal gate may be RNA, but the labeled strand of the signal gate typically comprises DNA. The inventors determined that RNA-DNA duplex cannot be transcribed by T7 RNA polymerase. Accordingly, configurations of cell-free sensors including a polymerase strand recycling component comprising a T7 RNA polymerase non-specific transcription step are configured such that the non-specific T7 RNAP target template comprises a DNA:DNA duplex region for T7 RNAP activity.

Considerations and aspects for fuel gate design comprise the following, without limitation: in some embodiments, the toehold from the fuel gate should be about ½ the length of the toehold from Flurophore/Quencher (signal gate). In some embodiments, the toehold of a fuel gate/signal gate hybrid is about ½ the length of the toe hold (e.g., overhangs) of a fuel gate duplex and about half the length of the toe hold of a signal gate duplex. (See e.g., FIG. 39). T7 RNAP does not transcribe DNA/RNA duplex to produce an RNA side product; methylating both ends of the fuel gate reduces the promoter-independent transcription activities; methylating both ends of the signal gate reduces the promoter-independent transcription activities; a 200-fold reduction of DNA template can be achieved using the disclosed T7 RNAP signal recycling scheme. In embodiments, toehold lengths (overhang) are 4-8 nucleotides; the hybridization region comprises about 15 nucleotides. T7 RNAP does not transcribe from a 3′ toehold of SS DNA if it is part of an DNA: RNA duplex; DNA:DNA duplex is preferred. SP6 RNAP does not have the same effect as T7 RNAP, and promoter-independent T7 RNAP activity is sensitive to the quality and source of T7 RNAP.

### Peptides, Polypeptides, and Proteins

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation), hydroxylation, iodination (e.g., of thyroid hormones), and phosphorylation (e.g., the addition of a phosphate group, usually to serine, tyrosine, threonine or histidine).

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may have one or more insertions, deletions, or substitution of at least one amino acid residue relative to a reference polypeptide.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Glu, Gln |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

In some embodiments of the disclosed compositions, systems, kits, and methods, the components may be substantially isolated or purified. The term “substantially isolated or purified” refers to components that are removed from their natural environment, and are at least 60% free, preferably at least 75% free, and more preferably at least 90% free, even more preferably at least 95% free from other components with which they are naturally associated.

Detection of Analytes and Target Molecules Using Regulated In Vitro Transcription with DNA Strand Displacement Circuits

Disclosed herein are compositions, systems, kits, and methods that relate to the detection of analytes and target molecules using regulated in vitro transcription. The disclosed compositions, systems, kits, and methods include and utilize components as described herein including components for forming DNA strand displacement circuits.

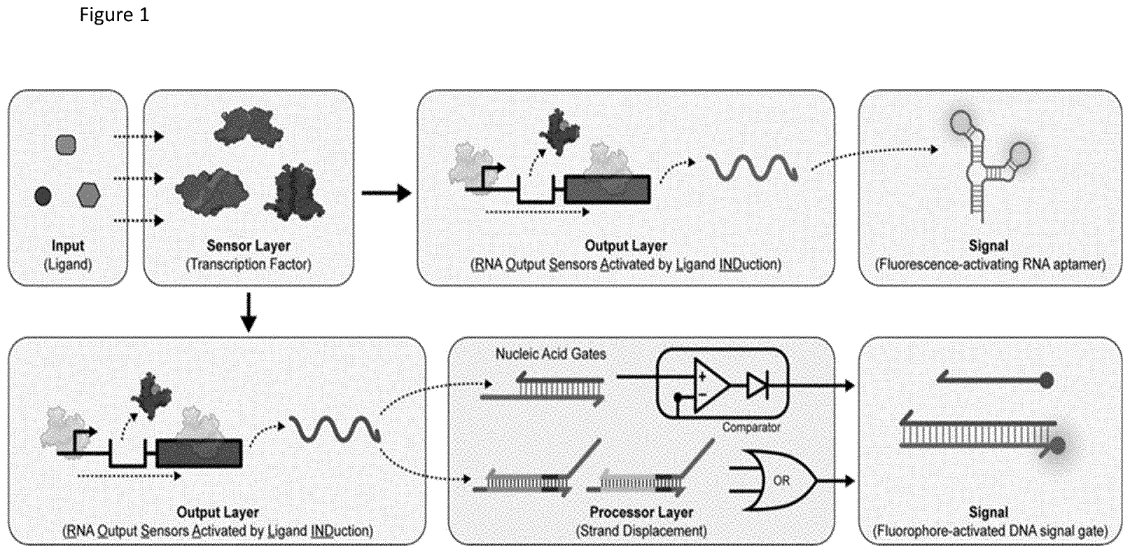

Disclosed herein is a generalizable strategy to enhance and expand the function of cell-free biosensors by introducing an information processing layer that can manipulate responses from the sensing layer before final signal generation (FIG. 1). Such information processing layers are a natural feature within biological organisms and are present in sophisticated genetic networks that enable cells to activate stress responses, alter physiology, guide development and make behavioral decisions based on intracellular and extracellular cues [8]. As such, genetic information processing layers have been extensively leveraged and engineered in synthetic cellular sense-and-respond systems [9, 10]. Similarly, it was recently shown that RNA-based circuits that implement genetic logic and feedback can be added to cell-free biosensing systems to improve their specificity and sensitivity without having to engineer the protein sensors [7]. However, these circuits still directly act on either the sensing or the output layer, limiting the ability to further expand the function of biosensing systems using this approach.

To create a more generalized information processing layer in a cell-free context, the inventors leveraged the development of toehold-mediated DNA strand displacement (TMSD)—a computationally powerful and versatile DNA nanotechnology platform that can be used for information processing in vitro [11]. In TMSD, single-stranded DNA (ssDNA) inputs interact with double-stranded DNA (dsDNA) ‘gates’ that are designed to exchange strands and produce ssDNA outputs. By configuring the DNA gates into different network architectures, a range of operations can be performed such as signal restoration [12], signal amplification [13] and logic computation [14, 15], much like a general chemical computational architecture [16]. The well-characterized thermodynamics of DNA base pairing enable large programmable networks to be built from relatively simple building blocks. In addition, the kinetics of these reactions can be precisely tuned by changing the strength of the ‘toeholds’— single-stranded regions within the DNA gates that initiate the strand displacement process [17]. TMSD has been used to create a range of devices including in vitro oscillators [18], catalytic amplifiers [19], autonomous molecular motors [20, 21] and reprogrammable DNA nanostructures [22, 23]. Furthermore, TMSD circuits capable of sophisticated molecular computations such as complex arithmetic [24] and even molecular neural networks that recognize chemical patterns [25] have been designed. Thus, there is a great potential in utilizing TMSD-based information processing to enhance and expand cell-free biosensor function.

The features of TMSD circuits have motivated the development of diagnostics that can detect nucleic acid targets such as microRNAs [26, 27] and human pathogens [28]. These circuits work by programming DNA gates to directly match the sequence complementarity of the desired nucleic acid input, which triggers strand exchange upon binding. However, there are currently no similar general design rules for triggering TMSD circuits with small molecules of varying size, shape and chemical properties. To leverage the power of TMSD circuits for small molecule chemical detection, an interface is needed that can convert the binding event of a chemical target to changes in nucleic acid sequence or structure that can trigger TMSD cascades. Allosteric transcription factors (aTFs) naturally create such an interface by activating the transcription of a programmable RNA sequence upon detection of a compound. However, there are significant challenges in creating an interface that allows aTFs and TMSD circuits to function together in situ, such as interference between RNA polymerase (RNAP) and nucleic acid gates [29], the lack of detailed experimental characterization of RNA-mediated TMSD circuits [30] and the need to develop design principles that insulate circuit function from the complexities of RNA folding.

As described herein, the inventors have overcome these challenges by interfacing the sensing layers of a previously developed cell-free biosensing platform called ROSALIND [7] with TMSD circuits to expand the platform's capabilities. The novel platform comprises a highly processive phage RNAP, an aTF and a DNA template that together regulate the synthesis of an invading RNA strand that can activate fluorescence from a DNA signal gate—a dsDNA consisting of a quencher strand and a fluorophore strand with a toehold region. In this way, this new platform combines TMSD with the biochemistry of aTFs and in vitro transcription (IVT) to enable TMSD circuits to serve as downstream signal processing units to a chemical ligand sensing reaction.

The inventors first show that the design of the DNA gate can be optimized to enable T7 RNAP driven in vitro transcription (IVT) and TMSD within the same reaction.

Next, the inventors systematically develop design principles for optimizing the secondary structure of the synthesized RNA to tune the reaction kinetics of TMSD, notably improving the biosensing response speed. The inventors also apply this principle to interface TMSD with several different aTFs to create biosensors for their cognate ligands.

The inventors then showcase programmability of the platform by building twelve different circuits that implement seven different logic functions (NOT, OR, AND, NOR, IMPLY, NIMPLY, NAND). Importantly, this required the development of additional RNA-level design principles such as fine-tuning of transcription efficiency and optimization of RNA secondary structure to efficiently interface RNA inputs with DNA-based TMSD circuits.

Finally, the inventors address a current limitation of cell-free biosensors by using a model-driven approach to design and build a multi-layer TMSD circuit that acts like an analog-to-digital converter to create a series of binary outputs that encode the concentration range of the target molecule being sensed. Taken together, this work demonstrates that the combination of TMSD and cell-free biosensing reactions can implement molecular computations to enhance the speed and utility of biosensors.

Applications of the disclosed technology include, but are not limited to: (i) Chemical testing; (ii) Chemical screening; (iii) Water quality testing; (iv) Environmental sensing; (v) Health marker sensing in human fluids (blood, urine, saliva, breast milk, etc.); (vi) Micronutrient diagnostics in water, soils, plants and animals; (vii) Drug testing; (viii) Drug discovery; (ix) Heavy metal testing; (x) Contaminant testing; (xi) Diagnostics; (xii) High-throughput screening; (xiii) Research (transcription factor screening, protein engineering); (xiv) Food testing; (xv) Beverage testing; (xvi) Agriculture; (xvii) Aquaculture; and (xviii) Animal health.

The advantages of the disclosed technology include, but are not limited to: (i) speed, where the methods can be performed within minutes; (ii) low cost, where the cost for performing the methods is less than a few dollars to pennies per sample; (iii) robustness, where the methods can be performed using a variety of samples; (iv) reproducibility, where the technology utilizes biochemically defined reactions; (v) ease of use, where the methods may be performed using handheld and portable components; (vi) methods are performed in vitro and do not involve replicating components (e.g., cells); and (vii) extensibility and adaptability, where the methods may be performed to detect a variety of target molecules and analytes.

The disclosed compositions, systems, kits, and methods may be utilized to detect an analyte or a target molecule in a sample. In some embodiments, the disclosed compositions, systems, kits, and methods comprise or utilize one or more components selected from: (a) an RNA polymerase; (b) an allosteric transcription factor (aTF), wherein the analyte or target molecule is a ligand to which the aTF binds; (c) an engineered transcription template; (d) a dsDNA signal gate molecule (e.g., a dsDNA molecule comprising a quencher strand hybridized to a fluorophore strand with a toehold region), (e) a dsDNA fuel gate molecule (e.g., a dsDNA molecule comprising a waste strand and a RecycleD strand); and/or a combination thereof. The transcription template typically comprises a promoter sequence for the RNA polymerase and an operator sequence for the aTF. The promoter sequence and operator sequence are operably linked to a sequence encoding an RNA, wherein the aTF modulates transcription of the encoded RNA when the aTF binds the analyte or target molecule as a ligand. The RNA that is transcribed from the transcription template may displace a strand of the dsDNA fuel molecule by hybridizing to the waste strand, and the RecycleD strand displaces a strand of the dsDNA signal gate whereby a signal is generated (e.g., a fluorescent signal), thereby indicating that the analyte or target molecule is present.

In some embodiments of the disclosed compositions, systems, or kits, the transcribed RNA displaces a nucleotide strand of a reporter molecule which comprises a fluorescently labeled double-stranded DNA signal gate molecule as disclosed herein. In other embodiments of the disclosed compositions, systems, or kits, the compositions, systems, or kits further comprise a second engineered transcription template, in which the second engineered transcription template comprises a promoter sequence for the RNA polymerase operably linked to a sequence encoding a second RNA. In these embodiments, the second transcribed RNA displaces a nucleotide strand of a reporter molecule which comprises a fluorescently labeled double-stranded DNA signal gate molecule as disclosed herein.

Suitable RNA polymerases for inclusion or use in the disclosed compositions, systems, kits, and methods may include, but are not limited to, RNA polymerases derived from bacteriophages. Suitable RNA polymerases may include but are not limited to T7 RNA polymerase, T3 RNA polymerase, SP6 RNA polymerase, and Syn5 RNA polymerase. Suitable RNA polymerases may include engineered RNA polymerases as contemplated herein. In some embodiments, the RNA polymerase is T7 RNA polymerase.

In the disclosed compositions, systems, kits, and methods, the allosteric transcription factor (aTF) modulates transcription from the engineered transcription template. In some embodiments, the aTF modulates transcription from the engineered transcription template when the aTF binds the operator sequence. In some embodiments, the aTF represses transcription from the engineered transcription template when the aTF binds the operator sequence. In other embodiments, the aTF activates, derepresses, and/or augments transcription from the engineered transcription template when the aTF binds the operator sequence.

In the disclosed compositions, systems, kits, and methods, the allosteric transcription factor (aTF) binds the analyte or target molecule as a ligand. In some embodiments, in the absence of the analyte or target molecule as a ligand the aTF binds to the operator sequence, and/or in the presence of the analyte or target molecule as a ligand the aTF does not bind to the operator sequence or binds to the operator sequence at a lower affinity than in the absence of the analyte or target molecule as a ligand. In other embodiments, in the presence of the analyte or target molecule as a ligand the aTF binds to the operator sequence, and/or in the absence of the analyte or target molecule as a ligand the aTF does not bind to the operator sequence or binds to the operator sequence at a lower affinity than in the presence of the analyte or target molecule as a ligand.

Allosteric transcription factors (aTFs) are known in the art. Suitable aTFs for the disclosed compositions, systems, kits, and methods may include, but are not limited to prokaryotic aTFs. Suitable aTFs may include but are not limited to TetR, MphR, QacR, OtrR, CtcS, SAR2349, MobR, and SmtB. The TetR family of aTFs include TetR, MphR, and QacR. The MarR family of aTFs include OtrR, CtcS, SAR2349, and MobR. Suitable aTF may also include the ArsR/SmtB family of aTFs.

Suitable aTFs may include engineered aTFs. For example an engineered aTF is a non-naturally occurring aTF having an amino acid sequence which has been engineered to include one or more of an insertion, a deletion, or a substitution relative to the amino acid sequence of a naturally occurring or wild-type aTF.

In some embodiments of the disclosed compositions, systems, kits, and methods, the analyte or target molecule that is a ligand for the aTF is a member of the tetracycline-family of antibiotics. Suitable analytes/target molecules as ligands for the aTF may include, but are not limited to tetracycline, anhydrotetracyline, oxytetracycline, chlortetracycline, and doxycycline.

In some embodiments of the disclosed systems and methods, the target molecule that is the ligand for the aTF is a member of the macrolide-family of antibiotics. Suitable target molecules/ligands for the aTF may include, but are not limited to erythromycin, azithromycin, and clarithromycin.

In some embodiments of the disclosed compositions, systems, kits, and methods, the analyte or target molecule that is a ligand for the aTF is a quaternary amine or salt thereof. Suitable quaternary amines may include but are not limited to alkyldimethylbenzylammonium salts.

In some embodiments of the disclosed compositions, systems, kits, and methods, the analyte that is a ligand for the aTF is a metal or a cation thereof. Suitable metals or cations thereof may include but are not limited to heavy metals and cations thereof. Suitable metals or cations thereof may include but are not limited to Zn, Pb, Cu, Cd, Ni, As, Mn (or Zn²⁺, Pb²⁺, Cu⁺, Cu²⁺, Cd²⁺, Ni²⁺, As³⁺, As⁵⁺, and Mn²⁺).

In some embodiments of the disclosed compositions, systems, kits, and methods, the analyte that is a ligand for the aTF is selected from salicylate, 3-hydroxy benzoic acid, narigenin, uric acid.

In the disclosed compositions, systems, kits, and methods, the RNA that is transcribed from the engineered transcription template additionally or alternatively displaces the RecycleD strand from the dsDNA fuel gate molecule which then allows the RecycleD strand to bind to a reporter molecule, and the binding to the reporter molecule results in a detectable signal being generated from the reporter molecule. Suitable reporter molecules may include dsDNA molecules which may be referred to as dsDNA signal gate molecules. In some embodiments of the disclosed compositions, systems, kits, and methods, the reporter molecule is a fluorescently labeled dsDNA molecule (e.g., which functions as an output gate) comprising a fluorophore and a quencher that quenches the fluorophore in the fluorescently labeled double-stranded nucleic acid, and a toehold region. In these embodiments, the RNA that is transcribed from the engineered transcription template displaces the RecycleD strand from the dsDNA fuel gate molecule which then displaces one of the strands of the fluorescently labeled double-stranded nucleic acid reporter molecule which results in dequenching of the fluorophore to generate the detectable signal.

In some embodiments of the disclosed compositions, systems, kits, and methods, RNA transcribed by off-target transcription by T7 RNA polymerase (T7 RNAP; (promoter independent T7 RNAP transcription) is used to recycle circuit inputs, creating a catalytic amplification effect by which one circuit input can activate multiple outputs to increase sensitivity, the molecules of which can be referred to as the dsDNA fuel gate molecule. In some embodiments, the dsDNA fuel gate molecule (e.g., a dsDNA molecule comprising a waste strand and a RecycleD strand) generates a free single-stranded DNA molecule (e.g., a RecycleD strand) which displaces a strand of the dsDNA signal gate molecule to produce a detectable signal and a new hybrid signal/fuel gate, and wherein RNA polymerase transcribes the hybrid signal/fuel gate to release the RecycleD strand of DNA which can then displace an additional dsDNA signal gate molecule, thus creating a positive feedback loop of signal amplification.

In some embodiments, suitable reporter molecules may include but are not limited to fluorescently labeled double-stranded DNA molecules (e.g., which function as an output gate) comprising a top strand having a fluorophore conjugated at its 3′-end and a bottom strand having a quencher conjugated at its 5′ end that quenches the fluorophore in the fluorescently labeled double-stranded DNA molecule and a toehold region. In these embodiments, the RecycleD strand comprises a sequence that is complementary to the full length of the top strand and the RecycleD displaces the bottom strand which results in dequenching of the fluorophore to generate the detectable signal. Typically these reporter molecules are configured such that, the top strand is longer than the bottom strand (e.g., by about 1, 2, 3, 4, 5, 6, 7, 8, 9, 10, 11, 12, 13, 14, 15 nucleotides or more). In this configuration, displacement of the bottom strand by the RecycleD strand is thermodynamically favored because the RecycleD strand comprises a sequence that permits additional base-pairing between the RecycleD strand and the top strand that is not presented between the top strand and the bottom strand. In some embodiments, the top strand could comprise the quencher and the bottom strand the fluorophore.

In some embodiments, the fuel molecule is a dsDNA molecule comprising a waste strand and a RecycleD strand. In some embodiments, the transcribed RNA is complementary to the waste strand and displaces the RecycleD strand. In such embodiments, the RecycleD strand is complementary to the top strand of the dsDNA signal gate molecule and displaces the bottom strand comprising the quencher. Therefore, binding of the transcribed RNA to the waste strand must be thermodynamically favored compared to binding of the RecycleD strand to the waste strand. Likewise, the binding of the RecycleD strand to the top strand of the dsDNA signal gate must be thermodynamically favored compared to the binding of the top strand to the bottom strand. Furthermore, in some embodiments, the full length of the RecycleD strand hybridizes to the top strand, but leaves one or more nucleotides unhybridized, i.e., a toehold, at the 3′ end of the top strand. In some embodiments, the toehold is 4-8 nucleotides in length. In some embodiments, the waste strand, the bottom strand, or both comprise methylated nucleotides to prevent leakage of transcription.

Optionally, the disclosed systems and methods further may comprise a non-labeled double-stranded DNA molecule (e.g., which functions as a threshold gate) comprising a top strand that comprises a nucleotide sequence that is identical to the nucleotide sequence of the top strand of the labeled double-stranded DNA molecule. Typically, the top strand of the non-labeled double-stranded DNA molecule is longer than the bottom strand of the non-labeled double-stranded DNA molecule (e.g., by about 1, 2, 3, 4, 5, 6, 7, 8, 9, 10, 11, 12, 13, 14, 15 nucleotides or more). Optionally, the bottom strand of the non-labeled double-stranded DNA molecule is shorter in length than the length of the bottom strand of the fluorescently labeled double-stranded DNA molecule (e.g., by about 1, 2, 3, 4, 5, 6, 7, 8, 9, 10, 11, 12, 13, 14, 15 nucleotides or more), such that displacement of the bottom strand of the non-labeled double-stranded DNA molecule is favored thermodynamically versus displacement of the bottom strand of the fluorescently labeled double-stranded DNA molecule.

In some embodiments of the disclosed compositions, systems, kits, and methods, multiple aTFs and/or multiple engineered transcription templates may be included and/or utilized. For example, multiple aTFs and/or multiple engineered transcription templates may be included and/or utilized in order to create logic gates.

The compositions, systems, kits, and methods disclosed herein further may include or utilize additional components, such as additional components for performing RNA transcription. Additional components may include but are not limited to one or more of ribonucleoside triphosphates, an aqueous butter system that includes a reducing agent such dithiothreitol (DTT), divalent cations such as Mg⁺⁺, spermidine, an inorganic pyrophosphatase, an RNase inhibitor, crowding agents, and monovalent salts (e.g., NaCl and K-glutamate).

The components of the disclosed compositions, systems, kits, and methods may be mixed. For example, the components of the disclosed compositions, systems, kits, and methods may be mixed as an aqueous solution and/or may be dried or lyophilized to prepare a dried mixture which may be reconstituted (e.g., to perform the methods disclosed herein).

The disclosed compositions, systems, and kits, and the components thereof may be utilized in methods for detecting an analyte or target molecule in a sample (e.g., by performing an RNA transcription reaction). The methods may include contacting one or more components of the disclosed compositions, systems, and kits with the sample and detecting a detectable signal, thereby detecting the analyte or target molecule in the sample.

### Exemplary Embodiments

The compositions, systems, methods, and kits disclosed herein are exemplified by the embodiments below. These exemplary embodiments are not intended to be limiting.

1. A first embodiment comprise a composition, system, or kit for detecting an analyte comprising one or more of the following components: (a) an RNA polymerase; (b) an allosteric transcription factor (aTF), wherein the analyte is a ligand to which the aTF binds; (c) an engineered transcription template, (d) a dsDNA signal gate molecule, wherein the engineered transcription template comprises a promoter sequence for the RNA polymerase and an operator sequence for the aTF operably linked to a sequence encoding an RNA, wherein the aTF modulates transcription of the encoded RNA when the aTF binds the analyte as a ligand and wherein the transcribed RNA displaces a strand of the dsDNA signal gate molecule and a detectable signal is generated.

2. The composition, system, or kit of embodiment 1, wherein dsDNA signal gate molecule is a fluorescently labeled double-stranded nucleic acid comprising a fluorophore and a quencher that quenches the fluorophore in the fluorescently labeled double-stranded nucleic acid and the transcribed RNA displaces one of the strands of the fluorescently labeled double-stranded nucleic acid which results in dequenching of the fluorophore to generate the detectable signal.

3. The composition, system, or kit of any of the previous embodiments, wherein the reporter molecule is a fluorescently labeled double-stranded DNA molecule comprising a top strand having a fluorophore conjugated at its 3′-end and a bottom strand having a quencher conjugated at its 5′ end that quenches the fluorophore in the fluorescently labeled double-stranded DNA molecule and the transcribed RNA displaces the bottom strand of the fluorescently labeled double-stranded DNA molecule which results in dequenching of the fluorophore to generate the detectable signal.

4. The composition, system, or kit of any of the previous embodiments, wherein the top strand is longer than the bottom strand and wherein the transcribed RNA comprises a sequence that is complementary to the full length of the top strand.

5. The composition, system, or kit of any of the previous embodiments, wherein the top strand comprises one or more non-natural modifications that prevent the top strand from being utilized as a template for transcription (e.g., 2′-O-methylation).

6. The composition, system, or kit of any of the previous embodiments, wherein the system further comprises a non-labeled double-stranded DNA molecule comprising a top strand that comprises a nucleotide sequence that is identical to the nucleotide sequence of the top strand of the labeled double-stranded DNA molecule.

7. The composition, system, or kit of any of the previous embodiments, wherein the top strand of the non-labeled double-stranded DNA molecule is longer than the bottom strand of the non-labeled double-stranded DNA molecule.

8. The composition, system, or kit of any of the previous embodiments, wherein the bottom strand of the non-labeled double-stranded DNA molecule is shorter in length than the length of the bottom strand of the fluorescently labeled double-stranded DNA molecule.

9. The composition, system, or kit of any of the previous embodiments, wherein the transcribed RNA does not form and/or is designed not to form an intramolecular secondary structure, and optionally an intramolecular structure comprising more than 3 consecutively paired nucleotides.

10. The composition, system, or kit of any of the previous embodiments, wherein the RNA polymerase is selected from T7 RNA polymerase, T3 RNA polymerase, SP6 RNA polymerase, and Syn5 RNA polymerase or the RNA polymerase is an engineered polymerase.

11. The composition, system, or kit of any of the previous embodiments, wherein the RNA polymerase is an engineered RNA polymerase.

12. The composition, system, or kit of any of the previous embodiments, wherein the aTF represses, blocks, or inhibits transcription from the engineered transcription template when the aTF binds the operator.

13. The composition, system, or kit of of any of the previous embodiments, wherein the aTF activates transcription from the engineered transcription template when the aTF binds the operator.

14. The composition, system, or kit of any of the previous embodiments, wherein in the absence of the analyte as a ligand the aTF binds to the operator sequence.

15. The composition, system, or kit of of any of the previous embodiments, wherein in the presence of the analyte as a ligand the aTF does not bind to the operator or binds to the operator at a lower affinity than in the absence of the analyte as a ligand.

16. The composition, system, or kit of any of the previous embodiments, wherein in the presence of the analyte as a ligand the aTF binds to the operator sequence.

17. The composition, system, or kit of any of the previous embodiments, wherein in the absence of the analyte as a ligand the aTF does not bind to the operator or binds to the operator at a lower affinity than in the presence of the analyte as a ligand.

18. The composition, system, or kit of any of the previous embodiments, wherein the aTF belongs to the TetR, MarR, or ArsR/SmtB class or family of transcription factors or the aTF is an engineered aTF.

19. The composition, system, or kit of any of the previous embodiments, wherein the aTF is selected from the group consisting of TetR, MphR, QacR, OtrR, CtcS, SAR2349, MobR, SmtB, CadC, CsoR, AdcR, TtgR, and HucR.

20. The composition, system, or kit of any of the previous embodiments, wherein the analyte that is a ligand for the aTF is a member of the tetracycline-family of antibiotics.

21. The composition, system, or kit of any of the previous embodiments, wherein the analyte that is a ligand for the aTF is a member of the macrolide-family of antibiotics.

22. The composition, system, or kit of any of the previous embodiments, wherein the analyte is a quaternary amine or salts thereof.

23. The composition, system, or kit of any of the previous embodiments, wherein the analyte is a metal or a cation thereof.

24. The composition, system, or kit of any of the previous embodiments, wherein the metal or the cation thereof is Zn, Pb, Cu, Cd, Ni, As, or Mn.

25. The composition, system, of any of the previous embodiments, wherein the analyte is selected from salicylate, 3-hydroxy benzoic acid, naringenin, and uric acid.

26. The composition, system, of any of the previous embodiments, further comprising (d) one or more components for preparing a reaction mixture for RNA transcription.

27. The composition, system, of any of the previous embodiments, wherein the components are mixed and form an aqueous solution for performing RNA transcription.

28. The composition, system, of any of the previous embodiments, wherein the components are mixed and form a dried mixture which may be reconstituted to form a reaction mixture for performing RNA transcription.

29. A method for detecting an analyte in a sample, the method comprising contacting the sample with one or more components of the composition, system, or kit of any of the foregoing embodiments and detecting signal.

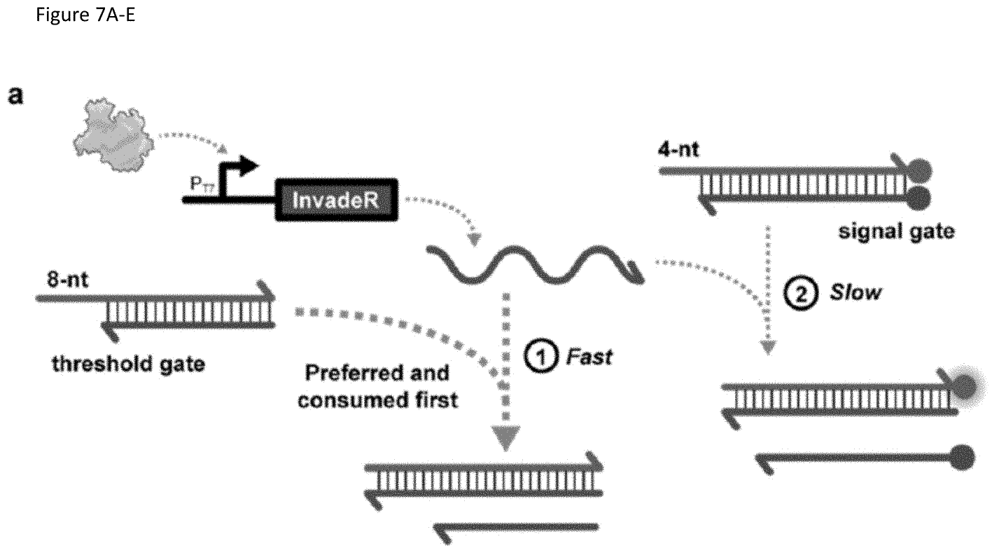

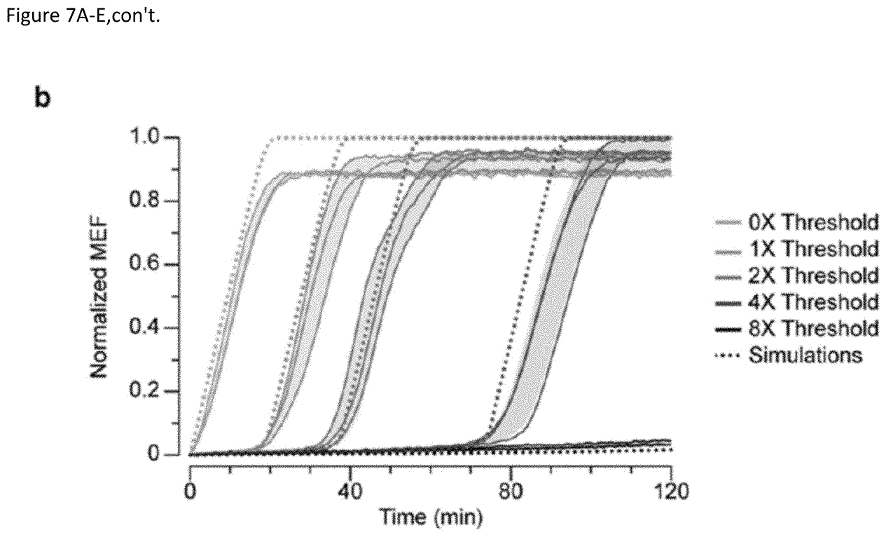

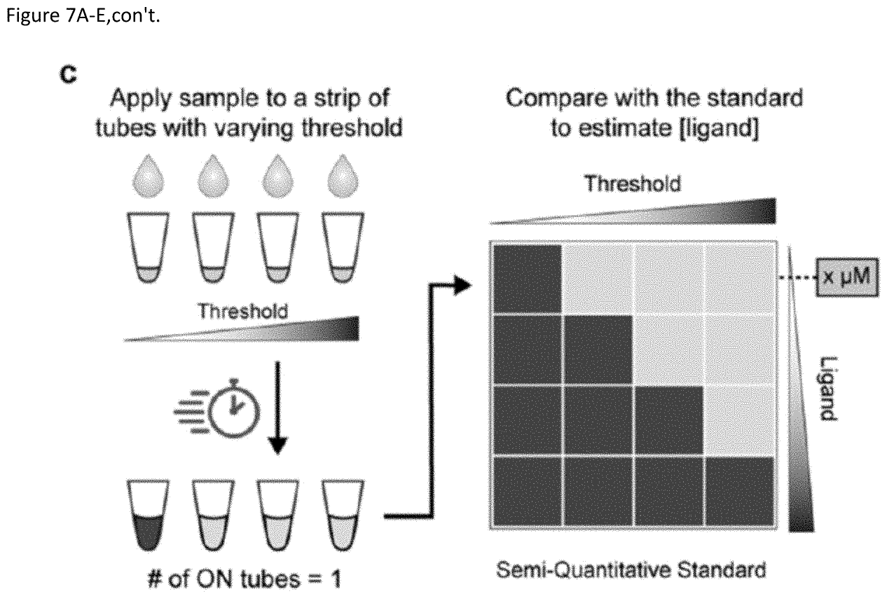

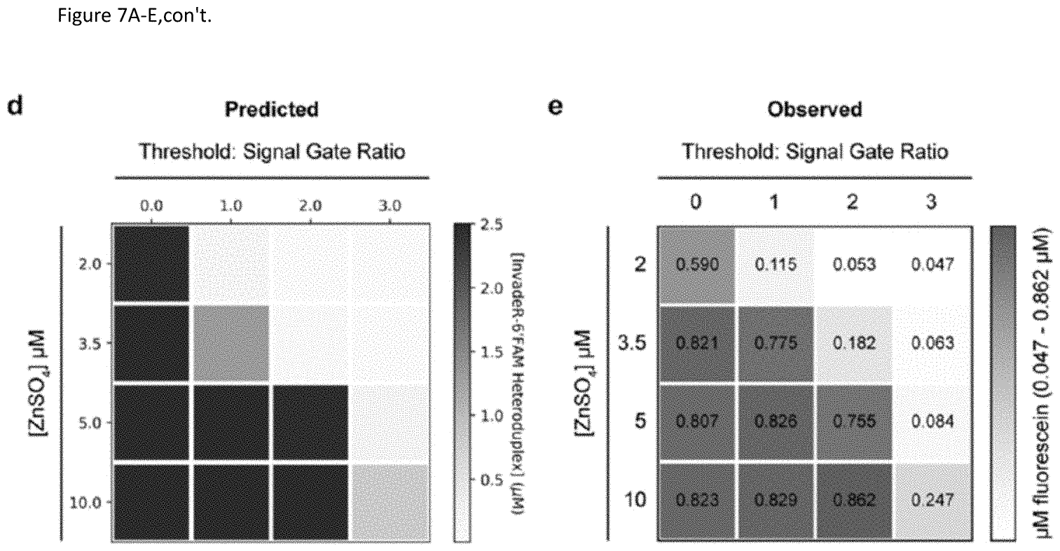

30. The composition, system, kit or method of any of the foregoing embodiments comprising and/or utilizing a plurality of RNA output sequences that are adapted to displacement multiple DNA strands in a dsDNA signal gate molecule, optionally wherein the composition, system, kit or method exhibits improved reaction kinetics for example as illustrated in FIG. 7 a, b, c.

31. The composition, system, kit or method of any of the foregoing embodiments which enables molecular computation between the sensing events and the reporting events optionally as illustrated in FIG. 1.

32. The composition, system, kit or method of any of the foregoing embodiments comprising and/or utilizing a non-labelled dsDNA gate with a longer toehold than the dsDNA signal gate molecule which functions as a kinetic “comparator” circuit which function to delay the temporal response of the reaction, optionally as illustrated in FIG. 7.

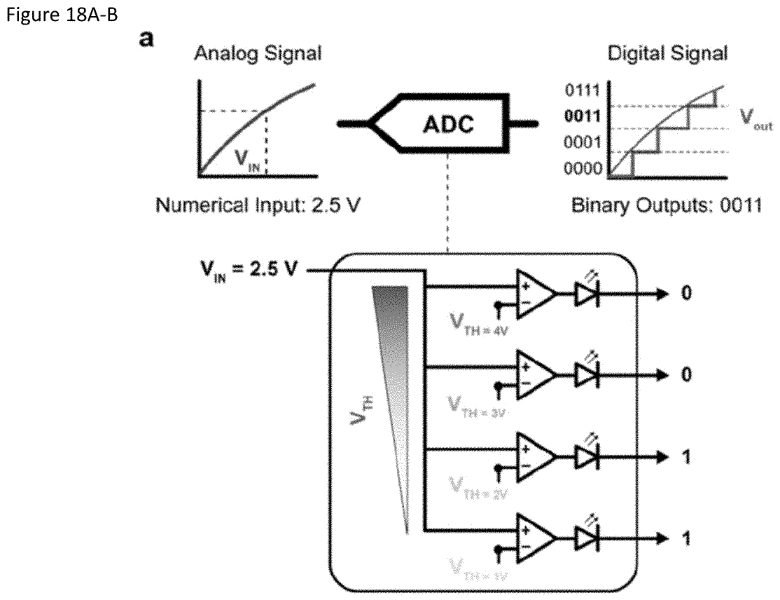

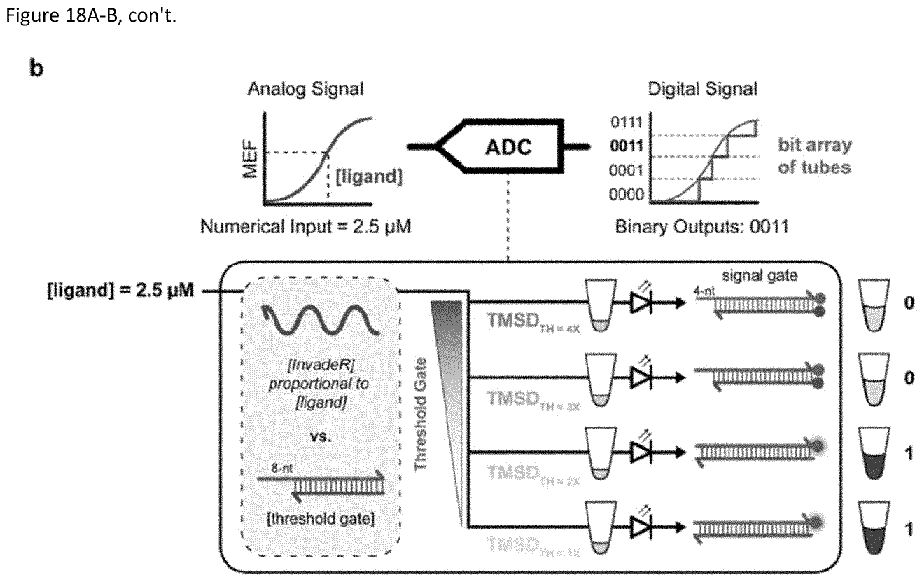

33. The composition, system, kit or method of embodiment 32, comprising and/or utilizing a plurality of comparator circuits in series which function to act as a genetic “analog-to-digital converter” (ADC) to enable target input quantification, optionally as illustrated in FIG. 18.

34. The composition, system, kit or method of embodiment 32 or 33, which is adapted to detect and/or quantify a range of compounds related to environmental contamination and human health, optionally as illustrated in FIG. 7.

35. The composition, system, kit or method of any of embodiments 32-34, in which the genetic ADC circuit receives an analog input concentration of a target compound and converts it to a digital binary output to indicate the concentration range of that compound.

36. The composition, system, kit or method of any of embodiments 32-35, wherein the target compound is zinc, optionally as illustrated in FIG. 7.

37. A method for predicting the functional characteristics of any of a composition, system, kit or method disclosed herein comprising utilizing one or more ordinary differential equations (ODEs) as disclosed herein.

38. The composition, system, or kit of any of embodiments 1-28, or 30-36, or the method of embodiment 29 or 37, wherein the composition, system, kit, or method is configured to detect the presence and/or absence of at least two analytes.

39. The composition, system, kit, or method of embodiment 38, comprising a first aTF and a second aTF, wherein the first aTF binds a first ligand, and wherein the second aTF binds a second ligand.

40. The composition, system, kit, or method of embodiment 39, comprising a first engineered transcription template and a second engineered transcription template, wherein the first engineered transcription template comprises a first operator for the first aTF, and wherein the second engineered transcription template comprises a second operator for the second aTF.

41. The composition, system, kit, or method of embodiment 40, wherein the first engineered transcription template encodes a first RNA, and wherein the second engineered transcription template encodes a second RNA, wherein (a) the first RNA and the second RNA are different, or (b) the first RNA and the second RNA are the same.

42. The composition, system, kit, or method of embodiment 41, comprising a first dsDNA signal gate molecule and a second ds DNA signal gate molecule, wherein one strand of the first ds DNA signal gate molecule is complementary to the first encoded RNA, and wherein one strand of the second ds DNA signal gate molecule is complementary to the second encoded RNA.

43. The composition, system, kit, or method of any one of embodiments 38-42, wherein the first and second aTFs bind the first and second operators on the first and second engineered transcription templates, respectively, in the presence of the first and second ligands.

44. The composition, system, kit, or method of any one of embodiments 38-42, wherein the first and second aTFs bind the first and second operators on the first and second engineered transcription templates, respectively, in the absence of the first and second ligands.

45. The composition, system, kit, or method of any one of embodiments 38-42, wherein the first aTF binds the first operator on the first engineered transcription template in the presence of the first ligand, and wherein the second aTF binds the second operator on the second engineered transcription template in the absence of the second ligand.

46. The composition, system, kit, or method of any embodiments 38-41, comprising a dsDNA signal gate molecule, wherein one strand of the ds DNA signal gate molecule comprises a first region complementary to the first encoded RNA, and a second region complementary to the second encoded RNA.

47. The composition, system, kit, or method of embodiment 46, wherein the first and second aTFs bind the first and second operators on the first and second engineered transcription templates, respectively, in the presence of the first and second ligands.

48. The composition, system, kit, or method of embodiment 46, wherein the first and second aTFs bind the first and second operators on the first and second engineered transcription templates, respectively, in the absence of the first and second ligands.

49. The composition, system, kit, or method of embodiment 46, wherein the when the first aTF binds the first operator on the first engineered transcription template in the presence of the first ligand, and wherein the second aTF binds the second operator on the second engineered transcription template in the absence of the first ligand.

50. The composition, system, kit, or method of embodiment 38, comprising a first engineered transcription template encoding a first RNA, and an unregulated transcription template encoding a second RNA; wherein the unregulated transcription template comprises a promoter sequence for RNA polymerase and wherein the encoded second RNA is different than the first encoded RNA.

51. The composition, system, kit, or method of embodiment 50, wherein the first encoded RNA hybridizes to the second encoded RNA.

52. A composition, system, or kit for detecting an analyte comprising one or more of the following components: (a) an RNA polymerase; (b) an allosteric transcription factor (aTF), wherein the analyte is a ligand to which the aTF binds; (c) an engineered transcription template, (d) a dsDNA signal gate molecule, (e) a dsDNA fuel molecule, wherein the engineered transcription template comprises a promoter sequence for the RNA polymerase and an operator sequence for the aTF operably linked to a sequence encoding an RNA, wherein the aTF modulates transcription of the encoded RNA when the aTF binds the analyte as a ligand and wherein the transcribed RNA displaces a strand of the DNA fuel molecule generating a free single-stranded DNA molecule which displaces a strand of the dsDNA signal gate molecule and a detectable signal is generated.

53. The composition, system, or kit of embodiment 52, wherein the dsDNA fuel molecule is a double-stranded nucleic acid comprising a waste strand and a RecycleD strand.

54. The composition, system, or kit of embodiment 52 or 53, wherein the dsDNA signal gate molecule is a fluorescently labeled double-stranded DNA molecule comprising a top strand having a fluorophore conjugated at its 3′-end and a bottom strand having a quencher conjugated at its 5′ end that quenches the fluorophore in the fluorescently labeled double-stranded DNA molecule and the RecycleD strand displaces the bottom strand of the fluorescently labeled double-stranded DNA molecule which results in dequenching of the fluorophore to generate the detectable signal and hybridization of the RecycleD strand to the top strand generates a double-stranded DNA molecule comprising a 3′ toehold on the top strand wherein the RNA polymerase transcribes the top strand and displaces the RecycleD strand and generates a fluorescent DNA/RNA hybrid.

55. The composition, system, or kit of any one of embodiments 52-54, wherein the top strand is longer than the bottom strand and wherein the transcribed RNA comprises a sequence that is complementary to the full length of the top strand.

56. The composition, system, or kit of any one of embodiments 52-55, wherein the waste strand or the bottom strand comprises one or more non-natural modifications that prevent the strand from being utilized as a template for transcription (e.g., 2′-O-methylation).

57. The composition, system, or kit of any one of embodiments 52-56, which is adapted to detect and/or quantify one or more compounds, or a range of compounds related to environmental contamination and/or human health.

### EXAMPLES

The following examples are illustrative and should not be interpreted to limit the scope of the claimed subject matter.

### Example 1—Programming Cell-Free Biosensors with DNA Strand Displacement Circuits

Engineering TMSD to be Compatible with In Vitro Transcription

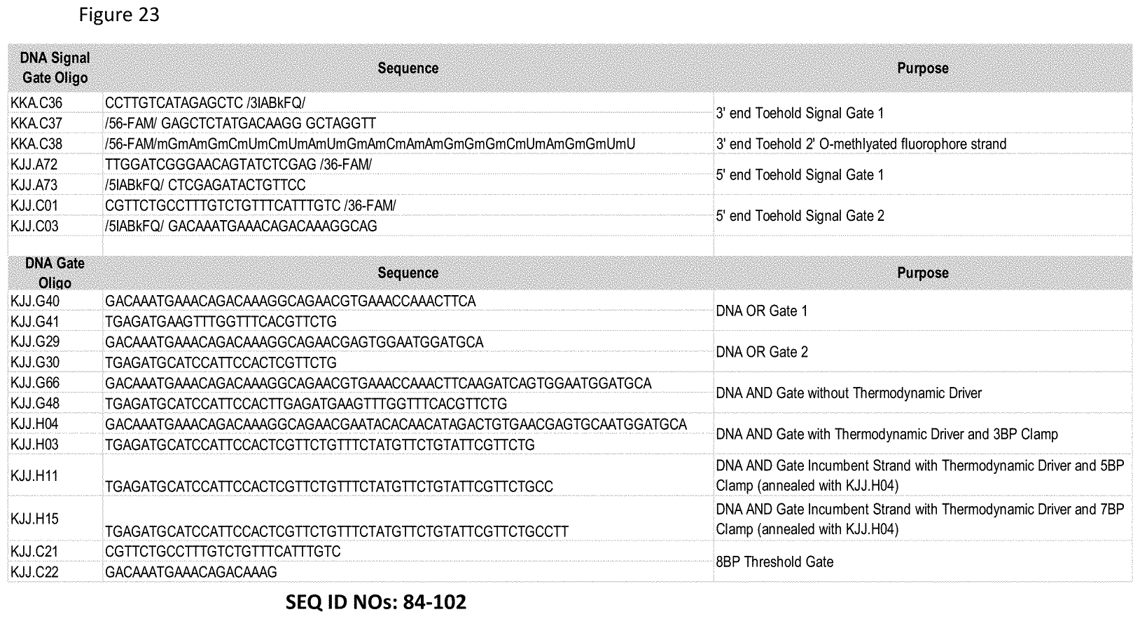

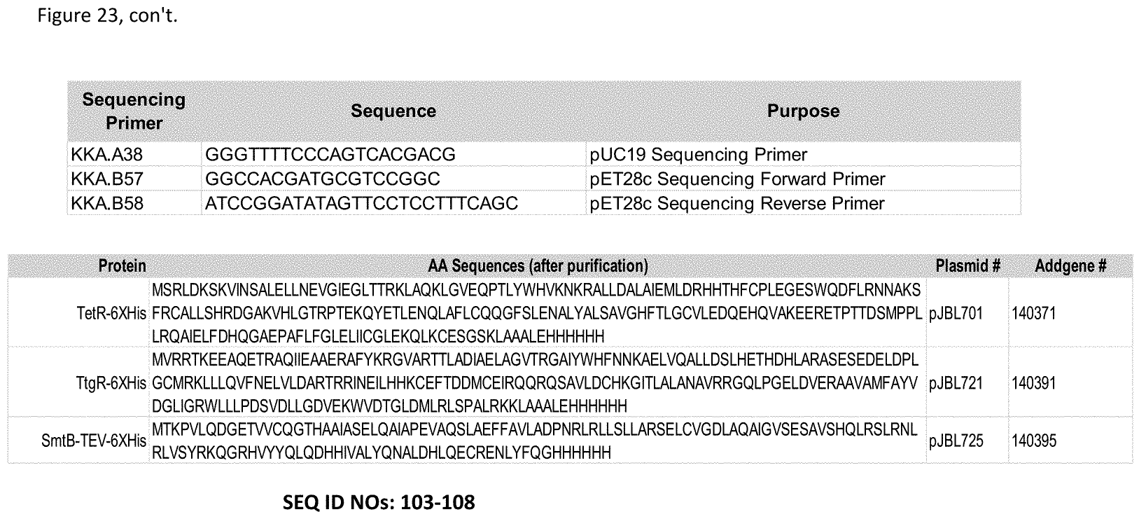

To interface aTFs with TMSD, we first sought to directly interface unregulated IVT reactions with the DNA gates used to generate signals in TMSD. This required us to validate that a single-stranded RNA can strand-displace a DNA signal gate. We adapted the design and the sequence of the DNA signal gate from a previous work [31] and created the gate by annealing two ssDNA strands: (1) a fluorophore strand consisting of a 24-nucleotide (nt) ssDNA modified with a 6′ FAM fluorophore on its 5′ end and (2) a quencher strand consisting of a 16-nt ssDNA strand complementary to the fluorophore strand and modified with an Iowa black quencher on its 3′ end (FIG. 23) [31]. Once annealed, the DNA signal gate has an 8-nt toehold on its 3′ end to initiate strand displacement. Following the TMSD design architecture, we designed an invading RNA strand (“InvadeR”) to be fully complementary to the 24-nt fluorophore strand so that it could bind to the toehold region and strand-displace the quencher strand to generate a fluorescent output.

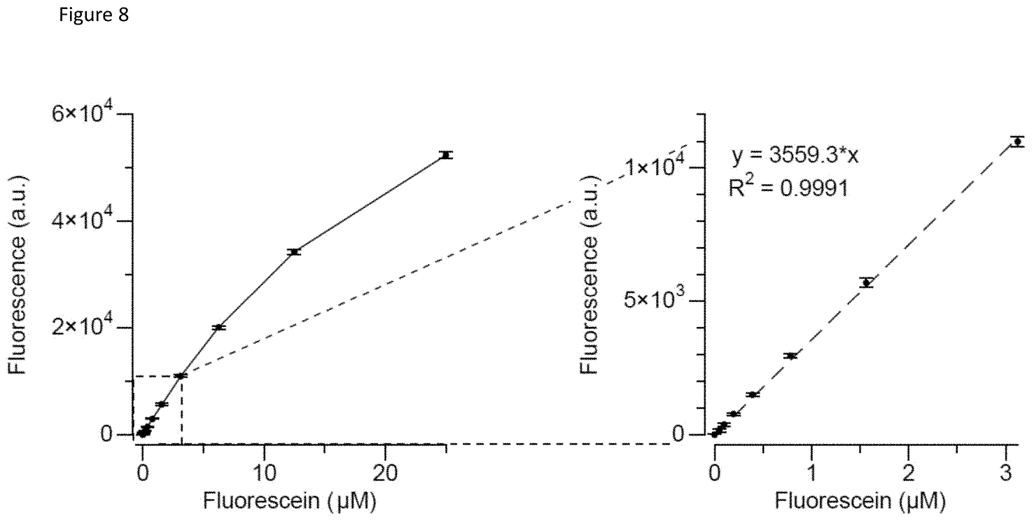

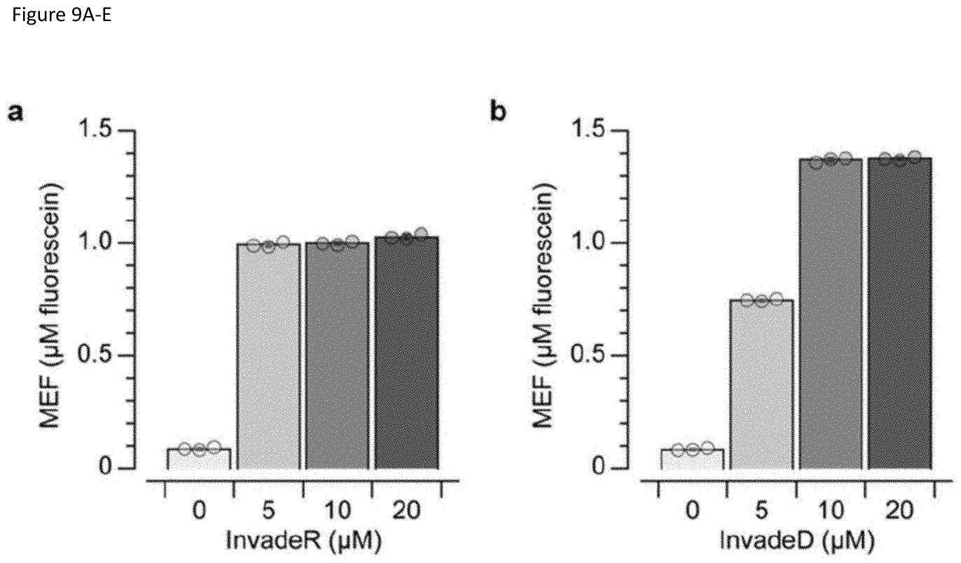

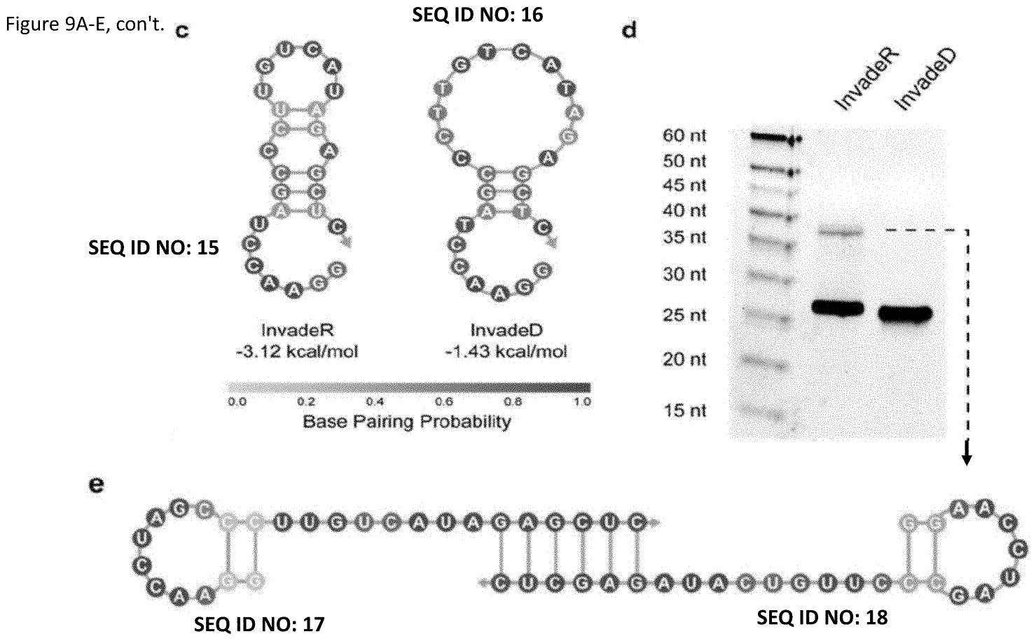

We tested the strand displacement efficiency of this DNA signal gate by adding purified InvadeR to the reaction and monitoring fluorescence, which was standardized to an external fluorescein standard (FIG. 8). Addition of purified InvadeR at varying concentrations resulted in significant fluorescence activation over a no InvadeR control (FIG. 9a). In this way, InvadeR behaved similarly to an invading ssDNA strand (“InvadeD”), though these molecules differed in the fluorescence dose response observed (FIG. 9a, b). Specifically, titration of InvadeR resulted in a plateau of fluorescence at lower concentrations than InvadeD. This could be due to differences in RNA and DNA folding thermodynamics. In particular, NUPACK [32] predicts that InvadeD has a less stable structure than InvadeR (−1.43 kcal/mol vs. −3.12 kcal/mol, respectfully) (FIG. 9c), and that InvadeR can bind to itself to form a duplex (FIG. 9d, e). This could inhibit binding and strand displacement of the DNA signal gate and lead to a much slower TMSD response even at a higher InvadeR concentration. While this result shows that RNA-initiated TMSD is possible, it also points to several RNA-specific challenges that need to be addressed.

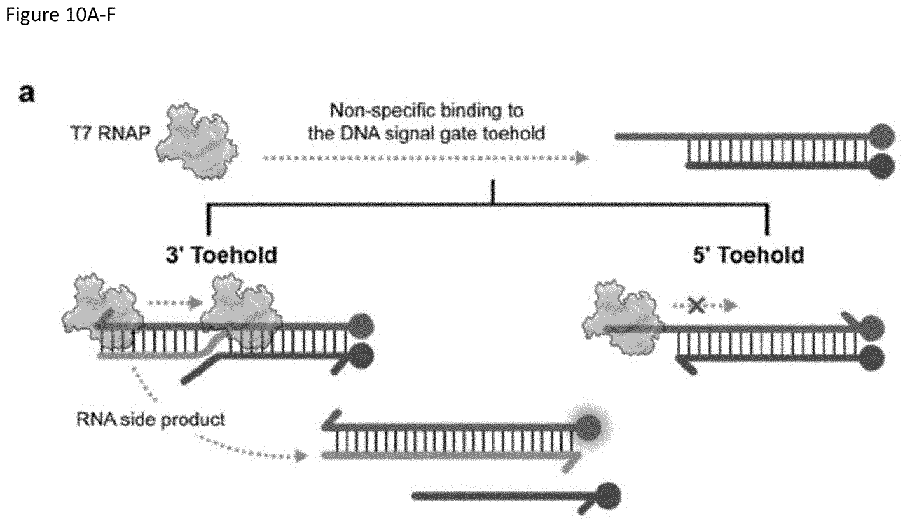

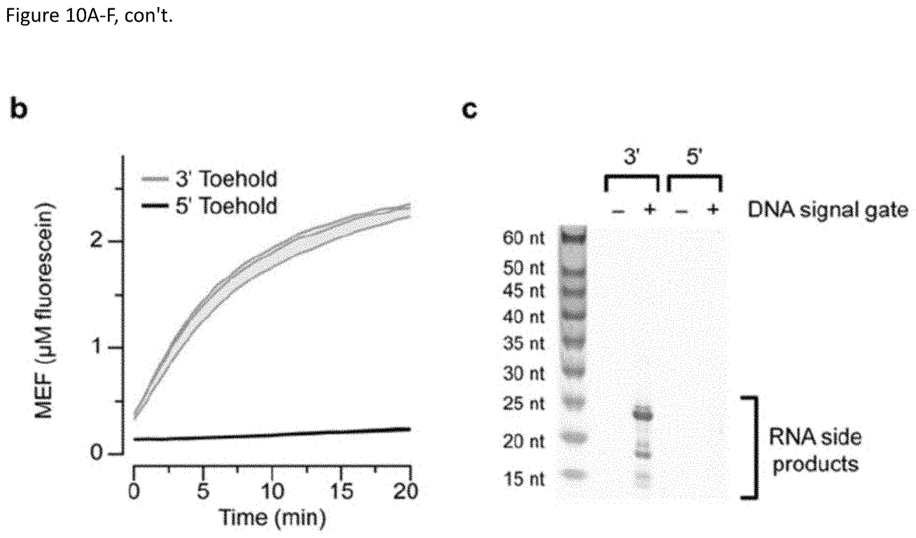

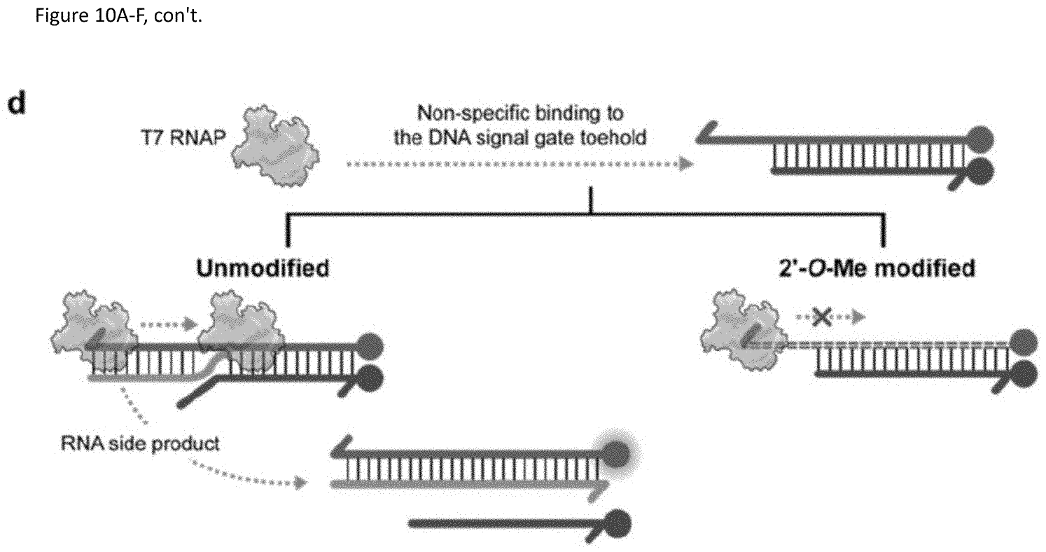

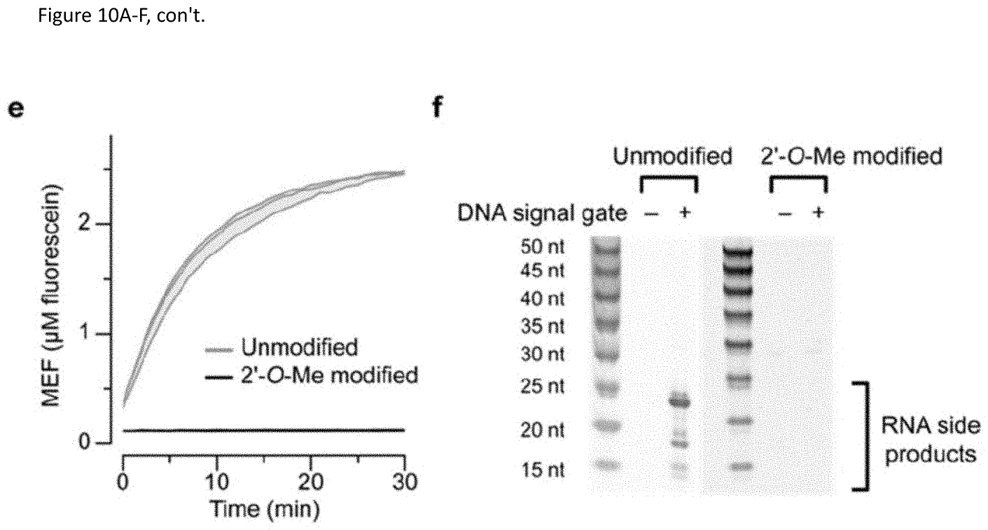

We next sought to determine if InvadeR can be transcribed in situ in the presence of the DNA signal gate to generate a fluorescent output. Following the ROSALIND platform design, we chose reaction conditions that use the fast phage polymerase, T7 RNAP. We configured the DNA template encoding InvadeR to consist of the minimal 17-base pair (bp) T7 promoter sequence followed by two initiating guanines and the InvadeR sequence. To begin, we tested whether adding T7 RNAP along with other in vitro transcription (IVT) reagents could interfere with the DNA signal gate. To our surprise, we observed an increase in fluorescence in the absence of a DNA template when only T7 RNAP, IVT buffer and NTPs were added to the DNA signal gate (FIG. 10b). Previous literature reported the ability of T7 RNAP to initiate promoter-independent transcription from exposed, linear ssDNA regions [29, 33-35]. Based on these observations, we hypothesized that T7 RNAP was initiating transcription from the 3′ toehold region of the DNA signal gate, causing strand displacement and signal generation (FIG. 10a). To test this hypothesis, we reversed the polarity of the DNA signal gate so that the toehold region is on its 5′ end while keeping its sequence the same. As expected, no fluorescence signal was observed from the 5′ toehold DNA signal gate in the absence of the DNA template encoding InvadeR (FIG. 10b). To confirm that the signal generation is due to transcription of the DNA signal gate, RNA species from each IVT reaction without the DNA template were extracted and run on a urea-PAGE gel. The resulting gel image shows that RNA side products were generated only from the reaction with the 3′ toehold DNA signal gate (FIG. 10c). We also designed a modified DNA signal gate where the fluorophore strand is modified with 2′-O-methlyation to prevent transcription while keeping the toehold on the 3′ end [35]. As expected, the signal remained at a basal level with the 3′ toehold 2′-O-methylated DNA signal gate, and no RNA side products were observed on a gel (FIG. 10d-f), further confirming our hypothesis.

Together, these results revealed several important design features required to interface TMSD with IVT reactions. In particular, the use of a 5′ toehold is an important design requirement of the DNA signal gate to prevent promoter-independent transcription by T7 RNAP.

Interfacing In Vitro Transcription with TMSD Outputs

We next sought to use TMSD to directly track the RNA outputs generated by T7 RNAP-driven IVT in situ. In particular, we focused on optimizing the design of InvadeR for rapid, robust signal generation. Based on our observations in the differences between InvadeR and InvadeD (FIG. 9), we hypothesized that the secondary structure of InvadeR would play a critical role in the strand displacement efficiency and thus the overall TMSD reaction kinetics. For example, an InvadeR strand that forms a stable secondary structure would have to overcome a greater energy barrier to unfold and displace the quencher strand from the DNA signal gate. A stable 3′ end structure of InvadeR could also interfere with the initial binding of the toehold region and hinder the TMSD branch migration process [36, 37].

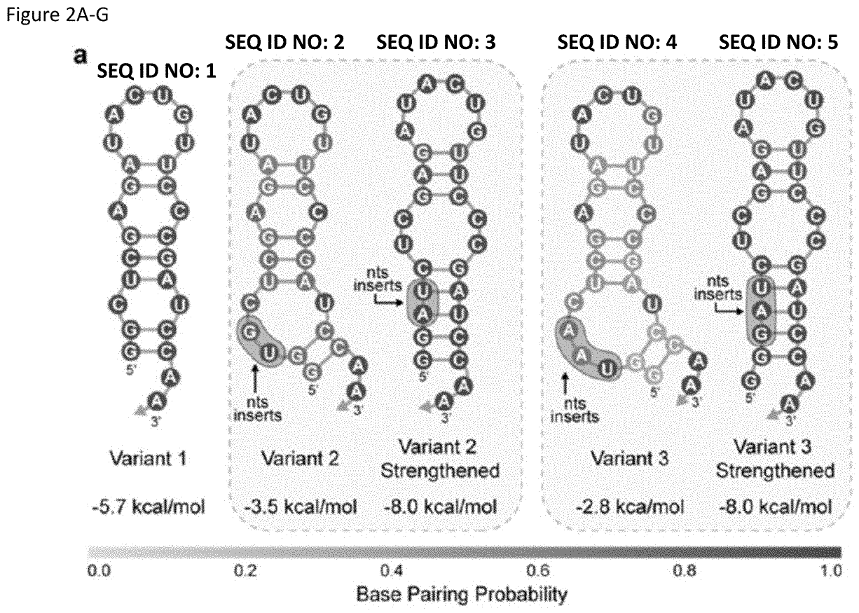

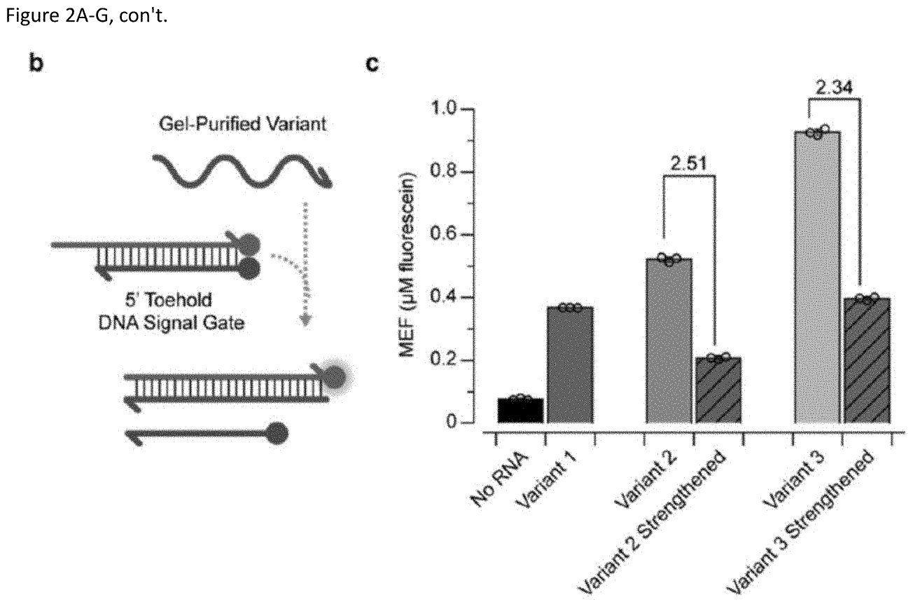

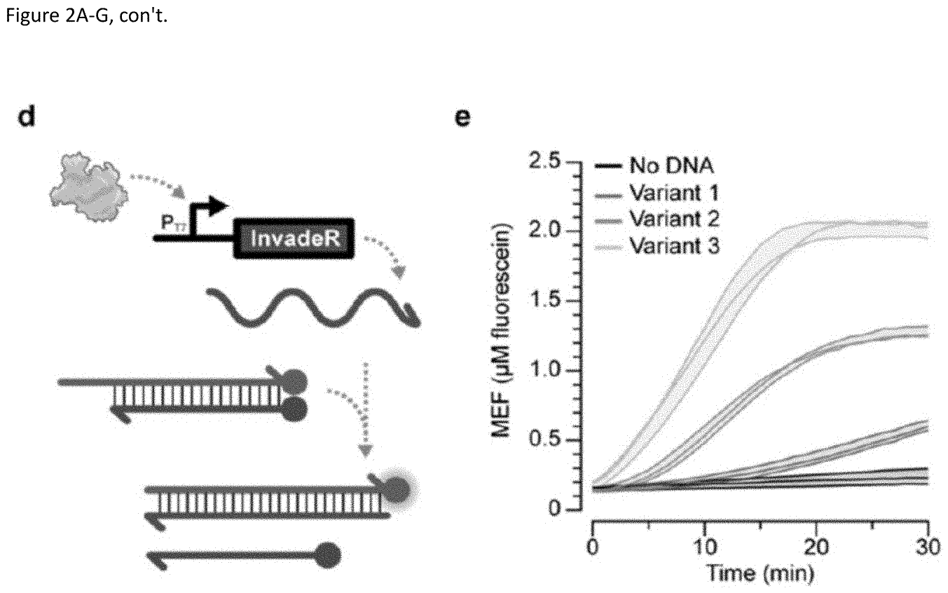

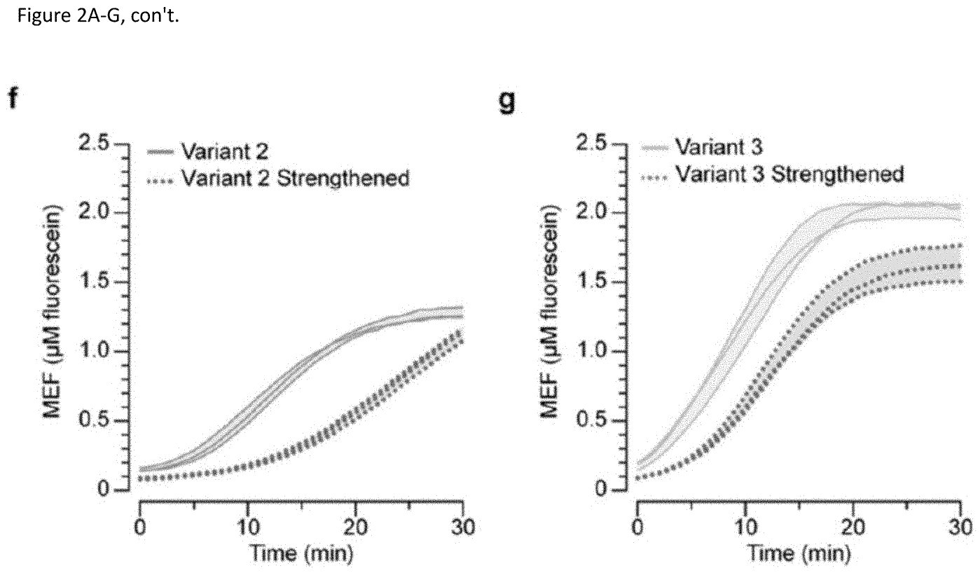

To test this hypothesis, we designed three different variants of InvadeR that can strand-invade the DNA signal gate optimized in the previous section (FIG. 2a). Variant 1, which is the reverse sequence of the InvadeR designed to strand-displace the 3′ toehold DNA signal gate shown in FIG. 10, consists of two initiating guanines followed by the sequence fully complementary to the fluorophore strand of the 5′ toehold DNA signal gate (for the rest of this work, all DNA signal gates used had 5′ toeholds). For variants 2 and 3, additional nucleotides were inserted between the initiating guanines and the InvadeR sequence to destabilize the predicted G-C base pairs on the 3′ end as well as the overall secondary structure. We also designed strengthened versions of variants 2 and 3 such that the additional nucleotides made the predicted secondary structures more stable with high base pairing probabilities on their 3′ ends. When an equimolar amount of each gel-purified InvadeR variant was added to the DNA signal gate (FIG. 2b), we observed that in general, the magnitudes of fluorescence signals were ordered according to the predicted minimum free energies of each variant, with the least stable variant 3 (−2.8 kcal/mol) showing the highest fluorescence and the most stable variant 1 (−5.7 kcal/mol) the lowest (FIG. 2c). Furthermore, each strengthened version showed significantly lower fluorescence than the corresponding un-strengthened variant (fold reduction of 2.51 and 2.34 for variants 2 and 3, respectively), confirming our hypothesis (FIG. 2c).

Next, we tested the strand displacement reaction kinetics of the variants transcribed in situ. Fifty nM of the DNA template encoding each InvadeR variant was added to a reaction mixture containing IVT buffer, T7 RNAP, NTPs and the DNA signal gate, and their fluorescence activation was measured (FIG. 2d). We observed fastest fluorescence activation from variant 3 followed by variants 2 and 1 (FIG. 2e), which agree with the previous experiment. When we compared the reaction kinetics of variant 2 and 3 to that of their respective strengthened versions, we observed slower responses from the strengthened versions, reaffirming our hypothesis (FIG. 2f, g).

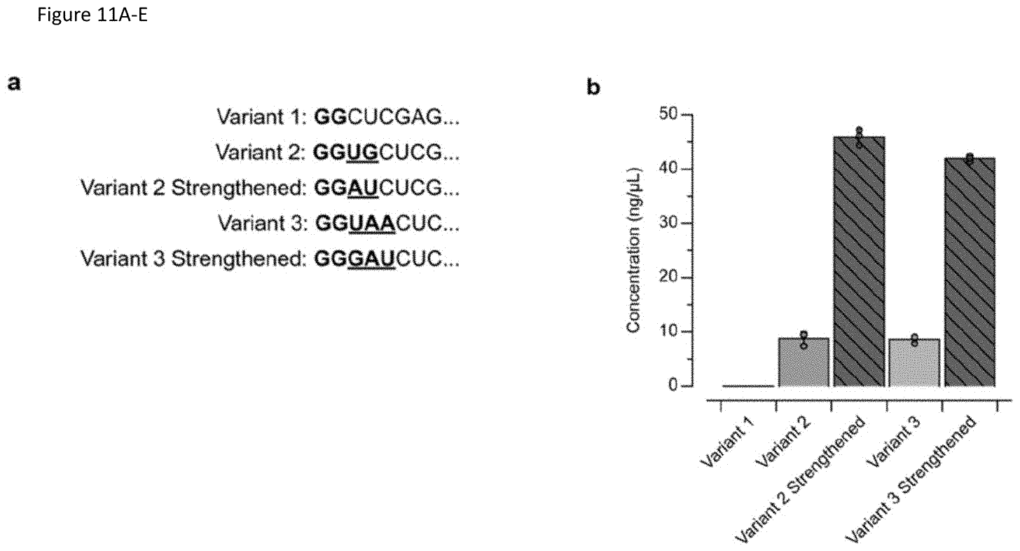

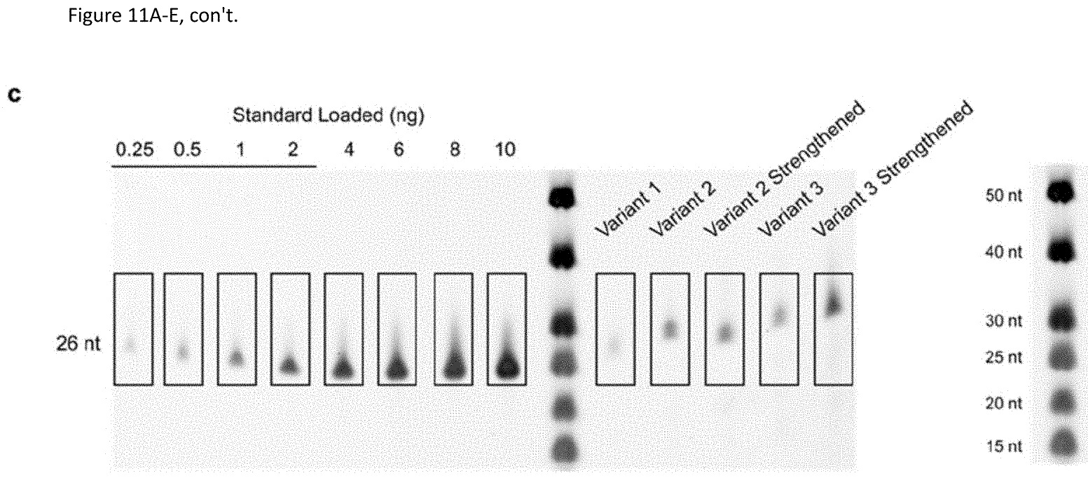

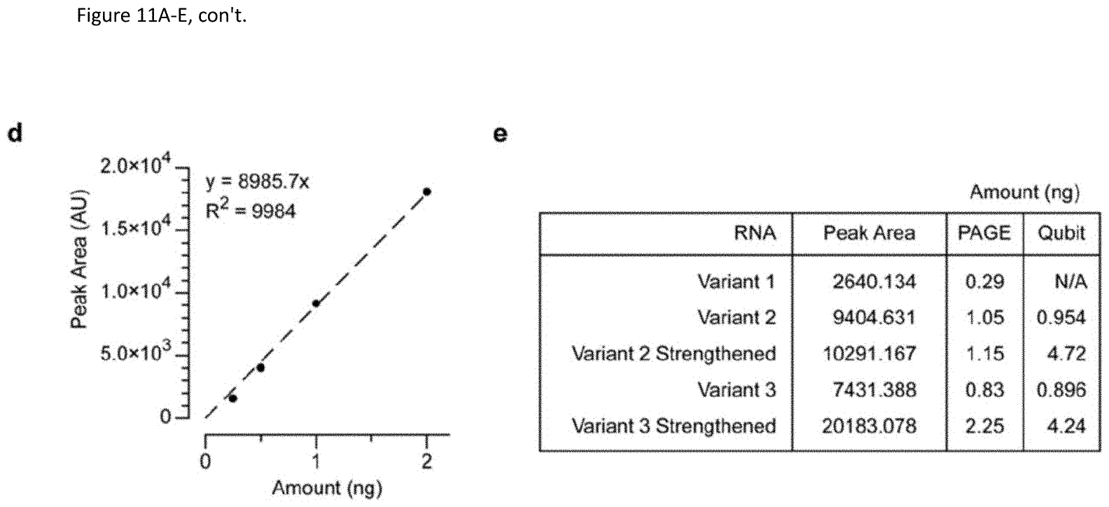

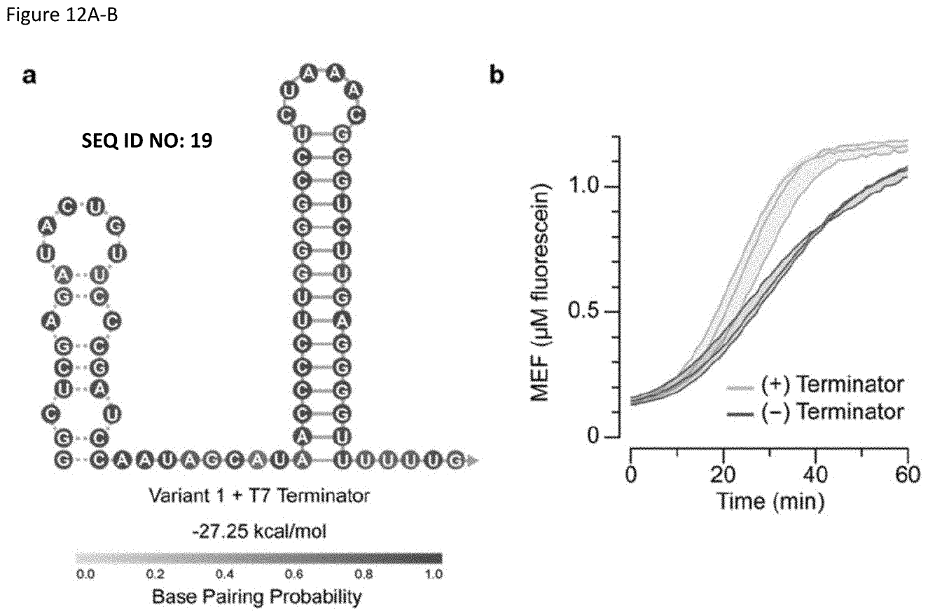

While the qualitative ordering of fluorescence kinetics and predicted thermodynamic stabilities of the InvadeR variants held, we did observe some discrepancies between the quantitative predicted secondary structure stabilities and the reaction kinetics of the strengthened variants. For instance, although NUPACK predicts lower minimum free energy values from the strengthened variants than variant 1, the strengthened variants show faster reaction kinetics than variant 1 (FIG. 2e-g). Furthermore, the endpoint fluorescence values reached by the strengthened variants in situ are higher than that of variant 1, conflicting with the results observed in FIG. 2c. We hypothesized that these discrepancies could be due to varying transcription efficiency of each DNA template, as it has been reported that the sequence of the initially transcribed region greatly impacts T7 RNAP transcription efficiency [38]. To test this hypothesis, RNA products from 30-minute long IVT reactions initiated with each DNA template were extracted, and their RNA concentrations were measured using both the RNA Qubit assay and a gel band intensity analysis from a urea-PAGE gel stained with SYBR gold (FIG. 11) [39]. In both cases, we observed the highest RNA concentrations from the strengthened variants (FIG. 11b, e), which explain the discrepancies described above. Despite having a low transcription efficiency, variant 3 still showed the fastest kinetics, indicating that the RNA secondary structure greatly affects the TMSD response speed. We also found that adding a T7 terminator sequence at the end of the DNA template speeds up the reaction [40], although not considerably (FIG. 12).

Together, these results show that both secondary structure and transcription efficiency impact the ability of RNA strands transcribed in situ to invade DNA signal gates and that these design principles can be leveraged to enhance reaction speed.

Interfacing Cell-Free Biosensors with TMSD Outputs

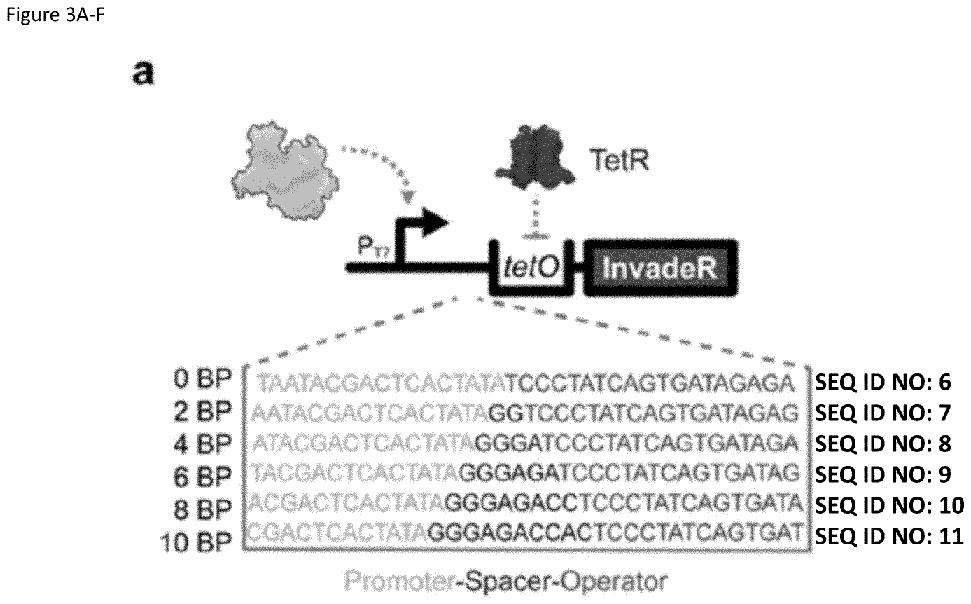

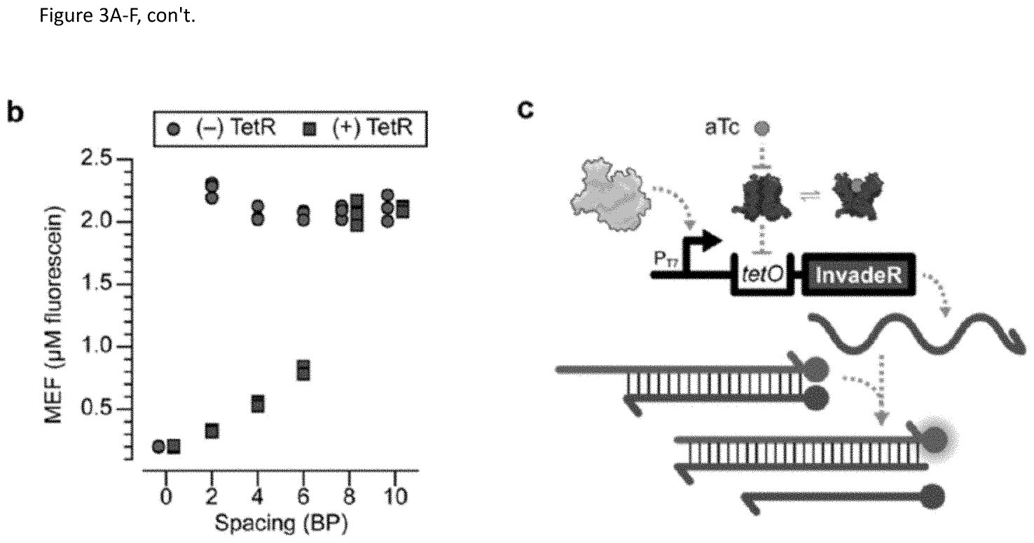

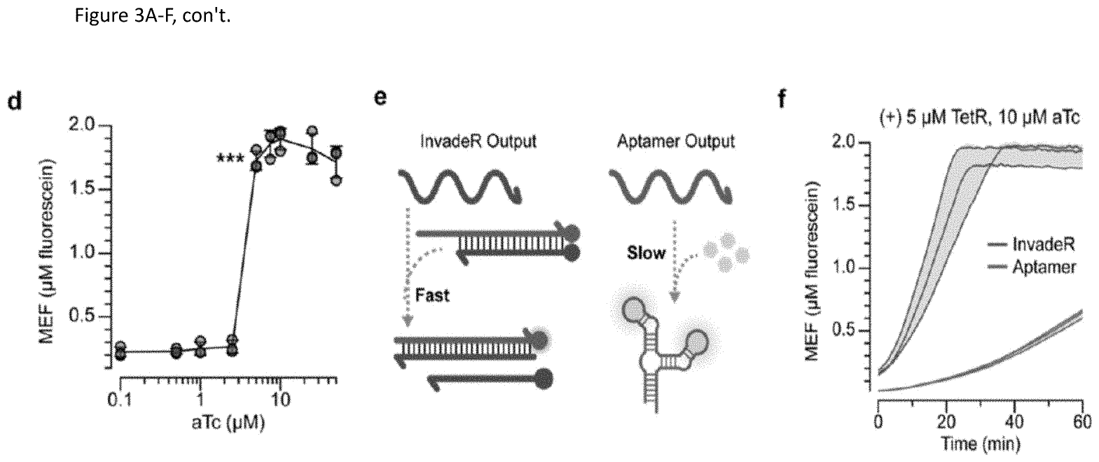

Next, we sought to determine whether the transcription of InvadeR can be regulated with an aTF, thus creating a ligand-responsive biosensor that uses TMSD outputs. This required us to insert an aTF operator sequence in between the T7 promotor and InvadeR sequence to allow an aTF to regulate transcription. We previously demonstrated that the spacing between the minimal 17-bp T7 promoter sequence and the aTF operator sequence is important for efficient regulation of IVT in ROSALIND reactions [7]. To test if this spacing remained important in the TMSD platform, we used TetR as our model aTF to determine the optimum spacing for efficient repression in the presence of TetR and efficient transcription in the absence of TetR [41]. We used the native sequence that follows the canonical T7 RNAP promoter as a spacer in 2-bp increments from 0 to 10-bp, immediately followed by the tetO sequence and InvadeR sequence (FIG. 3a). IVT reactions were set up using 50 nM DNA template with or without purified recombinant TetR protein in 100-fold excess of the DNA template. Consistent with our previously reported observation [7], the absence of any spacing resulted in no fluorescence activation in either the presence (regulated) or the absence (unregulated) of TetR (FIG. 3b). However, robust fluorescence signal was observed without TetR when using a 2-bp spacer, which was reduced to nearly baseline levels when regulated by TetR. Spacers longer than 2-bp resulted in T7 RNAP read-through, leading to fluorescence activation in the presence of TetR. We note, however, if an operator sequence starts with a guanine, thus acting as the initiating nucleotide for T7 RNAP, no spacing could still lead to transcription in the absence of the corresponding aTF.

Using the 2-bp spacer, we next determined whether TetR can be de-repressed with its cognate ligand, anhydrotetracycline (aTc) to allow transcription of InvadeR (FIG. 3c). When a range of aTc concentrations was added to reactions each containing T7 RNAP, 50 nM DNA template, 5 μM TetR dimer and 5 μM DNA signal gate, we observed a strong repression down to low micromolar amounts of aTc, with half-maximal induction between 2.5 μM and 5 of aTc. (FIG. 3d).

Due to the rapid speed of TMSD reactions [17], we hypothesized that the ligand-mediated induction speed of the InvadeR output would be much faster than the previously used fluorescence-activating RNA aptamer output (FIG. 3e). Specifically, the kinetic rate of TMSD of an 8-nt toehold is two orders of magnitude higher than the Spinach aptamer and DFHBI-1T binding kinetic rate [36, 42]. Furthermore, it has been recently discovered that fluorescence-activating RNA aptamers are prone to misfolding [43], possibly contributing to slower and reduced signal generation relative to the amount of RNA transcribed. As expected, we observed that the InvadeR platform activates fluorescence visible in ˜10 minutes which is approximately 5-times faster than the RNA aptamer platform when using the equimolar amounts of the DNA template, TetR and aTc (FIG. 3f).

Overall, these results demonstrate that an aTF-based biosensor can be successfully interfaced with TMSD outputs, leading to immediate improvements in reaction speed.

Optimizing Invading RNA Designs for Different Biosensor Families

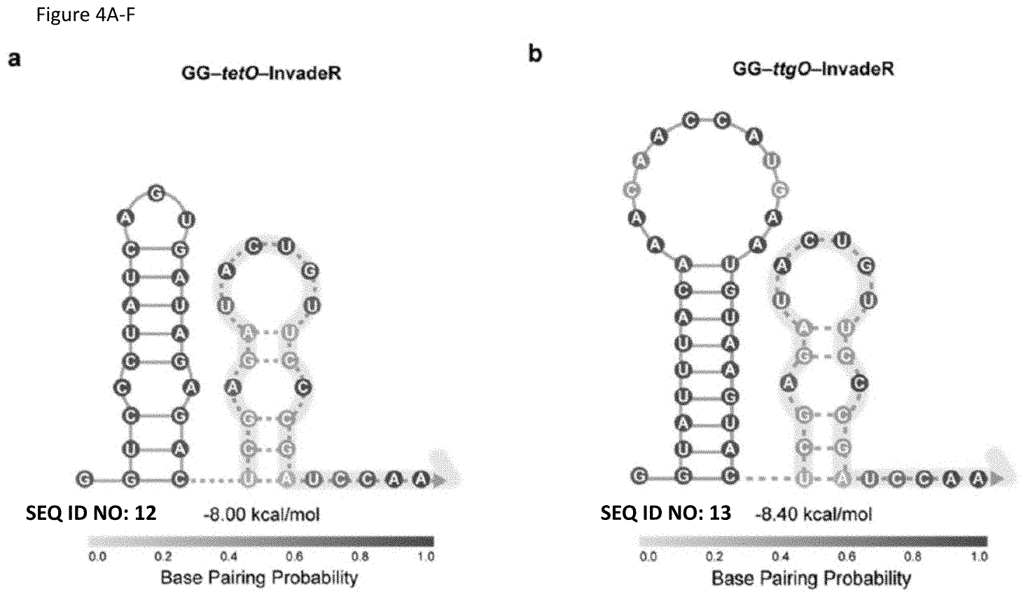

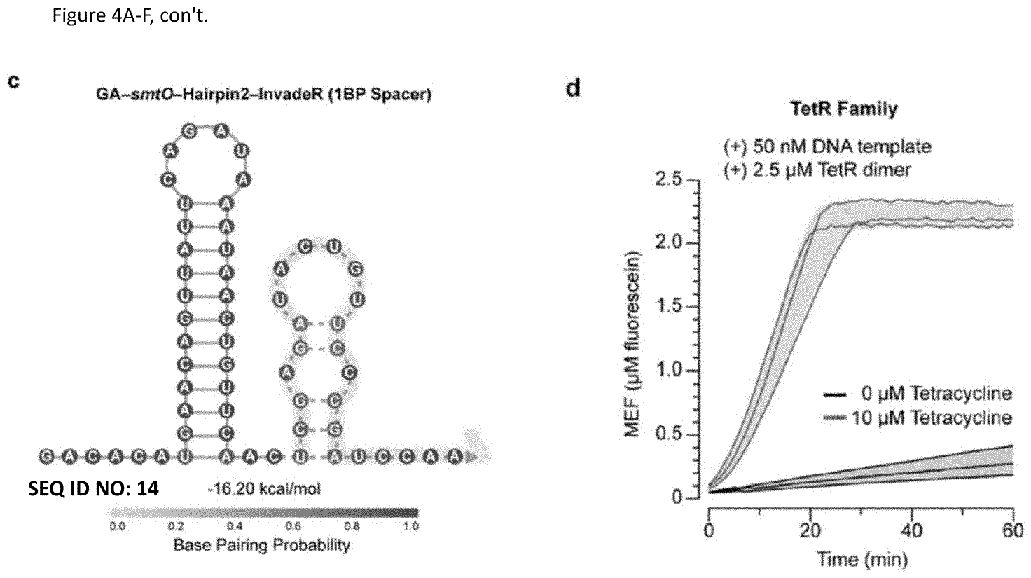

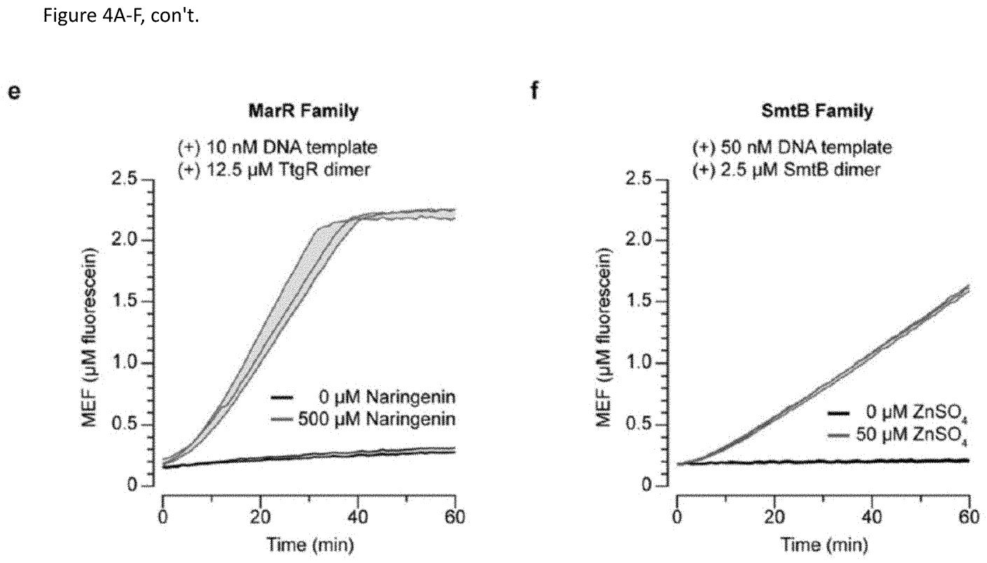

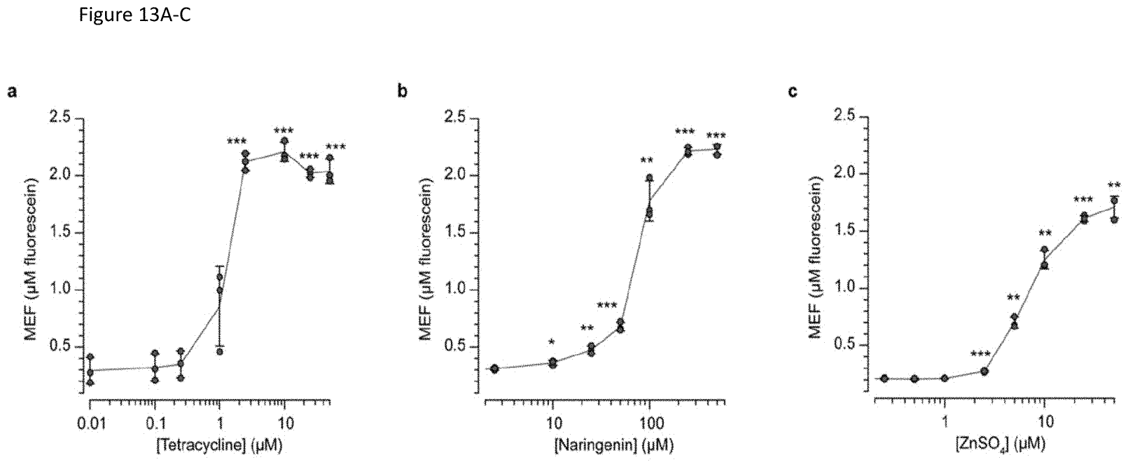

Having demonstrated the ability to regulate InvadeR with TetR, we next sought to determine whether the system is compatible with different families of aTFs to create biosensors for various classes of chemical contaminants. In addition to TetR, we chose TtgR [44] and SmtB [45] as representative aTFs of the MarR family [46] and SmtB/ArsR family [47], respectively. We placed the cognate operator sequence of each aTF 2-bp downstream of the T7 promoter and immediately upstream of the InvadeR sequences (FIG. 4a-c). When tetracycline was used to induce TetR-regulated reactions, we observed a strong and robust fluorescent signal visible in ˜10 minutes (FIG. 4d, FIG. 13a). Similarly, when a cognate ligand of TtgR, naringenin was added to TtgR-regulated reactions, we again saw robust fluorescence activation only in the presence of the ligand (FIG. 4e, FIG. 13b). We note that we observed a slight decrease in the induction speed from the TtgR-regulated reactions compared to that of the TetR-regulated reactions, likely due to the reduced ttgO DNA template concentration needed to accommodate TtgR's relatively weak binding affinity to its operator sequence [48].

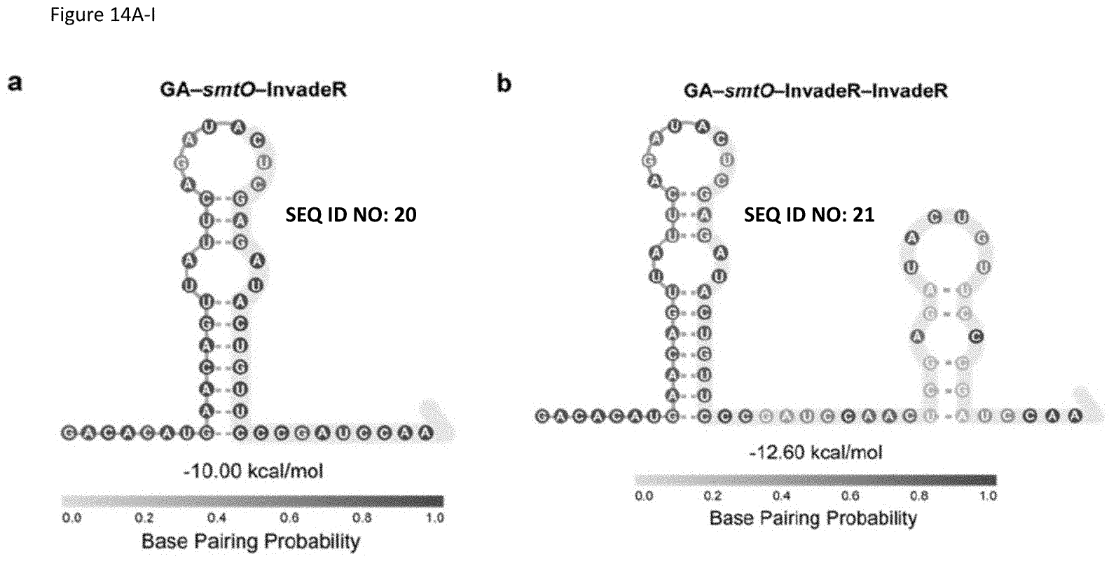

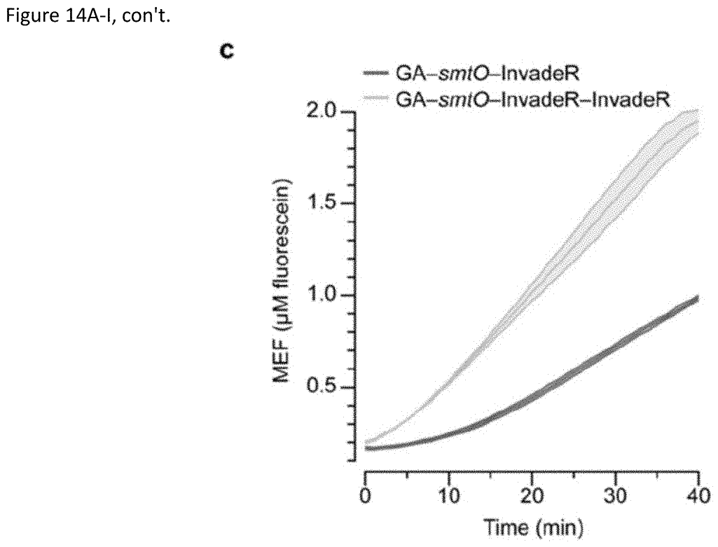

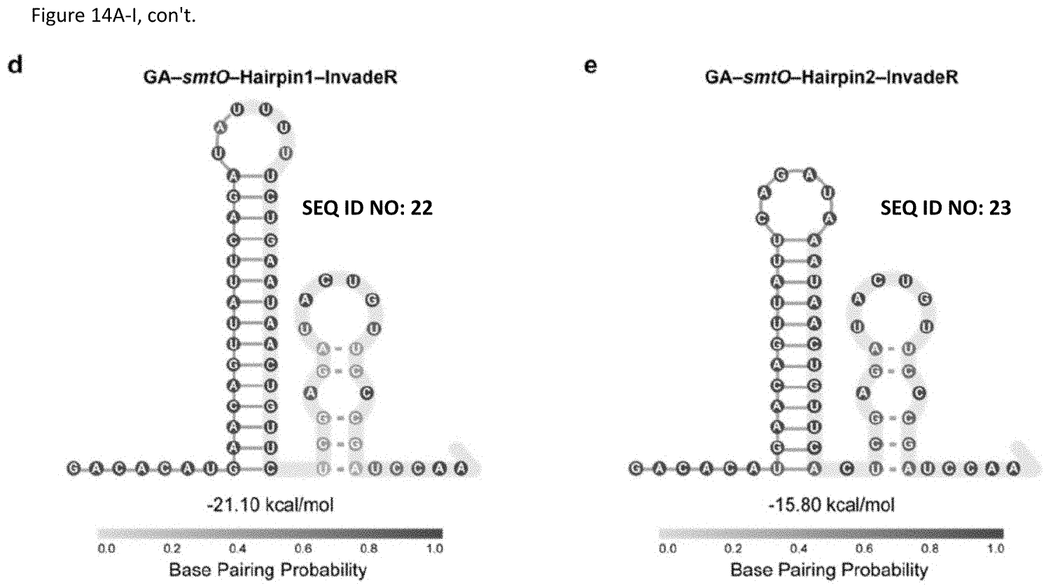

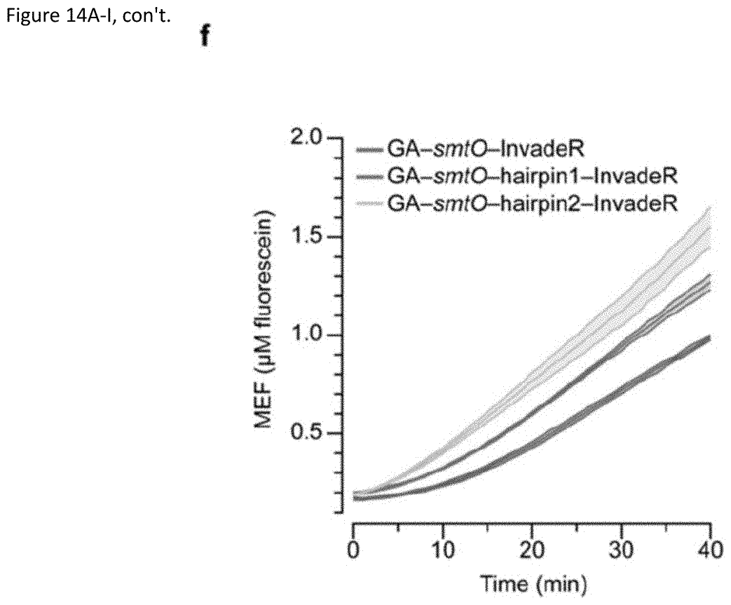

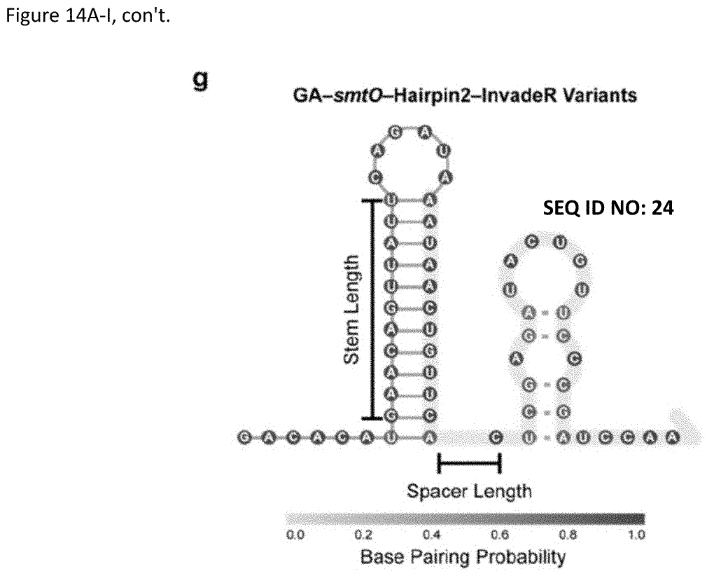

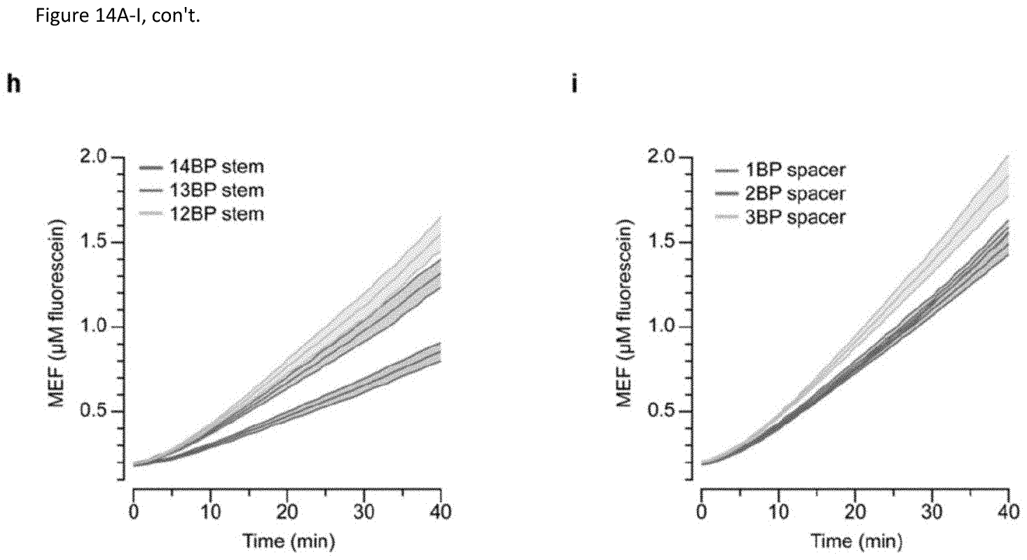

We were immediately successful in adapting the system to TetR and TtgR (FIG. 4a, b). In contrast, introducing the smtO sequence resulted in much slower reaction speeds, even in unregulated reactions (FIG. 14c). Interestingly, we observed that the smtO sequence is predicted to form a strong hairpin with the InvadeR sequence (FIG. 14a), which based on our results shown in FIG. 2 could impact reaction speeds. We hypothesized that adding extra sequences between the smtO and InvadeR sequences to prevent intramolecular smtO:InvadeR folding would improve the reaction kinetics. To test this hypothesis, we added several types of extra sequences between smtO and InvadeR, including an extra concatenated InvadeR sequence and several hairpin variants designed to directly sequester the transcribed smtO sequence (FIG. 14), and observed varying degrees of speed improvement from them (FIG. 14d-i). These results revealed that different design features of sequestering hairpins, such as the length of the predicted stem-loop of the hairpin or the spacer length between the hairpin and InvadeR may dictate the reaction kinetics. By optimizing these features, we created an improved design that showed clear activation of a SmtB-regulated reaction in the presence of ZnSO₄ with low background signal (FIG. 4c, f, FIG. 13c).

Together, these results demonstrate that the modularity of the ROSALIND platform is extensible to the TMSD platform (See Data Availability section for more information). They also reinforce that the secondary structures of the invading RNA strands play a critical role in determining reaction speed and revealed several design principles to improve the TMSD response speed.

Performing Logic Gate Computation with Cascaded TMSD Circuits

The interface of cell-free biosensors with TMSD creates a potentially powerful molecular computation paradigm for engineering devices that can perform programmed tasks in response to specific chemical inputs. This is especially true since TMSD circuits are much easier to program than protein-based circuits as a result of their simpler design rules [49], computational models that accurately predict their behavior [36, 37] and the emerging suite of design tools [24, 50]. We, therefore, sought to leverage these features of TMSD circuits to create an information processing layer for cell-free biosensors that could be used to expand their function.

As the first step towards this goal, we began with simple logic computation to process two different chemical ligands as inputs to the system. Previously, DNA-based TMSD circuits have demonstrated several approaches to building AND and OR logic gates. They typically involve engineering specific interactions between independent sequence domains to trigger a cascade of TMSD reactions—the final output strand then can interact with the DNA signal gate to activate fluorescence under the desired logic conditions with DNA inputs [12, 14, 15, 24]. We therefore thought to adapt this DNA-based logic gate architecture to build RNA-based TMSD circuits that can take chemical inputs, instead of nucleic acid inputs, to perform logic gate computation.

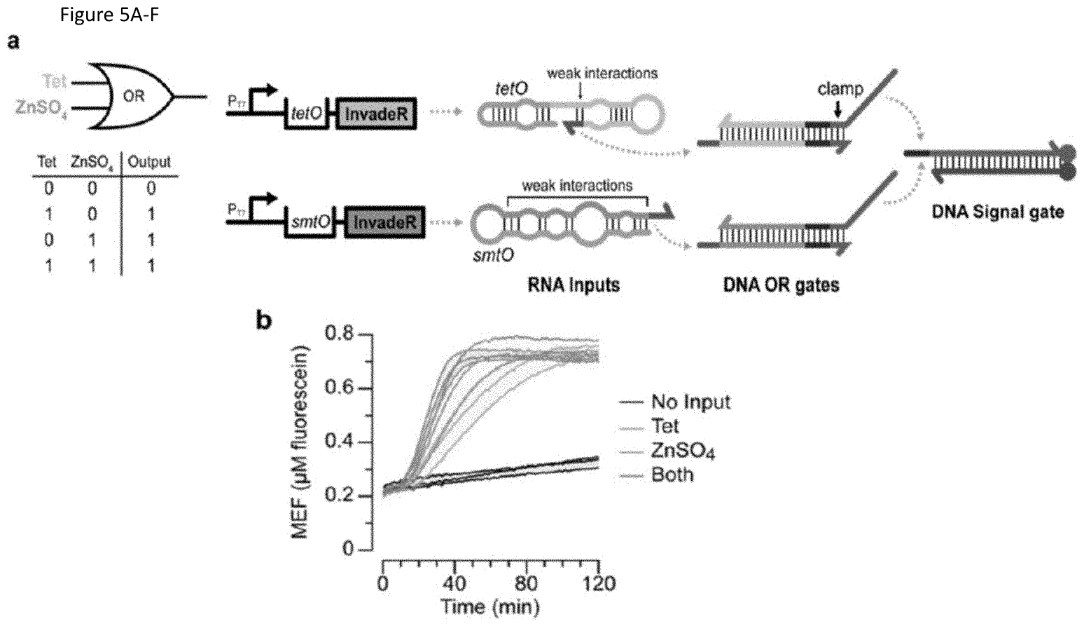

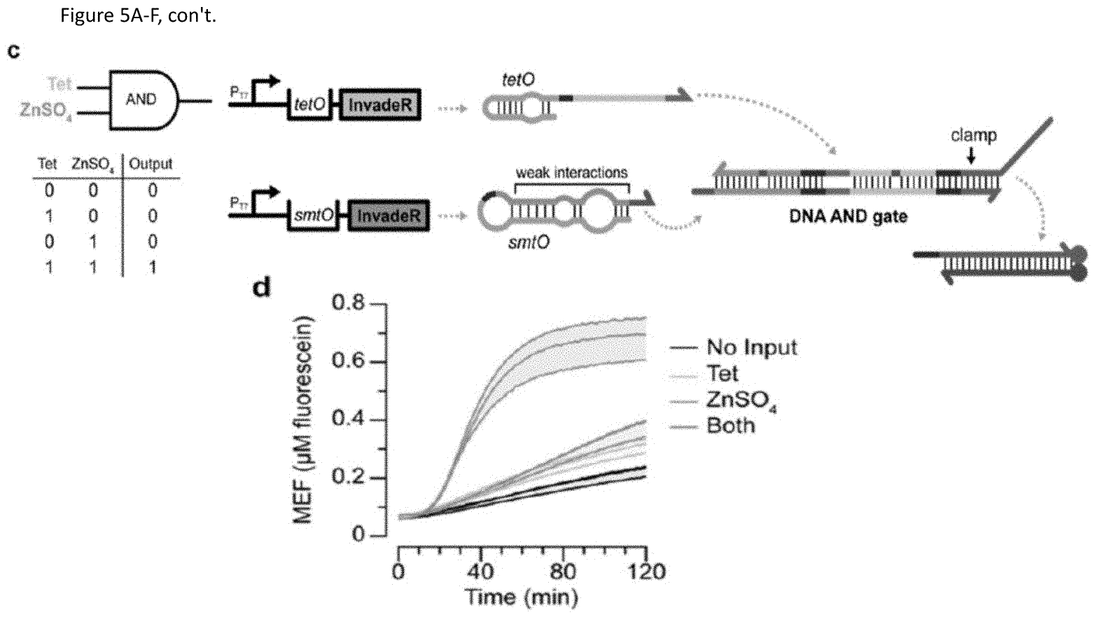

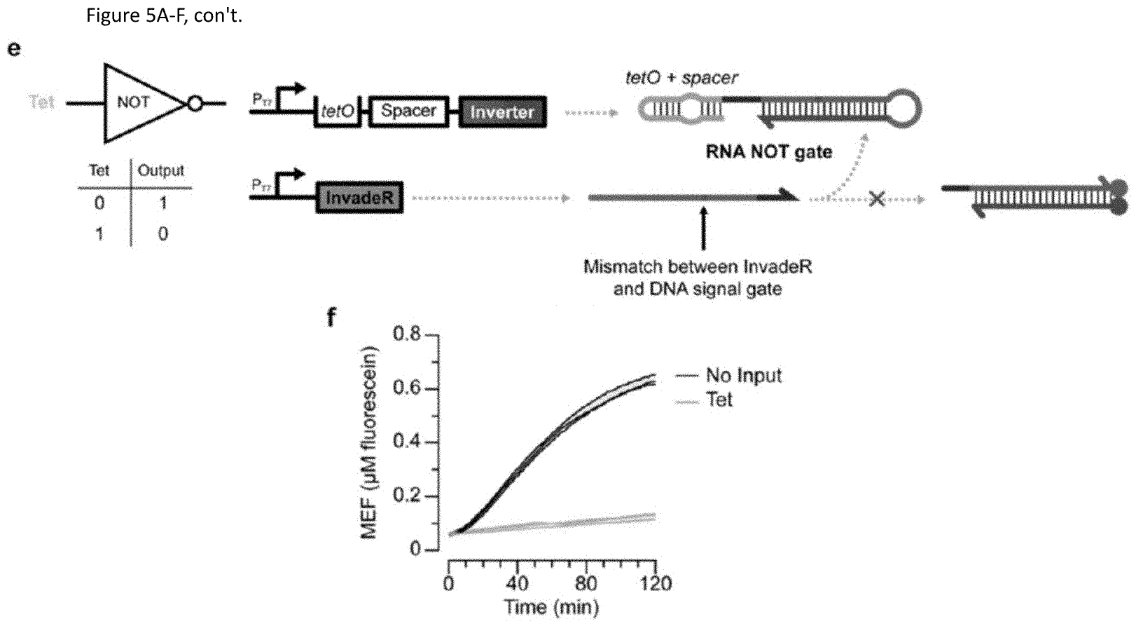

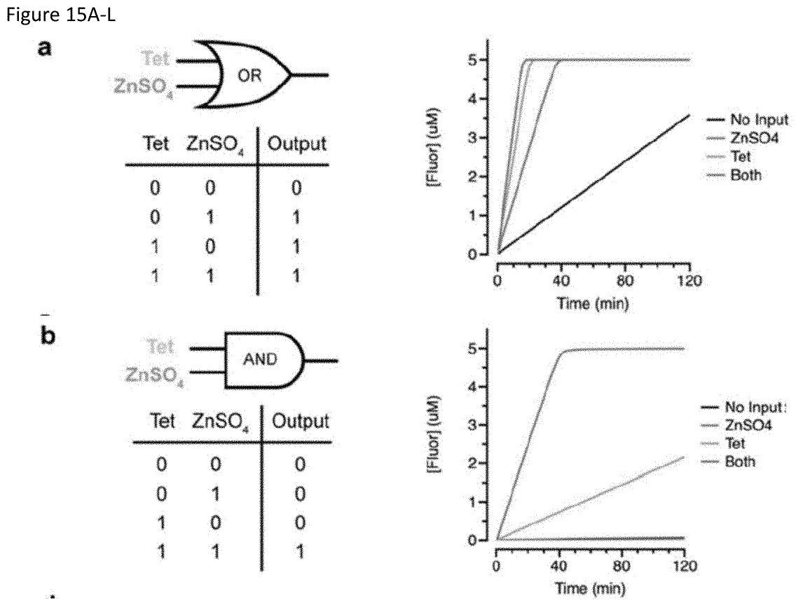

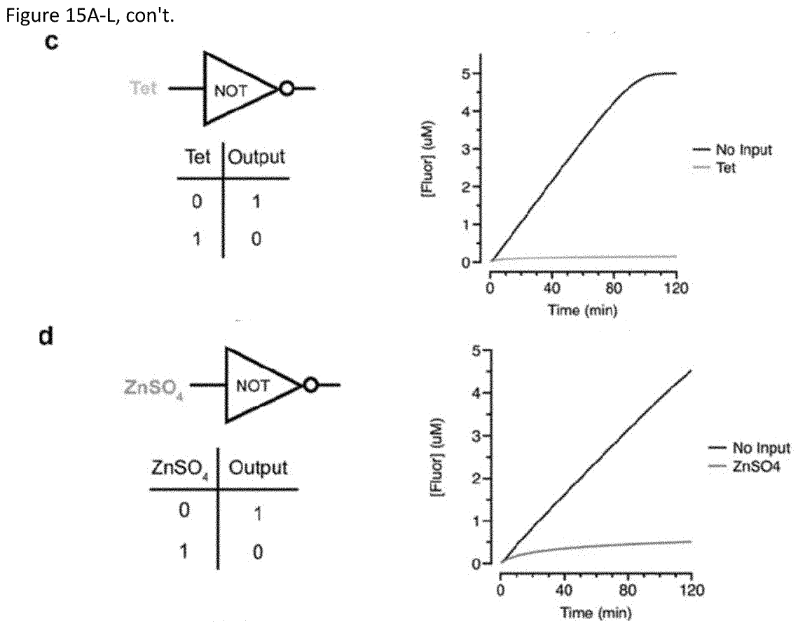

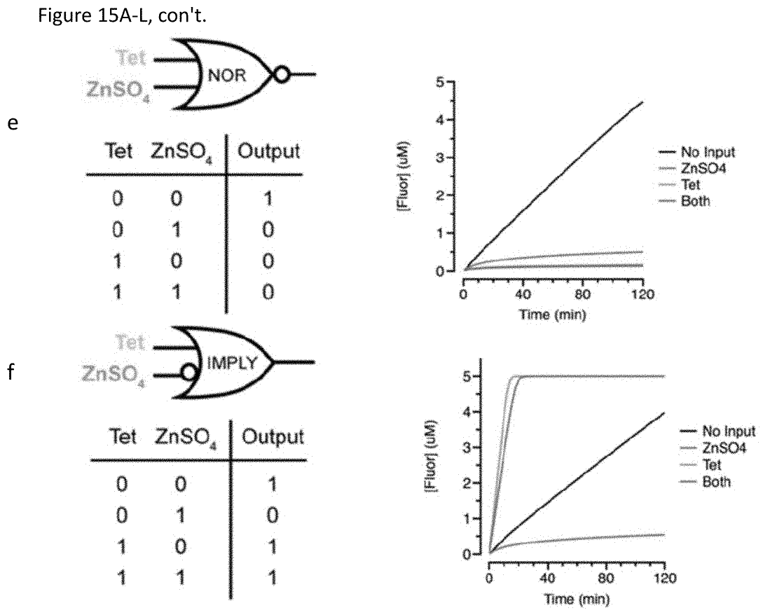

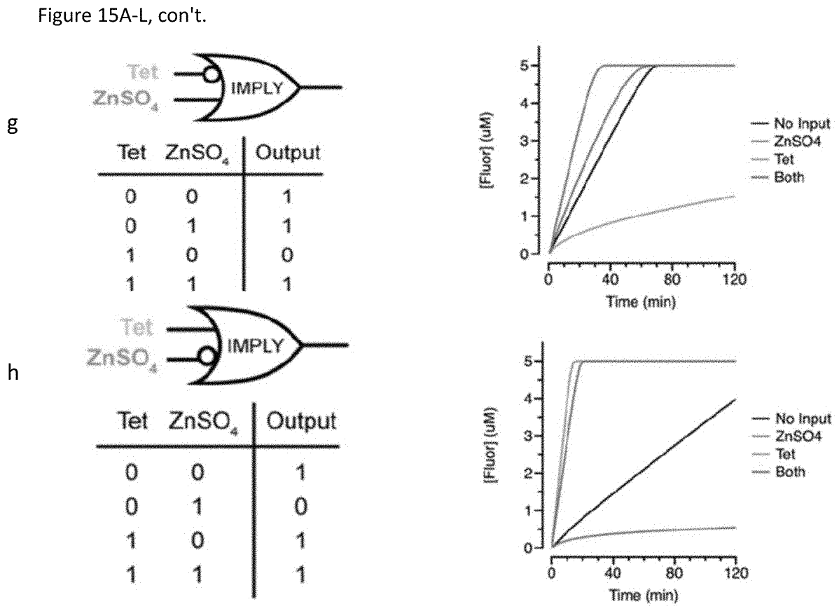

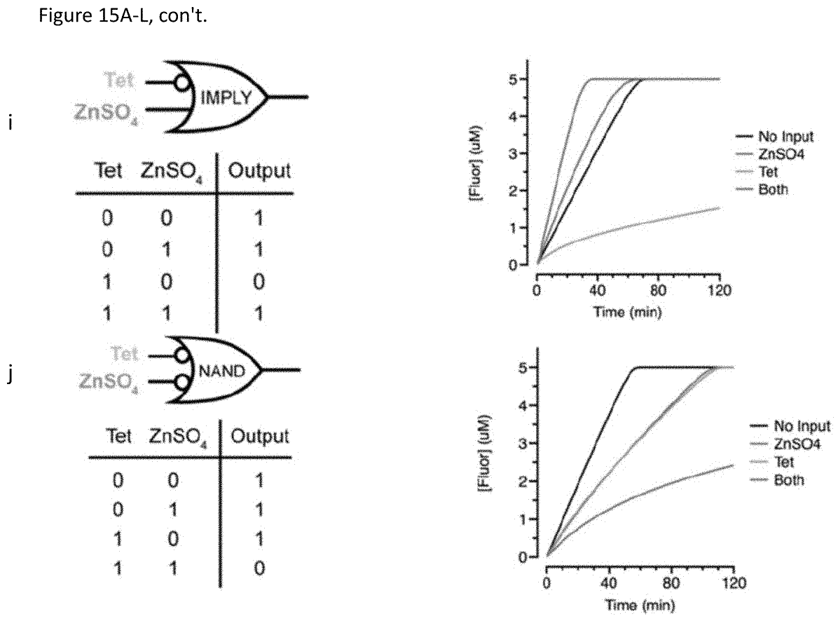

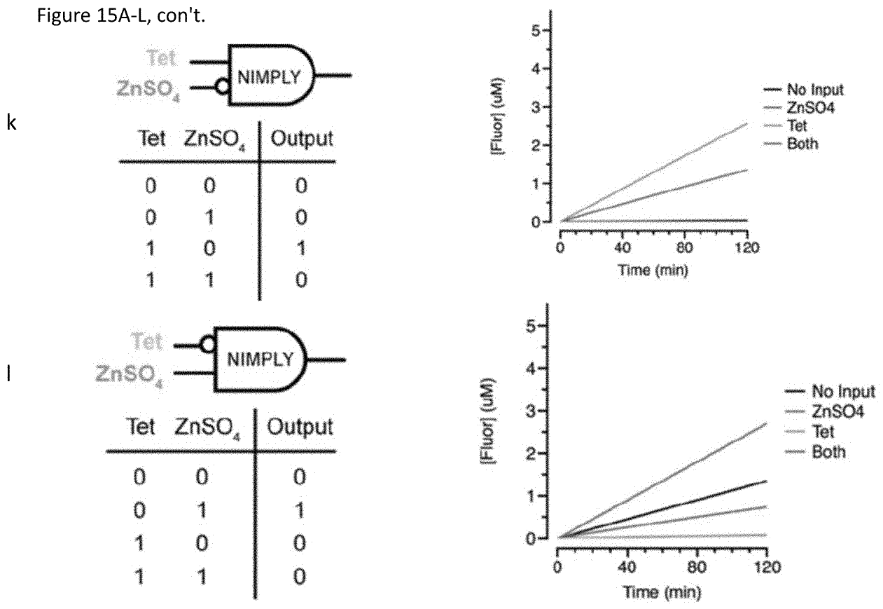

We started by constructing basic components of logic gates, namely OR, AND and NOT gates (FIG. 5). We implemented OR logic by designing DNA OR gates that act as an intermediate layer between InvadeR and the signal gate—once the transcription of InvadeR is triggered by a ligand input, InvadeR can perform TMSD on the corresponding DNA OR gate to release the top strand, which can subsequently invade the DNA signal gate to produce a signal (FIG. 5a). The sequences of the OR gates were either taken from a previous DNA-based TMSD work [24] or designed using NUPACK [32] The two DNA OR gates share the same output domain (green), but are activated by InvadeR strands regulated by different aTFs (TetR or SmtB). Including these DNA OR gates alongside DNA templates, TetR, SmtB and the signal gate led to fast signal generation except when no ligand inputs are introduced to the system, thus confirming OR logic computation (FIG. 5b). We note that the speed of fluorescence activation is slightly slower when only tetracycline is added likely due to the InvadeR secondary structure in the toehold region introduced by the tetO sequence (FIG. 5a). Modeling the OR gate using a set of ordinary differential equations (ODEs) that describe the reactions (IVT and TMSD) in the system (see Supplementary Method, below, for details on the ODE model used) matched the experimental results (FIG. 15a).

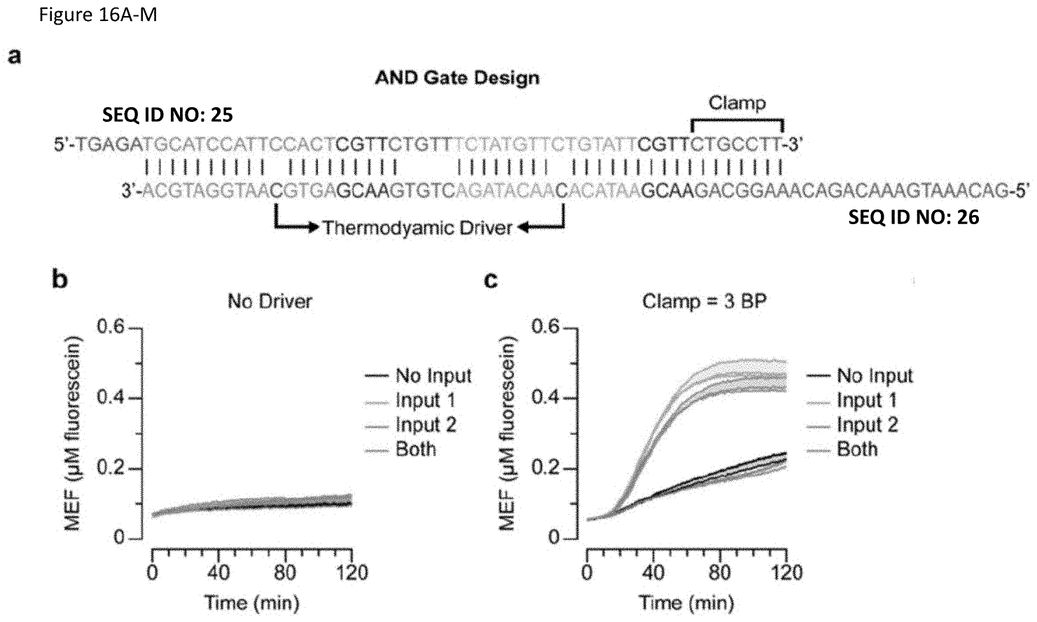

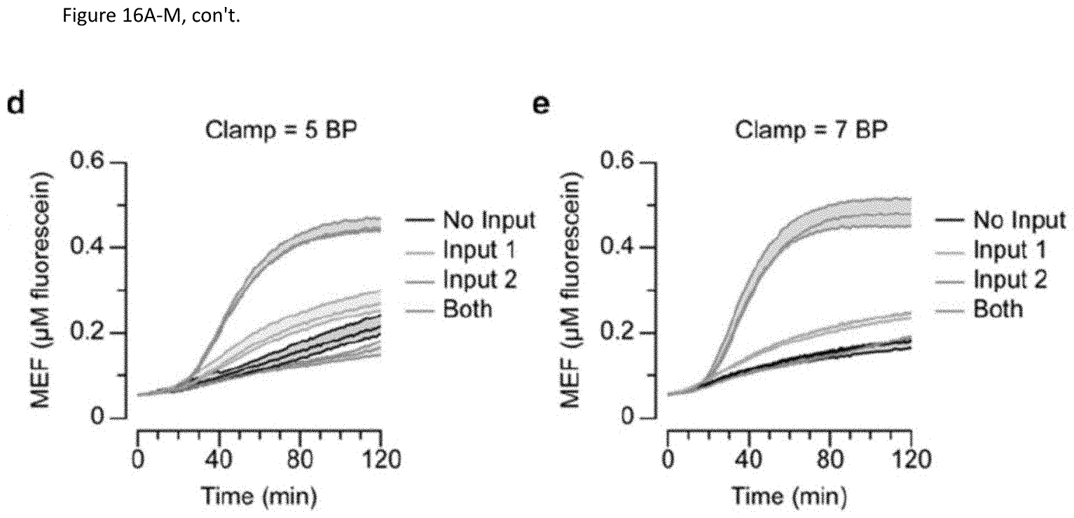

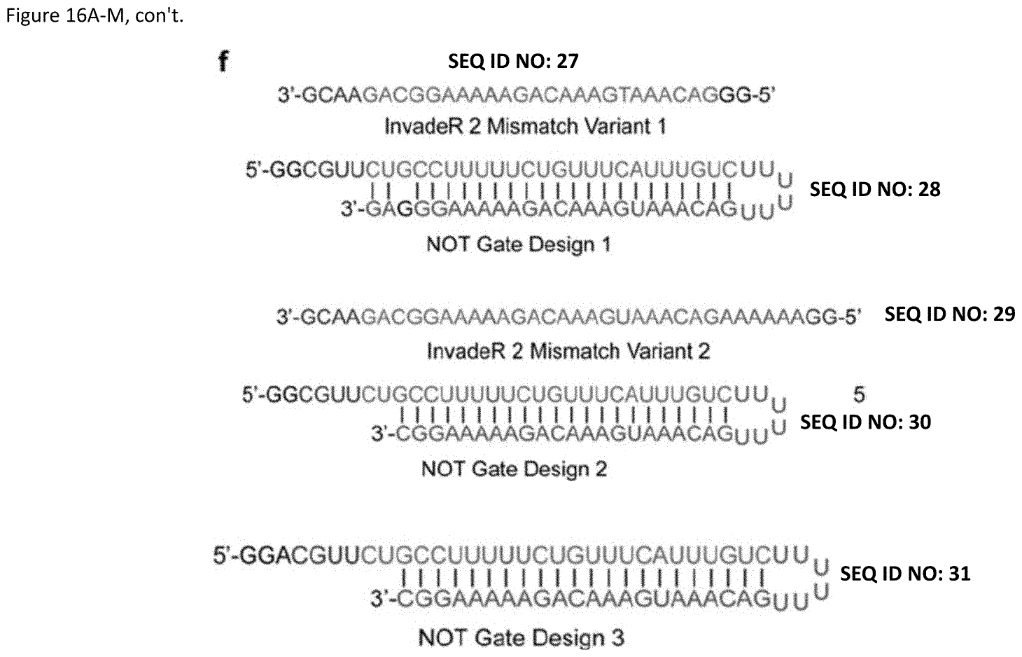

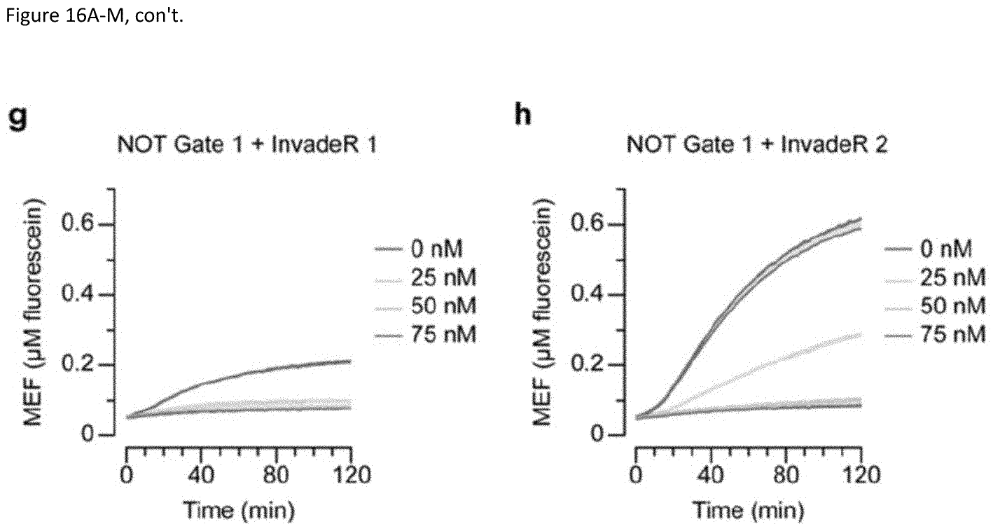

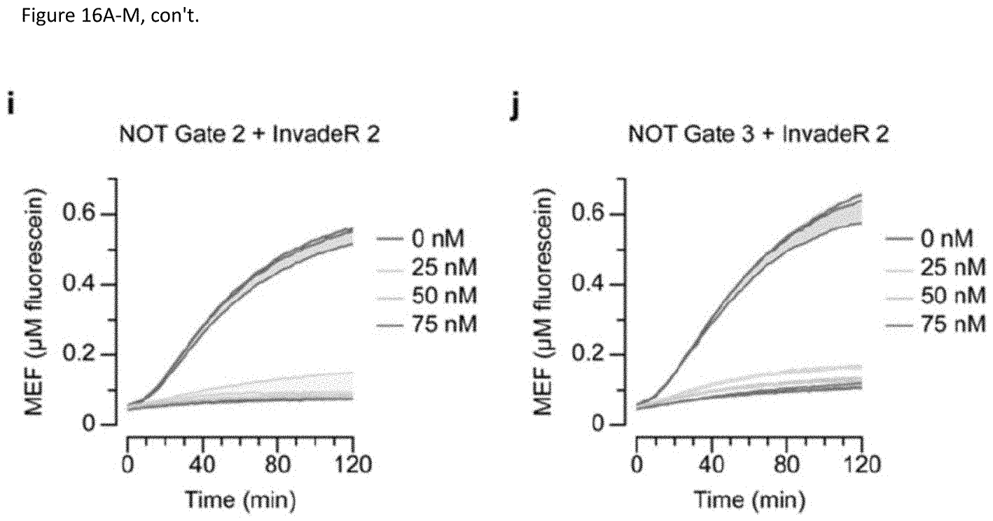

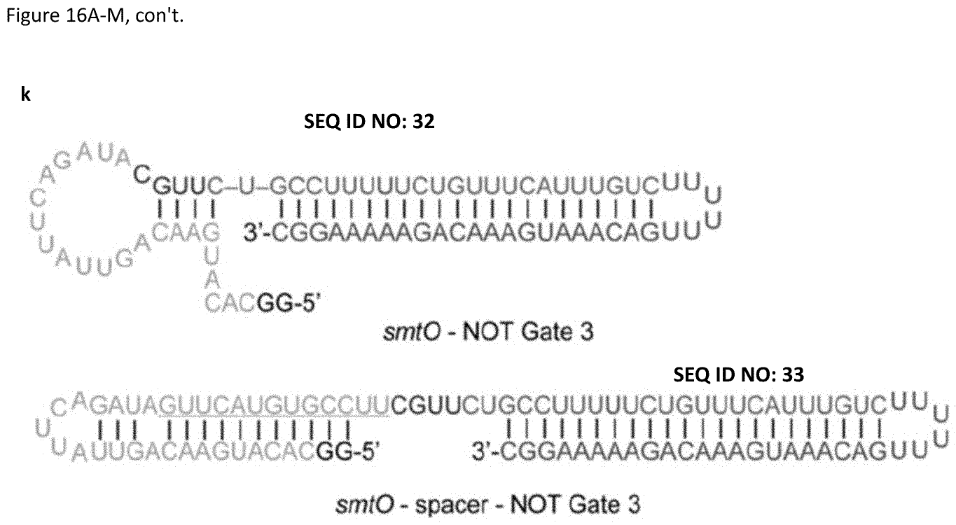

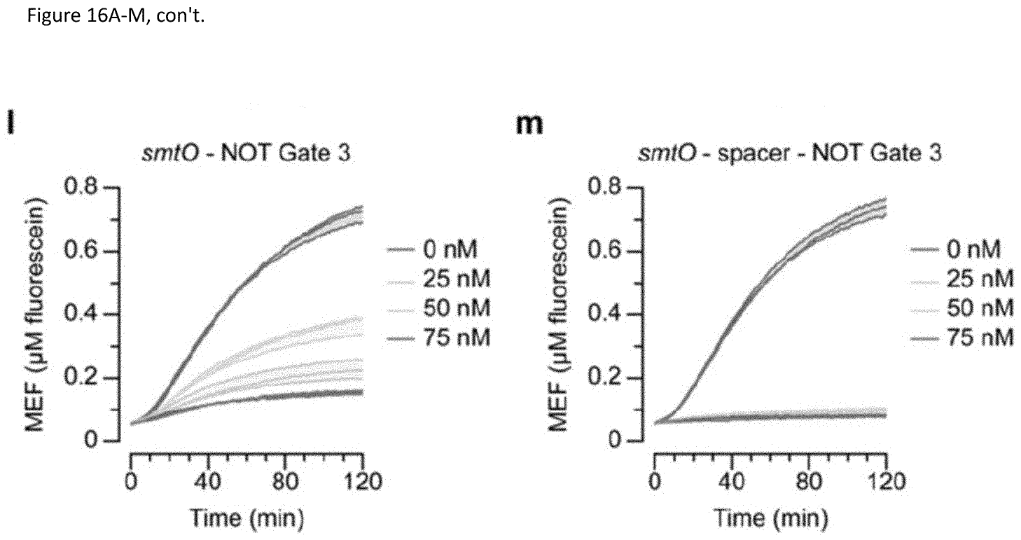

Next, we designed an AND gate by adapting a recently reported DNA AND gate design [30]. In this architecture, the AND gate includes three domains: domain 1 complementary to InvadeR 1 controlled by one aTF (blue), domain 2 complementary to InvadeR 2 controlled by the second aTF (orange) and domain 3 complementary to the DNA signal gate (green) (FIG. 5c). InvadeR strands that interact with the AND gate were designed to have different toehold sequences to minimize unwanted binding of the toeholds to the incorrect gate domain. Implementation of this architecture in sensing reactions using TetR and SmtB initially led to no fluorescence activation in any condition (FIG. 16b). To fix this, we incorporated two additional design features based on previous work—(1) mismatches in the AND gate that act as thermodynamic drivers for TMSD by the fully complementary InvadeR [51] and (2) a clamp in domain 3 of the AND gate which reduces leak caused by partial TMSD in the presence of Input 1 only [52] (FIG. 16a). With the thermodynamic driver mismatches installed, we observed signal activation but significant leak with the Input 1 only condition (FIG. 16c). By increasing the clamp length to 7 bp, we built an AND gate that requires both tetracycline and ZnSO₄ inducible InvadeR strands for signal generation and observed the expected computation pattern (FIG. 5d, FIG. 15b, FIG. 16e).

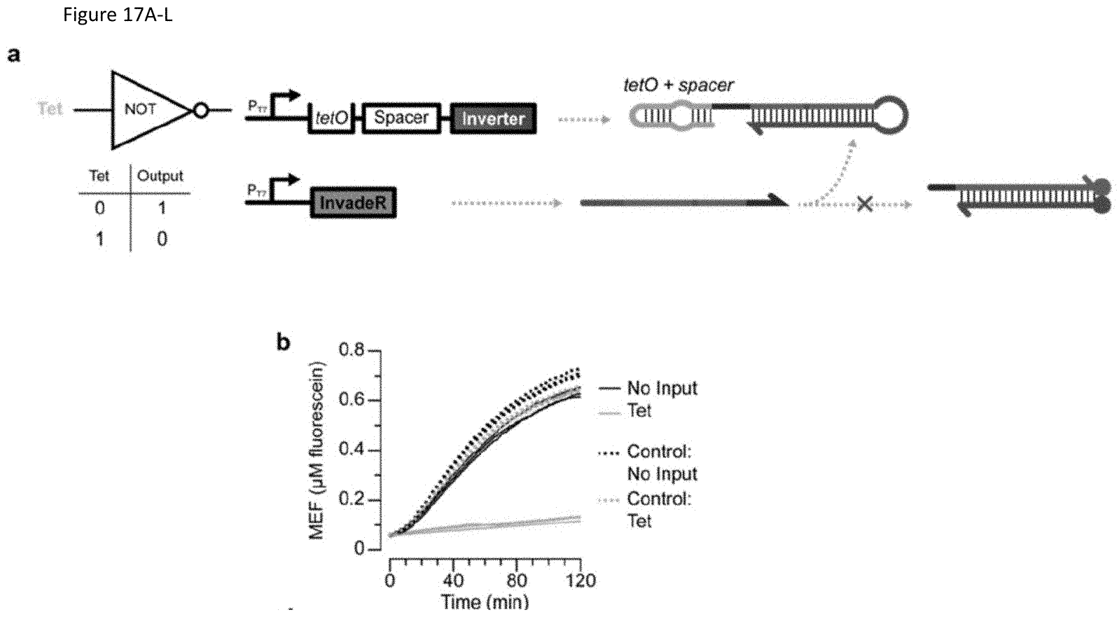

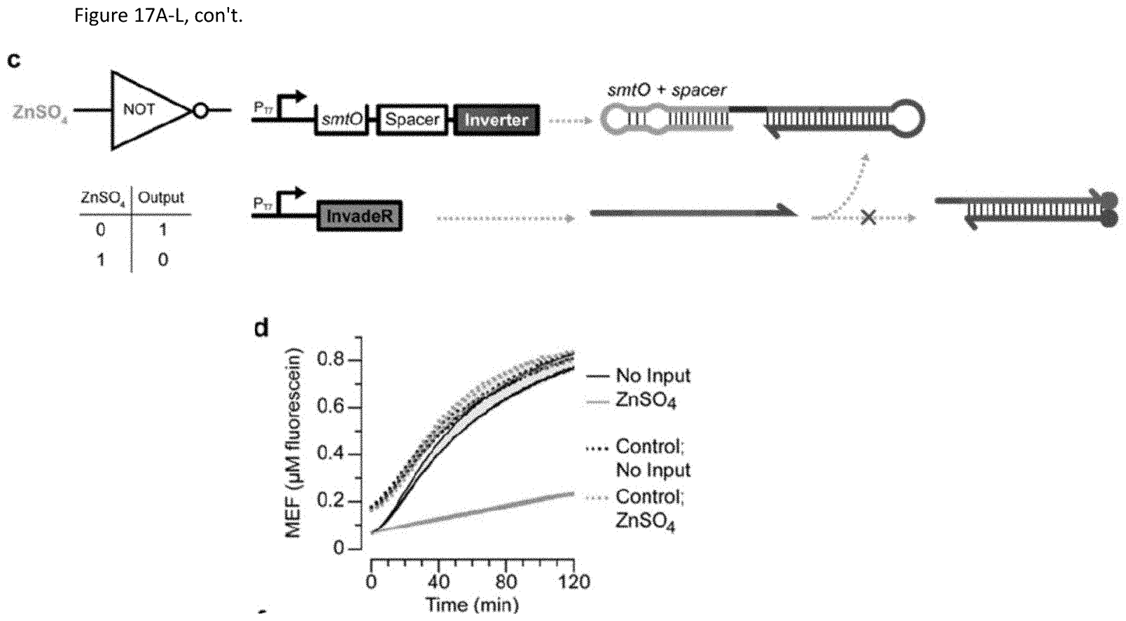

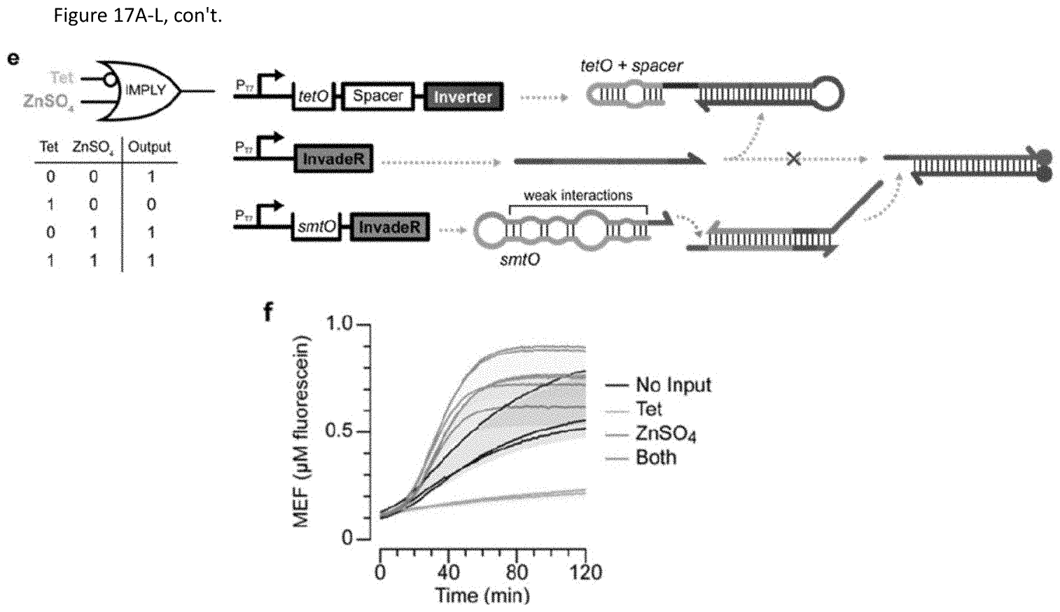

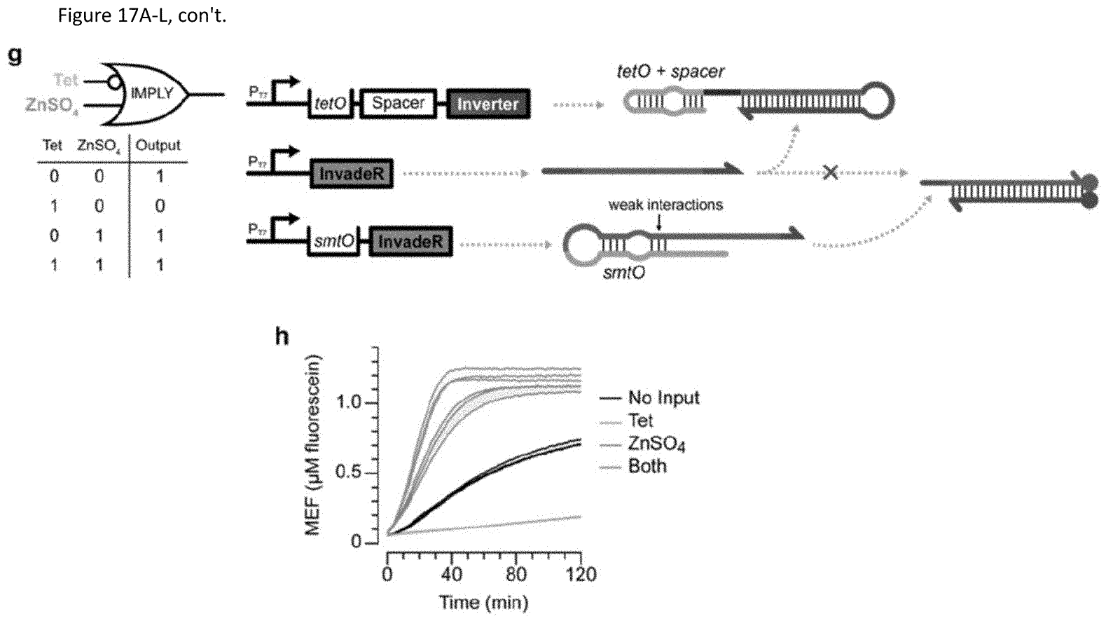

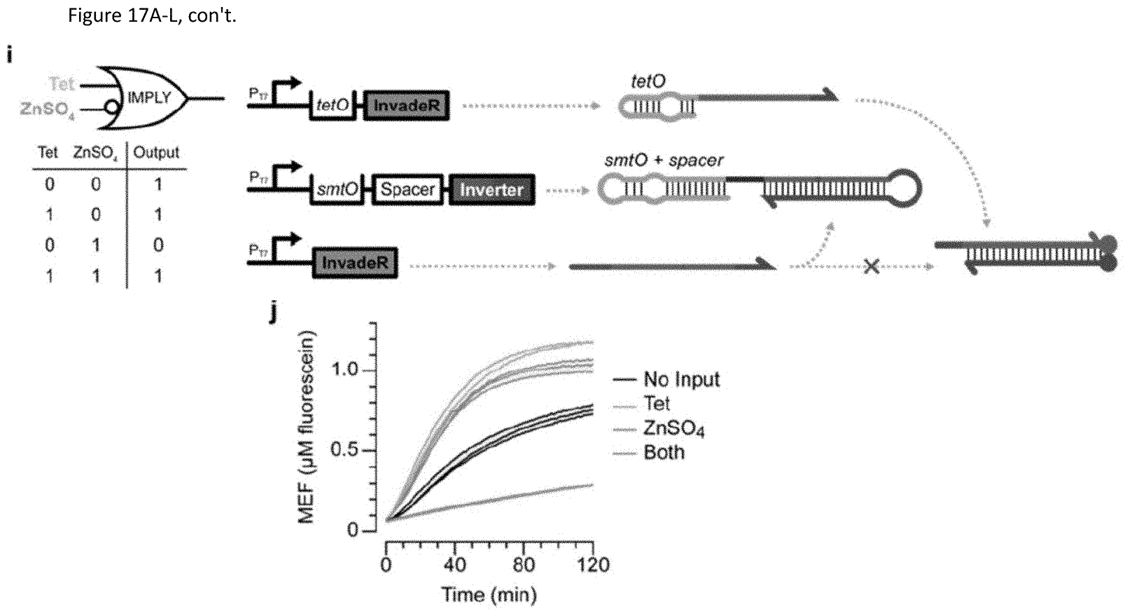

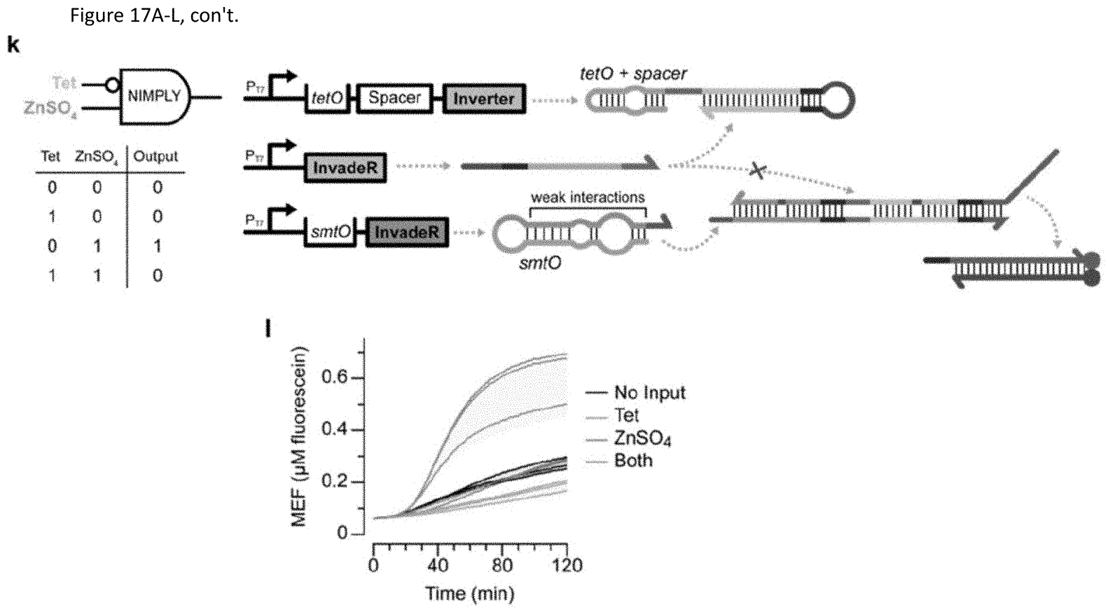

While AND and OR are important logic components, more complex logic gate computation requires a NOT circuit, which blocks signal in the presence of a ligand input. Such computation is a basic component of several logic gates including NOR and NAND, but it has not been extensively explored or applied in the context of TMSD circuits. To achieve signal inversion, we designed an RNA NOT gate that is capable of sequestering InvadeR away from the DNA signal gate (FIG. 5e). Adapted from a previous work of RNA gates [30, 53], this RNA NOT gate folds into a hairpin structure that mimics the DNA signal gate so that it can bind to and sequester InvadeR (FIG. 5e). To bias InvadeR binding to the RNA NOT gate, we included three design features: (1) a longer exposed toehold on the RNA NOT gate than on the DNA signal gate, (2) additional nucleotides within the loop of the RNA NOT gate that interact with InvadeR nucleotides and (3) a mismatch (highlighted in red) between InvadeR and the DNA signal gate (FIG. 16f-j). With this set of optimized design features, we incorporated an operator sequence in the DNA template encoding the RNA NOT gate to regulate its transcription with a ligand input. As discussed above, RNA-based TMSD poses unique challenges as the operator sequence introduces secondary structure that can prevent efficient TMSD reactions. To minimize the structural disruption of the RNA NOT gate, a spacer sequence was designed to form a hairpin with the tetO sequence (FIG. 5e). When 100 nM of the DNA template encoding the tetracycline-inducible RNA NOT gate was included with 25 nM of the DNA template encoding InvadeR, we observed a significant signal reduction in the presence of 20 tetracycline (FIG. 5f, FIG. 15c). To ensure that the signal reduction is not due to resource limitations from transcribing an extra DNA template, we designed and tested a control template whose sequence is shuffled from the TetR-regulated RNA NOT gate DNA template and observed no signal inversion (FIG. 17a, b). The same design architecture was applied to build a ZnSO₄-inducible RNA NOT gate (FIG. 15d, FIG. 16k-m, FIG. 117c, d).

These results demonstrate that with additional RNA-level design considerations, DNA-based TMSD logic gate architectures can be adapted to accommodate RNA strands whose transcription is induced by small molecule inputs in situ, thereby establishing a basis for building cascaded TMSD circuits for more complex logic gate computation.

Layering Gate Components to Perform Complex Logic Computation

With the three basic logic components established (OR, AND, NOT), we next sought to layer these components to enable more complex logic gate computation that form the basis of more sophisticated circuits, including NOR, NAND, IMPLY and NIMPLY.

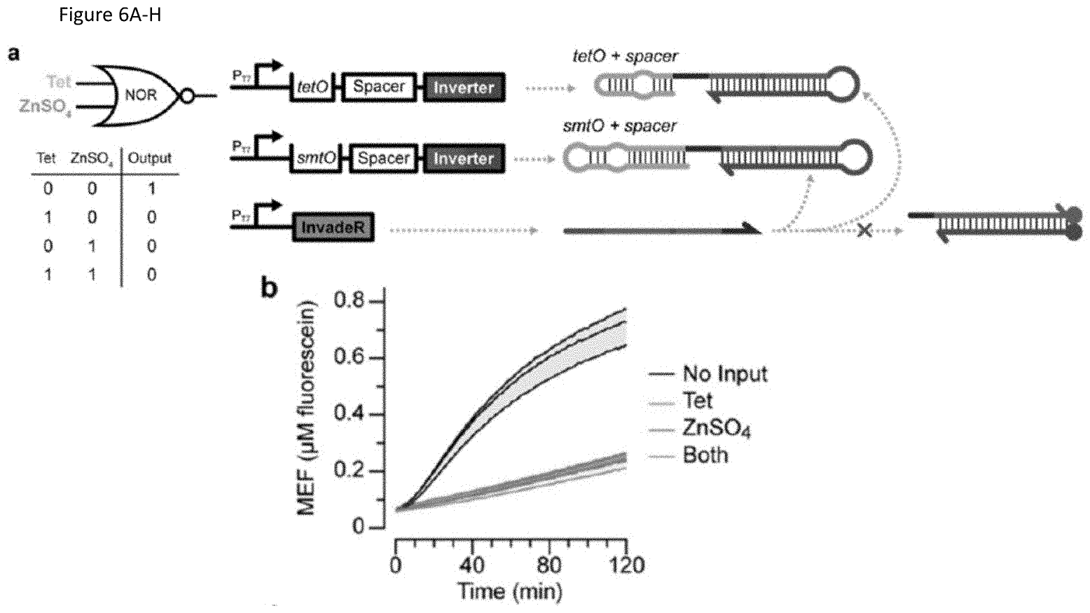

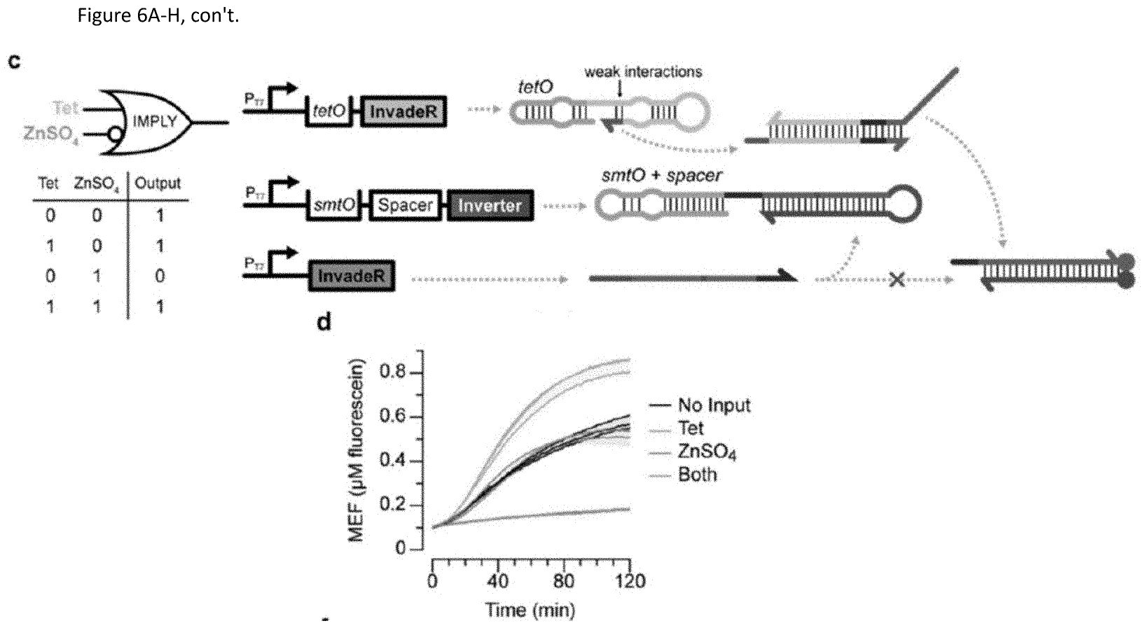

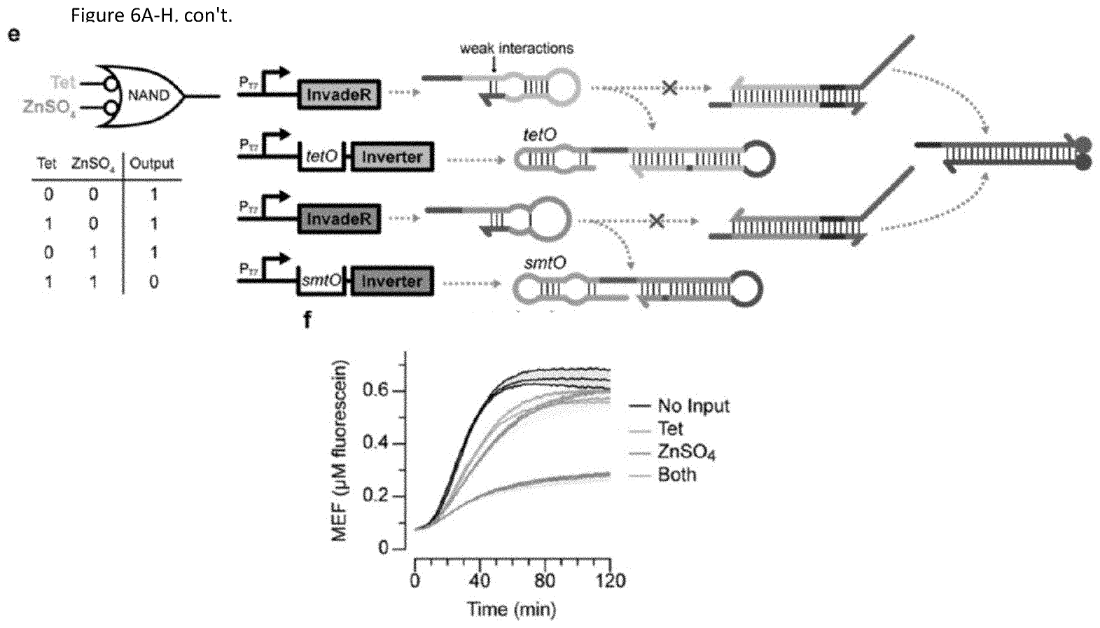

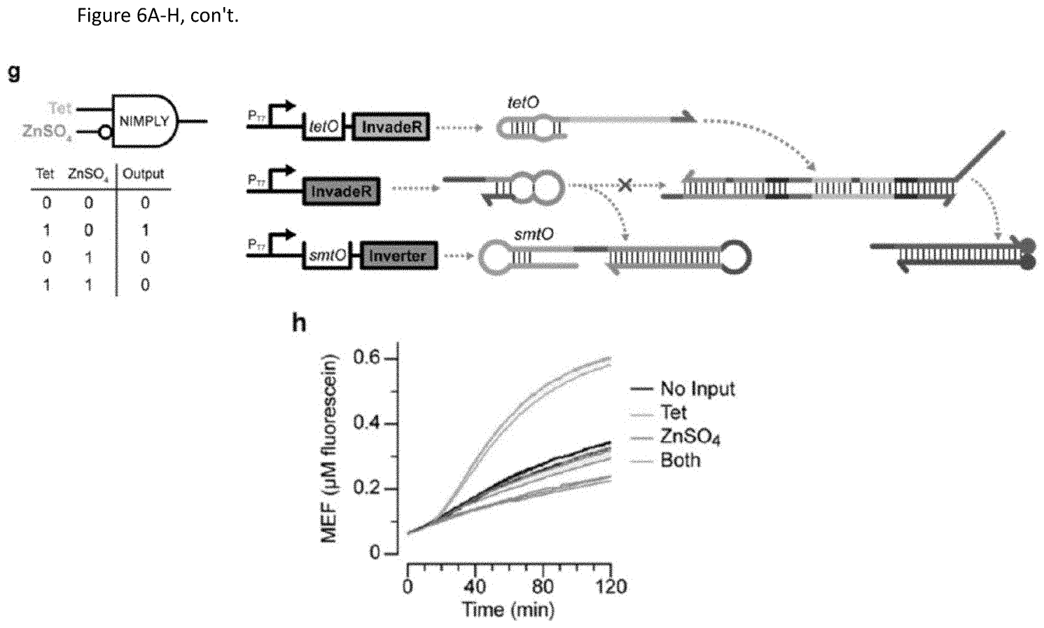

We began with NOR, an inversion of the OR gate that only generates signal when all inputs are absent, by combining two RNA NOT gates each regulated by TetR or SmtB (FIG. 6a). The NOT gates were designed to share the same domain as the DNA signal gate so that either ligand would produce an RNA NOT gate that can sequester constitutively expressed InvadeR molecules. With this architecture, we observed fluorescence activation only in the absence of both inputs, as expected (FIG. 6b, FIG. 15e).

Next, we focused on the A IMPLY B architecture, which has a truth table whose output is always on except under the condition in which A is present and B is absent. The ZnSO₄ IMPLY tetracycline gate was built by layering the tetracycline-induced DNA OR gate with the ZnSO₄-induced RNA NOT gate (FIG. 6c). When implemented, the gate generated signal in all input conditions except when only ZnSO₄ is present as expected (FIG. 6d, FIG. 15f). However, we observed differences in kinetics of fluorescence activation where the tetracycline only condition produced a faster and greater fluorescent signal than the other input conditions that generate signal. This is likely due to the built-in mismatch between the constitutively expressed InvadeR strand and the DNA signal gate (highlighted in red), making its TMSD inefficient compared to that of the tetracycline-induced OR gate output strand. Similar effects were observed from the tetracycline IMPLY ZnSO₄ gate as well, though to a lesser degree (FIG. 15g, FIG. 17e, f). Interestingly, IMPLY gates can also be built without the DNA OR gate, which allows direct interactions between the ligand-induced InvadeR strand and the DNA signal gate (FIG. 15h, i, FIG. 17g-j). This design enables faster signal generation when both inputs are present as it eliminates the need for two cascaded TMSD reactions. The ability to design alternative logic gate architectures highlights the platform's programmability and modularity when building circuits for downstream signal processing.

We then constructed a NAND gate which combines NOT and AND gates to produce signals in all conditions except when both inputs are present. We explored two design options for the NAND gate: (1) inversion of an AND gate output strand (A NAND B=NOT (A AND B)) and (2) the combination of two RNA NOT gates being integrated as inputs into a DNA OR gate (NOT (A AND B)=NOT A OR NOT B). The first design scheme requires the AND gate output strand to form a NOT gate hairpin structure upon being strand-displaced by both InvadeR strands. This poses a sequence constraint where the two domains on the AND gate need to be complementary to each other. Because of this complexity, we instead chose to build the NAND gate using the second design option (FIG. 6e). This design involves the DNA signal gate, two DNA OR gates and four different transcription templates—two unregulated templates each encoding InvadeR for each DNA OR gate and two regulated templates each encoding the RNA NOT gate capable of sequestering its respective InvadeR. The RNA NOT gates were built the same way as previously described with one design change. Instead of introducing a bp mismatch between InvadeR and the DNA signal gate, we built in a thermodynamic driver in the RNA NOT gate (highlighted in red) to favor the TMSD reaction of InvadeR with the RNA NOT gate over that with the DNA signal gate (FIG. 6e). This change prevents a slower response speed caused by the mismatch between InvadeR and the DNA signal gate as observed in the IMPLY gate architecture. In this architecture, both tetracycline and ZnSO₄ are required to prevent signal generation from the unregulated RNA inputs, which matches the pattern we observed (FIG. 6f, FIG. 15j).

Finally, we designed the A NIMPLY B gate, which combines AND and NOT gates to implement A AND NOT B logic, producing an output only when input A is present alone. The specific NIMPLY gate design shown in FIG. 6g uses an RNA NOT gate regulated by the ZnSO₄ input alongside a DNA AND gate that requires both unregulated InvadeR and tetracycline-induced InvadeR for activation. When implemented, both the ZnSO₄ NIMPLY tetracycline gate as well as the tetracycline NIMPLY ZnSO₄ gate performed the expected logic gate computations (FIG. 6g, FIG. 15k, 1, FIG. 17k).

Together, these results show that basic logic gate components can be combined and layered to perform more complex molecular computation using small molecules as inputs to the system. Specifically, the novel development of an RNA NOT gate architecture enabled the constructions of four different logic gates, namely NOR, IMPLY, NAND and NIMPLY.

Using a TMSD Circuit Processing Layer to Quantify Biosensor Outputs

Two-input logic gate computations with small molecule inputs are a powerful demonstration of the platform's programmability for information processing. To demonstrate a practical application of such an information processing layer, we next chose to focus on quantifying biosensor outputs. In typical cell-free biosensing systems, the sensor layer is wired to the output layer (FIG. 1), thus directly coupling the amount of output signal to the properties of the biosensor. In many cases, this results in an output signal that is generated above a specific detection threshold which is determined by the aTF-ligand and aTF-DNA binding constants [54], making it difficult to obtain information about the input target compound concentration. One approach to solving this challenge is to configure reactions to generate a proportional output response [3], though this system requires users to judge output intensity or hue to estimate the input target concentration which can lead to uncertainty. Alternatively, we chose to create a system similar to an analog-to-digital converter (ADC) circuit—widespread in electronic systems that interface sensors to information processing modules [55]— that creates a series of binary outputs that encode the analog input concentration of the target compound (FIG. 18).

To construct a genetic ADC circuit, we first needed to create a comparator circuit—a building block of ADCs that produces a “True” binary output when the input is above a pre-defined threshold. ADC circuits can then be built by creating a series of comparators, each with different thresholds. Previously, this concept of thresholding was implemented in in vitro DNA-only TMSD circuits to act as a low-level noise filter [12, 24]. Thresholding can be implemented in TMSD because the reaction kinetics of strand displacement can be precisely increased by lengthening DNA gate toehold regions [17] (FIG. 7a). Specifically, each additional nt added to a toehold region enhances strand displacement kinetics by 10-fold [36]. As a result, additional DNA gates with longer toeholds can be designed to preferentially react with inputs, thus only allowing DNA signal gates to be activated when the input strand completely consumes the longer-toehold DNA gates (FIG. 7a).

Our first step was to build a similar thresholding circuit but using input RNA strands generated in situ. The DNA threshold gate was designed to contain two strands: an identical strand to the fluorophore strand of the signal gate and a shortened complementary strand to allow a longer 8-nt toehold compared to the 4-nt toehold of the signal gate (FIG. 7a). Additionally, the threshold gate lacked the fluorophore and quencher modifications. In this design, InvadeR should react preferentially with the threshold gate with orders of magnitude increased rates, preventing InvadeR from interacting with the signal gate. Only after the threshold gate is completely exhausted can InvadeR efficiently strand-invade the signal gate to generate a fluorescent signal. We reasoned that by tuning the amount of the threshold gate present in the reaction, we can precisely control the time at which InvadeR activates fluorescence from the signal gate. Modeling this kinetic behavior using a set of ODEs showed that this is indeed the predicted behavior of the setup (FIG. 7b). We then tested these reactions experimentally and observed quantitative agreement with the model predictions (FIG. 7b). In this way, a thresholded TMSD reaction acts as a “kinetic” comparator circuit—for a given input, the time at which signal generation occurs is proportional to the amount of the threshold gate added to the reaction.

Next, we sought to create a series of biosensing TMSD comparator circuits to act as an ADC for ligand concentration. Specifically, we prepared a strip of reactions where each tube contains a different amount of the threshold gate. By adding the same input ligand concentration to each tube and observing the output at a specific time point, a user can observe which tubes in the series were activated to obtain semi-quantitative information about ligand concentration (FIG. 7c). For example, since a higher ligand concentration is required to overcome a higher threshold value, we expected to observe different numbers of activated tubes depending on the input concentration—the higher the input, the greater the number of activated tubes in the series.

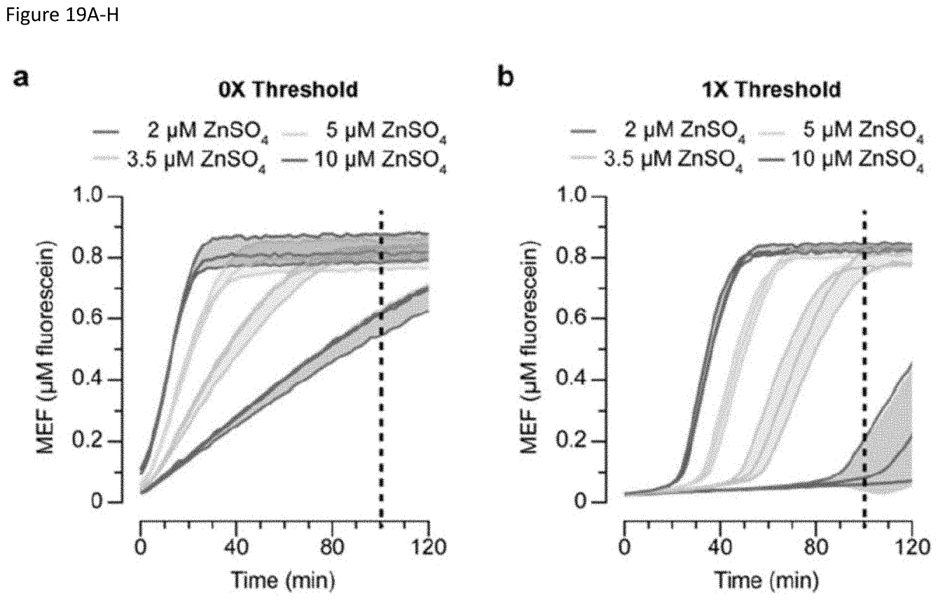

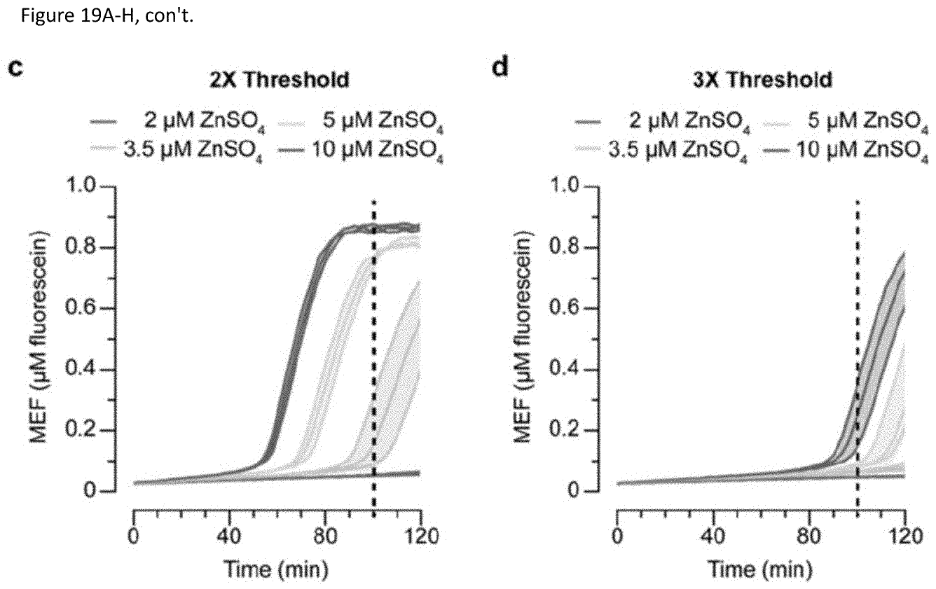

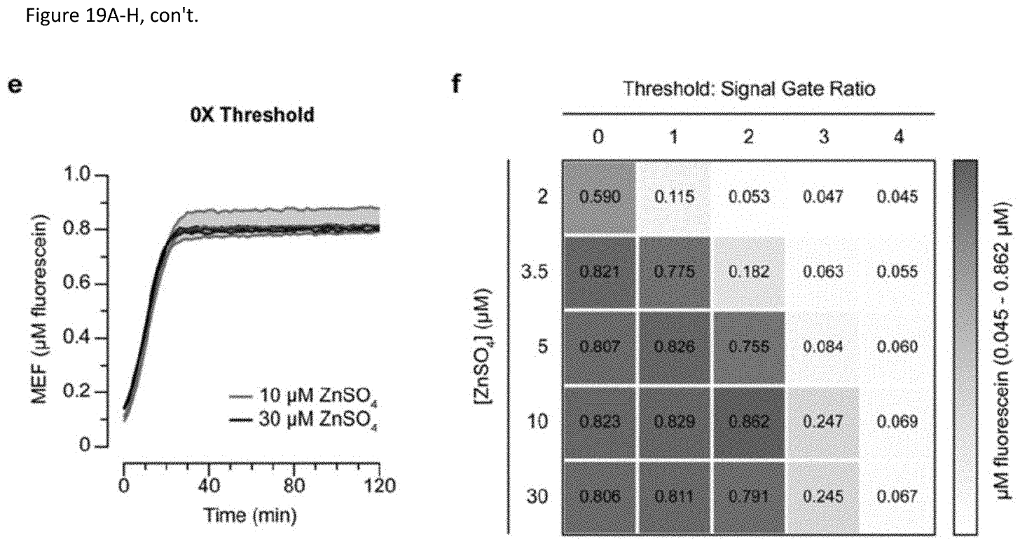

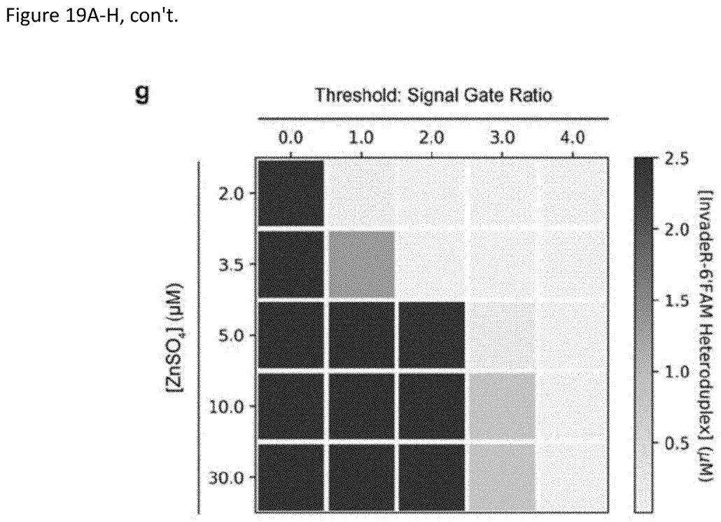

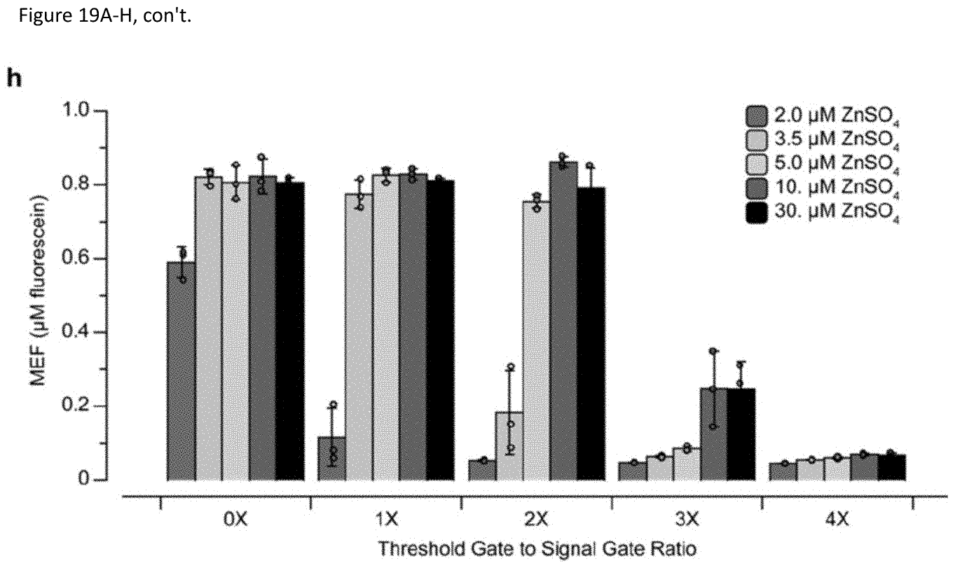

We first built a model for the system to determine the feasibility of the approach using the same set of ODEs used in FIG. 7b with the addition of aTF-DNA and aTF-ligand binding kinetics, focusing on zinc sensing with SmtB because of its relevance in municipal water supplies [56]. We used simulations to determine the threshold gate concentrations needed to activate one, two, three or four tubes after 100 minutes corresponding to zinc concentrations of 2 μM, 3.5 μM, 5 μM and 10 respectively (FIG. 7d). Here, we define a tube with its MEF value higher than 0.5 as “True” or “ON” since the visible threshold is around the indicated value. We then proceeded to build this genetic ADC circuit with the SmtB-regulated TMSD reactions. Four ADC reaction sets were built using the threshold gate concentrations simulated in FIG. 7d, and each set was tested with either 2 μM, 3.5 μM, 5 μM or 10 μM ZnSO₄. When the reactions were run for 100 minutes, we saw the expected pattern of signals where the number of activated tubes in the series increased with higher input ZnSO₄ concentrations (FIG. 7e, FIG. 19a-d). This implementation allows a user to determine the unknown input zinc concentration range by directly reading out the sequence of activated tubes.

While simple, this demonstration represents the potential of TMSD circuits as an information processing layer to expand the functionality of cell-free biosensors where the circuits transform an analog input signal into a digital readout to increase ease of interpretation and information content of the output signals

### DISCUSSION

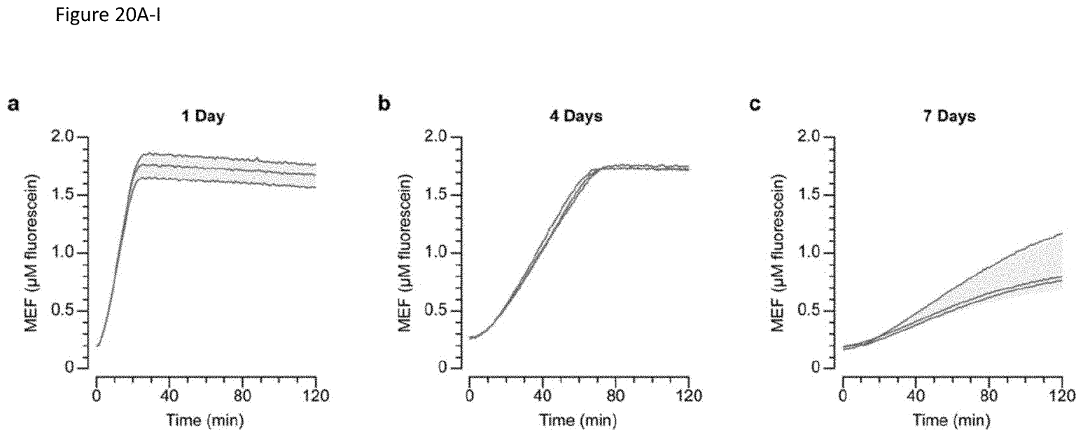

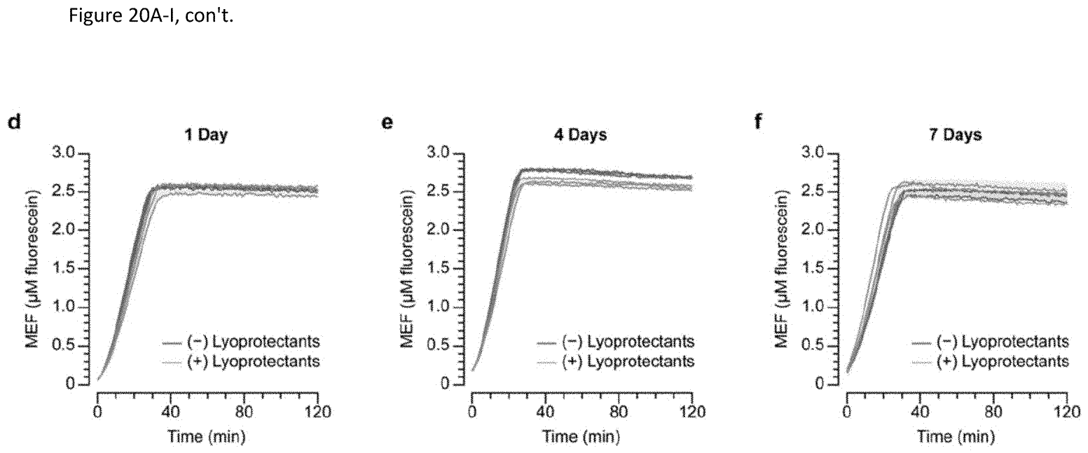

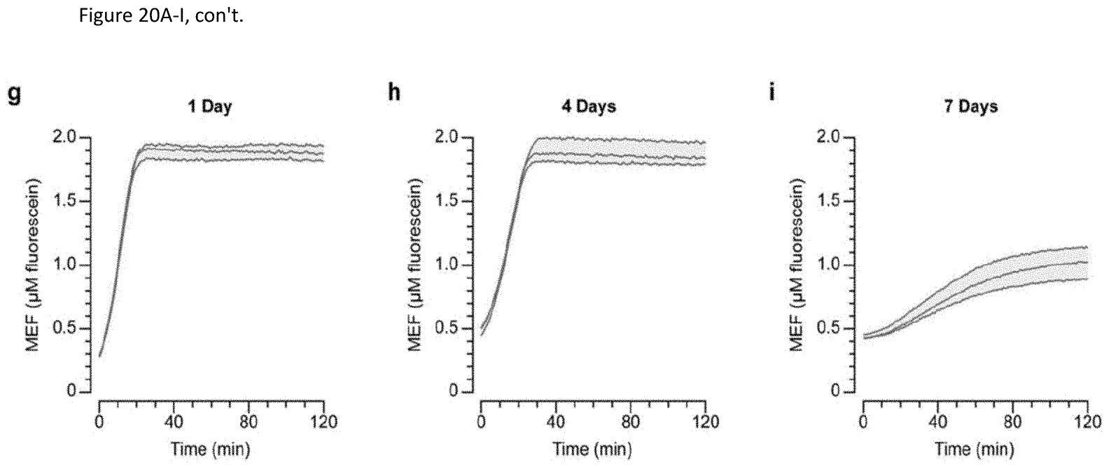

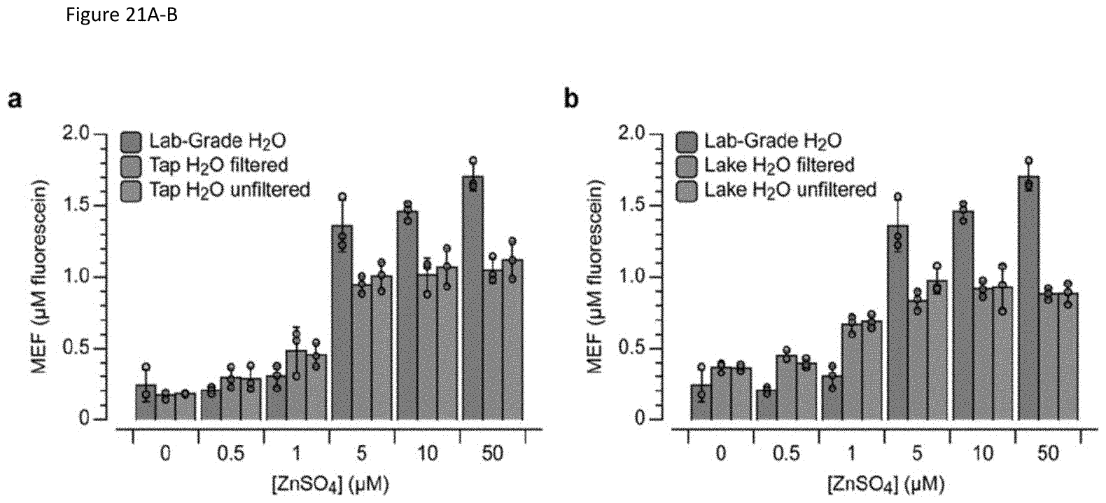

In this study, we show that nucleic acid strand displacement circuits can be interfaced with IVT to act as an information processing layer for cell-free biosensors. We found that the speed of DNA strand displacement outputs led to a significant enhancement of output signal generation speed, with visible outputs being produced in ˜10 minutes compared to ˜50 minutes for fluorescent RNA aptamer outputs (FIG. 3f). More significantly, we found that the simple and defined nature of ROSALIND, combined with the computational power of TMSD and the ability to model TMSD reactions with ODE simulations, enabled us to layer multiple RNA-DNA gates to build thirteen different circuits that implement seven different logic functions (FIGS. 5 and 6). Importantly, the ability to use RNA NOT gates to invert signals allowed us to create some of the first NOR, IMPLY, NAND and NIMPLY logic gates using TMSD circuits. Harnessing this high programmability of the platform, we also designed and validated a circuit that can estimate the concentration range of an unknown target compound within a sample (FIG. 7). Finally, this platform is amenable to lyophilization (FIG. 20) and can function with unprocessed real-world sample matrices (FIG. 21).

While simple in concept, we found that the combination of TMSD with cell-free biosensing reactions did not work immediately. This was due to several factors including the incompatibility of 3′ toehold overhangs in DNA gates with T7 RNAP-driven IVT reactions [50]. A careful analysis of the issues determined that this incompatibility is due to undesired transcription of these 3′ toehold overhangs by T7 RNAP, which can be solved by changing toehold overhangs to be on the 5′ ends (FIG. 10a-c), or by modifying the DNA gates to incorporate 2′-O-methylated nucleotides (FIG. 10d-f) [35]. In addition to resolving challenges with the compatibility of DNA gates with IVT, we needed to develop RNA engineering strategies to insulate our components against interfering RNA structures that can slow down TMSD. For example, in some cases, we observed that the transcribed aTF operator sequence interacted with the InvadeR sequence (FIG. 3, FIG. 14, 16k-m). We found that nucleic acid design tools such as NUPACK can be used to manipulate or add sequence regions predicted to minimize these structural interferences and therefore enhance compatibility with TMSD. Using this approach, we were able to freely design different InvadeR strands with minimum secondary structures on their toehold binding regions to improve the kinetics for both unregulated (FIG. 3) and aTF-regulated reactions (FIG. 14, 16k-m). Finally, T7 transcription efficiency can be altered with the initially transcribed sequences of an InvadeR strand [38], which can be used to tune and optimize transcription-driven TMSD (FIG. 11, 16f-j). Collectively, these elements—DNA gate compatibility with T7 RNAP, RNA structural interferences with TMSD and transcription efficiency tuning—represent additional design principles that were developed to create an interface between aTF biosensing and TMSD circuits for building information processing platforms into sensing reactions.

One of the major limitations of the platform is its cost. Despite the significant decrease in cost of DNA synthesis, chemically modified oligos with purification can still cost ˜$100 USD or more, though a single batch can be used to make hundreds of reactions. Furthermore, DNA gates often need to be gel-purified after hybridization to eliminate any unbound ssDNA strands, which can be time-intensive and laborious. This challenge can be partially solved by designing DNA signal gate sequences to minimize fluorophore quenching by the base adjacent to the modification [57]. Additionally, invading RNA strands can be designed to minimize intra- and intermolecular interactions to ensure that all TMSD reactions go to completion to maximize a fluorescent signal from the amount of a DNA signal gate used. We note, however, that the cost of chemical dyes for fluorescence-activating RNA aptamer reporting systems is not insignificant, and the advantages provided by the TMSD system such as the improved response speed and computational power outcompete its limitations.

The key feature of this study was demonstrating the potential of TMSD circuits to expand the function of cell-free biosensors by acting as additional information processing layers. While a similar approach was recently developed to interface aTF-based biosensing with TMSD through endonuclease-mediated TMSD cascades [58], no programmable molecular computation beyond simple contaminant detection was presented. As in natural organisms, information processing layers significantly expand the function of cell-free sensors by enabling systems to manipulate output signals, perform logic operations and make decisions. As a demonstration, we modeled, designed and validated several layered TMSD circuits capable of performing complex logic gate computation with chemical inputs (FIG. 5, 6, 15-17). These logic gates adapt principles of DNA-based [12, 15, 24] and co-transcriptional RNA-based [30, 53] TMSD logic circuits to interface the aTF-mediated small molecular sensing with a suite of cascaded TMSD circuits. We also demonstrate multiple different design architectures for a single type of logic gate, showcasing the platform's flexibility and modularity in implementing TMSD circuits (FIG. 17).

To further highlight the platform's capability for information processing, we developed a genetic ADC circuit that can be used to estimate an input ligand concentration at a semi-quantitative level (FIG. 7). In particular, this genetic ADC circuit uses thresholding computation to convert an analog signal of an input target molecule concentration into a digital output of the number of activated tubes. A key feature of TMSD that enabled this development is its ability to precisely tune reaction rates based on the toehold length. We note, however, this genetic ADC circuit is different from an electrical ADC circuit in that its result depends on time of activation and not activation level, because the circuit relies on thresholding reaction kinetics rather than strictly input concentrations. As a result, this ADC strategy is best suited to distinguishing between ligand concentrations that cause differences in output kinetics. (FIG. 19e-g).

We believe that this platform opens the door to enabling other types of molecular computation in cell-free systems. For example, an amplification circuit such as a catalytic hairpin assembly [59] could be applied to ROSALIND with TMSD for amplifying signals and making a sensor ultrasensitive. Beyond thresholding, other operations demonstrated in DNA seesaw gate architectures could be ported to this platform for various computations [24]. For instance, logic gate operations can be extended to develop a general strategy to fix aTF ligand promiscuity [7]. In addition, since virtually any aTF that functions in an in vitro context can be used [7], multiple DNA gates with different reporters could be added for multiplexing. The fundamental role that ADC circuits play in interfacing analog and digital electronic circuitry also holds promise for adopting additional electronic circuit designs to biochemical reactions.

Together, these results show that establishing an interface between small molecule biosensing and TMSD circuits is a promising first step towards creating a general molecular computation platform to enhance and expand the function of cell-free biosensing technologies.

Materials and Methods

Strains and Growth Medium

E. coli strain K12 (NEB Turbo Competent E. coli, New England Biolabs #C2984) was used for routine cloning. E. coli strain Rosetta 2(DE3)pLysS (Novagen #71401) was used for recombinant protein expression. Luria Broth supplemented with the appropriate antibiotic(s) (100 μg/mL carbenicillin, 100 μg/mL kanamycin and/or 34 μg/mL chloramphenicol) was used as the growth media.

DNA Gate Preparation

DNA signal gates used in this study were synthesized by Integrated DNA technologies as modified oligos. They were generated by denaturing a 6-FAM (fluorescein) modified oligonucleotide and the complementary Iowa Black® FQ quencher modified oligonucleotide (FIG. 23) at 95° C. separately for 3 minutes and slow cooling (−0.1° C./s) to room temperature in annealing buffer (100 mM potassium acetate and 30 mM HEPES, pH 8.0). Annealed oligonucleotides where then purified by resolving them on 20% native PAGE-TBE gels, isolating the band of expected size and eluting at 4° C. overnight in annealing buffer. The eluted DNA gate was then ethanol precipitated, resuspended in MilliQ ultrapure H₂O and concentration quantified using the Thermo Scientific™ NanoDrop™ One Microvolume UV-Vis spectrophotometer. The DNA threshold gate used in FIG. 7 was prepared using the same method but by annealing two complementary oligonucleotides without any modifications.

Plasmids and Genetic Parts Assembly

DNA oligonucleotides for cloning and sequencing were synthesized by Integrated DNA Technologies. Genes encoding aTFs were synthesized either as gBlocks (Integrated DNA Technologies) or gene fragments (Twist Bioscience). Protein expression plasmids were cloned using Gibson Assembly (NEB Gibson Assembly Master Mix, New England Biolabs #E2611) into a pET-28c plasmid backbone and were designed to overexpress recombinant proteins as C-terminus His-tagged fusions. A construct for expressing SmtB additionally incorporated a recognition sequence for cleavage and removal of the His-tag using TEV protease. Gibson assembled constructs were transformed into NEB Turbo cells, and isolated colonies were purified for plasmid DNA (QIAprep Spin Miniprep Kit, Qiagen #27106). Plasmid sequences were verified with Sanger DNA sequencing (Quintara Biosciences) using the primers listed in FIG. 23.

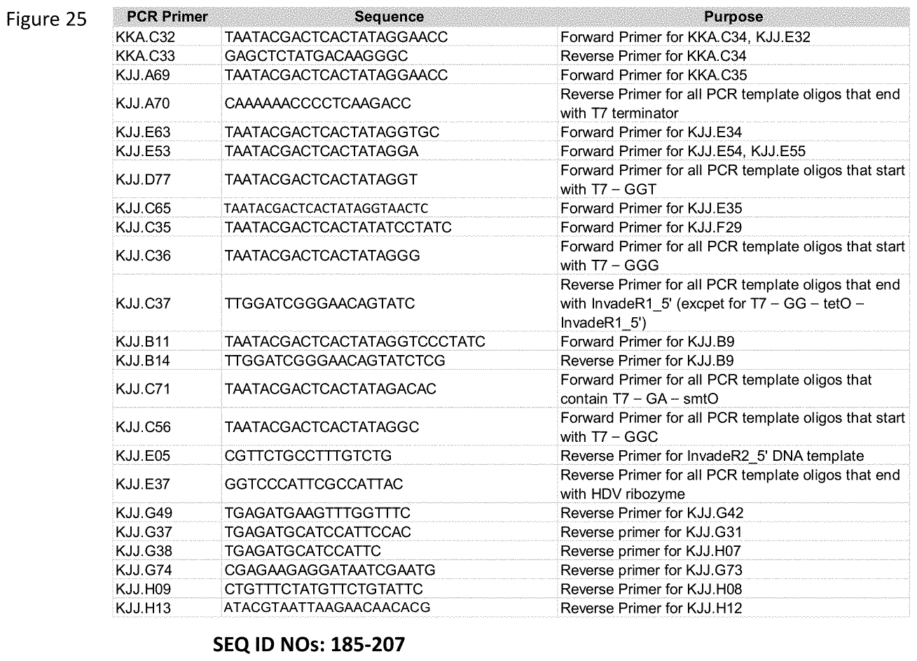

All transcription templates except for the templates encoding InvadeR variant 1 in FIG. 3, smtO-InvadeR-InvadeR in FIG. 14b, all RNA NOT gates presented in this study and InvadeR in FIG. 7 were generated by PCR amplification (Phusion High-Fidelity PCR Kit, New England Biolabs #E0553) of an oligo that includes a T7 promoter, an optional aTF operator site, the InvadeR coding sequence and an optional T7 terminator using the primer sets listed in FIG. 25. Here, we define the T7 promoter as a minimal 17-bp sequence (TAATACGACTCACTATA) excluding the first G that is transcribed. Amplified templates were then purified (QIAquick PCR purification kit, Qiagen #28106), verified for the presence of a single DNA band of expected size on a 2% TAE-Agarose gel, and concentrations were determined using the Qubit dsDNA BR Assay Kit (Invitrogen #Q32853). The templates encoding InvadeR variant 1 in FIG. 3, smtO-InvadeR-InvadeR in FIG. 14b, and all RNA NOT gates presented in this study and InvadeR in FIG. 7 were generated using the same method described in DNA gate preparation but with two complementary oligonucleotides that include a T7 promoter and the InvadeR or RNA NOT gate coding sequences.

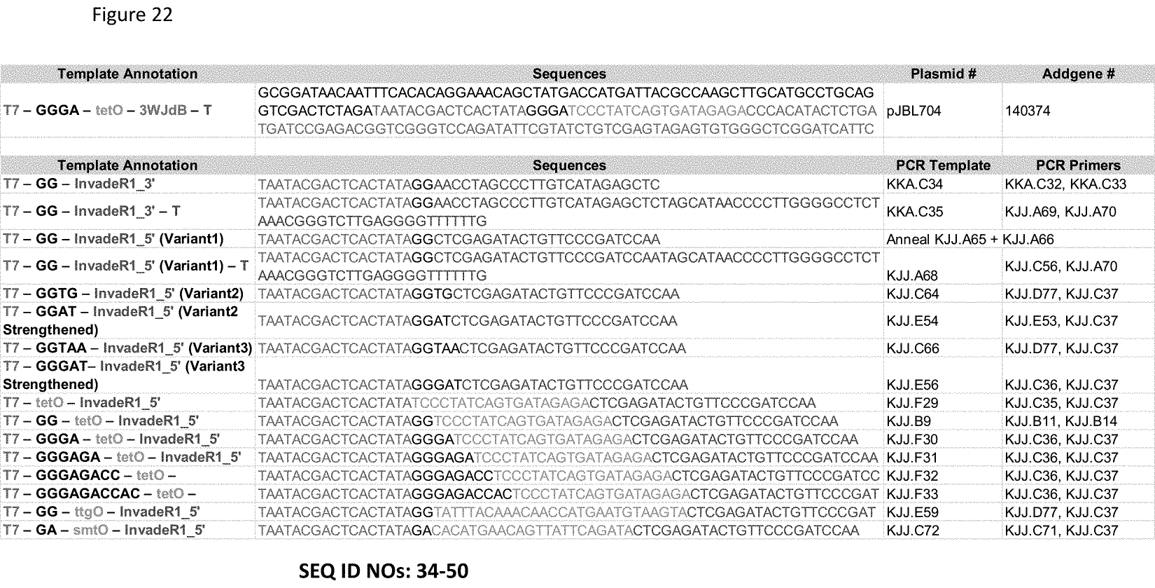

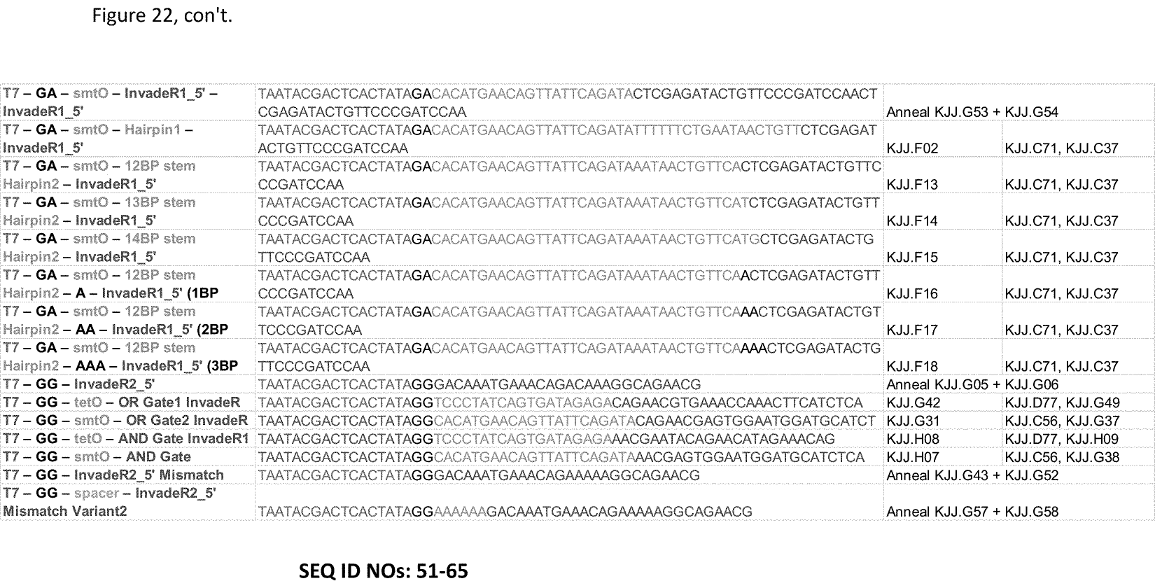

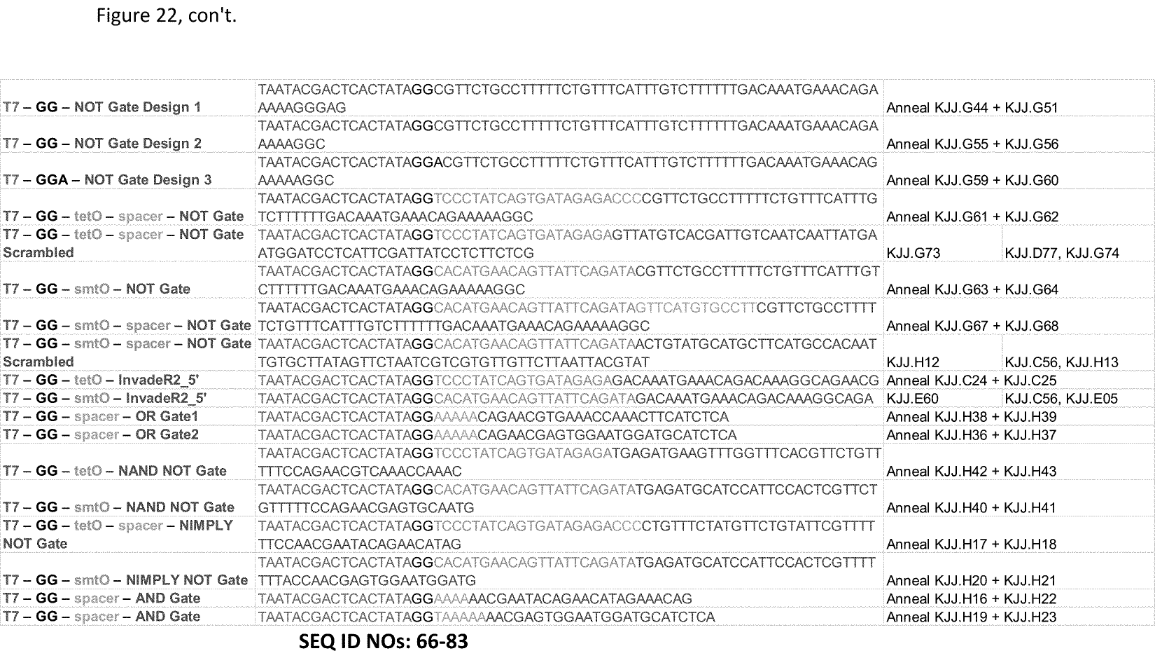

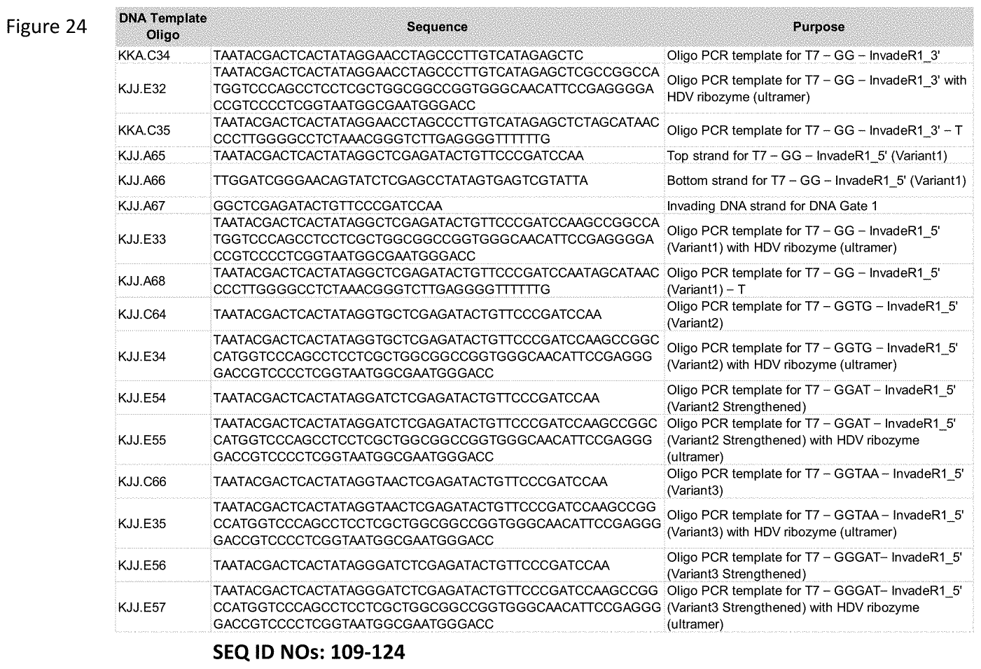

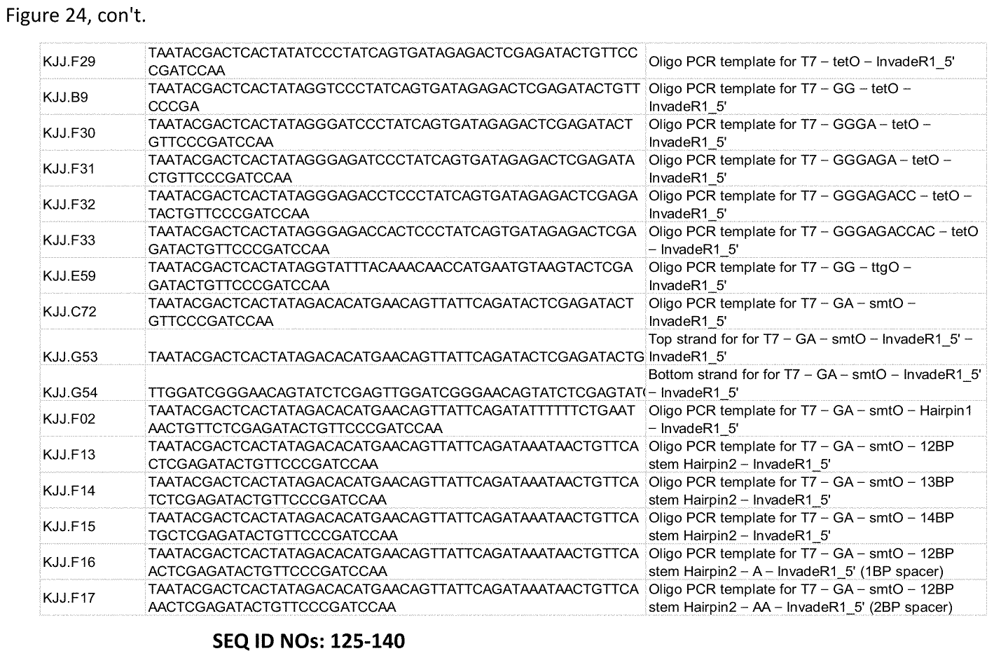

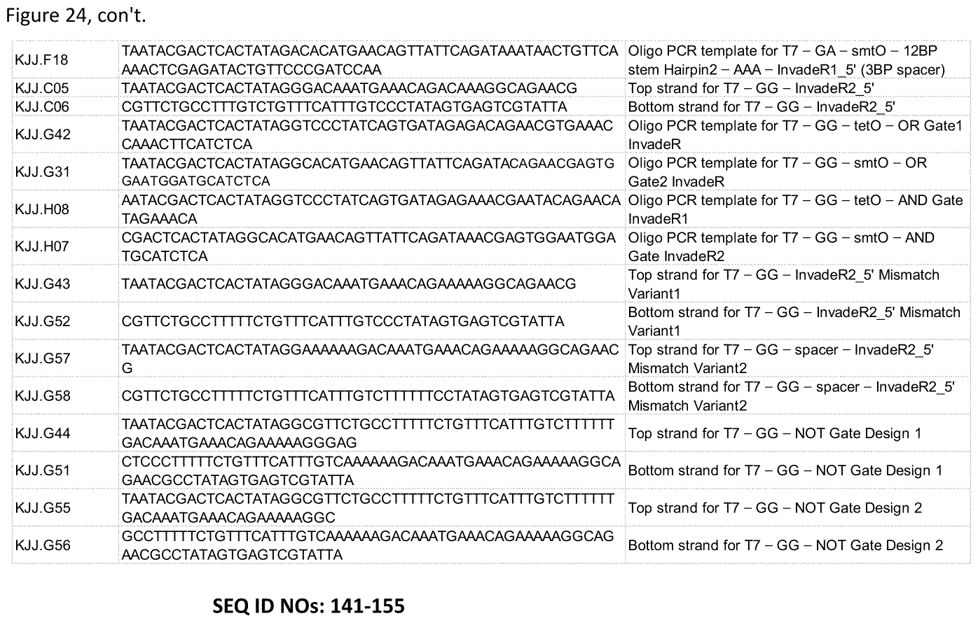

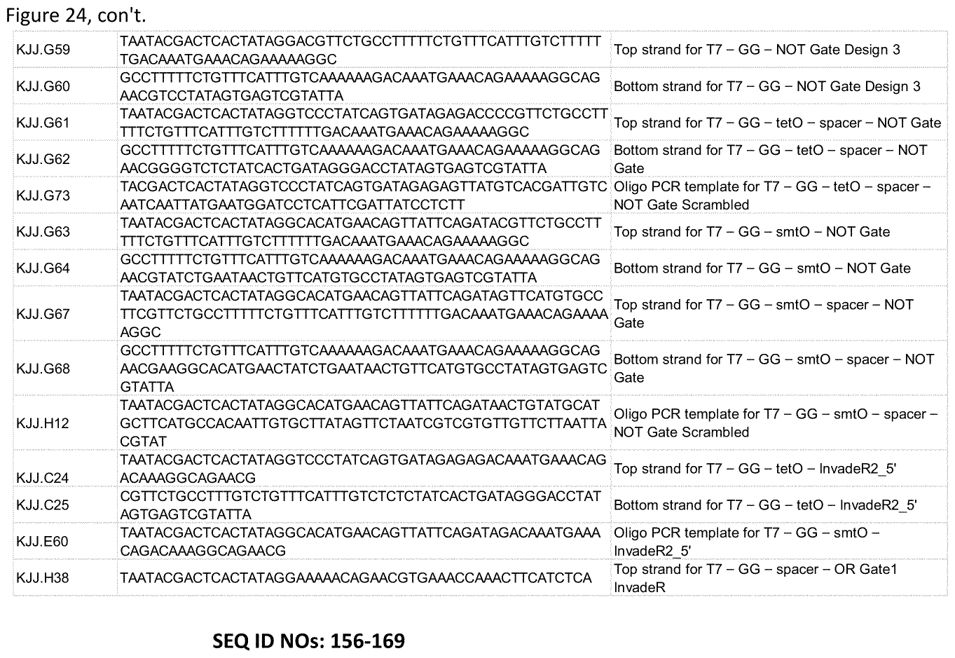

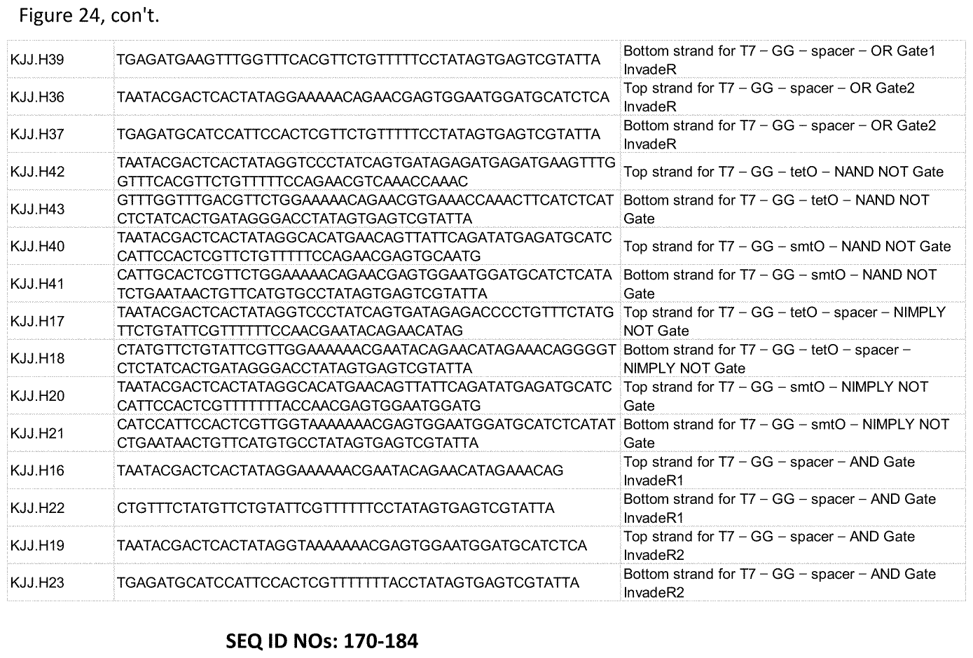

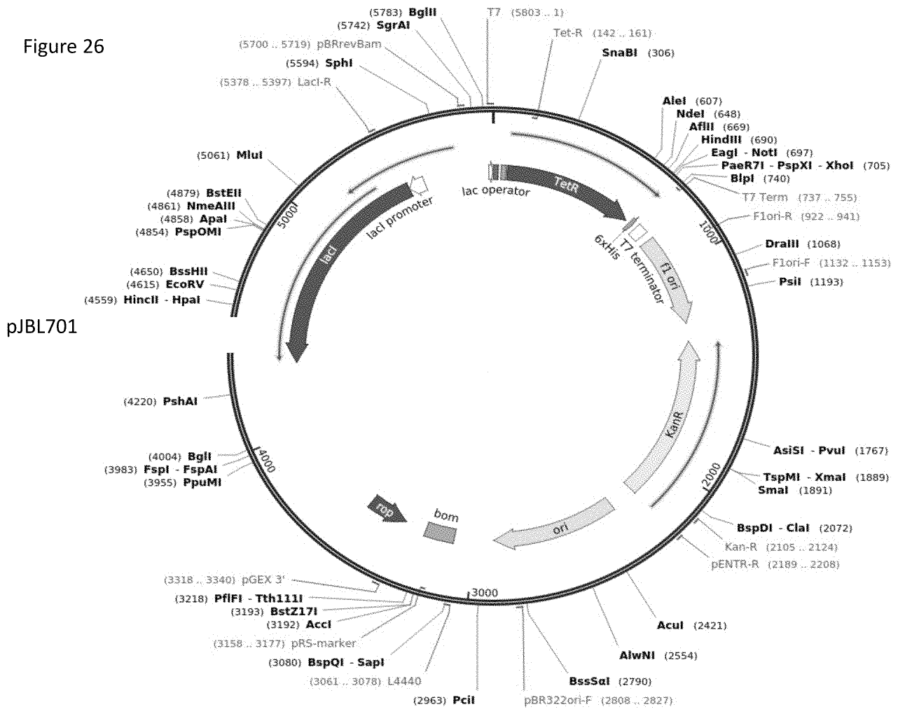

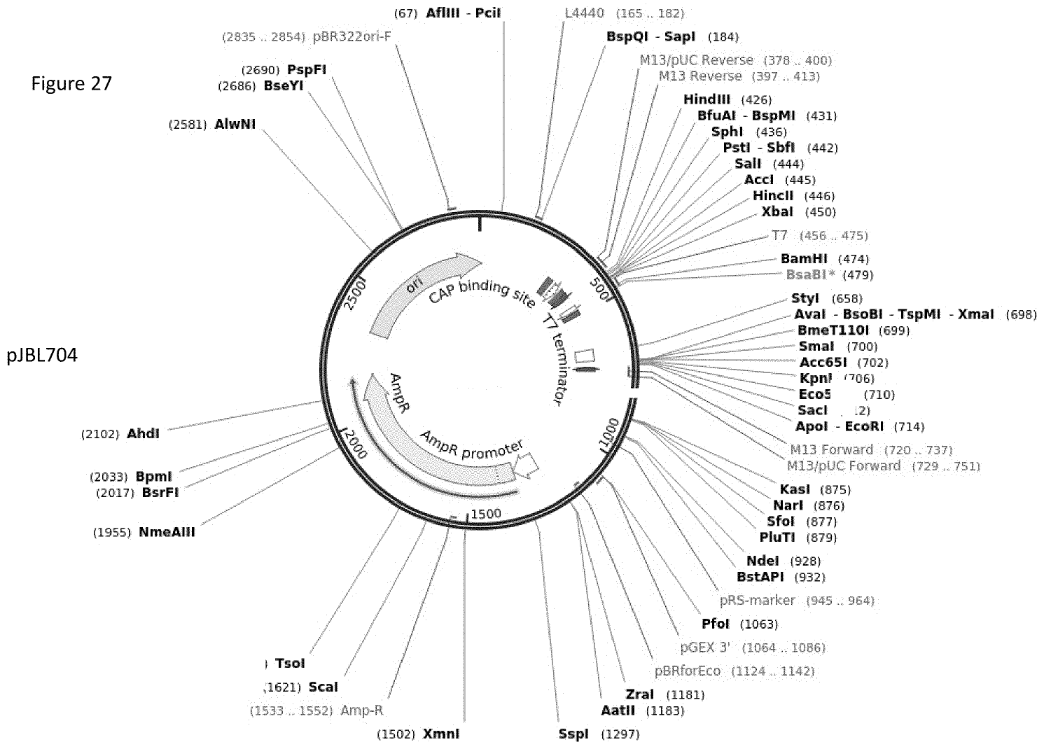

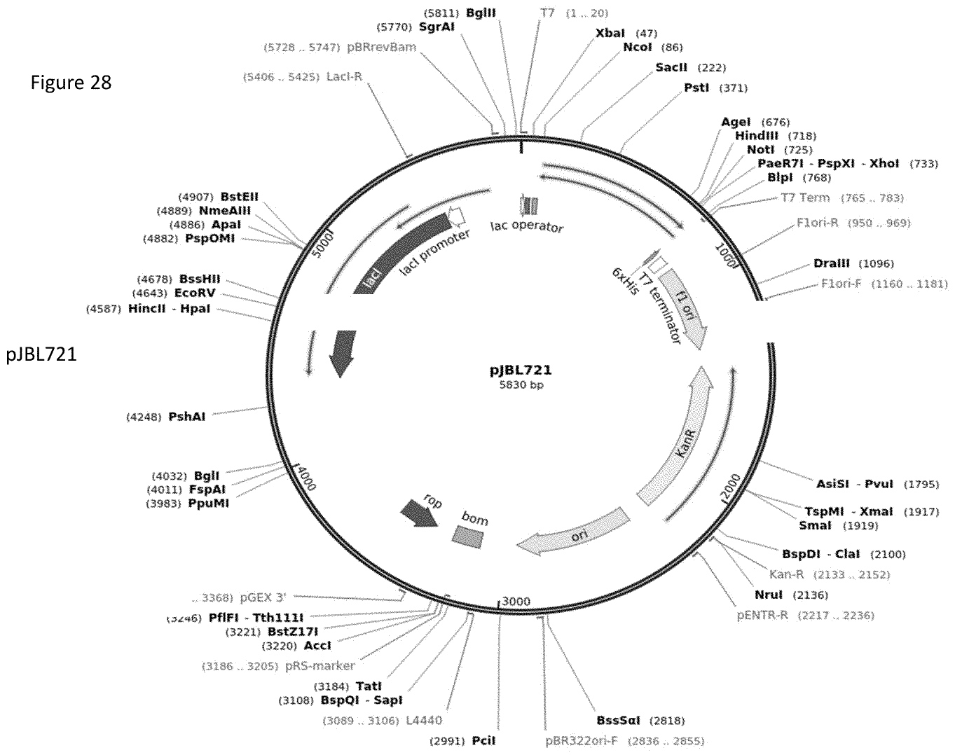

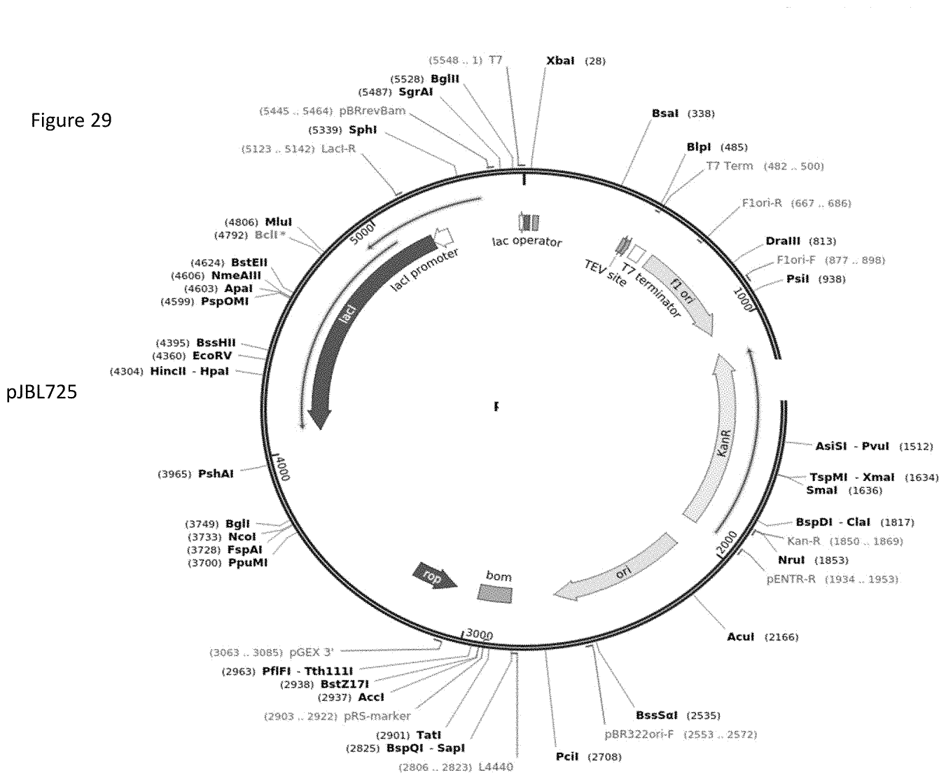

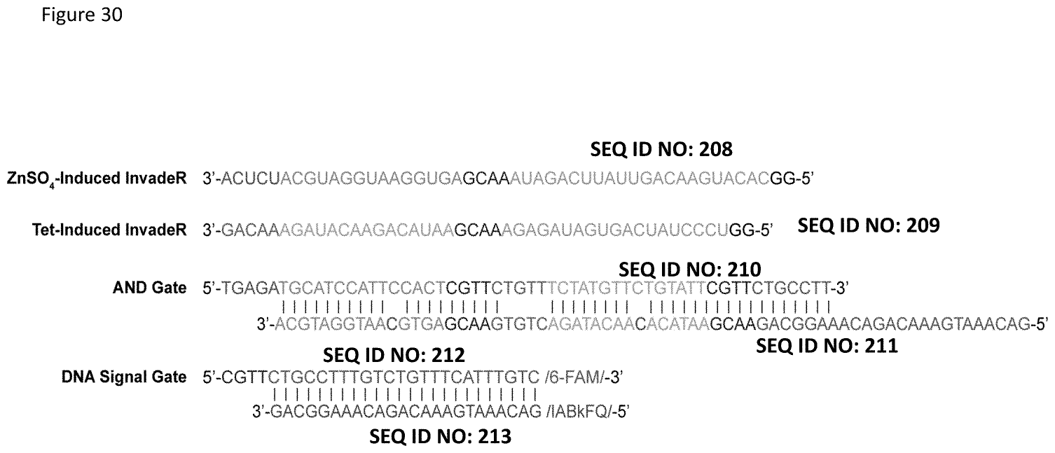

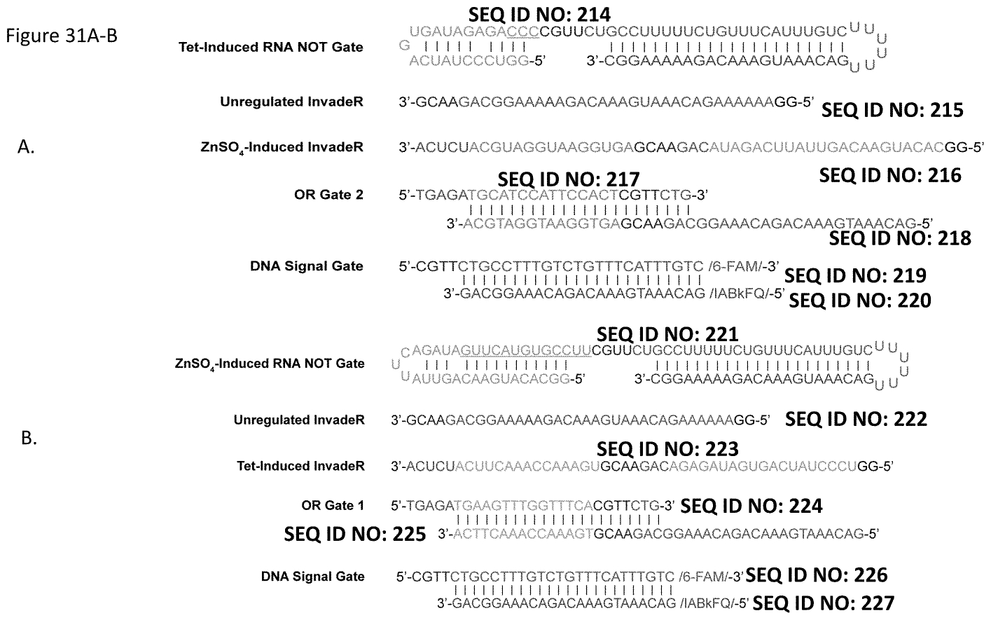

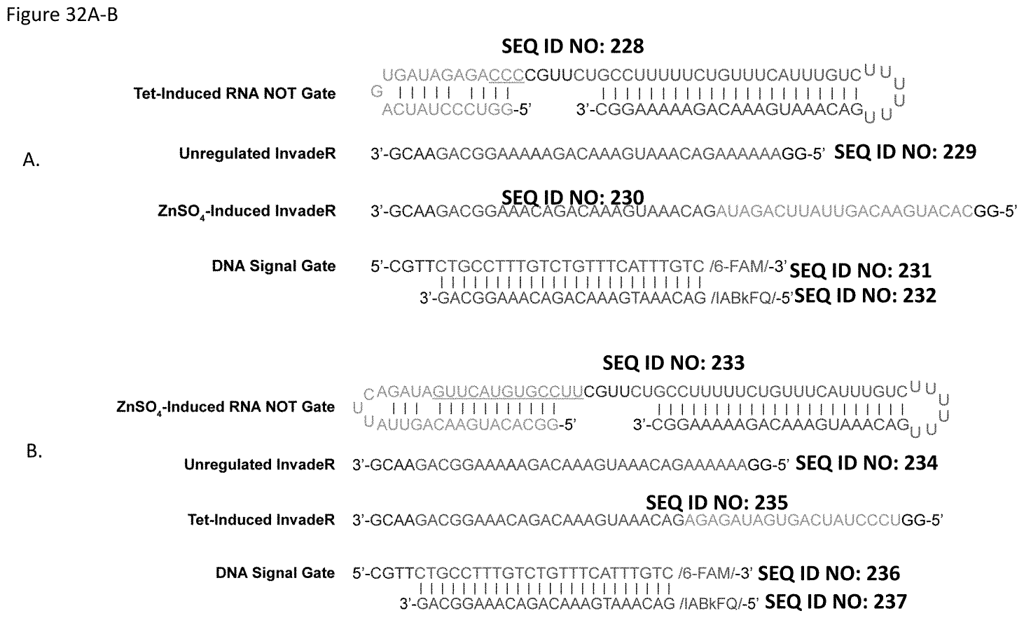

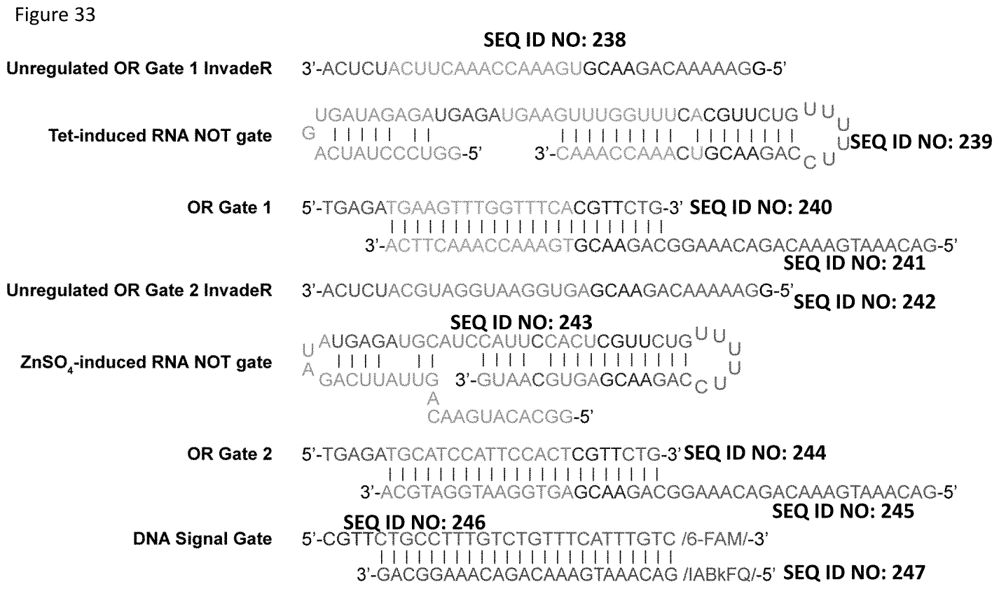

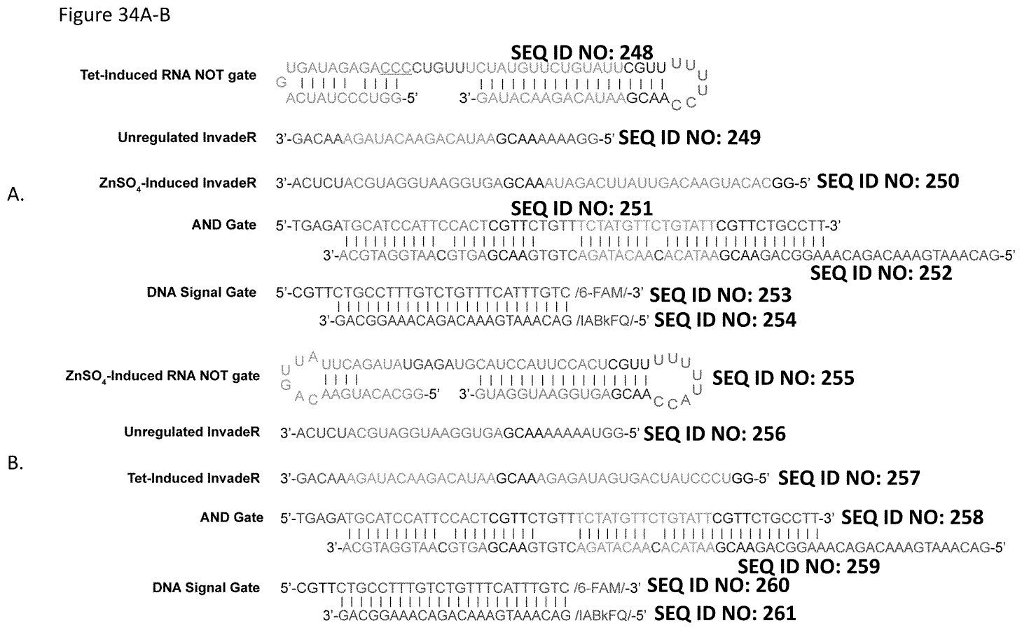

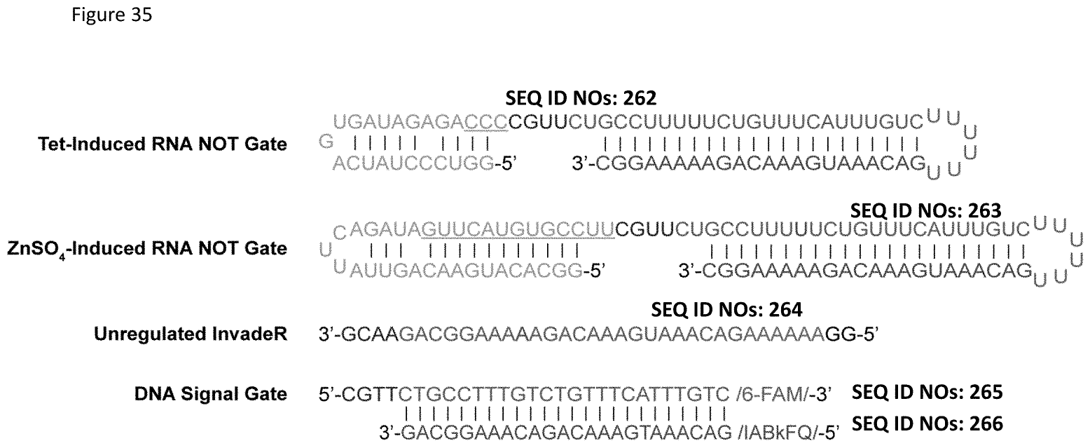

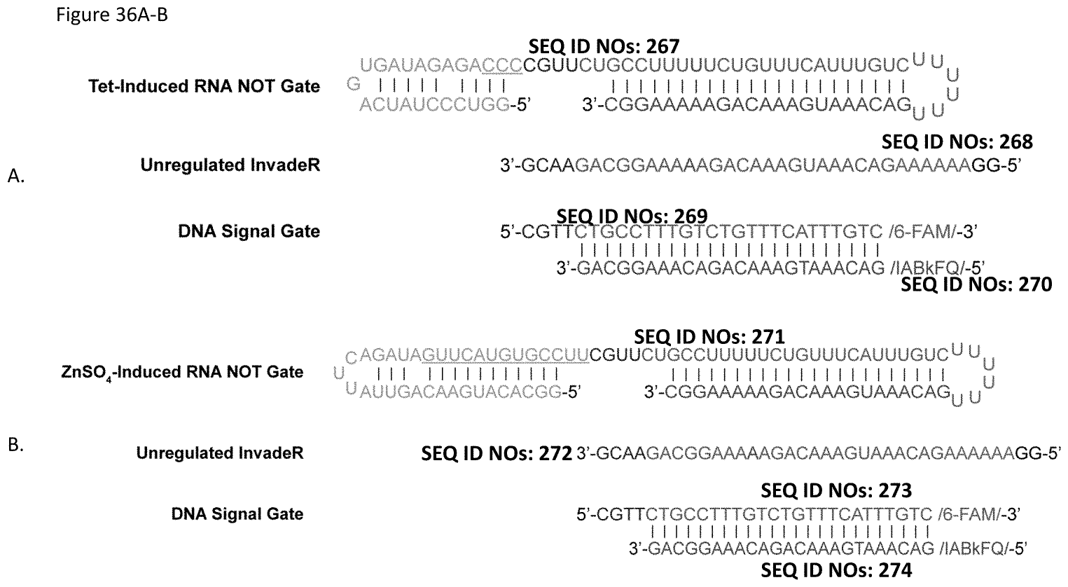

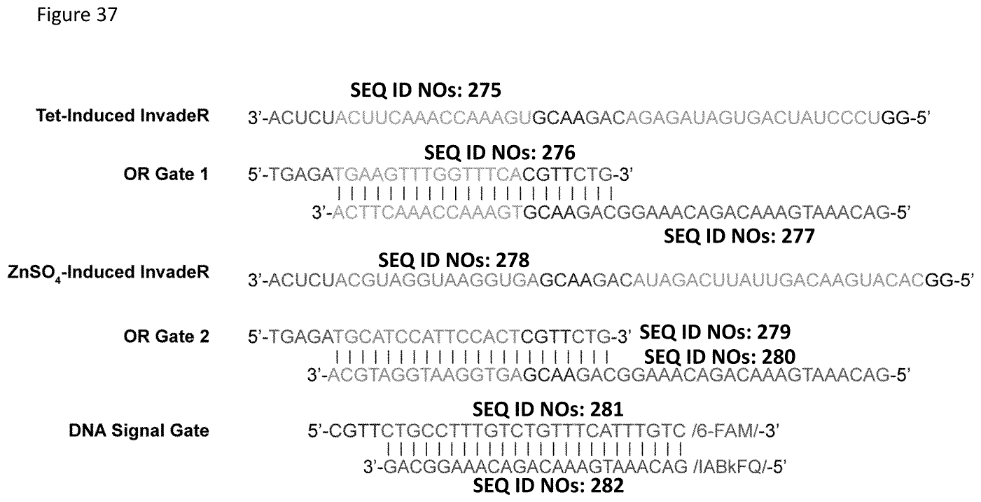

All plasmids and DNA templates were stored at 4° C. until usage. A listing of the sequences of the oligos and plasmids described in this document are provided in FIGS. 22-29.

RNA Expression and Purification

InvadeR variants used for the purified oligo binding assays were first expressed by an overnight IVT at 37° C. from a transcription template encoding a cis-cleaving Hepatitis D ribozyme on the 3′ end of the InvadeR sequence with the following components: IVT buffer (40 mM Tris-HCl pH 8, 8 mM MgCl₂, 10 mM DTT, 20 mM NaCl, and 2 mM spermidine), 11.4 mM NTPs pH 7.5, 0.3 U thermostable inorganic pyrophosphatase (#M0296S, New England Biolabs), 100 nM transcription template, 50 ng of T7 RNAP and MilliQ ultrapure H₂O to a total volume of 500 The overnight IVT reactions were then ethanol-precipitated and purified by resolving them on a 20% urea-PAGE-TBE gel, isolating the band of expected size (26-29 nt) and eluting at 4° C. overnight in MilliQ ultrapure H₂O. The eluted InvadeR variants were ethanol precipitated, resuspended in MilliQ ultrapure H₂O, quantified using the Qubit RNA BR Assay Kit (Invitrogen #Q10211) and stored at −20° C. until usage.

aTF Expression and Purification

aTFs were expressed and purified as previously described [7]. Briefly, sequence-verified pET-28c plasmids were transformed into the Rosetta 2(DE3) pLysS E. coli strain. 1-2 L of cell cultures were grown in Luria Broth at 37° C., induced with 0.5 mM of IPTG at an optical density (600 nm) of ˜0.5 and grown for 4 additional hours at 37° C. Cultures were then pelleted by centrifugation and were either stored at −80° C. or resuspended in lysis buffer (10 mM Tris-HCl pH 8, 500 mM NaCl, 1 mM TCEP, and protease inhibitor (complete EDTA-free Protease Inhibitor Cocktail, Roche)) for purification. Resuspended cells were then lysed on ice through ultrasonication, and insoluble materials were removed by centrifugation. Clarified supernatant containing TetR was then purified using His-tag affinity chromatography with a Ni-NTA column (HisTrap FF 5 mL column, GE Healthcare Life Sciences) followed by size exclusion chromatography (Superdex HiLoad 26/600 200 pg column, GE Healthcare Life Sciences) using an AKTAxpress fast protein liquid chromatography (FPLC) system. Clarified supernatants containing TtgR and SmtB were purified using His-tag affinity chromatography with a gravity column charged with Ni-NTA Agarose (Qiagen #30210). The eluted fractions from the FPLC (for TetR) or from the gravity column (for TtgR and SmtB) were concentrated and buffer exchanged (25 mM Tris-HCl, 100 mM NaCl, 1 mM TCEP, 50% glycerol v/v) using centrifugal filtration (Amicon Ultra-0.5, Millipore Sigma). Protein concentrations were determined using the Qubit Protein Assay Kit (Invitrogen #Q33212). The purity and size of the proteins were validated on a SDS-PAGE gel (Mini-PROTEAN TGX and Mini-TETRA cell, Bio-Rad). Purified proteins were stored at −20° C.

In Vitro Transcription (IVT) Reactions

Homemade IVT reactions were set up by adding the following components listed at their final concentration: IVT buffer (40 mM Tris-HCl pH 8, 8 mM MgCl₂, 10 mM DTT, 20 mM NaCl, and 2 mM spermidine), 11.4 mM NTPs pH 7.5, 0.3 U thermostable inorganic pyrophosphatase (#M0296S, New England Biolabs), transcription template, DNA gate(s) and MilliQ ultrapure H₂O to a total volume of 20 μL. Regulated IVT reactions additionally included a purified aTF at the indicated concentration and were equilibrated at 37° C. for ˜10 minutes. Immediately prior to plate reader measurements, 2 ng of T7 RNAP and, optionally, a ligand at the indicated concentration were added to the reaction. Reactions were then characterized on a plate reader as described in Plate reader quantification and micromolar equivalent fluorescein (MEF) standardization.

RNA Extraction from IVT Reactions

For RNA products shown on the gel images of FIG. 10c, f and FIGS. 11b and c, IVT reactions were first set up as described above. Then, phenol-chloroform extraction followed by ethanol precipitation was performed to remove any proteins. The reactions were then rehydrated in 1×TURBO™ DNase buffer with 2 U of TURBO™ DNase (Invitrogen #QAM2238) to a total volume of 20 μL and incubated at 37° C. for 30 minutes to remove the DNA gates and the transcription templates. Then, phenol-chloroform extraction followed by ethanol precipitation was performed again to remove DNase and rehydrated in MilliQ ultrapure H₂O. The concentrations of the extracted RNA products were measured using the Qubit RNA HS assay kit (Invitrogen #Q32852) and stored in −20° C. until further analysis such as PAGE.

Freeze-Drying

Prior to lyophilization, PCR tube caps were punctured with a pin to create three holes. Lyophilization of ROSALIND reactions was then performed by assembling the components of IVT (see above) with the addition of 50 mM sucrose and 250 mM D-mannitol. Assembled reaction tubes were immediately transferred into a pre-chilled aluminum block and placed in a −80° C. freezer for 10 minutes to allow slow-freezing. Following the slow-freezing, reaction tubes were wrapped in Kimwipes and aluminum foil, submerged in liquid nitrogen and then transferred to a FreeZone 2.5 L Bench Top Freeze Dry System (Labconco) for overnight freeze-drying with a condenser temperature of −85° C. and 0.04 millibar pressure. Unless rehydrated immediately, freeze-dried reactions were packaged as follows. The reactions were placed in a vacuum-sealable bag with a desiccant (Dri-Card Desiccants, Uline #S-19582), purged with Argon using an Argon canister (ArT Wine Preserver, Amazon #8541977939) and immediately vacuum-sealed (KOIOS Vacuum Sealer Machine, Amazon #TVS-2233). The vacuum-sealed bag then was placed in a light-protective bag (Mylar open-ended food bags, Uline #S-11661), heat-sealed (Metronic 8 inch Impulse Bag Sealer, Amazon #8541949845) and stored in a cool, shaded area until usage.

Plate Reader Quantification and Micromolar Equivalent Fluorescein (MEF) Standardization

A NIST traceable standard (Invitrogen #F36915) was used to convert arbitrary fluorescence measurements to micromolar equivalent fluorescein (MEF). Serial dilutions from a 50 μM stock were prepared in 100 mM sodium borate buffer at pH 9.5, including a 100 mM sodium borate buffer blank (total of 12 samples). The samples were prepared in technical and experimental triplicate (12 samples×9 replicates=108 samples total), and fluorescence values were read at an excitation wavelength of 495 nm and emission wavelength of 520 nm for 6-FAM (Fluorescein)-activated fluorescence, or at an excitation wavelength of 472 nm and emission wavelength of 507 nm for 3WJdB-activated fluorescence on a plate reader (Synergy H1, BioTek). Fluorescence values for a fluorescein concentration in which a single replicate saturated the plate reader were excluded from analysis. The remaining replicates (9 per sample) were then averaged at each fluorescein concentration, and the average fluorescence value of the blank was subtracted from all values. Linear regression was then performed for concentrations within the linear range of fluorescence (0-3.125 μM fluorescein) between the measured fluorescence values in arbitrary units and the concentration of fluorescein to identify the conversion factor. For each plate reader, excitation, emission and gain setting, we found a linear conversion factor that was used to correlate arbitrary fluorescence values to MEF (FIG. 8).

To characterize reactions, 19 μL of reactions were loaded onto a 384-well optically-clear, flat-bottom plate using a multichannel pipette, covered with a plate seal and measured on a plate reader (Synergy H1, BioTek). Kinetic analysis of 6-FAM (Fluorescein)-activated fluorescence was performed by reading the plate at 1 minute intervals with excitation and emission wavelengths of 495 nm and 520 nm, respectively, for two hours at 37° C. Kinetic analysis of 3WJdB-activated fluorescence was performed by reading the plate at 3-minute intervals with excitation and emission wavelengths of 472 nm and 507 nm, respectively, for four hours at 37° C. Arbitrary fluorescence values were then converted to MEF by dividing with the appropriate calibration conversion factor.

Except for the data in FIG. 7b, no background subtraction was performed when analyzing outputs from any reaction. An example of this standardization procedure is shown in FIG. 8.

Fluorescence Data Normalization (FIG. 7b Only)

Data shown in FIG. 7b were generated as above and then normalized by the following method in order to compare experimental observations to ODE simulations. Raw fluorescence values were first standardized to MEF (μM fluorescein) using the method described above. Then, the maximum MEF value was determined among all of the reactions run (5 conditions×3 replicates=15 reactions). Each MEF value at every time interval was then normalized using the following formula:

$$f ( x ) = \frac{{MEF}_{t = x} - {MEF}_{t = 0}}{Max MEF - {MEF}_{t = 0}}$$

where x is a given time point (0≤x≤120)

Background subtraction was performed to account for the non-zero fluorescence observed for the quenched DNA signal gate. Once all data were normalized according to the formula above, n=3 replicates per condition were averaged, and the corresponding standard deviation value per condition was calculated.

Gel Image Analysis

See Data availability section for uncropped, unprocessed gel images presented in FIG. 9d, FIG. 10c, f and FIG. 11c (deposited in Mendeley Data (doi: 10.17632/hr3j3yztxb.1)). The band intensity from a SYBR gold stained urea-PAGE gel in FIG. 11c was calculated using Fiji-ImageJ using the traditional lane-profile method as previously described [60]. Briefly, a region of interest in every lane was registered using a rectangle of the same dimension. Then, the uneven background was accounted for by drawing a straight line at the bottom of each peak, and the peak area in each lane was calculated using the wand tool. The peak areas of the RNA standard were then plotted against the total amounts loaded to create the standard curve in FIG. 11d (a linear range: 0.25-2 ng). Using the conversion factor from the standard curve, the concentrations of InvadeR variants were estimated from the peak area values obtained from the wand tool.

Tap and Lake Water Sampling

For ZnSO₄—spiked tap water from Evanston, IL, two bottles of approximately 50 ml of the water samples were collected from a drinking fountain. One of the bottles was then filtered at 0.22 μm using a Steriflip-GP sterile vacuum filtration system (MilliPore Sigma Cat. #SCGP00525). Both the filtered and unfiltered water samples were spiked using either 10 mM, 1 mM or 0.1 mM ZnSO₄ solution that has been diluted from the 2 M ZnSO₄ solution stock (Sigma Cat. #83265). Upon rehydration, fluorescence measurements of the reactions were performed by a plate reader (see “Plate reader quantification and MEF standardization”). For ZnSO₄—spiked Lake Michigan water from Evanston, IL, the same sampling method was applied.

Statistics and Reproducibility

The number of replicates and types of replicates performed are described in the legend to each figure. Individual data points are shown, and where relevant, the average±standard deviation is shown; this information is provided in each figure legend. The type of statistical analysis performed in FIG. 2c and FIG. 13 is described in the legend to each figure. See Data Availability section for exact P-values along with degrees of freedom computed from the statistical analysis.

Data Availability

All data presented in this document are deposited in Mendeley Data (doi: 10.17632/hr3j3yztxb.1). All plasmids used in this manuscript are available in Addgene with the identifiers 140371, 140374, 140391 and 140395.

Code Availability

The Python code used in FIG. 15 and FIG. 7 is available in GitHub at https://git.io/Jtlh1. The ODE model used in this manuscript is described in the Supplementary Methods below.

Supplementary Methods

ODE Model of TMSD Thresholding Circuit

Here, we use the kinetic rates of T7 RNAP-DNA binding, SmtB-srntO binding, SmtBZn binding, TMSD reactions and T7 RNAP-mediated IVT reactions to simulate ROSALIND Reactions. The following variables will be used:

### Abbreviations

| Abbreviation | Description |
|---|---|
| D | DNA template |
| RNAP | T7 RNA Polymerase |
| RD | T7 RNAP and DNA template bound complex |
| m | InvadeR |
| TF | Unbound, free SmtB tetramer |
| TFD | SmtB tetramer bound to one smtO |
| I | Unbound, free zinc ions |
| TFI | One zinc ion bound to SmtB tetramer |
| RQ | Signal gate |
| SD | InvadeR and 6′FAM heteroduplex |
| Q | guencher DNA strand |
| Th | Threshold gate |
| SDTh | InvadeR and threshold heteroduplex |
| QTh | Incumbent strand from threshold gate |

In this model, we assume:

1. One-One-to-one binding of T7 RNAP and T7 promoter on the DNA template.

2. The DNA template can be bound to either RNAP or TF, but not both.

3. One-to-one binding of SmtB tetramer and smtO on the DNA template (see footnote a).

4. One-to-one binding of SmtB tetramer and a zinc ion (see footnote a).

5. SmtB tetramer can be bound to either smtO on the DNA template or zinc, but not both.

6. All TMSD reactions are irreversible.

7. Fraying within each gate is ignored.

With these assumptions, we have the following reactions and ODEs in the system:

Reactions:

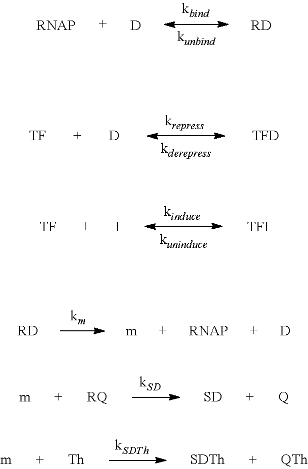

ODEs:

$$\frac{d [ RNAP ]}{dt} = {k}_{unbind} [ RD ] - {k}_{bind} [ RNAP ] [ D ] + {k}_{m} [ RD ]$$

$$\frac{d [ RD ]}{dt} = {k}_{bind} [ RNAP ] [ D ] - {k}_{unbind} [ RD ] - {k}_{m} [ RD ]$$

$$\frac{d [ D ]}{dt} = {k}_{unbind} [ RD ] - {k}_{bind} [ RNAP ] [ D ] + {k}_{derepress} [ TFD ] - {k}_{repress} [ TF ] [ D ] + {k}_{m} [ RD ]$$

$$\frac{d [ m ]}{dt} = {k}_{m} [ RD ] - {k}_{SD} [ RQ ] [ m ] - {k}_{SDTh} [ Th ] [ m ]$$

$$\frac{d [ TF ]}{dt} = {k}_{derepress} [ TFD ] - {k}_{repress} [ TF ] [ D ] - {k}_{induce} [ TF ] [ I ] + {k}_{uninduce} [ TFI ]$$

$$\frac{d [ TFD ]}{dt} = {k}_{repress} [ TF ] [ D ] - {k}_{derepress} [ TFD ]$$

$$\frac{d [ I ]}{dt} = {k}_{uninduce} [ TFI ] - {k}_{induce} [ TF ] [ I ]$$

$$\frac{d [ TFI ]}{dt} = {k}_{induce} [ TF ] [ I ] - {k}_{uninduce} [ TFI ]$$

$$\frac{d [ RQ ]}{dt} = - {k}_{SD} [ RQ ] [ m ]$$

$$\frac{d [ SD ]}{dt} = {k}_{SD} [ RQ ] [ m ]$$

$$\frac{d [ Q ]}{dt} = {k}_{SD} [ RQ ] [ m ]$$

$$\frac{d [ Th ]}{dt} = - {k}_{SDTh} [ Th ] [ m ]$$

$$\frac{d [ SDTh ]}{dt} = {k}_{SDTh} [ Th ] [ m ]$$

$$\frac{d [ QTh ]}{dt} = {k}_{SDTh} [ Th ] [ m ]$$

This set of ODEs was then run using an ODE solver function, odeint from the Scipy. Integrate package in Python 3.7.6. using the rate parameters shown below. For the unregulated reactions shown in FIG. 6b, the initial concentrations of TF, TFD, TFI and I were set to zero.

Parameters:

| Parameter | Value | Reference and Note |
|---|---|---|
| kₘ for T7-GG-InvR2 | 0.1/sec | [1] see footnote b |
| kₘ for T7-GG-smtO-InvR2 | 0.03/sec | [1] see footnote b |
| k_{bind} | 56/μM-sec | [2] |
| k_{unbind} | 0.2/sec | [2] |
| k_{SD} | 0.04/μM-sec | [3] for 4-nt toehold |
| k_{SDTh} | 4.0/μM-sec | [3] for 8-nt toehold |
| kᵣₑₚᵣₑₛₛ | 3.0/μM-sec | see footnote c |
| k_{derepress} | 0.18/sec | see footnote c |
| k_{induce} | 80/μM-sec | [4] see footnote d |
| k_{uninduce} | 0.1/sec | [4] see footnote d |

Notes:
a. We have found conflicting evidence from literature that reports different DNASmtB-zinc binding mechanisms. For instance, Kar, S. R., et al reports that SmtB predominantly forms a dimer and binds two zinc ions per subunit (therefore, one SmtB dimer binding 4 total zinc ions) [5]. On the other hand, VanZile, M. L. et al reports that SmtB binds one zinc ion per monomer [6]. A more recent literature 3 from Busenlenher, L. S. et al reports that two SmtB dimers tightly bind a single 12-2-12 inverted repeat of the smtO sequence [7]. We have found that following the mechanism from the most recent literature [7] where SmtB forms a tetramer to bind a single smtO site matches our experimental observations the best. We also suspect that while a single SmtB tetramer can bind multiple zinc ions at a time, not all zinc ions are needed to induce the transcription regulated by the SmtB tetramer. Following this logic, we used a model where one zinc ion can bind to the SmtB tetramer to induce the transcription of the invading RNA, which precisely matched our experimental observations.
b. These values were adjusted from the reported rate to accommodate for different initially transcribed nucleotides in each template. For instance, pT7-GGGA has a much greater transcription efficiency than pT7-GGCA [8].
c. There was no literature with this information available to the best of our knowledge. We have decided to estimate these values based on a reasonable range of a Kd value for a transcriptional repressor, which is typically in the nanomolar range. Currently, the rate constants are set so that the Kd = kderepress krepress_60 nM.
d. These values have been estimated to fit the association constants of SmtB-Zinc reported in VanZile, et al [4].

### REFERENCES FOR SUPPLEMENTARY METHODS

1. McClure, W. R., Rate-limiting steps in RNA chain initiation. PNAS, 1980. 77(10): p. 5634-8.
2. Ujvari, A. and C. T. Martin, Thermodynamics and kinetic measurements of promoter biding by T7 RNA polymerase. Biochemistry, 1996. 35(46): p. 14574-82.
3. Srinivas, N., et al., On the biophysics and kinetics of toehold-mediated DNA strand displacement. Nucleic Acids Research, 2013. 41(22): p. 10641-58.
4. VanZile, M. L., et al., Structural characterization of distinct a3N and a5 metal sites in the cyanobacterial zinc sensor SmtB. Biochemistry, 2002. 41(31): p. 9765-75.
5. Kar, S. R., et al., The cyanobacterial repressor SmtB is predominantly a dimer and binds two Zn2+ ions per subunit. Biochemistry, 1997. 36(49): p. 15343-8.
6. VanZile, M. L., et al., The Zinc Metalloregulatory Protein Synechococcus PCC7942 SmtB Binds a Single Zinc Ion per Monomer with High Affinity in a Tetrahedral Coordination Geometry. Biochemistry, 2000. 39(38): p. 11818-29.
7. Busenlehner, L. S., et al., The SmtB/ArsR family of metalloregulatory transcriptional repressors: structural insights into prokaryotic metal resistance. FEMS Microbiology Reviews, 2003. 27: p. 131-143.
8. Conrad, T., et al., Maximizing transcription of nucleic acids with efficient T7 promoters. Commun. Biol., 2020. 3(1): p. 439.

### Example 2. Polymerase Strand Recycling to Create an Intermediate Layer in Cell-Free Systems to Increase Biosensor Sensitivity

Polymerase Strand Recycling (PSR) is a DNA toehold-mediated strand displacement circuit that exploits promoter-independent transcription by T7 RNA polymerase. In this reaction, off-target transcription by T7 RNA polymerase (T7 RNAP) can be used to recycle circuit inputs, creating a catalytic amplification effect by which one circuit input can activate multiple outputs to increase sensitivity. We showed that we can generate higher fluorescent signal from the combination of controlled T7 RNAP off-target transcription and toehold-mediated strand displacement than for circuits that solely rely on strand displacement. This work establishes incorporating T7 RNAP side-reactions as an intended design choice into engineered TMSD circuits and has the potential to enhance existing molecular diagnostics systems as well as increase circuit design possibilities by creating an intermediate layer in cell-free systems that amplifies signal.

A cell-free biosensor (ROSALIND) typically activates when a target compound (input) binds to a protein transcription factor (sensor layer) that is configured to activate expression of a reporter construct (output layer). The single-stranded RNA output is capable of activating toehold-mediated strand displacement (TMSD) circuits that generate signal. Adding a downstream amplification layer before signal generation can recycle the single-stranded RNA output and decrease the amount of input required to fully activate a DNA signal gate.

Advantages to the disclosed technology include, but are not limited to, using T7 RNAP as a recycler for DNA strands to achieve signal recycling and amplification; the need for smaller amounts of one or more of the following components to achieve a detectable signal: (1) InvadeR; (2) DNA template; (3) aTF; (4) ligand. The system, including PSR (e.g., including a fuel gate and T7 RNAP as disclosed herein), achieves more sensitive detection of small molecule ligands as compared to the same system lacking the PSR components. The system including the PSR components as disclosed herein provides for greater sensitivity and lower detection limits (detection of lower concentrations and/or amounts) of small molecule ligands as compared to the same system without the PSR components.

In some embodiments, the sensitivity of embodiments including PSR is increased by greater than 10 fold, 20 fold, 30 fold, 40 fold, 50 fold, 60 fold, 70 fold, 80 fold, 90 fold, 100 fold, 150 fold, or greater than 200 fold compared to embodiments without PSR. In some embodiments, the amount of DNA template input can be reduced by a factor of 10 fold, 20 fold, 30 fold, 40 fold, 50 fold, 60 fold, 70 fold, 80 fold, 90 fold, 100 fold, 150 fold, or greater than 200 fold compared to embodiments without PSR. In some embodiments, the amount of aTF input can be reduced by a factor of 10 fold, 20 fold, 30 fold, 40 fold, 50 fold, 60 fold, 70 fold, 80 fold, 90 fold, 100 fold, 150 fold, or greater than 200 fold compared to embodiments without PSR.

In some embodiments, the TMSD circuit design incorporating T7 RNAP promoter-independent transcription activities comprises one or more chemically modified nucleic acids in the TMSD circuit component design.

In the presence of in vitro transcription components, T7 RNAP can non-specifically bind to the toehold region of the DNA signal gate. When the toehold is on the 3′ endo fo the gate, this leads to transcription of the unwanted RNA side products that can displace the quencher strand. This process is blocked when the toehold is on the 5′ endo fhte gate.

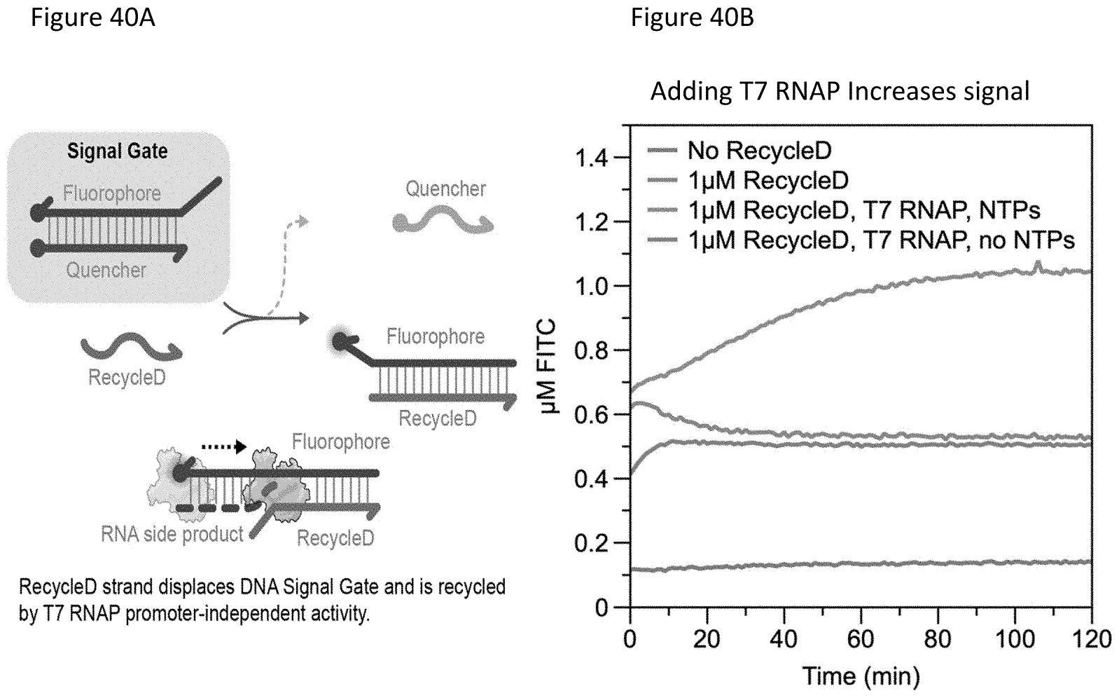

In some embodiments, the ligand binds to the allosteric transcription factor (aTF) allowing T7 RNA polymerase to transcribe the InvadeR strand which is complementary to the waste strand of the fuel gate and binds to the waste strand and displaces the RecycleD strand (FIG. 38). The RecycleD strand is complementary to the fluorophore strand of the signal gate and displaces the quencher strand of the signal gate, generating a fluorescent signal under appropriate conditions for the generation of fluorescence. Non-exclusive example fuel and signal gate designs are provided in FIG. 39 comprising SEQ ID NOs 283 and 284 and SEQ ID NOs 285 and 286, respectively. The dsDNA molecule comprising the fluorophore strand and the Recycle D strand comprises a toehold on the 3′ end of the fluorophore strand which allows for binding of T7 RNA polymerase, transcription of the fluorophore strand, and displacement of the RecycleD strand (FIG. 40A). Thus, the quencher strand is not likely to re-hybridize to the fluorophore strand because the RNA side product has higher affinity for the fluorophore strand than the quencher strand. In addition, the RecycleD strand is free to displace another quencher strand from a fluorophore strand generating further fluorescent signal and further perpetuating the cycle of transcribing the fluorophore strand and displacing the RecycleD strand, thereby increasing the signal generated downstream of a single InvadeR transcript, sometimes called a “two-way fuel gate” (FIG. 40B). T7 polymerase can displace a range of 3′ toehold lengths (data not shown).

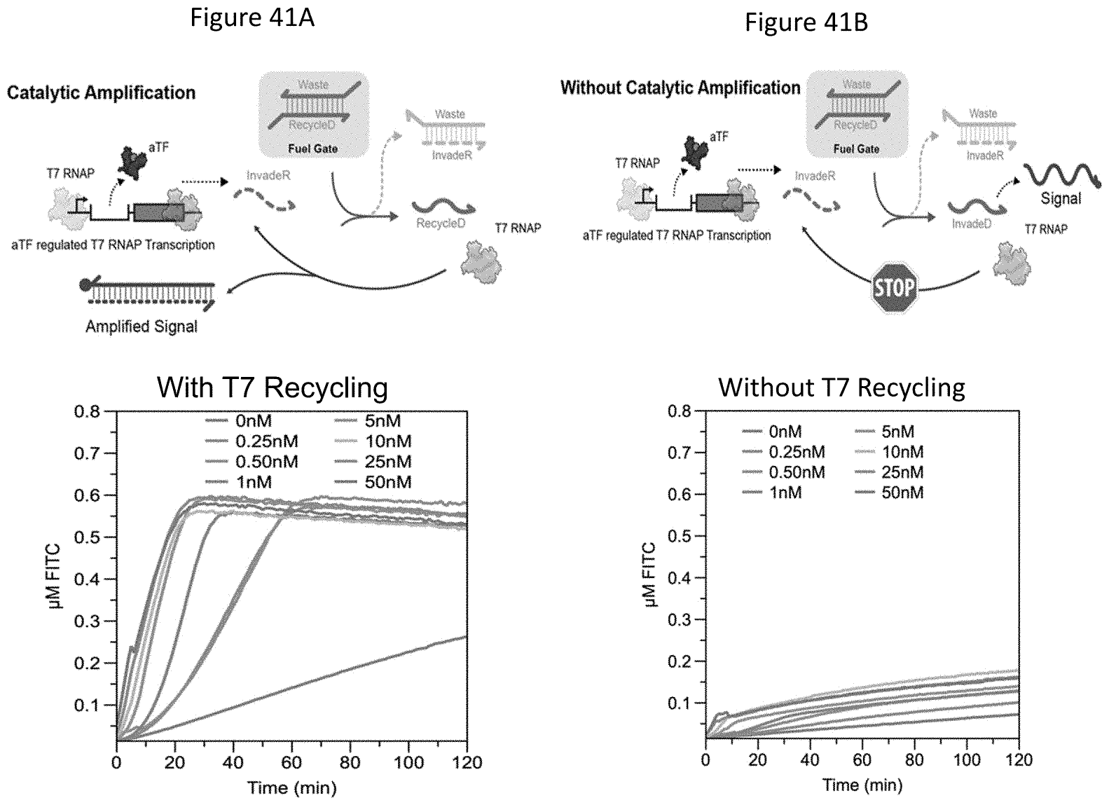

Using the embodiments described herein, DNA template inputs needed for the amplification system can be reduced by 100- to 200-fold (FIG. 41). Catalytic amplification of fluorescence (signal) with T7 recycling (FIG. 41A) has shown to produce greater florescence (signal) when compared to fluorescence (signal) production without catalytic amplification nor T7 recycling (FIG. 41B).

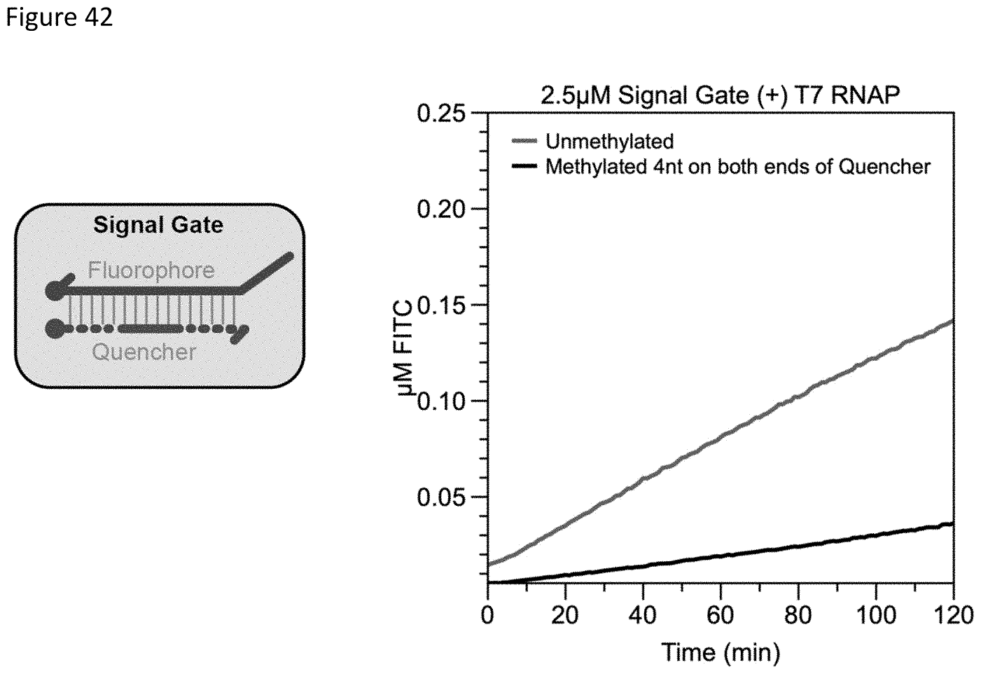

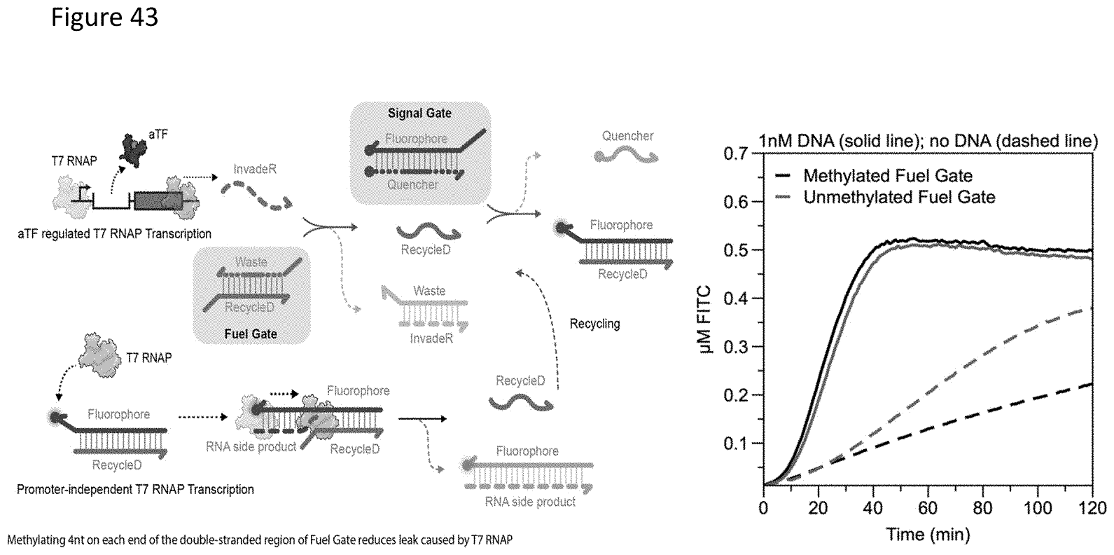

High leak signal proves to be a significant challenge in building a catalytic amplifier in the presence of T7 RNAP. However, methylation of the quencher strand of the signal gate reduces leak from the T7 RNAP amplifier and improves signal/noise ratio (FIG. 42). Methylation of the fuel gate further and/or alternatively reduces leak (FIG. 43). Methylation may be from one or both ends of the fuel gate and/or signal gate. Methylation of one end of either the fuel gate and/or signal gate may be preferred.

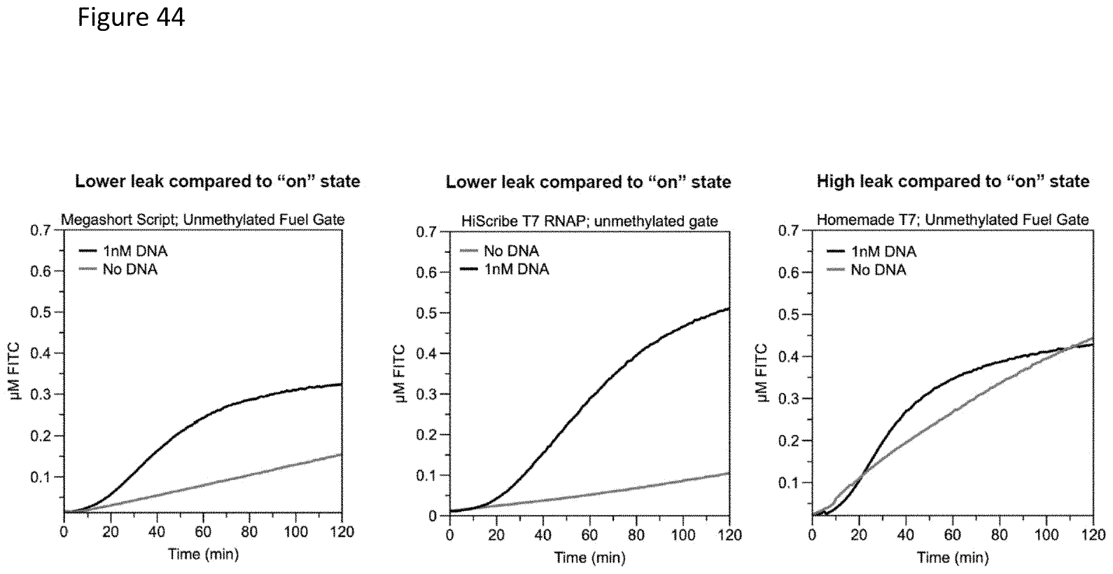

The present disclosure reveals that T7 RNAP activity is sensitive to the quality and source of T7 RNAP (FIG. 44) with respect to leak. For example, Megashort Script and HiScribe T7 RNAP have lower leak compared to “on” state while Homemade T7 has higher leak compared to “on” state (FIG. 44). Accordingly, while various T7 RNAPs will work in the present systems and methods (including the Homemade T7), in some embodiments, higher resolution may be desired, and alternate polymerases may be used. Interestingly, SP6 polymerase cannot displace 3′ toeholds (data not shown), and RNA-DNA duplex cannot be transcribed by 77 RNAP (data not shown).

In some embodiments, toehold lengths of the fuel gate may be between about 1-15 nt, between about 2-10 nt, or between about 4-8 nt. In some embodiments, the double stranded region of the fuel gate may be greater than 15 nt, such as 16, 17, 18, 19, 20, 21, 22, 23, 24, 25, 26, 27, 28, 29, 30, or more nucleotides.

A three-way fuel gate is disclosed herein, wherein the InvadeR strand displaced the DNA fuel gate and exposes a 3′ toehold for T7 RNAP to bind. In this system, T7 RNAP promoter-independently transcribes from the 3′ toehold and releases RecycleD which then activated the Signal Gate and is recycled by a second round of T7 RNAP promoter-independent transcription to amplify signal.

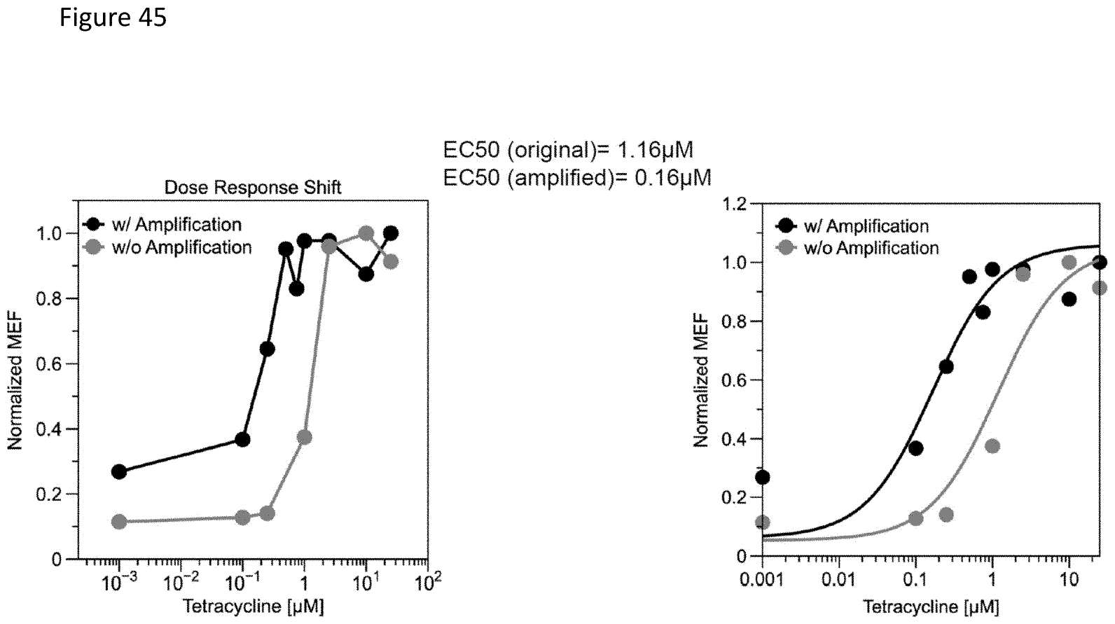

The present disclosure exhibits that tetracycline sensor sensitivity can be increased by PSR. As illustrated in FIG. 45, the EC₅₀ of tetracycline is decreased by greater than 80% (e.g., 86%) with PSR amplification (FIG. 45).

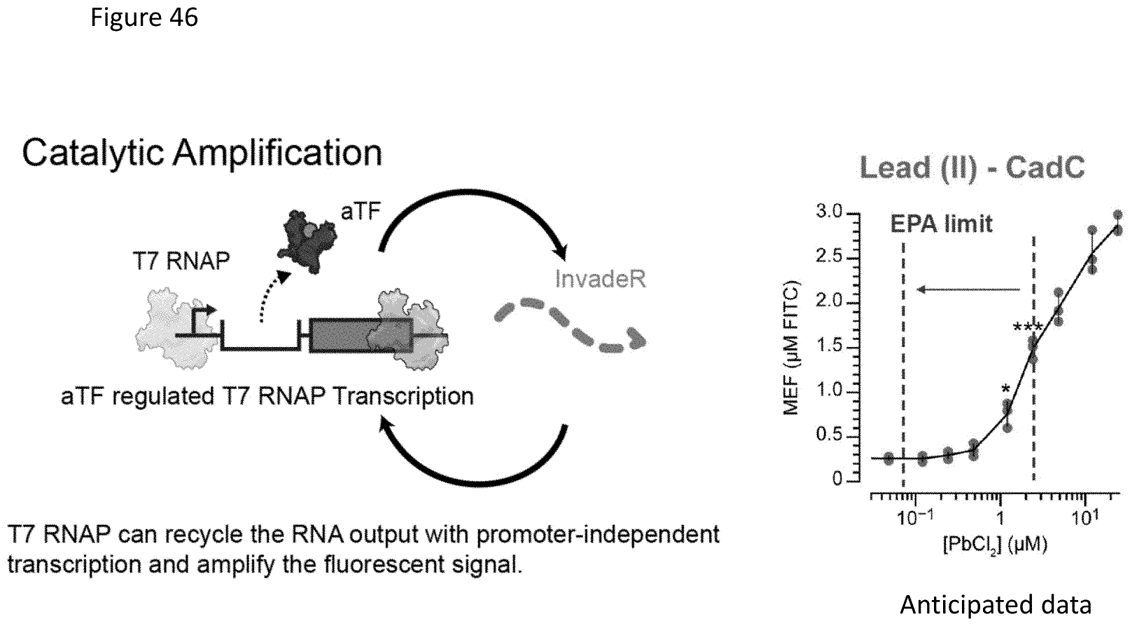

By way of example, and not by way of limitation, in some embodiments, the use of a fuel gate will reduce the amount of transcription factor needed for a ROSALIND reaction and increase the sensitivity of small molecule sensors, including, for example, lead and zinc small molecule sensors. For example, T7 RNAP may be able to recycle the RNA output with promoter-independent transcription and amplify the fluorescent signal of aTF-regulated transcription. For example, the Environmental Protection Agency's limit for lead contamination is below the sensitivity limit of some small molecule sensors. The inventors predict that using the embodiments of the present disclosure, small molecule sensors will be able to detect lower levels of ligands, such as, without limitation lead, at levels mandated by regulatory agencies or below (see e.g., FIG. 46).

### REFERENCES

1. Thavarajah, W., et al., Point-of-Use Detection of Environmental Fluoride via a Cell-Free Riboswitch-Based Biosensor. ACS Synth Biol, 2020. 9(1): p. 10-18.
2. Pardee, K., Perspective: Solidifying the impact of cell free synthetic biology through lyophilization. Biochem Eng J, 2018. 138: p. 91-97.
3. McNerney, M. P., et al., Point-of-care biomarker quantification enabled by sample-specific calibration. Sci Adv, 2019. 5(9): p. eaax4473.
4. Wen, K. Y., et al., A Cell-Free Biosensor for Detecting Quorum Sensing Molecules in P. aeruginosa-Infected Respiratory Samples. ACS Synth Biol, 2017. 6(12): p. 2293-2301.
5. Grawe, A., et al., A paper-based, cell-free biosensor system for the detection of heavy metals and date rape drugs. PLoS One, 2019. 14(3): p. e0210940.
6. Silverman, A. D., et al., Design and Optimization of a Cell-Free Atrazine Biosensor. ACS Synth Biol, 2020. 9(3): p. 671-677.
7. Jung, J. K., et al., Cell free biosensors for rapid detection of water contaminants. Nat Biotechnol, 2020. 38(12): p. 1451-1459.
8. Alon, U., An introduction to systems biology: design principles of biological circuits. Second edition. ed. 2019, Boca Raton, Fla.: CRC Press. pages cm.
9. Nielsen, A. A., et al., Genetic circuit design automation. Science, 2016. 352(6281): p. aac7341.
10. Wan, X., et al., Cascaded amplifying circuits enable ultrasensitive cellular sensors for toxic metals. Nat Chem Biol, 2019. 15(5): p. 540-548.
11. Zhang, D. Y. and G. Seelig, Dynamic DNA nanotechnology using strand-displacement reactions. Nat Chem, 2011. 3(2): p. 103-13.
12. Winfree, L. Q. a. E., A simple DNA gate motif for synthesizing large-scale circuits. Journal of the Royal Society Interface, 2011. 8(62): p. 1281-1297.
13. Zhang, D. Y., et al., Engineering entropy-driven reactions and networks catalyzed by DNA. Science, 2007. 318(5853): p. 1121-5.
14. Li, W., et al., Three-input majority logic gate and multiple input logic circuit based on DNA strand displacement. Nano Lett, 2013. 13(6): p. 2980-8.
15. Seelig, G., et al., Enzyme free nucleic acid logic circuits. Science, 2006. 314(5805): p. 1585-8.
16. Hjelmfelt, A., E. D. Weinberger, and J. Ross, Chemical implementation of neural networks and Turing machines. Proc Natl Acad Sci USA, 1991. 88(24): p. 10983-7.
17. Zhang, D. Y. and E. Winfree, Control of DNA strand displacement kinetics using toehold exchange. J Am Chem Soc, 2009. 131(47): p. 17303-14.
18. Kim, J. and E. Winfree, Synthetic in vitro transcriptional oscillators. Mol Syst Biol, 2011. 7: p. 465.
19. Jiang, Y. S., et al., Real-time detection of isothermal amplification reactions with thermostable catalytic hairpin assembly. J Am Chem Soc, 2013. 135(20): p. 7430-3.
20. Yurke, B., et al., A DNA fuelled molecular machine made of DNA. Nature, 2000. 406(6796): p. 605-8.
21. Dirks, R. M. and N. A. Pierce, Triggered amplification by hybridization chain reaction. Proc Natl Acad Sci USA, 2004. 101(43): p. 15275-8.
22. Yin, P., et al., Programming biomolecular self-assembly pathways. Nature, 2008. 451(7176): p. 318-22.
23. Green, L. N., et al., Autonomous dynamic control of DNA nanostructure self-assembly. Nat Chem, 2019. 11(6): p. 510-520.
24. Qian, L. and E. Winfree, Scaling up digital circuit computation with DNA strand displacement cascades. Science, 2011. 332(6034): p. 1196-201.
25. Cherry, K. M. and L. Qian, Scaling up molecular pattern recognition with DNA-based winner-take-all neural networks. Nature, 2018. 559(7714): p. 370-376.
26. Hu, X., et al., Single-molecule catalytic hairpin assembly for rapid and direct quantification of circulating miRNA biomarkers. Anal Chim Acta, 2018. 1042: p. 109-115.
27. Jiang, Y. S., et al., Robust strand exchange reactions for the sequence-specific, real-time detection of nucleic acid amplicons. Anal Chem, 2015. 87(6): p. 3314-20.
28. Jiang, Y. S., et al., Portable platform for rapid in-field identification of human fecal pollution in water. Water Res, 2018. 131: p. 186-195.
29. Schaffter, S. W., et al., T7 RNA polymerase non-specifically transcribes and induces disassembly of DNA nanostructures. Nucleic Acids Res, 2018. 46(10): p. 5332-5343.
30. Schaffter, S. W. and E. A. Strychalski, Co-transcriptional RNA strand displacement circuits. bioRxiv, 2021: p. 2021.07.20.450530.
31. Bhadra, S. and A. D. Ellington, Design and application of cotranscriptional non-enzymatic RNA circuits and signal transducers. Nucleic Acids Res, 2014. 42(7): p. e58.
32. Zadeh, J. N., et al., NUPACK. Analysis and design of nucleic acid systems. J Comput Chem, 2011. 32(1): p. 170-3.
33. Sharmeen, L. and J. Taylor, Enzymatic synthesis of RNA oligonucleotides. Nucleic Acids Res, 1987. 15(16): p. 6705-11.
34. Krupp, G., Unusual promoter-independent transcription reactions with bacteriophage RNA polymerases. Nucleic Acids Res, 1989. 17(8): p. 3023-36.
35. Marras, S., et al., Real-time measurement of in vitro transcription. Nucleic acids research, 2004. 32: p. e72.
36. Srinivas, N., et al., On the biophysics and kinetics of toehold-mediated DNA strand displacement. Nucleic Acids Res, 2013. 41(22): p. 10641-58.
37. Sulc, P., et al., Modelling toehold-mediated RNA strand displacement. Biophys J, 2015. 108(5): p. 1238-47.
38. Conrad, T., et al., Maximizing transcription of nucleic acids with efficient T7 promoters. Commun Biol, 2020. 3(1): p. 439.
39. Schindelin, J., et al., Fiji: an open-source platform for biological-image analysis. Nat Methods, 2012. 9(7): p. 676-82.
40. Rong, M., R. K. Durbin, and W. T. McAllister, Template strand switching by T7 RNA polymerase. J Biol Chem, 1998. 273(17): p. 10253-60.
41. Cuthbertson, L. and J. R. Nodwell, The TetR family of regulators. Microbiol Mol Biol Rev, 2013. 77(3): p. 440-75.
42. Han, K. Y., et al., Understanding the photophysics of the spinach-DFHBI RNA aptamer-fluorogen complex to improve live-cell RNA imaging. J Am Chem Soc, 2013. 135(50): p. 19033-8.
43. Mitra, J. and T. Ha, Nanomechanics and co-transcriptional folding of Spinach and Mango. Nat Commun, 2019. 10(1): p. 4318.
44. Rogers, J. K., et al., Synthetic biosensors for precise gene control and real-time monitoring of metabolites. Nucleic Acids Res, 2015. 43(15): p. 7648-60.
45. VanZile, M. L., X. Chen, and D. P. Giedroc, Allosteric negative regulation of smt O/P binding of the zinc sensor, SmtB, by metal ions: a coupled equilibrium analysis. Biochemistry, 2002. 41(31): p. 9776-86.
46. Deochand, D. K. and A. Grove, MarR family transcription factors: dynamic variations on a common scaffold. Crit Rev Biochem Mol Biol, 2017. 52(6): p. 595-613.
47. Busenlehner, L. S., M. A. Pennella, and D. P. Giedroc, The SmtB/ArsR family of metalloregulatory transcriptional repressors: Structural insights into prokaryotic metal resistance. FEMS Microbiol Rev, 2003. 27(2-3): p. 131-43.
48. Liu, X., et al., De novo design of programmable inducible promoters. Nucleic Acids Research, 2019. 47(19): p. 10452-10463.
49. Krishnan, Y. and F. C. Simmel, Nucleic acid based molecular devices. Angew Chem Int Ed Engl, 2011. 50(14): p. 3124-56.
50. Schaffter, S. W. and R. Schulman, Building in vitro transcriptional regulatory networks by successively integrating multiple functional circuit modules. Nature Chemistry, 2019. 11(9): p. 829-838.
51. Haley, N. E. C., et al., Design of hidden thermodynamic driving for non-equilibrium systems via mismatch elimination during DNA strand displacement. Nat Commun, 2020. 11(1): p. 2562.
52. Wang, B., et al., Effective design principles for leakless strand displacement systems. Proc Natl Acad Sci USA, 2018. 115(52): p. E12182-E12191.
53. Bae, W., G. V. Stan, and T. E. Ouldridge, In situ Generation of RNA Complexes for Synthetic Molecular Strand-Displacement Circuits in Autonomous Systems. Nano Lett, 2021. 21(1): p. 265-271.
54. Mannan, A. A., et al., Fundamental Design Principles for Transcription-Factor-Based Metabolite Biosensors. ACS Synth Biol, 2017. 6(10): p. 1851-1859.
55. Ida, N., Sensors, actuators, and their interfaces: a multidisciplinary introduction. 2014, Edison, NJ: SciTech Publishing. 761 pages.
56. World Health Organization., Guidelines for drinking-water quality. 4th ed. 2011, Geneva: World Health Organization. xxiii, 541 p.
57. Nazarenko, I., et al., Effect of primary and secondary structure of oligodeoxyribonucleotides on the fluorescent properties of conjugated dyes. Nucleic Acids Res, 2002. 30(9): p. 2089-195.
58. Rodriguez-Serrano, A. F. and I. M. Hsing, Allosteric Regulation of DNA Circuits Enables Minimal and Rapid Biosensors of Small Molecules. ACS Synth Biol, 2021.
59. Liu, J., et al., Applications of Catalytic Hairpin Assembly Reaction in Biosensing. Small, 2019. 15(42): p. e1902989.
60. Kenji Ohgane, H. Y., Quantification of Gel Bands by an Image J Macro, Band/Peak Quantification Tool. protocols.io, 2019.
61. Zadeh, J. N., et al., NUPACK. Analysis and design of nucleic acid systems. J Comput Chem, 2011. 32(1): p. 170-3.
62. Schindelin, J., et al., Fiji: an open-source platform for biological-image analysis. Nat Methods, 2012. 9(7): p. 676-82.
63. Conrad, T., et al., Maximizing transcription of nucleic acids with efficient T7 promoters. Commun Biol, 2020. 3(1): p. 439.
64. Pardee, K. et al. Paper-Based Synthetic Gene Networks. Cell 1-22 (2014). doi:10.1016/j.cell.2014.10.004
65. Pardee, K. et al. Rapid, Low-Cost Detection of Zika Virus Using Programmable Biomolecular Components. Cell 165, 1-25 (2016).
66. Salehi, A. S. M. et al. Biosensing estrogenic endocrine disruptors in human blood and urine: A RAPID cell-free protein synthesis approach. Toxicol. Appl. Pharmacol. 345, 19-25 (2018).
67. Peter L Voyvodic, Amir Pandi, Mathilde Koch, Jean-Loup Faulon, Jerome Bonnet. Plug-and-Play Metabolic Transducers Expand the Chemical Detection Space of Cell-Free Biosensors. doi: https://doi.org/10.1101/397315
68. Soltani, M., Davis, B. R., Ford, H., Nelson, J. A. D. & Bundy, B. C. Reengineering cell-free protein synthesis as a biosensor: Biosensing with transcription, translation, and protein-folding. Biochemical Engineering Journal 138, 165-171 (2018).
69. Heili, J. M. et al. Real-Time Visualization of in Vitro Transcription of a Fluorescent RNA Aptamer: An Experiment for the Upper-Division Undergraduate or First-Year Graduate Laboratory. Journal of Chemical Education 95, 1867-1871 (2018).
70. Hofer, K., Langejurgen, L. V. & Jäschke, A. Universal aptamer-based real-time monitoring of enzymatic RNA synthesis. J. Am. Chem. Soc. 135, 13692-13694 (2013).
71. Paige, J. S., Wu, K. Y. & Jaffrey, S. R. RNA mimics of green fluorescent protein. 333, 642-646 (2011).
72. Filonov, G. S., Moon, J. D., Svensen, N. & Jaffrey, S. R. Broccoli: rapid selection of an RNA mimic of green fluorescent protein by fluorescence-based selection and directed evolution. J. Am. Chem. Soc. 136, 16299-16308 (2014).
73. Dolgosheina, E. V. et al. RNA mango aptamer-fluorophore: a bright, high-affinity complex for RNA labeling and tracking. ACS Chem Biot 9, 2412-2420 (2014).
74. Gootenberg, J. S. et al. Nucleic acid detection with CRISPR-Cas13a/C2c2. Science 356, 438-442 (2017).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A composition, system, or kit for detecting an analyte comprising as components:
(a) an RNA polymerase;
(b) an allosteric transcription factor (aTF), wherein the analyte is a ligand to which the aTF binds;
(c) an engineered transcription template;
(d) a double-stranded DNA (dsDNA) signal gate molecule; and
(e) a dsDNA fuel gate molecule;
wherein the engineered transcription template comprises a promoter sequence for the RNA polymerase and an operator sequence for the aTF operably linked to a sequence encoding an RNA,
wherein the aTF modulates transcription of the encoded RNA when the aTF binds the analyte as a ligand and wherein the transcribed RNA displaces a strand of the dsDNA fuel gate molecule generating a free single-stranded DNA molecule which displaces a strand of the dsDNA signal gate molecule to produce a detectable signal and a new hybrid signal/fuel gate, and wherein RNA polymerase transcribes the hybrid signal/fuel gate to release the strand of DNA which can then displace an additional dsDNA signal gate molecule, thus creating a positive feedback loop of signal amplification.

2. The composition, system, or kit of claim 1, wherein the dsDNA fuel gate molecule is a double-stranded nucleic acid comprising a waste strand and a RecycleD strand.

3. The composition, system, or kit of claim 2, wherein the dsDNA signal gate molecule is a fluorescently labeled double-stranded DNA molecule comprising a top strand having a fluorophore conjugated at its 3′-end and a bottom strand having a quencher conjugated at its 5′ end that quenches the fluorophore in the fluorescently labeled double-stranded DNA molecule and the RecycleD strand displaces the bottom strand of the fluorescently labeled double-stranded DNA molecule which results in dequenching of the fluorophore to generate the detectable signal and hybridization of the RecycleD strand to the top strand generates a double-stranded DNA molecule comprising a 3′ toehold on the top strand wherein the RNA polymerase transcribes the top strand and displaces the RecycleD strand and generates a fluorescent DNA/RNA hybrid, and wherein the released RecycleD strand can displace an additional signal gate generating a positive feedback loop of signal amplification.

4. The composition, system, or kit of claim 3, wherein the top strand is longer than the bottom strand and wherein the transcribed RNA comprises a sequence that is complementary to the full length of the top strand.

5. The composition, system, or kit of claim 3, wherein the waste strand or the bottom strand comprises one or more non-natural modifications that prevent the strand from being utilized as a template for transcription (e.g., 2′-O-methylation).

6. The composition, system, or kit of claim 3, further comprising a non-labeled double-stranded DNA molecule comprising a top strand that comprises a nucleotide sequence that is identical to the nucleotide sequence of the top strand of the labeled double-stranded DNA molecule.

7. The composition, system, or kit of claim 6, wherein the top strand of the non-labeled double-stranded DNA molecule is longer than the bottom strand of the non-labeled double-stranded DNA molecule.

8. The composition, system, or kit of claim 6, wherein the bottom strand of the non-labeled double-stranded DNA molecule is shorter in length than the length of the bottom strand of the fluorescently labeled double-stranded DNA molecule.

9. The composition, system, or kit of claim 1, wherein the transcribed RNA does not form and/or is designed not to form an intramolecular secondary structure.

10. The composition, system, or kit of claim 1, wherein the RNA polymerase is selected from T7 RNA polymerase, T3 RNA polymerase, SP6 RNA polymerase, and Syn5 RNA polymerase or the RNA polymerase is an engineered polymerase.

11. The composition, system, or kit of claim 1, wherein the aTF represses, blocks, or inhibits transcription from the engineered transcription template when the aTF binds the operator.

12. The composition, system, or kit of claim 1, wherein the aTF activates transcription from the engineered transcription template when the aTF binds the operator.

13. The composition, system, or kit of claim 1, wherein in the presence of the analyte as a ligand the aTF does not bind to the operator sequence or binds to the operator at a lower affinity than in the absence of the analyte as a ligand.

14. The composition, system, or kit of claim 1, wherein in the absence of the analyte as a ligand the aTF does not bind to the operator sequence or binds to the operator at a lower affinity than in the presence of the analyte as a ligand.

15. The composition, system, or kit of claim 1, wherein the aTF belongs to the TetR, MarR, or ArsR/SmtB class or family of transcription factors or the aTF is an engineered aTF.

16. The composition, system, or kit of claim 1, wherein the aTF is selected from the group consisting of TetR, MphR, QacR, OtrR, CtcS, SAR2349, MobR, SmtB, CadC, CsoR, AdcR, TtgR, and HucR.

17. The composition, system, or kit of claim 1, wherein the analyte that is a ligand for the aTF is an antibiotic.

18. The composition, system, or kit of claim 17, wherein the analyte that is a ligand for the aTF is a member of the macrolide-family of antibiotics or member of the tetracycline-family of antibiotics.

19. The composition, system, or kit of claim 1, wherein the analyte is a quaternary amine or salts thereof.

20. The composition, system, or kit of claim 1, wherein the analyte is a metal or a cation thereof.

21. The composition, system, or kit of claim 20, wherein the metal or the cation thereof is Zn, Pb, Cu, Cd, Ni, As, or Mn.

22. The composition, system, or kit of claim 1, wherein the analyte is selected from salicylate, 3-hydroxy benzoic acid, naringenin, and uric acid.

23. The composition, system, or kit of claim 1, further comprising (f) one or more components for preparing a reaction mixture for RNA transcription.

24. A method for detecting an analyte in a sample, the method comprising contacting the sample with the composition, system, or kit of claim 1 and detecting a signal.

25. The composition, system, or kit of claim 1, wherein the composition, system, or kit is adapted to detect and/or quantify a range of compounds related to environmental contamination and human health.
